SEQUENCE LISTING (122 sequences)

SEQ ID NO: 1 - DNA, 3280 bp, Homo sapiens
gggggagggatgactaaagacaacggctgtaagagaactccacaagagagtcagaacgaaaatgtgcaacaagtgcggcggctcctaccatggcaggtgattcgaggactcagcacgaggcgagagtagggaccaggaagagccggaaaacccgcctgtgattggccgtccacgggtatcggtcgttgtgattgggtgaggcccagacagacagctgcgttttgaaccgcgtagggttctgggtagcaaaggccttgcaaggctcttaaccgaaagggggagggggaaggtcgccaacaaacggctgagctcacaatcctggccggggcgtccccctcccccatggagagagccagacccgagccgccgcctcagccgcgcccgttgcgtcccgctccgcccccgctgccggtcgagggcacctccttttgggcagcagccatggagccccctccgtcgtctcccacactgagcgcggcagccagtgcgaccttggcctcgtcgtgcggggaggcagtggcgtccggcttacagcccgcggtgcggcggctgctgcaggtgaagccagagcaggtgttgctgctaccacagcctcaggcccagaacgaggaagccgctgcctcgtccgcgcaggcgcggctgttgcagttcaggcccgacctgcggctcctgcagccgccgacagcgtcagacggcgccacctccaggcccgagttgcacccggtgcagcccctggcgctgcatgtcaaggccaagaagcagaagctggggcccagcctggatcagtcagtggggcctcgaggggccgtcgaaaccggtcctagagcctccagggtggtcaagttggaaggccccgggccggccctcggctacttccgaggggacgagaagggcaagctggaggcggaggaggtcatgagagactcgatgcaaggcggggcaggcaaaagcccggcagccatccgagaaggtgtgatcaaaacggaggaacccgagagactcctcgaggactgcaggctcggcgcggagcccgcgtccaatggcctggttcatggcagcgcggaggtcatcttggccccaacgtccggtgcctttgggccgcaccagcaagaccttaggatccctttgacgctccacacggtcccccctggggcccggatccagtttcagggagctccgccttcagagctgataagattgaccaaggtccccctgacaccagtgcctactaaaatgcagtccctactggagccttctgtaaaaattgaaaccaaagatgtcccgctcaccgtgttgccctcagatgcaggcataccagatactcccttcagtaaggacagaaatggtcatgtgaagcgacccatgaacgcatttatggtttgggcaaggatccaccgaccagcactagccaaagctaacccagcagccaacaatgcagaaatcagtgtccagcttgggttagagtggaacaaacttagtgaagaacaaaagaaaccctattacgatgaagcacaaaagattaaggaaaagcacagagaggaatttcctggttgggtttatcagcctcgtccagggaagcgaaaacgattccctctaagtgtttccaatgtattttctggtaccacacagaatatcatctctacaaatcctacaacagtttatccttaccgctcacctacgtactctgtggtaattcccagcctacagaatcccatcactcatccagttggtgaaacctcacctgctatccagctgcccacacctgcagtccagagcccaagccctgtcacacttttccagcccagcgtctccagtgctgctcaggtggctgtccaggatccaagtctacctgtctatccagcactcccaccccaacgctttactgggccttcccaaacagacactcatcagctgcattctgaagccactcacactgtgaagcaacccactcctgtctctctagagagcgccaacaggatttcaagtagtgcaagtactgcccatgccagatttgcaacttcgaccatccaacctcctagggagtattccagcgtttccccttgtcccagaagtgctccaatcccccaggcttctcccattccacacccacatgtctaccagccccctccccttggccatccagccacactgttcgggacaccaccaagattctcttttcatcacccttacttcctacccggacctcactacttcccatcaagtacatgcccttacagtcggcctccctttggctatggaaattttccgagttcaatgccagaatgccttagttattatgaagacaggtacccaaaacatgagggtatcttttcaactttaaatagagactattcttttagagactactcaagtgaatgcacacacagtgaaaattctcggagttgtgagaacatgaatggaacttcttactataacagtcatagccacagtggggaagaaaacttaaaccctgtgcctcagctggacattggaaccttggagaatgtcttcacagccccgacatcaactccttctagcatccagcaagtcaatgtcaccgacagtgatgaggaggaagaagaaaaagtgctcagggatttataattttaaaacaaatatgcacagaaaataaacatttcttaaaatatattctgggtcagttggtatgagaaaaaaaaaagcctagaattctttgttgaaagttttcagtcgtgatttgaggagttaaaaccaaatgcaatttatgtcttcataaaattttgattagtgaaactagagtctggatgtttcattgtaggaatatttaagttattaagtagtttaattttaatggctgaaatttgcatcaacatgtattattattactttatcctggaacatgcaaaatactgaagcctcacagttgtatgtgaggggaaaggggaaataaatctagcatagtgtgatttttattttatctcaggatacattttttaaatgattttttgtttgctttttatgtaatacttatggatgttgtcaatttttgatgtaacattttgaaagtattttgacaactcctagtgaacttggacttggttgctaaatttaacttacactaataaccaattataagttccaaatgtgttttaatggcacctgggtgattcttcagctaaatttagtcatttctgtttctaaatatttttatcattttaaaatattttttttccatttggcatacatcgttctttgttgtaattaaataaacatagataaaattgttaaaaaaaaaaaaaaa

SEQ ID NO: 2 - DNA, 1479 bp, Homo sapiens
aggcgagggaaactgagggcgaaagttgtgtgtcgtgttggcaggagggcctagaagggaaagactgtgcgttgataccaaactgcccccactccgtgggcgggtcaggagaggcctttggaagagcgtctcaactcggactggagcctcttctctcccaccgcggtctagtgggacaatgtcatattataaatttggaatgctgaatagaaaattatagattttgatattgaaggaaatgaagcgaagcctaaatgaaaattcagctcgaagtacagcaggctgtttgcctgttccgttgttcaatcagaaaaagaggaacagacagccattaacttctaatccacttaaagatgattcaggtatcagtaccccttctgacaattatgattttcctcctctacctacagattgggcctgggaagctgtgaatccagagttggctcctgtaatgaaaacagtggacaccgggcaaataccacattcagtttctcgtcctctgagaagtcaagattctgtctttaactctattcaatcaaatactggaagaagccagggtggttggagctacagagatggtaacaaaaataccagcttgaaaacttggaataaaaatgattttaagcctcaatgtaaacgaacaaacttagtggcaaatgatggaaaaaattcttgtccagtgagttcgggagctcaacaacaaaaacaattaagaatacctgaacctcctaacttatctcgcaacaaagaaaccgagctactcagacaaacacattcatcaaaaatatctggctgcacaatgagagggctagacaaaaacagtgcactacagacacttaagcccaattttcaacaaaatcaatataagaaacaaatgttggatgatattccagaagacaacaccctgaaggaaacctcattgtatcagttacagtttaaggaaaaagctagttctttaagaattatttctgcagttattgaaagcatgaagtattggcgtgaacatgcacagaaaactgtacttctttttgaagtattagctgttcttgattcagctgttacacctggcccatattattcgaagacttttcttatgagggatgggaaaaatactctgccttgtgtcttttatgaaatcgatcgtgaacttccgagactgattagaggccgagttcatagatgtgttggcaactatgaccagaaaaagaacattttccaatgtgtttctgtcagaccggcgtctgtttctgaacaaaaaactttccaggcatttgtcaaaattgcagatgttgagatgcagtattatattaatgtgatgaatgaaacttaagtagtgataaaaggaagtttagcataaattatagcagttttctgttattgcttaatttaccatctccatagttttatagctactattgtatttcacttgttgaattaaagtatttgaattcttttaaatgtggaaaaaaaaaaaaaaaa

SEQ ID NO: 3 - DNA, 1731 bp, Homo sapiens
ttagggcgggagcccggcgagggcgccggtgctttgttctgtctgaggccaggaagtttgaccgcgctgccatgccgaaccgtaaggccagccggaatgcttactatttcttcgtgcaggagaagatccccgaactacggcgacgaggcctgcctgtggctcgcgttgctgatgccatcccttactgctcctcagactgggcgcttctgagggaggaagaaaaggagaaatacgcagaaatggctcgagaatggagggccgctcagggaaaggaccctgggccctcagagaagcagaaacctgttttcacaccactgaggaggccaggcatgcttgtaccaaagcagaatgtttcacctccagatatgtcagctttgtctttaaaaggtgatcaagctctccttggaggcattttttattttttgaacatttttagccatggcgagctacctcctcattgtgaacagcgcttcctcccttgtgaaattggctgtgttaagtattctctccaagaaggtattatggcagatttccacagttttataaatcctggtgaaattccacgaggatttcgatttcattgtcaggctgcaagtgattctagtcacaagattcctatttcaaattttgaacgtgggcataaccaagcaactgtgttacaaaacctttatagatttattcatcccaacccagggaactggccacctatctactgcaagtctgatgatagaaccagagtcaactggtgtttgaagcatatggcaaaggcatcagaaatcaggcaagatctacaacttctcactgtagaggaccttgtagtggggatctaccaacaaaaatttctcaaggagccctctaagacttggattcgaagcctcctagatgtggccatgtgggattattctagcaacacaaggtgcaagtggcatgaagaaaatgatattctcttctgtgctttagctgtttgcaagaagattgcgtactgcatcagtaattctctggccactctctttggaatccagctcacagaggctcatgtaccactacaagattatgaggccagcaatagtgtgacacccaaaatggttgtattggatgcagggcgttaccagaagctaagggttgggagttcaggattctctcatttcaactcttctaatgaggaacaaagatcaaacacacccattggtgactacccatctagggcaaaaatttctggccaaaacagcagcgttcggggaagaggaattacccgcttactagagagcatttccaattcttccagcaatatccacaaattctccaactgtgacacttcactctcaccttacatgtcccaaaaagatggatacaaatctttctcttccttatcttaatgatggtactcttttcaatttctgaaaacagtaacaggcccaacttccttcttactacagtcatattaaacagatcacatcaatgacaaatgtcactactataaaaactacttaatttgtaaggaaattgtttcatagatttaaaaaaattgtggttggagagcatcttggcatttgtgctttttttcttgagggattgttctgcttcctggctgtatgatgggtatatcattaaagtttggagtcctatatgaacaaaactgacatttttagagttgtacttttgggaatgttatagattgatcattctttctcctgataataaaggtattgaatatctgttatgaaaggttct

SEQ ID NO: 4 - DNA, 531 bp, Homo sapiens
acgagcactggagcttgcgttacttggcctcacctcacctgtgctgtccacgcctggctttgtctcacctgacgcgatatgcctctcctgcgtgggcgctgtcctgcccgccgccactaccgccgcttggccctgctcggcctgcagcccgctccccgcttcgcccactcggggcccccgcgccagcggcccctgtctgccgcggaaatggctgttggacttgtggtgttttttacgaccttcttaacaccagctgcatatgtgctaggcaacctgaagcagttcagaaggaattagatggaagatgatgttgaacagctgttaacgtccaaaaaactttcagaaaaagctgtgtttttgttaacgagcaaaattgcctagttgagttgatgcaaccattgtggtattcactttcctcatgtttatgatgaatattttgcacttttttagtactgtgcattatatagatgtatagtcaaaaatgttctgcttaagtgttaaataaaacggaaacacttattcgtgcttggt

SEQ ID NO: 5 - DNA, 2652 bp, Homo sapiens
tcccttgacttgcttgcggagggagcggccggcggagggagcggcaggtggagggagtggcacgaggcatgcggagggagctgcaccgacatcacataaacgcactgggcagctcgcaggcgccattcgctcttcagacgccggagacgtaggagtgggtcttcagactccaaaggggttggactaatggcggatgctgaggcgagggctgagttcccggaggaggccagacctgacaggggcaccttgcaggtgttgcaagatatggccagccgcttgcgaatccattccatcagggccacatgctccacgagctccggccaccctacatcatgtagcagttcttctgagatcatgtctgtgctgttcttctacatcatgaggtacaagcagtcagatccagagaatccggacaacgaccgatttgtcctcgcaaagagactgtcgtttgtggatgtggcaacaggatggctcggacaaggactgggagttgcatgtggaatggcatatactggcaagtacttcgacagggccagctaccgggtgttctgcctcatgagtgatggcgagtcctcagaaggctctgtctgggaggcaatggcctttgcttcctactacagtctggacaatcttgtggcaatctttgatgtgaaccgcctgggacacagtggtgcattgcccgccgagcactgcataaacatctatcagaggcgctgcgaagcctttgggtggaacacttatgtggtggacggccgggacgtggaggcactgtgccaggtattctggcaggcttctcaggtgaagcacaagcccactgctgtggtggccaagaccttcaagggccggggcaccccaagtattgaggatgcagaaagttggcatgcaaagccaatgccgagagaaagagcagatgccattatcaaattaattgagagccagatacagaccagcaggaatcttgacccacagccccccattgaggactcacctgaagtcaacatcacagatgtaaggatgacctctccacctgattacagagttggtgacaagatagctactcggaaagcatgcggtctggctctggctaagctgggctacgcgaacaacagagtcgttgtgctggatggtgacaccaggtactctactttctctgagatattcaacaaggagtaccctgagcgcttcatcgagtgctttatggctgaacaaaacatggtgagcgtggctctgggctgtgcctcccgtggacggaccattgcttttgctagcacctttgctgcctttctgactcgagcatttgatcacatccggataggaggcctcgctgagagcaacatcaacattattggttcccactgtggggtatctgttggtgacgatggtgcttcccagatggccctggaggatatagccatgttccgaaccattcccaagtgcacgatcttctacccaactgatgccgtctccacggagcatgctgttgctctggcagccaatgccaaggggatgtgcttcattcggaccacccgaccagaaactatggttatttacaccccacaagaacgctttgagatcggacaggccaaggtcctccgccactgtgtcagtgacaaggtcacagttattggagctggaattactgtgtatgaagccttagcagctgctgatgagctttcgaaacaagatatttttatccgtgtcatcgacctgtttaccattaaacctctggatgtcgccaccatcgtctccagtgcaaaagccacagagggccggatcattacagtggaggatcactacccgcaaggtggcatcggggaagctgtctgcgcagccgtctccatggatcctgacattcaggttcattcgctggcagtgtcgggagtgccccagagtgggaagtccgaggaattgctggatatgtatggaattagtgccagacatatcatagtggccgtgaaatgcatgttgctgaactaaaatagctgttagctttggtcttttggcctctttaccctgtgtttatgtttgttccaaaaccatcatttaaatctctactgtcacattttgtttcttaaaagcaaagccagctaacaccttcattcatccctagttcggaaattcaagctaactacttaccctttaaactgtcactgcatatgcaagtaccgctctaatttttggatcattaaagggagttacacaacttttaagtgaaaaaaataggtaacaaaacaaccacctgatagtaagttttctgataagactatagataagtggtagaggtaatcaattcttccgaagtgtttccttcgtgaataactggtagaggtaatagttttttcaatgtatttccttcatgagtaaagaaaatgtggattgaagtatagattccagtagcctagtttccacagcacgataacaccatgacgcctactgctgttcccaccttgggattctgtgtgctgccatcccacctgcagctgccctggaattcccttcgctgtttgccttcatctccctccacgtttgagaggctgtcaggcagcagcgaaagcttgttaggatgtcctgtgctgcttgtgatgagagcctccacactgtactgttcaagtcaatgttaataaagcatttcaaaaccagctgctttattca

SEQ ID NO: 6 - DNA, 2521 bp, Homo sapiens
aaacgagtggagacacgaggaccagcgcgagcggtcccggtgggctaccctccccctgcgacgaccccccctcgctctgaccgactggtcccctaaacggtggcggcggtttttggtcgttgggccccgggatttaggaccaacatttgaagacccgaaggggaactgcaaccatgaatgaagaaaatatagatggaacaaatggatgcagtaaagttcgaactggtattcagaatgaagcagcattacttgctttgatggaaaagactggttacaacatggttcaggaaaatggacaaaggaaatttggcggtcctcctccaggttgggaaggtccacctccacctagaggctgtgaagtttttgtaggaaaaatacctcgtgatatgtatgaagatgagttagttcctgtatttgaaagagctgggaagatatatgaatttcgacttatgatggaatttagtggtgaaaatcgaggttatgcttttgtgatgtacactacaaaagaagaagcccaattagccatcagaattcttaataattatgaaattcgaccagggaagtttattggtgtgtgtgtaagcctggataattgtagattatttattggagctattcccaaggaaaagaagaaagaagaaattttagatgaaatgaagaaagttacagaaggagttgtagatgtcattgtttatccaagtgcaactgataagaccaaaaatcgtggttttgcatttgtggaatatgaatctcacagagctgctgctatggcaaggaggaaactaattccaggaacattccaactatggggccacaccattcaggtagattgggctgacccagagaaagaggtggatgaggaaaccatgcagagagttaaagttctttatgtaagaaatttaatgatctcaactacagaggaaacaattaaagcagaattcaataaatttaagcctggtgcagttgaacgggtaaagaaacttagagattatgcttttgttcactttttcaaccgagaagatgcagtggctgccatgtctgttatgaatggaaaatgcattgatggagcaagtattgaggtaacactagctaaaccagtaaataaagaaaacacttggagacagcatcttaatggtcagattagtccaaattctgaaaatctgattgtgtttgctaacaaagaagagagccacccaaaaactctaggcaagctgccaactcttcctgctcgtctcaatggtcagcatagcccaagtccgcctgaagttgaaagatgcacttaccctttttatcctggaacaaagcttactccaattagtatgtattctttaaaatccaatcattttaattctgcagtaatgcatttggattattactgcaacaaaaataactgggcaccaccagaatattatttatattcaacaacaagtcaagatgggaaagtactcttggtgtataagatagttattcctgctattgcaaatggatcccagagttacttcatgccagacaaactctgtactacgttagaagatgcaaaggaactggcagcccagtttacattacttcatttggactacaatttccatcgcagctcaataaatagtctttcccctgttagtgctaccctctcttctgggactcccagcgtgcttccttatacttcaaggccttattcttatccaggctatcctttgtcaccaacaatatcacttgctaatggcagccatgttggacagcggctatgtatctccaatcaggcctccttcttctgaagaaaatactaacattagtatgaaaatttgtgtaaatttgtagtatgaaaacttgcaaattaaaatattgttttattttagaatcgggtttgcatatttggttttaaaaaggtatttattccaaagtactaaacatcagctataattcagaataacatggagttgtagaatttataaaaatgcaaagtttaaaaagttattcagtggtttctcttgataaaggtacagcaaactactattctttttaaacttctaggattttcttctactttctgagtgggcaatagaacctagtcatttatgtttttttttttttttgcataattttactaaatagtatttcacaaatattaaagcacttgaagacaatggttatagtagatttgattaccaaggatcactatctgtactggagattagaacaattatatgaccagaagcatctaaccattatgtaaaaagaaatgatgagacaaaaagattaagatacaaattttgtgcagtactaaagaaaaagcagtctaccattgtggtccttgaaaataactatagatatttttgttatttgttagacacaaattataattttgttgttaatgtatttaagcattttatagttatgctttgtgtttttgatattctttgtattgttaataacaagtgttatgggtttttaatgttgaaatcatgtgttaatttttgtacttgaattcaaattttttgacattaaatatgtgatgcttcta

SEQ ID NO: 7 - DNA, 1949 bp, Homo sapiens
aataaaggggtctgagccggtcgcctgagcctgaaaagtgctgtcacgtcagcggaaggaggcgtcccagatcttctcagctgtcttggtgccagccttcctagtcttcctacccacactcctacctgctgtcacaggccacagccatcatgcctcggggtcacaagagtaagctccgtacctgtgagaaacgccaagagaccaatggtcagccacagggtctcacgggtccccaggccactgcagagaagcaggaagagtcccactcttcctcatcctcttctcgcgcttgtctgggtgattgtcgtaggtcttctgatgcctccattcctcaggagtctcagggagtgtcacccactgggtctcctgatgcagttgtttcatattcaaaatccgatgtggctgccaacggccaagatgagaaaagtccaagcacctcccgtgatgcctccgttcctcaggagtctcagggagcttcacccactggctctcctgatgcaggtgtttcaggctcaaaatatgatgtggctgccaacggccaagatgagaaaagtccaagcacttcccatgatgtctccgttcctcaggagtctcagggagcttcacccactggctcgcctgatgcaggtgtttcaggctcaaaatatgatgtggctgccgagggtgaagatgaggaaagtgtaagcgcctcacagaaagccatcatttttaagcgcttaagcaaagatgctgtaaagaagaaggcgtgcacgttggcgcaattcctgcagaagaagtttgagaagaaagagtccattttgaaggcagacatgctgaagtgtgtccgcagagagtacaagccctacttccctcagatcctcaacagaacctcccaacatttggtggtggcctttggcgttgaattgaaagaaatggattccagcggcgagtcctacacccttgtcagcaagctaggcctccccagtgaaggaattctgagtggtgataatgcgctgccgaagtcgggtctcctgatgtcgctcctggttgtgatcttcatgaacggcaactgtgccactgaagaggaggtctgggagttcctgggtctgttggggatatatgatgggatcctgcattcaatctatggggatgctcggaagatcattactgaagatttggtgcaagataagtacgtggtttaccggcaggtgtgcaacagtgatcctccatgctatgagttcctgtggggtccacgagcctatgctgaaaccaccaagatgagagtcctgcgtgttttggccgacagcagtaacaccagtcccggtttatacccacatctgtatgaagacgctttgatagatgaggtagagagagcattgagactgagagcttaaggcagggctggcactatttccttggccagggtaccttatggggccatatcctacagatcctcccatttctagggaggtctgaagtagaattttcactttatgttagaagagagtagtgagctttctaagtagtgcagtatagtagaggctggagggaacaagatatgtatctttcttttgttacacatgagtaacttgcagatttatgttttatctctgtcagttatcaacattgttcctgttaagtgaaggtttattttgcttcagattatacaattatcaataacatagctctcacattcatggctgtttaaccaatctgaaagttacggtttgggaattaataaaacaaagtcatacaacacattttctttgtaattgagaactagataacatggtaacagagaattgattttcatatgaatcttaactccacagtaaaatagttgacatcataatatgaagagaaagaaaaggaaaaacagaaatgtaaaagttgtttaattcttggtttgcctaattcgttttcctatttcttttcatacaaataaaggatacctggatttatttaggtta

SEQ ID NO: 8 - DNA, 2499 bp, Homo sapiens
cttctaattctgttattgcaactgcagaccgttacctggtacgctggctgctacctccctcactcttgtcagagtcggagctacaggcagtgccttcagctctgagctcaggcatcccggtccctgtttttgcggttaaggactctaaagtgttgtgtcgtgttcatcaactttttctcaacttccctggctctacctcttctgccacaaacgtcagcatggtggtatctgccgaccctttgtccagcgagagggcagagatgaacatcctagaaatcaaccaggaattgcgctcgcagctggcagagagcaatcagcagttccgagacctcaaagagaaattccttataactcaagctactgcctactccctggccaaccagctgaagaaatacaagtgtgaagagtacaaagacatcatagactctgtgctgagggatgaactgcagtccatggagaagctggcagagaagctcaggcaagctgaggagctcaggcagtataaagccctggttcactctcaggcaaaagagctgacccagttacgggagaagttacgggaagggagagatgcctcccgctggctgaacaagcatctgaaaaccctcctcactcctgatgaccctgacaagtcccagggtcaggacctccgagagcagctggctgaggggcacaggctggcagagcaccttgttcacaagctgagcccagaaaatgatgaagatgaagatgaggatgaagacgacaaagacgaggaggttgagaaagtacaggaatcacctgcccccagagaggtgcagaagactgaagaaaaggaagtccctcaggactcactggaggaatgtgctgtcacttgttcaaatagtcacaacccttctaactccaaccagcctcacaggagcaccaaaatcacatttaaggaacacgaagtcgactctgctctggttgtagagagtgaacaccctcatgatgaagaggaggaagctctaaacattcccccagaaaatcaaaatgaccatgaggaggaggaggggaaagcgccagtgccccccagacaccatgacaagtccaactcttaccggcatcgtgaagtctctttcttggcattggatgaacagaaagtttgctccgctcaggatgttgccagggattactccaatcccaaatgggatgaaacctcacttggcttcctcgaaaagcaaagtgatcttgaagaggtgaaaggacaagaaacagttgctcccaggctcagcaggggaccgctgagagtggacaagcatgaaatcccccaggagtcactggatggatgttgcttgactccttccatccttcctgacctgactccctcctaccacccttattggagcactttgtactcttttgaagacaagcaagtcagcttggctcttgtagacaaaattaaaaaggatcaagaggagatagaagaccaaagcccaccatgccccaggctcagccaggagctgccagaggtgaaggagcaggaagtcccagaggactctgtgaatgaagtttacttgactccctcagttcaccatgacgtgtctgactgccaccagccttatagcagcaccttgtcctcattggaggatcagcttgcctgctctgctctggatgtagcctcccccaccgaggcggcctgtccccaagggacttggagtggagacttgagccaccaccagtcagaggtgcaagtttcacaggcacagctggaaccaagcaccctggtgcccagttgtctgcgactacagctggatcaagggttccactgtgggaacggcttggcccagcggggcctttcctccaccacctgcagcttctcagccaatgctgattctgggaaccaatggcccttccaagagctggttttagagccctctctggggatgaagaaccctccccagctggaagatgatgcacttgaaggctcagcaagcaacacacaagggcgtcaagtcactggccggattcgtgcctcccttgtcctgatactgaagaccatcagaagaagactcccgttcagcaagtggagactggcattcagattcgctggcccgcatgctgagagcgcagagataccaaatactgctggaaggacgcaaaggatggcaggatgaaagaatgtcacaaaaagcagcttttccacttgataaaaacaactaaaacagcaaagcaagtttaagtccaaacacaatactgcaggggtccttcactgaggattgaatttcagacacagaatactcttgatgacttcaagccactatgctcctttgatttgagaagccacattccatccccctccaattgtgatcaatacctagggagaccaatgcccagatggacaaatagcattgaccggcgttagccctgtttctcaattcccatcgtgtagagaacaggagtccgcagctgctggcaggagacagcatgtcagccgggactctgccagggcagagtatgagcaatgccatgttcttgctg

SEQ ID NO: 9 - DNA, 1405 bp, Homo sapiens
atcttaagaggcgttcctttttgcatagttcccatgagcatgagagaagaagcaatgcacgctccggcagattcctaggaaccaaatacctctgaggagcaccagatttcagcttatgggatgctttgattgctctgtggctgcatttaggagaaggaagctgcagtcatgcgtcatcactgccagcctcacatctcttgacagttaaagccttagggtggagcaagggaaaatttaaaataacaaatgaagcaaaagcaagaggtgatgttccaaagcagaggaaggctaagtttatatatacaaatgtcaagtgtgtatagtgcaaaactaggaccagttggtggaatctgtggtcaaaaacaaaagccttcctttttttttttcaaggcccagtcccaagacgcaagaccacttgcgccagcagcgtgcatcagcaagatagcaaaagcaggacgagagctgcccggaagacatctacctggccagaagacacctaccctggccggaagacatgtacccctgaagatagagaaagaggccatcgtgtactacgtagcagtcatgtcagactgggacacttcctgtttacagaggactataaaacccctgtcctgtcctcacttggggctgacgccatcttaggcctcagcccgcctgcagccaggcgttcgttaaaacagcatgttgctccacaccgccttgtattgtttgttggtcccactctctgggctcgaaccaatacaagcacctttcaagcagtatattcttcagtgtcttgatcctccaaataactctcttctaattcctcctgaccacaaaaagcacttatactctaggatgactgattccagcccagtggcctggcaagggtgaattacaccttgcatatcacactcttgacatttgtgtgcgctagcataagaattataattgaaacagggatttaagtatctcctctctaggtgcctaccctccttggactcaggtcaaatttattaaaggaagttttgtttctagataggttgtttgaaataaaataacagaatgttcaagtaacacagtgtacctacagcttttaacaaaattgaggacttgggtctcgaaacaatttcctttgattttcaggtattttatctataaaaagggagataaagcattagttcataggacagttatatgtttaaatgtgataatgtatattaaccaccttgcatgtattcaaatgtgttttgaaatctaacgtctacattttgatagtttaactcttctacataagtgacttacaacaggcattaaatattgtttggcattttcatatatctgtaactgtatcttaatctacaatgagcttaattttaagtgtagcataaaacagaaccttcaataaagtggtaatattagg

SEQ ID NO: 10 - DNA, 3848 bp, Homo sapiens
agcgttgctgctgccttgcagtttgatctcagactgctgtgctagcaatcagcgagactccgtgggcgtaggaccctccaagccaggtgaaagagcttatgatcctaagcacttccataatagggtcagtagaatcatgatcgatgatcataatgtccccactctacgggagatggtagcattctccaaggaagtgttggagtggatggctcaagattctgaaaacatcgtagtgattcactgtaaaggaggcaaagaatagatatgttggatattttgcacaagtgaaacatagctacaactggaatctccctccaagaaaaacactgtttataaaaagattcgttatttattcgattcatggtgttggaacaggcgatggatatgatctaaaagtccaaatagtaatgaagaaaaagattgtcttttcctgtacttccttaaagaattgtcgggtatttcatgacactgaaacagacagggtaataactgatgtgttcaactgtccacctctgtatgatgatgtgaaagtgcaagcttcctcttcaagagaagagggcagcacacctcgcagggctaactggaagggggagccatccaggagacctgtgctcaactgatgggtggggagcaatagcgagaacgagggagggacctgagagtggaagcctttattgggatgtaaggtgttacctgagcaggtttcctacggggaggtctaactggtggatttaatgcaagcagtcatgagttccatggagtcatgctgtgactgagaggtggtcattgatatatccacatggtccatgcagagtacgggggtctgtagggaggttatatctagctgtcccataatgaagtagtcaccaacagaaggttgtataaggcagatactgggatcagtcacattgagaaacctggaggaggtgaactggaaactgtcaagggtgactgaaccctgcttctgatatcagaaagtccaatttatatttgaaagggatgctgaggcacaaaaaaattgtaagaattcactacaaaaatacttggctatatataagcataggtccttagtagattctgtttagcactatctaaaccagattcaaatttcagcatttaaattaaatatctatcatggaaaataaactattccttgaaaattttggtagaaacagcaagagaaagcaatagcattttcttaagcctcctcctctgtgtcttgagtgtgttattatagaatgcagagtgctacctattgaatggttataattatttgataaatatataaaggaataaaggaaggaactttgatttctttggaatgatagttcttggcatcaattttacttttaaaatatttttttttctttttaggattttcctaaatactatcacaactacccttttttcttctggtttaacacatctttaatacaaaataacgggtatggatataatatcaacccatagaaacaacctaatcttcaatgtctatgtataagatgtaatggcaagtcttttgctggttgtcataagcttaatttatagaaaacaaaaaatccttgagccaccattgttcattgccttactccttttacgttggctattttaaaaatacagttgttcttgagacccccagttgcagtatcctcaaggtccatgccataggactgtgttatgagctcaaaagtattataatcagatcttaagtgtggaagtaaattcctcccagagaagttcaatatgaatctgctcagtaccttcaacatgtcaggtcctcagtaggtgctgatttaccaatgacgaaccaccaccaaattttgtgctaaagtaagggaggacctagggaagcttcagctagctgaaaagctgactgacacacttatatctaggagaagttacaagacacagtaagtattaagaaatacagctaaaaaatcattaaaattggtagtctcccatttaaacatgggtttctaataactgaattgggaaaactttcttaaaaactattaattggaggctgggtgtggtggctcatgcctgtaatcctagcactttgggaggctgaggcgggcggatcacctaaggttggaagttcgagactagcctggccaacatggtaaaactccgtctctactaaaaatacaaaaatcagccaggcgtggtggcacatgcctgtaatcccagctactcaggaggctgagccagtagaatcgcttgaacccaggaggcagattgcagtgagccgagatcgcaccactacactccagcctgggcgacagagtgagactctgtctaaagaaaaaagcaaaaaaacagaacaactattacttggatttggagattattgttcccagaaaaccttctgccatatttggaaacttatttctcagtctagaagttctccactttaagtagcatttgttctgtgctggtgaaaaactgagatttttttgtattaaccatactcttcaatacaaaaggagaaaatatttttaaaatgcttcaggtcacagttgaggcagttgctatgattgcatgtggcatgaattggtagttattgttacaaccagttctagtcttttcttcaaatctgagctggatctaataactccttaagtccagcaaggcaacagtaaattaaacctctggtctacacacttgcaatacatacacatttaatagattttgatagagtgaactttggattggatggaaattttttaaaaatttgtttcttggatgcatacaaacaataagctttgactcctaacatgagcaaagtccctcaattgtgagagctgggtggagcttcatttgttgctgctcctcaaattgattcttggtaaaggatacagatttttcctttgaaacaccatgttcattttggggaagcaataagttagatcacctttattttcacttttatataaatttctaaagatttctgtaatatttaaatttatatactattggtaaagctgtttttcttagttgtgaaattgttgtttagccaaaaatgccaacttctgtcttttagaacactaggcataaatgggttaaccaatttatgcctagtgttccattattggaatgctaagcatgtgggatttatttatatcctactgctcaaggtcatcgccaagggctgtttgcaaaaattcaaaaaattgcaacctcaggcataaattaaaagagatatagtattttattattgggttttgatacatgtctaatcagactgatttctgtcacatatagaaatttagatactgtattaaacctggatgtcattaattccataaaaagcaacgttaaaagaatcagtagcatgtgttactgatgtgttgctgaagattaagatatttttaagtctcaccgaaaaggtagaaggagccaactgagacacaaaaaggggctgaggttctattcatggtgagcaagtctttttttttgtttgtttcttcaagctctaacaagggtgcctactacatggcttttcagttagccccaaaataagatgtaacaatttttttttctattcttaggctttatctacaaagaaatgaattggataatcttcataaacaaaaaacatggaaaatttatcaaccagaatatgcagtagagatatattttaatgagaaatgacttaagttatgttgtaactggtagctgattaagtatagttccctgcaccccttctgggaaagaattatgttctttctaaccctgccacatagttatatgttctaaatcttccttgctggtacatctatattgatatatgtatacacatgttctttataaatctattaaatatatacagaaaaaaaaaaaaaaaa

SEQ ID NO: 11 - DNA, 1756 bp, Homo sapiens
gcttgggggcggaaaagccgtggcgcccccttgcgtggcgcgtcggtctcagagtcgcgtgacttcaaccccctcttcgggaggctgggtcgtcatgatccggaccccattgtcggcctctgcccatcgcctgctcctcccaggctcccgcggccgacccccgcgcaacatgcagcccacgggccgcgagggttcccgcgcgctcagccggcggtatctgcggcgtctgctgctcctgctactgctgctgctgctgcggcagcccgtaacccgcgcggagaccacgccgggcgcccccagagccctctccacgctgggctcccccagcctcttcaccacgccgggtgtccccagcgccctcactaccccaggcctcactacgccaggcacccccaaaaccctggaccttcggggtcgcgcgcaggccctgatgcggagtttcccactcgtggacggccacaatgacctgccccaggtcctgagacagcgttacaagaatgtgcttcaggatgttaacctgcgaaatttcagccatggtcagaccagcctggacaggcttagagacggcctcgtgggtgcccagttctggtcagcctccgtctcatgccagtcccaggaccagactgccgtgcgcctcgccctggagcagattgacctcattcaccgcatgtgtgcctcctactctgaactcgagcttgtgacctcagctgaaggtctgaacagctctcaaaagctggcctgcctcattggcgtggagggtggtcactcactggacagcagcctctctgtgctgcgcagtttctatgtgctgggggtgcgctacctgacacttaccttcacctgcagtacaccatgggcagagagttccaccaagttcagacaccacatgtacaccaacgtcagcggattgacaagctttggtgagaaagtagtagaggagttgaaccgcctgggcatgatgatagatttgtcctatgcatcggacaccttgataagaagggtcctggaagtgtctcaggctcctgtgatcttctcccactcagctgccagagctgtgtgtgacaatttgttgaatgttcccgatgatatcctgcagcttctgaagaagaacggtggcatcgtgatggtgacactgtccatgggggtgctgcagtgcaacctgcttgctaacgtgtccactgtggcagatcactttgaccacatcagggcagtcattggatctgagttcatcgggattggtggaaattatgacgggactggccggttccctcaggggctggaggatgtgtccacatacccagtcctgatagaggagttgctgagtcgtagctggagcgaggaagagcttcaaggtgtccttcgtggaaacctgctgcgggtcttcagacaagtggaaaaggtgagagaggagagcagggcgcagagccccgtggaggctgagtttccatatgggcaactgagcacatcctgccactcccacctcgtgcctcagaatggacaccaggctactcatctggaggtgaccaagcagccaaccaatcgggtcccctggaggtcctcaaatgcctccccataccttgttccaggccttgtggctgctgccaccatcccaaccttcacccagtggctctgctgacacagtcggtccccgcagaggtcactgtggcaaagcctcacaaagccccctctcctagttcattcacaagcatatgctgagaataaacatgttacacatggaaaaaaaaaaaaaaaaaaa

SEQ ID NO: 12 - DNA, 1009 bp, Homo sapiens
gggcagaggccaagtgggcaccggatagcgccagccccgcccagagagcgaaatcatggagccttccaagaccttcatgagaaacctgccaatcacaccaggctatagcggctttgtgccattcctcagctgccaaggaatgtccaaggaggatgacatgaaccactgtgtgaaaaccttccaggagaaaacacagcgctataaagaacagctgcgggaattgtgctgcgcagtggccactgccccgaaactgaaacctgtcaactccgaggagacggtcctgcaggccctgcaccagtacaatctgcagtaccaccccctgatcctggaatgcaaatatgtaaagaaacctctccaggagcccccgatccctggctgggcaggctacctgccgagagccaaggtcactgaatttggctgtggcacgagatacactgtcatggccaaaaactgctacaaggacttcctggagatcacggagagggccaagaaggcacatctgaaaccatatgaagagtgaggagaaatgtctctttccttcctactaccgttttaaaaaggggatgaaatgtttgcagtggcctttctgcttagctgggccagctccctgcaactcacacggacggttcctctcctagatggaagctgccctgcccttggaaggcccctgagagaggaccccaaaactccgctgacatgtggctgtgctcagaggccaagtataccatgcagtgggaagatgtatctagagccactgtcctccgcaaagtatgcagaaggctagaagcgcagagtctcccaaggaggtgaactttaagtggggcttccaaaacctgccattctcatgttggaatcacgcccagtgagcaataaagaaatttagtaacaagaattttttaactgccgcctgcatcctgagtggttgacggttgcatgtcattaatgataaagaccgttttttgtcatgtgggaataaagaggctgcttctccgcaaaaaaaaaaaaaaaa

SEQ ID NO: 13 - DNA, 3185 bp, Homo sapiens
aaaaaagtttgggattcccagtctacaaaaccccacagctcctaggaattctctcaccacccttgtgcctttaggcttcggtagattgcaaatgacctgctttctttcggatcccgggctgctttcggacacctgtcgaatagtaaatcccaagtaaggtacctgcggtcgtcggcagatctgaattttcttcttggacacctaatacccacagtcctccagagaccgagaggttgatgtcactcccaatatcggaggaagtatacacccccgtgtgagatggtccttaataatattccacggcggagggggtgatatgactacatacatggcagaaagtggaaacctcccagggatattgttcccacgatcctggagggaagaagatgatgttactttcaatatgacagaaggtggatgaagtggtggactgcccctccacacctgtggacacgcccccactgatattccttctaattgcagcgtgggagaggaggatatgacacgcgatatcgcagggagtagaaacacccctgtgatactgttcttaatattcagggaggatgaggatgatattactcccaatacagacgggtgtacaccctctgtacaccgagggtgtacacctgtctgtgaaagagttcgtaatctccagagggggagatgatattactcacaatatggtaaagaggctgtgagtccacggaggatcctcagagccagcgggggaagaggggctggctctcagtccccgcctcgcgggggtgactccccccagtgcgatgggggtcctaagagccagtgggggaagaggggctggctctgagaccccgcctcgtggggggtgcctccccgccctgtgatgggggtccgaagagccagaaggcttagaggggctggctctcagtccccgcctcgcggggggtgcctcctccccctgcgatgggggtcgtaagagccactgggggaataggggcttgctctcagtcaccgcctcgcggggggtgcctcctttcctttctacatagacacagtgacagtctgatctctctttcttttccctacagatggacacgcccccactgatattgtttttaatgcagcgtgggaggggaggatatgacacgcgatatcgcaggtagtagaaacacccctttgatagtgttcttaatattcagggaggaagaagatgatgttactcccaatacagacggatgtacaccctctgtacaccgagggtgtacacccgtctgtgaaggagttcgtaatctccagaggtggagatgatattactcacaatatggtaaacaggctgtgagtccatcgcggatcctcagagccaggtggggaagaggggctggctgtcagtcccctcctcgcggggggtgcctcccccactgctatggggatcccaagagccagtgggggaagaggtgctggctctcagtctccgcctcgcgaggtgcctctccaccctgcgatcggggtccgaagagccaggggggaagaggggctggctctcttcgtggatgattctttttccattctcaggcagttttcttttttctttctttctttttttttttttttgagactgagtcttgctctgttgcccatgctttgctcgatctcgggtgactgcaaccactgcctcccaggttcaagagattctcctgcctcagcctcctgagtagctgggactagaggcgtgtgtcaccacacccagctaatttttgtatttttagtagagatggggtttcaccatgtttgccaggatggtctctatctcctgcccatgtgatccacccacctcagcctcccaaagtgctgggattgcaggtgcgagccaccgggtccagcctctcaggcgattttcatacctgcatactctggtcactactctgttaaacagtcaaggagggtaagtattatcttcagatttccagagctctgtctctgtacagccctctcctcctcaatattctgccctatgaattctagccacattggccttcccaggctcacagttctgtcttctcaactcaggaagatctctgagttccatctgcattctttcttcctgtgctgtggcctggaaagttttctaaggtgttagggaggtcaattgtggggctagcctcatttgtttctcatctcttgaggatcactgccctttgatgcttgattccagtgattgattccctttgttgcttgagggccatagtttcatatattttgtccagtagttttgttgttttaggtcagaaagtaattttggtctctgttactctatcttggccagaagtgtaagacctaagcatttacacatcaaaatactgcacacataattttagtttaagctactttttaaaaaatctccttcattttccatttagcattctatttagggtattacattggtttttttgaaattctgttattggcagtttctattgcctatcaatcccatttaaagatagtgcatagggtattctaaaatagctgttaagcaaagagaaaattgggcctgatagggtgagaatcacagctctaatacctagagtgaccttataatgtattgtccaaaggagatatttttgacagtgaaagagggtgttgttagtaattatatcaggaccatggcctaaaccaggactatcccaggaagcctgggacatatttgtaccccatctctatttaatgcctttatacaattctttacttaattctaccagcctttattgagcctgctttctttgtctagctgagtgccacgtgctgacgtcactaagatcaatacagcaaactctgaaagatggacagagagacaggagatggtcctttataatgcagtgtgatctgtgctgcaatagagttttggaagctagaagttcaaaacgaggtgttggcagcaccatgctctctctgaagatgctaggaagaatctgctccatgcctttccattcgcttctggggtttcctgcaagccctgacattccttggcttgtagatgcatcaccccagtttccgcccccatcatcacatggcctcctctctgtgtgtgcctctgcgttccctctattcttcttctaaggacaccgacaccagtcatagtggattaagtgtccacccctaaccaattacatctgcaacaaccctatttccaaataaagtcacattctaag

SEQ ID NO: 14 - DNA, 2106 bp, Homo sapiens
agcggatcgtctcttgccgctgccatgaaaggagtgctttttgcctcccaccatgattctgaggcctccccagccatgtagaattagccagcctgtttttgatgaagagcaactgtactggagagaaagcctgtattcatattcatcgtcatcaggcaaatcgaggagctgacttgcttaggatcatctgctggttgaatccatggatgcagaaccccagattcggagggctgaatgtactttcgataggttcctgagcagcagtgattatagtcacagcagactggacagtttggtgagcaagttcaggggctggcttagtgattcatagttctttcttcctcctcccccagcagttttgtttcagcagtgatttggtcaaatgtgaaccacaaaccagcagcaaaagcattttctaggaacttgttaaaactggaaggcccaaggttgcactcaggcttactgagtcagtcactccaagtggaccccatccctaaaccttgagactcactgctcctcttttgatctgggagagaaacagaaaaaagaaggaaaaaaaatccccaatgtgagtatgctggaaagtaaatttgaatagctttttatattgggtacttactgtgggtaatttacaaactattggaaggaaccacaaattcaaacacagccaaatttaaggtctctttagcagaaatcaatcttacatcatggcagttctcccattctccagtctttcacttgatagatcacttcctcattaggatgacctacagcaattctacatgaactgctctttacatttaaacagtgctttctgtcctaacatgtcaaaatgcttgacaagcaatcaaaaggtagctcactaattttttcaactcatggcagtaaatagcctgcatctaccatcactgctgtttactggtgatttcttggcagggaaactttaaatgccttagcaatgtctagaaagtcaatgactaaagaaaatattcattttggcttccatagggctgtatttgatgaactgctcactaacttctattattacgtctcaagaatagataatgatactcagcaagggcaaatcccaaagaatagtctatgctttccttgattgaatctgatttttgagaaattacatggcccaatctacatttaatatgatacaaatgctacttaactccctgttatttctggttgcatataattgatactacttgaagagtaagaagcagagtctcactacactggggaccctttgagagccaagtctgtgtcttagtcatctttgatgccttagtgaccagtaccatacctggaatagtatatgtgatcagtaactcaaatgtatgtatttcttgaatgtatggtgcatgaatgagtcaagatacctttagcgttggaagcaatctgtcatggagtaggaggagaaaaagtagtgaaaaaacttctaaattctcaaaacctagtgctgaactgaatgctgataataacttgacaacgtcctttggcattgcagatgttgagataccaacatctaatccgccaagaaagaatttcagaattaaaatttgggtgtgttcttgcgctggggatttctctgacctaccttctgatagaactttgaacccgagtcaatagtcaatatactagttttttacttaaaactgtaacattttaatctaattttggggacgtgaaataaaactaatgggaaactcttgaaatttttatcactggaataacctaattttgaaaacactgatggttagtttcttgaaatattaatattactacaagtcataagtaaaagcattctatcttaagtgagaaactataaagttggataattactatttgagtttgtggcttggtttgaataaacacttgcttgttttaagtaaaagttcagctgaagtgacaatcaacctttaatcttgtaaagcttctgtgttagatattttctatctctaacatgccaaacatgcatattaaactgagtttttttgcatgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - DNA, 459 bp, Homo sapiens
atggctcgtacgaagcaaacagctcgcaagtctaccggcggcaaagctccgcgcaagcagcttgctactaaagcagcccgtaagagcgctccggccaccggtggcgtgaagaaacctcatcgctaccgcccgggcaccgtggccttgcgcgaaatccgtcgctaccagaagtccaccgagctgctgatccggaagctgccgttccagcgcctggtgcgagaaatcgcccaggacttcaaaaccgacctgcgtttccagagctctgcggtgatggcgctgcaggaggcttgtgaggcctacctggtgggactcttcgaagacaccaatctgtgcgctattcacgctaaacgcgtcaccatcatgcccaaagatatccagctggcacgtcgcatccgtggggaaagggcataagtctgcccgtttcttcctcattgaaaaggctcttttcagagccactca

SEQ ID NO: 16 - DNA, 3591 bp, Homo sapiens
ttactgggcgtatggcgtacagacacgaggccggcgcccgggaggcggtgttcatccgcccgggaaaagagcgcctgttgctcgctgcccgcgtgtccctggctctctcgggaacccagcgccgaaggcgaggtgggcgcgggccgaaggaggtcctgggaggtcggcggcgcggagggatctccgcgggagccgttggggctgttggcctcgggctgaggtgcaaggaccaggactagggcgagggcagcggtccaagaaatagaaaacaatgactgggagagcccgagccagagccagaggaagggcccgcggtcaggagacagcgcagctggtgggctccactgccagtcagcaacctggttatattcagcctaggcctcagccgccaccagcagagggggaattatttggccgtggacggcagagaggaacagcaggaggaacagccaagtcacaaggactccagatatctgctggatttcaggagttatcgttagcagagagaggaggtcgtcgtagagattttcatgatcttggtgtgaatacaaggcagaacctagaccatgttaaagaatcaaaaacaggttcttcaggcattatagtaaggttaagcactaaccatttccggctgacatcccgtccccagtgggccttatatcagtatcacattgactataacccactgatggaagccagaagactccgttcagctcttctttttcaacacgaagatctaattggaaagtgtcatgcttttgatggaacgatattatttttacctaaaagactacagcaaaaggttactgaagtttttagtaagacccggaatggagaggatgtgaggataacgatcactttaacaaatgaacttccacctacatcaccaacttgtttgcagttctataatattattttcaggaggcttttgaaaatcatgaatttgcaacaaattggacgaaattattataacccaaatgacccaattgatattccaagtcacaggttggtgatttggcctggcttcactacttccatccttcagtatgaaaacagcatcatgctctgcactgacgttagccataaagtccttcgaagtgagactgttttggatttcatgttcaacttttatcatcagacagaagaacataaatttcaagaacaagtttccaaagaactaataggtttagttgttcttaccaagtataacaataagacatacagagtggatgatattgactgggaccagaatcccaagagcacctttaagaaagccgacggctctgaagtcagcttcttagaatactacaggaagcaatacaaccaagagatcaccgacttgaagcagcctgtcttggtcagccagcccaagagaaggcggggccctggggggacactgccagggcctgccatgctcattcctgagctctgctatcttacaggtctaactgataaaatgcgtaatgattttaacgtgatgaaagacttagccgttcatacaagactaactccagagcaaaggcagcgtgaagtgggacgactcattgattacattcataaaaacgataatgttcaaagggagcttcgagactggggtttgagctttgattccaacttactgtccttctcaggaagaattttgcaaacagaaaagattcaccaaggtggaaaaacatttgattacaatccacaatttgcagattggtccaaagaaacaagaggtgcaccattaattagtgttaagccactagataactggctgttgatctatacgcgaagaaattatgaagcagccaattcattgatacaaaatctatttaaagttacaccagccatgggcatgcaaatgagaaaagcaataatgattgaagtggatgacagaactgaagcctacttaagagtcttacagcaaaaggtcacagcagacacccagatagttgtctgtctgttgtcaagtaatcggaaggacaaatacgatgctattaaaaaatacctgtgtacagattgccctaccccaagtcagtgtgtggtggcccgaaccttaggcaaacagcaaactgtcatggccattgctacaaagattgccctacagatgaactgcaagatgggaggagagctctggagggtggacatccccctgaagctcgtgatgatcgttggcatcgattgttaccatgacatgacagctgggcggaggtcaatcgcaggatttgttgccagcatcaatgaagggatgacccgctggttctcacgctgcatatttcaggatagaggacaggagctggtagatgggctcaaagtctgcctgcaagcggctctgagggcttggaatagctgcaatgagtacatgcccagccggatcatcgtgtaccgcgatggcgtaggagacggccagctgaaaacactggtgaactacgaagtgccacagtttttggattgtctaaaatccattggtagaggttacaaccctagactaacggtaattgtggtgaagaaaagagtgaacaccagattttttgctcagtctggaggaagacttcagaatccacttcctggaacagttattgatgtagaggttaccagaccagaatggtatgacttttttatcgtgagccaggctgtgagaagtggtagtgtttctcccacacattacaatgtcatctatgacaacagcggcctgaagccagaccacatacagcgcttgacctacaagctgtgccacatctattacaactggccaggtgtcattcgtgttcctgctccttgccagtacgcccacaagctggcttttcttgttggccagagtattcacagagagccaaatctgtcactgtcaaaccgcctttactacctctaacctgcagaagacgatgcagccgcttttctttttgaaatgactttgggatttttttaagcttttatttactttttttttaactgttatctttctggatgaaacttgggaaggggattaggagatctagcattttatttctagcattgctattcaccggcttccttattttatacgtaaaaattaagattttatattttatcttcttgtttctcatagatattttgtgagcatttttttgtttattttgaagaaatgtggataagatacttggtagtataaaacagactctctgagagtatttgaaatgtgtttggagatttacttaaacgtactttcaggagtgagcaagtcctacttataaacctatattaactttatttttgagatacctgttttgaatttaaaggagataagaggcgtaaagtaggatgctcactacaaccataggtggggtttcagctcatatcttaaagataaaaggtactattatataacctatacacaagatacaggagaaaatatgcttgatttttatttggcaggggggctaggttgtatgggagtaaaaaaaacattgaaaatttttaaattgtccaaagaaacattttaagactctttaacaaaaaaggccatgagtaaatctctatattaacattactatttattttgttttggaactgggacatgattctatttgttataaaataaaattgatgtgattgtcaccttatttg

SEQ ID NO: 17 - DNA, 819 bp, Homo sapiens
gtgatgctccctgggcctcctgaccgcgccctcgcctgggaggcggggcgggccgggttctctctgtgacgtcacaaaggccccgccatgcctctggctttgacccttctgctgctctcgggcttgggcgcccccggaggctggggctgcctgcagtgcgaccccttggtgctggaggccctgggtcacctgcgctccgccctcatccccagtcgcttccagttggagcagctgcaggcgcgcgccggggccgtgctgatgggcatggaggggcctttcttccgggactacgcgctgaacgtgtttgtggggaaagtggagacaaatcaactggaccttgtggcgtcctttgtcaagaaccaaacgcagcacttaatgggtaactctctgaaagatgagcctctgctggaagagctggtgaccctcagggcgaatgtgatcaaggaattcaagaaagttttaatttcatatgaattaaaagcctgcaaccccaaactttgccgcttgctaaaagaagaggtgttggactgtttacattgccagaggatcactcccaagtgtatccacaaaaagtactgctttgtcgaccggcaaccccgcgtggccctgcagtaccagatggacagcaaatacccgaggaaccaggcgctgttgggcatcctcatttctgtgtctctggctgtctttgtcttcgtggtcatcgtggtctcggcttgtacatacagacaaaaccgaaaactcctgctgcagtaggacggtggtttgggggtaaggagaaaggaaaataaatttaataaaattggtgacaaatccaaaaaa

SEQ ID NO: 18 - DNA, 5120 bp, Homo sapiens
ggactcgcactcggcggttgttccagaagaaagagacagcgatggcggcagaggcttcgaagactgggccttctaggtcttcctaccagcgaatggggaggaagagtcagccctggggtgccgctgaaatccagtgcaccaggtgtggaaggagggtatccagatcatccggtcaccattgtgaacttcaatgtggacatgctttttgtgaactatgcttgttaatgactgaagaatgcaccacaattatatgccctgattgtgaggttgctacagctgtaaatactagacaacgctactacccaatggctggatatattaaggaagactccataatggaaaaactgcagcctaagacgataaagaattgttctcaggactttaagaagactgctgatcagctaactactggtttagaacgttcagcctccacagacaagactcttttgaactcatcagctgtaatgttggacactaatactgcagaagaaattgatgaagcattgaatacagcacaccatagtttcgaacagttaagcattgctggaaaagcacttgaacacatgcagaagcaaacgatagaggaaagagaaagagttatagaagttgtggagaaacagtttgaccaacttttggctttttttgattccaggaaaaagaacctgtgtgaagaatttgcaagaactactgatgattatctatcaaatttaataaaggctaaaagctacattgaagagaaaaaaaataatttgaatgcagctatgaacatagcaagagcattacaattatcgccttctctaagaacatactgtgacctgaatcagattatccggactttgcagttaacttcagatagtgaattagcacaagttagttctccacaactaaggaaccctcccaggttgagtgtgaattgcagtgagatcatctgtatgttcaacaatatgggaaagattgaatttagggactcaacaaaatgttatccccaagaaaatgaaattagacagaatgttcaaaagaaatataataacaaaaaggaactttcttgttacgatacatacccaccgctagaaaagaaaaaggttgacatgtctgtcctaaccagtgaagcaccaccacctcctttgcaacctgagacaaatgatgtacatttagaagcaaaaaacttccagccacagaaagacgttgcaacagcatcccctaaaaccattgctgtgttacctcagatgggatctagccctgatgtgataattgaagaaattattgaagacaacgtggaaagttctgcagagctagtttttgtaagccatgtaatagatccttgccatttctacattcggaagtattcacaaataaaagacgccaaagtactggagaagaaggtgaatgaattttgcaataggagttcacaccttgatccttcagacattttggaactaggtgcaagaatatttgtcagcagtattaaaaatggaatgtggtgtcgaggaactatcacagaattaattccaatagagggtagaaataccagaaaaccttgtagtccaaccagattatttgtccatgaagttgcactaatacaaatattcatggtagattttggaaattctgaagtcctgattgtcactggagttgttgatacccatgtgagaccagaacactctgctaagcaacatattgcactaaatgatttatgtctggttctaaggaaatctgaaccatatactgaagggctgctaaaagacatccagccattagcacaaccatgctcattgaaagacattgttccacagaattcaaatgaaggctgggaagaggaagctaaagtggaatttttgaaaatggtaaataacaaggctgtttcaatgaaagtttttagagaagaagatggtgtgcttattgtagatctgcaaaaaccaccaccgaataaaataagcagtgatatgcctgtgtctcttagagatgcgctagtttttatggaactagcaaagtttaagtcacaatcactaagaagtcactttgaaaaaaatactactttacactatcatccacctattttgcctaaagaaatgacagatgtttcagtaacggtttgtcatataaatagtcctggagatttctatcttcagttgatagagggcctggatattttatttctattaaagacaatcgaggaattctataaaagtgaagatggagaaaatctggaaatcctctgtccagttcaagatcaagcctgtgtagctaaatttgaagatggaatttggtaccgagcaaaagttatcggattgcctggacatcaggaagttgaagttaaatatgtggactttggtaatactgcaaaaataacaatcaaagacgtgcgtaaaataaaggatgagtttctgaatgccccagagaaggcaattaaatgtaagttggcctatattgaaccatataaaaggacaatgcagtggtccaaagaagctaaagaaaaatttgaagaaaaggctcaagataaatttatgacatgttcagttatcaaaattctggaagataatgtgctcttagttgagcttttcgattctcttggtgctcctgaaatgactactactagtattaatgaccagctagttaaagagggcctagcatcttatgaaataggatacatcctcaaagataattctcaaaagcatattgaagtttgggatccttctccagaagaaattatttcaaatgaagtacacaacttaaatcctgtgtctgcaaaatctctacctaatgagaattttcagtcactttataataaggaattgcctgtgcatatctgtaatgtaatatctcctgagaagatttatgttcagtggttgttaactgaaaacttacttaatagtttagaagaaaagatgatagctgcttatgaaaactcaaaatgggaacctgttaaatgggaaaatgatatgcactgtgctgttaagatccaagataaaaatcagtggcgaagaggccagatcatcagaatggttacagacacattggtagaggtcttgctgtatgatgtgggtgttgaactagtagtgaatgttgactgtttaagaaaacttgaagaaaatctaaagacaatgggaagactctctttggaatgttctctggttgacataagaccagctggtgggagtgacaagtggacagcaacagcttgtgactgtctttcattgtacctgactggagctgtagcaactataatcttacaggtggatagtgaggaaaacaacacaacatggccattacctgtgaaaattttctgcagagatgaaaaaggagagcgtgttgatgtttctaaatatttgattaaaaagggtttggctttgagagaaaggagaattaataacttagataacagccattcattatctgagaagtctctggaagtccccctggaacaggaagattcagtagttactaactgtattaaaactaactttgaccctgacaagaaaactgctgacataatcagtgaacagaaagtgtctgaatttcaggagaaaattctagaaccaagaaccactagagggtataagccaccagctattcctaacatgaacgtatttgaggcaacagtcagctgtgttggtgatgatggaactatatttgtagtacctaaactatcagaatttgagctaataaaaatgacaaatgaaattcaaagtaatttaaaatgccttggtcttttggagccttatttctggaaaaaaggagaagcatgtgcagtaagaggatccgatactctgtggtatcgtggcaaggtgatggaggttgtaggtggcgctgtcagagtacaatatttagatcatggattcactgaaaagattccgcagtgccatctttaccctattttgctgtatcctgatataccccagttttgtattccttgtcagctccataataccacacctgttgggaatgtctggcaaccagatgcaatagaagttcttcaacaactgctttcaaagagacaggtggacattcacattatggagttacctaaaaatccatgggagaaattgtctattcacctctattttgatggaatgtcactttcttattttatggcatactataaatactgtacttctgaacatactgaggagatgttgaaagaaaaaccaagatcagatcatgataaaaagtatgaagaggaacaatgggaaataaggtttgaggaattgctttcggctgaaacagacactcctcttttaccaccatatttgtcttcatctctgccttccccaggagaactctatgctgttcaagttaagcacgttgtctcacctaatgaagtgtatatttgccttgattctatagaaacttctaaccagtctaaccagcatagtgacacagatgatagtggagtcagcggggaatcagaatccgagagccttgatgaagcactgcagagggttaataagaaggtagaggcgcttcctcctctgacggattttagaacagaaatgccttgccttgcagaatatgatgatggcttatggtatagagcgaagattgttgccattaaagaatttaatcctttatctatcttagtacaatttgttgattatggatcaactgcaaagctgacattaaacagactgtgccaaattccttctcatcttatgcggtatccagctcgagccataaaggttctcttggcagggtttaaacctcccttaagggatctaggggagacaagaataccatattgtcccaaatggagcatggaggcactgtgggctatgatagactgtcttcaaggaaaacaactctatgctgtgtccatggctccagcaccagaacagatagtgacattatatgacgatgaacagcatccagttcatatgccgttggtagaaatggggcttgcagataaagatgaataagtgcctaagtgtatacagtgagagcatctatagaagcctagaagaattctgttatgtttagactatgtcttatctttagactatttcaggcttaattttcctaacttgttcagcactagtgctttacctctcatttttaattgaactgttaggaattgtgtggggaaaaaaagtaaataaatgttcgcttccaaaaaaaaaaaaaaa

SEQ ID NO: 19 - DNA, 1738 bp, Homo sapiens
gatgtcatcaggctttgtgagggggactgtactgccctttgagtactactgtgtggccccgcaacccagcacaaccaggtatctgcttggaacccagccaccataaagcctgctagctaaaaaaaattttacatctctcagttcattcggcacagacccctgcctcattcagctgtgactctgcttggaaaattcatcagttacaaagcagccaatgcaattatctcaagggaaattgaaaaatggacctttgaaaatgctagatttacaatgagaaatgccataattcaaggtttattctatgggtccttgacatttgggatctggacagctctgttattcatatatttgcaccataatcatgtgagcagctggcagaagaaaagccaggagcctctgtcagcttggtcccctggaaaaaaagtgcatcagcaaattatctatggctcagagcaaataccaaaacctcatgtaatagtcaaaaggactgatgaagataaagcaaagtctatgttaggtacagattttaaccatacaaacccagaacttcataaagaacttttaaaatatggatttaatgtgattatcagtagaagcttgggcatcgaaagagaagtgccagataccaggagtaaaatgtgtcttcaaaaacattacccagcccgcctcccgactgccagcattgtcatttgcttctataatgaagaatgtaatgccttgtttcagaccatgtccagtgtcacgaacctcacgccacactattttcttgaagaaattattttggtagatgacatgagcaaagttgatgatttgaaagaaaaactagactatcacctggaaacttttcggggaaaggttaaaataataagaaacaaaaagagagaggggctgattcgagcaaggctgattggagcttctcatgcttcaggggatgttctggtgttcctggacagccactgtgaggtgaacagagtatggctggagcccctgctgcatgccattgccaaggaccccaaaatggtggtgtgccccctgatagatgtcattgatgatagaactctggagtataagccctctcctcttgtaaggggaacttttgattggaacctacaatttaaatgggataatgttttctcttatgagatggatggaccagaaggatctactaaaccaatccggtcacctgcaatgtctggaggaatttttgctatacgtcggcattattttaatgaaattggacagtatgacaaggatatggatttttggggaagagaaaatttggaactttcactaaggatctggatgtgtggaggccaactctttataatcccctgctctcgagtaggacatatcagtaagaaacaaactggaaaaccttctacaatcatcagtgctatgacacataactacctaagactggtgcacgtttggctggatgaatataaggagcagttttttcttcgaaagcctggtctgaaatatgtcacctacggaaatattcgcgagcgtgttgagttaaggaaacgactgggttgcaagtcatttcagtggtatttggataatgtcttcccagagttggaggcatctgtgaacagcctgtgaaaggaaaacaaatcactttcattaataaagggttaaaagtctcctagtcattcaacatagtgtcacaagagtgtaagtttggaacatcgtggaattacgtgaaatgcaattaaaaaaatatgaccagacgtgaaaa

SEQ ID NO: 20 - DNA, 3623 bp, Homo sapiens
tctagcacaggggatccccaaacatcaggacttttggggggcgcctgtgctgtccatgggaagagcatgcattgtgggttactggaggaacccgacatggattccacagagagctggattgaaagatgtctcaacgaaagtgaaaacaaacgttattccagccacacatctctggggaatgtttctaatgatgaaaatgaggaaaaagaaaataatagagcatccaagccccactccactcctgctactctgcaatggctggaggagaactatgagattgcagagggggtctgcatccctcgcagtgccctctatatgcattacctggatttctgcgagaagaatgatacccaacctgtcaatgctgccagctttggaaagatcataaggcagcagtttcctcagttaaccaccagaagactcgggacccgaggacagtcaaagtaccattactatggcattgcagtgaaagaaagctcccaatattatgatgtgatgtattccaagaaaggagctgcctgggtgagtgagacgggcaagaaagaagtgagcaaacagacagtggcatattcaccccggtccaaactcggaacactgctgccagaatttcccaatgtcaaagatctaaatctgccagccagcctgcctgaggagaaggtttctacctttattatgatgtacagaacacactgtcagagaatactggacactgtaataagagccaactttgatgaggttcaaagtttccttctgcacttttggcaaggaatgccgccccacatgctgcctgtgctgggctcctccacggtggtgaacattgtcggcgtgtgtgactccatcctctacaaagctatctccggggtgctgatgcccactgtgctgcaggcattacctgacagcttaactcaggtgattcgaaagtttgccaagcaactggatgagtggctaaaagtggctctccacgacctcccagaaaacttgcgaaacatcaagttcgaattgtcgagaaggttctcccaaattctgagacggcaaacatcactaaatcatctctgccaggcatctcgaacagtgatccacagtgcagacatcacgttccaaatgctggaagactggaggaacgtggacctgaacagcatcaccaagcaaaccctttacaccatggaagactctcgcgatgagcaccggaaactcatcacccaattatatcaggagtttgaccatctcttggaggagcagtctcccatcgagtcctacattgagtggctggataccatggttgaccgctgtgttgtgaaggtggctgccaagagacaagggtccttgaagaaagtggcccagcagttcctcttgatgtggtcctgtttcggcacaagggtgatccgggacatgaccttgcacagcgcccccagcttcgggtcttttcacctaattcacttaatgtttgatgactacgtgctctacctgttagaatctctgcactgtcaggagcgggccaatgagctcatgcgagccatgaagggagaaggaagcactgcagaagtccgagaagagatcatcttgacagaggctgccgcaccaaccccttcaccagtgccatcgttttctccagcaaaatctgccacatctgtggaagtgccacctccctcttcccctgttagcaatccttcccctgagtacactggcctcagcactacaggagcaatgcagtcttacacgtggtctctaacatacacagtgacgacggctgctgggtccccagctgagaactcccaacagctgccctgtatgaggaacactcatgtgccttcttcctccgtcacacacaggataccagtttatccccacagagaggaacatggatacacgggaagctataactatgggagctatggcaaccagcatcctcaccccatgcagagccagtatccggccctccctcatgacacagctatctctgggccactccactatgccccttaccacaggagctctgcacagtacccttttaatagccccacttcccggatggaaccttgtttgatgagcagtactcccagactgcatcctaccccagtcactccccgctggccagaggtgccctcagccaacacgtgctacacaagcccgtctgtgcattctgcgaggtacggaaactctagtgacatgtatacacctctgacaacgcgcaggaattctgaatatgagcacatgcaacactttcctggctttgcttacatcaacggagaggcctctacaggatgggctaaatgactgctatcataggcatccatatttaatattaataataataattaataataataataaacccaacacccatcccccagaagactttatctctatacattgtaactcatgggctattcctaagtgcccattttcctaatgaacatgaggatgggatcaatgtgggatgaataaactttagttcagaaacaggacttactaaaagtcagtgggactgggtttctgtagccaagccagacttgactgtttctgtagagcactatctcgggcaggccattctgtgccttttccctctgttccatgactttgctttgtgttggcaaccacttctagtaagctactgattttcctgttgacaaaatctctttagtcttgaaggatggatactggagacagaatctggtttgtgttcttggatgggcacataatttaccaagagcattcaccttgccatctgtcttgtcattgtactgtacaaggaacagccctcagacgtgttctgcacatcccttcttcctggtggtaccatccctatttcctggagcaccagggctaaatggggagctatctggaaactctagattttctgtcatacccacatctgtcacagtacctgcattgtcttggaatgtaagcactgtcttgagggaaggaagaggtctgttctgtattgccttaagttgattgaggtttgtaggagactggttcttctacatacaaggatttgtcttaagtttgcacaatggctagtgtcagcaaaaggcaggagagggtttttgttttttttttaagttctatgagaatgtggatttatggcattgagtatcacactcagctctgctgtgttaactttgtgaaactggatggaacaaactttaacttaccaagcaccaagtgtgaaagtgactttcacggttccttcataaaactataataatatccgacactttgatagaaaaaaattcaaagctgtgcctttgagcctatactatactgtgtatgtgtggaaataaaaatgtattgtacttttggagaattttttgtaggcatttttctgtcagatttgtagtaatttgtgaggtttgttagagattaatataggttttctttctgtattataaaatgcaccaagcaattatggtggacctattaccctatgggtaagaaataaatggaaatatgacatcggatgtttcagcaactgttctgtaaataaaatctttgatcacaccactcagtgtgataattgtgtctacagctaaaatggaaatagttttatctgtacagttgtgcaagatatgaatggtttcacactcaaataaaaaatattgaaacgaaaaaaaaaaaaaaaa

SEQ ID NO: 21 - DNA, 1099 bp, Homo sapiens
gctgcattacagacacagacctgcaaacatctatggttgtgacagagtttctttctgacacctgagtctttctcctgctgcacggaaagcttgctgggaggggcttggaatctggcatgaagccaaagggcatctctgagttgcagcatttaaatgatcccactcagagattcacacagaagactggacacaattccgaagagctgcccagaaggagagaacaatgtcatcactacccagtggcagacacctttcaccccagctacacaagagggggcagatgtgtgaggatcactgcagtccaggagttcgatgtttcagtgagctgtgattgcaccactgcatatcagcctgggtgacagagcaagaccctatctcaaaaatacagaaaaatcatcaaccacttgcagtcgtcgtagaaatcaatcattccctccagttatgtccctgacccacaggcttcatttgtgcaagtactggggctgtgctgtcagtaatgtgtgccgcttctgggaaggacgtccattgcccttgatgattgtggtaccatacacactgcctgtttccttgcctgttggttcgtgcgtgataatcacagggacaccgatcctcacttttgtcaaggacccacagctggaggtgaatttctacactgggatggatgaggactcagatattgctttccaattccgactgcactttggtcatcctgcaatcatgaacagttgtgtgtttggcatatggagatatgaggagaaatgctactatttaccctttgaagatggcaaaccatttgagctgtgcatctatgtgcgtcacaaggaatacaaggtaatggtaaatggccaacgcatttacaactttgcccatcgattcccgccagcatctgtgaagatgctgcaagtcttcagagatatctccctgaccagagtgcttatcagcgattgagggagatgatcagactcctcattgttgaggaatccctctttctacctgaccatgggattcccagagcctactaacagaataatccctcctcaccccttcccctacacttgatcattaaaacagcaccaaacttcaaaaaaaaaaaaaaaaa

SEQ ID NO: 22 - DNA, 1660 bp, Homo sapiens
ggtgcactagcaaaacaaacttattttgaacactcagctcctagcgtgcggcgctgccaatcattaacctcctggtgcaagtggcgcggcctgtgccctttataaggtgcgcgctgtgtccagcgagcatcggccaccgccatcccatccagcgagcatctgccgccgcgccgccgccaccctcccagagagcactggccaccgctccaccatcacttgcccagagtttgggccaccgcccgccgccaccagcccagagagcatcggcccctgtctgctgctcgcgcctggagatgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactgaaaccagatgaaataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtctataatggaaacatttacaataaatattctgcatggaaatactgttaaaaaaaaaaa

SEQ ID NO: 23 - DNA, 1504 bp, Homo sapiens
ggttgaggtcaagtagtagcgttgggctgcggcagcggaggagctcaacatgcgtgagtgtatctctatccacgtggggcaggcaggagtccagatcggcaatgcctgctgggaactgtactgcctggaacatggaattcagcccgatggtcagatgccaagtgataaaaccattggtggtggggacgactccttcaacacgttcttcagtgagactggagctggcaagcacgtgcccagagcagtgtttgtggacctggagcccactgtggtcgatgaagtgcgcacaggaacctataggcagctcttccacccagagcagctgatcaccgggaaggaagatgcggccaataattacgccagaggccattacaccatcggcaaggagatcgtcgacctggtcctggaccggatccgcaaactggcggatctgtgcacgggactgcagggcttcctcatcttccacagttttgggggtggcactggctctgggttcgcatctctgctcatggagcggctctcagtggattacggcaagaagtccaagctagaatttgccatttacccagccccccaggtctccacggccgtggtggagccctacaactccatcctgaccacccacacgaccctggaacattctgactgtgccttcatggtcgacaatgaagccatctatgacatatgtcggcgcaacctggacatcgagcgtcccacgtacaccaacctcaatcgcctgattgggcagatcgtgtcctccatcacggcctccctgcgatttgacggggccctgaatgtggacttgacggaattccagaccaacctagtgccgtacccccgcatccacttccccctggccacctacgccccggtcatctcagccgagaaggcctaccacgagcagctgtccgtggctgagatcaccaatgcctgcttcgagccagccaatcagatggtcaagtgtgaccctcgccacggcaagtacatggcctgctgcatgttgtacaggggggatgtggtcccgaaagatgtcaacgcggccatcgccaccatcaagaccaagcgcaccatccagtttgtagattggtgcccaactggatttaaggtgggcattaactaccagccccccacggtggtccctgggggagacctggccaaggtgcagcgggctgtgtgcatgctgagcaacaccacggccatcgcggaggcctgggctcgcctggaccataagttcgatctcatgtatgccaagcgggcctttgtgcactggtacgtgggagaaggcatggaggagggggagttctctgaggcccgcgaggacctggcagctctggagaaggattatgaagaggtgggcgtggattccgtggaagccgaggctgaagaaggtgaagaatactgaggggagggtgtggtgggttctccactccactgccacccccagcgtggctgctttcaagttctttgcaattaaaggttctgtataaaaaaaaaaaaaaaaaaa

SEQ ID NO: 24 - protein, 753 aa, Homo sapiens [sequence omitted]

SEQ ID NO: 25 - protein, 363 aa, Homo sapiens
MKRSLNENSARSTAGCLPVPLFNQKKRNRQPLTSNPLKDDSGISTPSDNYDFPPLPTDWAWEAVNPELAPVMKTVDTGQIPHSVSRPLRSQDSVFNSIQSNTGRSQGGWSYRDGNKNTSLKTWNKNDFKPQCKRTNLVANDGKNSCPVSSGAQQQKQLRIPEPPNLSRNKETELLRQTHSSKISGCTMRGLDKNSALQTLKPNFQQNQYKKQMLDDIPEDNTLKETSLYQLQFKEKASSLRIISAVIESMKYWREHAQKTVLLFEVLAVLDSAVTPGPYYSKTFLMRDGKNTLPCVFYEIDRELPRLIRGRVHRCVGNYDQKKNIFQCVSVRPASVSEQKTFQAFVKIADVEMQYYINVMNET

SEQ ID NO: 26 - protein, 434 aa, Homo sapiens
MPNRKASRNAYYFFVQEKIPELRRRGLPVARVADAIPYCSSDWALLREEEKEKYAEMAREWRAAQGKDPGPSEKQKPVFTPLRRPGMLVPKQNVSPPDMSALSLKGDQALLGGIFYFLNIFSHGELPPHCEQRFLPCEIGCVKYSLQEGIMADFHSFINPGEIPRGFRFHCQAASDSSHKIPISNFERGHNQATVLQNLYRFIHPNPGNWPPIYCKSDDRTRVNWCLKHMAKASEIRQDLQLLTVEDLVVGIYQQKFLKEPSKTWIRSLLDVAMWDYSSNTRCKWHEENDILFCALAVCKKIAYCISNSLATLFGIQLTEAHVPLQDYEASNSVTPKMVVLDAGRYQKLRVGSSGFSHFNSSNEEQRSNTPIGDYPSRAKISGQNSSVRGRGITRLLESISNSSSNIHKFSNCDTSLSPYMSQKDGYKSFSSLS

SEQ ID NO: 27 - protein, 72 aa, Homo sapiens
MPLLRGRCPARRHYRRLALLGLQPAPRFAHSGPPRQRPLSAAEMAVGLVVFFTTFLTPAAYVLGNLKQFRRN

SEQ ID NO: 28 - protein, 596 aa, Homo sapiens
MADAEARAEFPEEARPDRGTLQVLQDMASRLRIHSIRATCSTSSGHPTSCSSSSEIMSVLFFYIMRYKQSDPENPDNDRFVLAKRLSFVDVATGWLGQGLGVACGMAYTGKYFDRASYRVFCLMSDGESSEGSVWEAMAFASYYSLDNLVAIFDVNRLGHSGALPAEHCINIYQRRCEAFGWNTYVVDGRDVEALCQVFWQASQVKHKPTAVVAKTFKGRGTPSIEDAESWHAKPMPRERADAIIKLIESQIQTSRNLDPQPPIEDSPEVNITDVRMTSPPDYRVGDKIATRKACGLALAKLGYANNRVVVLDGDTRYSTFSEIFNKEYPERFIECFMAEQNMVSVALGCASRGRTIAFASTFAAFLTRAFDHIRIGGLAESNINIIGSHCGVSVGDDGASQMALEDIAMFRTIPKCTIFYPTDAVSTEHAVALAANAKGMCFIRTTRPETMVIYTPQERFEIGQAKVLRHCVSDKVTVIGAGITVYEALAAADELSKQDIFIRVIDLFTIKPLDVATIVSSAKATEGRIITVEDHYPQGGIGEAVCAAVSMDPDIQVHSLAVSGVPQSGKSEELLDMYGISARHIIVAVKCMLLN

SEQ ID NO: 29 - protein, 533 aa, Homo sapiens
MNEENIDGTNGCSKVRTGIQNEAALLALMEKTGYNMVQENGQRKFGGPPPGWEGPPPPRGCEVFVGKIPRDMYEDELVPVFERAGKIYEFRLMMEFSGENRGYAFVMYTTKEEAQLAIRILNNYEIRPGKFIGVCVSLDNCRLFIGAIPKEKKKEEILDEMKKVTEGVVDVIVYPSATDKTKNRGFAFVEYESHRAAAMARRKLIPGTFQLWGHTIQVDWADPEKEVDEETMQRVKVLYVRNLMISTTEETIKAEFNKFKPGAVERVKKLRDYAFVHFFNREDAVAAMSVMNGKCIDGASIEVTLAKPVNKENTWRQHLNGQISPNSENLIVFANKEESHPKTLGKLPTLPARLNGQHSPSPPEVERCTYPFYPGTKLTPISMYSLKSNHFNSAVMHLDYYCNKNNWAPPEYYLYSTTSQDGKVLLVYKIVIPAIANGSQSYFMPDKLCTTLEDAKELAAQFTLLHLDYNFHRSSINSLSPVSATLSSGTPSVLPYTSRPYSYPGYPLSPTISLANGSHVGQRLCISNQASFF

SEQ ID NO: 30 - protein, 407 aa, Homo sapiens
MPRGHKSKLRTCEKRQETNGQPQGLTGPQATAEKQEESHSSSSSSRACLGDCRRSSDASIPQESQGVSPTGSPDAVVSYSKSDVAANGQDEKSPSTSRDASVPQESQGASPTGSPDAGVSGSKYDVAANGQDEKSPSTSHDVSVPQESQGASPTGSPDAGVSGSKYDVAAEGEDEESVSASQKAIIFKRLSKDAVKKKACTLAQFLQKKFEKKESILKADMLKCVRREYKPYFPQILNRTSQHLVVAFGVELKEMDSSGESYTLVSKLGLPSEGILSGDNALPKSGLLMSLLVVIFMNGNCATEEEVWEFLGLLGIYDGILHSIYGDARKIITEDLVQDKYVVYRQVCNSDPPCYEFLWGPRAYAETTKMRVLRVLADSSNTSPGLYPHLYEDALIDEVERALRLRA

SEQ ID NO: 31 - protein, 638 aa, Homo sapiens
MVVSADPLSSERAEMNILEINQELRSQLAESNQQFRDLKEKFLITQATAYSLANQLKKYKCEEYKDIIDSVLRDELQSMEKLAEKLRQAEELRQYKALVHSQAKELTQLREKLREGRDASRWLNKHLKTLLTPDDPDKSQGQDLREQLAEGHRLAEHLVHKLSPENDEDEDEDEDDKDEEVEKVQESPAPREVQKTEEKEVPQDSLEECAVTCSNSHNPSNSNQPHRSTKITFKEHEVDSALVVESEHPHDEEEEALNIPPENQNDHEEEEGKAPVPPRHHDKSNSYRHREVSFLALDEQKVCSAQDVARDYSNPKWDETSLGFLEKQSDLEEVKGQETVAPRLSRGPLRVDKHEIPQESLDGCCLTPSILPDLTPSYHPYWSTLYSFEDKQVSLALVDKIKKDQEEIEDQSPPCPRLSQELPEVKEQEVPEDSVNEVYLTPSVHHDVSDCHQPYSSTLSSLEDQLACSALDVASPTEAACPQGTWSGDLSHHQSEVQVSQAQLEPSTLVPSCLRLQLDQGFHCGNGLAQRGLSSTTCSFSANADSGNQWPFQELVLEPSLGMKNPPQLEDDALEGSASNTQGRQVTGRIRASLVLILKTIRRRLPFSKWRLAFRFAGPHAESAEIPNTAGRTQRMAG

SEQ ID NO: 32 - protein, 70 aa, Homo sapiens
MKQKQEVMFQSRGRLSLYIQMSSVYSAKLGPVGGICGQKQKPSFFFFKAQSQDARPLAPAACISKIAKAG

SEQ ID NO: 33 - protein, 522 aa, Homo sapiens [sequence omitted]

SEQ ID NO: 34 - protein, 513 aa, Homo sapiens
MIRTPLSASAHRLLLPGSRGRPPRNMQPTGREGSRALSRRYLRRLLLLLLLLLLRQPVTRAETTPGAPRALSTLGSPSLFTTPGVPSALTTPGLTTPGTPKTLDLRGRAQALMRSFPLVDGHNDLPQVLRQRYKNVLQDVNLRNFSHGQTSLDRLRDGLVGAQFWSASVSCQSQDQTAVRLALEQIDLIHRMCASYSELELVTSAEGLNSSQKLACLIGVEGGHSLDSSLSVLRSFYVLGVRYLTLTFTCSTPWAESSTKFRHHMYTNVSGLTSFGEKVVEELNRLGMMIDLSYASDTLIRRVLEVSQAPVIFSHSAARAVCDNLLNVPDDILQLLKKNGGIVMVTLSMGVLQCNLLANVSTVADHFDHIRAVIGSEFIGIGGNYDGTGRFPQGLEDVSTYPVLIEELLSRSWSEEELQGVLRGNLLRVFRQVEKVREESRAQSPVEAEFPYGQLSTSCHSHLVPQNGHQATHLEVTKQPTNRVPWRSSNASPYLVPGLVAAATIPTFTQWLC

SEQ ID NO: 35 - protein, 154 aa, Homo sapiens
MEPSKTFMRNLPITPGYSGFVPFLSCQGMSKEDDMNHCVKTFQEKTQRYKEQLRELCCAVATAPKLKPVNSEETVLQALHQYNLQYHPLILECKYVKKPLQEPPIPGWAGYLPRAKVTEFGCGTRYTVMAKNCYKDFLEITERAKKAHLKPYEE

SEQ ID NO: 36 - protein, 120 aa, Homo sapiens
MGVLRARGEVGLALSPRLVGGASPPCDGGPESRGRKRGCLLSPCLVGVASPSCDDGPKSQRGKRGWLSVPASLGVPPPPAIGVLRSRGGRGAGSQSPPRGGCLPPCDGGPESRERKRGCL

SEQ ID NO: 37 - protein, 76 aa, Homo sapiens
MTDVETTYADFIASGRTGRRNAIHDILVSSASGNSNELALKLAGLDINKTEGEEDAQRSSTEQSGEAQGEAAKSES

SEQ ID NO: 38 - protein, 70 aa, Homo sapiens
MARTKQTARKSTGGKAPRKQLATKAARKSAPATGGVKKPHRYRPGTVALREIRRYQKSTELLIRKLPFQR

SEQ ID NO: 39 - protein, 861 aa, Homo sapiens
MTGRARARARGRARGQETAQLVGSTASQQPGYIQPRPQPPPAEGELFGRGRQRGTAGGTAKSQGLQISAGFQELSLAERGGRRRDFHDLGVNTRQNLDHVKESKTGSSGIIVRLSTNHFRLTSRPQWALYQYHIDYNPLMEARRLRSALLFQHEDLIGKCHAFDGTILFLPKRLQQKVTEVFSKTRNGEDVRITITLTNELPPTSPTCLQFYNIIFRRLLKIMNLQQIGRNYYNPNDPIDIPSHRLVIWPGFTTSILQYENSIMLCTDVSHKVLRSETVLDFMFNFYHQTEEHKFQEQVSKELIGLVVLTKYNNKTYRVDDIDWDQNPKSTFKKADGSEVSFLEYYRKQYNQEITDLKQPVLVSQPKRRRGPGGTLPGPAMLIPELCYLTGLTDKMRNDFNVMKDLAVHTRLTPEQRQREVGRLIDYIHKNDNVQRELRDWGLSFDSNLLSFSGRILQTEKIHQGGKTFDYNPQFADWSKETRGAPLISVKPLDNWLLIYTRRNYEAANSLIQNLFKVTPAMGMQMRKAIMIEVDDRTEAYLRVLQQKVTADTQIVVCLLSSNRKDKYDAIKKYLCTDCPTPSQCVVARTLGKQQTVMAIATKIALQMNCKMGGELWRVDIPLKLVMIVGIDCYHDMTAGRRSIAGFVASINEGMTRWFSRCIFQDRGQELVDGLKVCLQAALRAWNSCNEYMPSRIIVYRDGVGDGQLKTLVNYEVPQFLDCLKSIGRGYNPRLTVIVVKKRVNTRFFAQSGGRLQNPLPGTVIDVEVTRPEWYDFFIVSQAVRSGSVSPTHYNVIYDNSGLKPDHIQRLTYKLCHIYYNWPGVIRVPAPCQYAHKLAFLVGQSIHREPNLSLSNRLYYL

SEQ ID NO: 40 - protein, 221 aa, Homo sapiens
MPLALTLLLLSGLGAPGGWGCLQCDPLVLEALGHLRSALIPSRFQLEQLQARAGAVLMGMEGPFFRDYALNVFVGKVETNQLDLVASFVKNQTQHLMGNSLKDEPLLEELVTLRANVIKEFKKVLISYELKACNPKLCRLLKEEVLDCLHCQRITPKCIHKKYCFVDRQPRVALQYQMDSKYPRNQALLGILISVSLAVFVFVVIVVSACTYRQNRKLLLQ

SEQ ID NO: 41 - protein, 1623 aa, Homo sapiens
MAAEASKTGPSRSSYQRMGRKSQPWGAAEIQCTRCGRRVSRSSGHHCELQCGHAFCELCLLMTEECTTIICPDCEVATAVNTRQRYYPMAGYIKEDSIMEKLQPKTIKNCSQDFKKTADQLTTGLERSASTDKTLLNSSAVMLDTNTAEEIDEALNTAHHSFEQLSIAGKALEHMQKQTIEERERVIEVVEKQFDQLLAFFDSRKKNLCEEFARTTDDYLSNLIKAKSYIEEKKNNLNAAMNIARALQLSPSLRTYCDLNQIIRTLQLTSDSELAQVSSPQLRNPPRLSVNCSEIICMFNNMGKIEFRDSTKCYPQENEIRQNVQKKYNNKKELSCYDTYPPLEKKKVDMSVLTSEAPPPPLQPETNDVHLEAKNFQPQKDVATASPKTIAVLPQMGSSPDVIIEEIIEDNVESSAELVFVSHVIDPCHFYIRKYSQIKDAKVLEKKVNEFCNRSSHLDPSDILELGARIFVSSIKNGMWCRGTITELIPIEGRNTRKPCSPTRLFVHEVALIQIFMVDFGNSEVLIVTGVVDTHVRPEHSAKQHIALNDLCLVLRKSEPYTEGLLKDIQPLAQPCSLKDIVPQNSNEGWEEEAKVEFLKMVNNKAVSMKVFREEDGVLIVDLQKPPPNKISSDMPVSLRDALVFMELAKFKSQSLRSHFEKNTTLHYHPPILPKEMTDVSVTVCHINSPGDFYLQLIEGLDILFLLKTIEEFYKSEDGENLEILCPVQDQACVAKFEDGIWYRAKVIGLPGHQEVEVKYVDFGNTAKITIKDVRKIKDEFLNAPEKAIKCKLAYIEPYKRTMQWSKEAKEKFEEKAQDKFMTCSVIKILEDNVLLVELFDSLGAPEMTTTSINDQLVKEGLASYEIGYILKDNSQKHIEVWDPSPEEIISNEVHNLNPVSAKSLPNENFQSLYNKELPVHICNVISPEKIYVQWLLTENLLNSLEEKMIAAYENSKWEPVKWENDMHCAVKIQDKNQWRRGQIIRMVTDTLVEVLLYDVGVELVVNVDCLRKLEENLKTMGRLSLECSLVDIRPAGGSDKWTATACDCLSLYLTGAVATIILQVDSEENNTTWPLPVKIFCRDEKGERVDVSKYLIKKGLALRERRINNLDNSHSLSEKSLEVPLEQEDSVVTNCIKTNFDPDKKTADIISEQKVSEFQEKILEPRTTRGYKPPAIPNMNVFEATVSCVGDDGTIFVVPKLSEFELIKMTNEIQSNLKCLGLLEPYFWKKGEACAVRGSDTLWYRGKVMEVVGGAVRVQYLDHGFTEKIPQCHLYPILLYPDIPQFCIPCQLHNTTPVGNVWQPDAIEVLQQLLSKRQVDIHIMELPKNPWEKLSIHLYFDGMSLSYFMAYYKYCTSEHTEEMLKEKPRSDHDKKYEEEQWEIRFEELLSAETDTPLLPPYLSSSLPSPGELYAVQVKHVVSPNEVYICLDSIETSNQSNQHSDTDDSGVSGESESESLDEALQRVNKKVEALPPLTDFRTEMPCLAEYDDGLWYRAKIVAIKEFNPLSILVQFVDYGSTAKLTLNRLCQIPSHLMRYPARAIKVLLAGFKPPLRDLGETRIPYCPKWSMEALWAMIDCLQGKQLYAVSMAPAPEQIVTLYDDEQHPVHMPLVEMGLADKDE

SEQ ID NO: 42 - protein, 443 aa, Homo sapiens
MRNAIIQGLFYGSLTFGIWTALLFIYLHHNHVSSWQKKSQEPLSAWSPGKKVHQQIIYGSEQIPKPHVIVKRTDEDKAKSMLGTDFNHTNPELHKELLKYGFNVIISRSLGIEREVPDTRSKMCLQKHYPARLPTASIVICFYNEECNALFQTMSSVTNLTPHYFLEEIILVDDMSKVDDLKEKLDYHLETFRGKVKIIRNKKREGLIRARLIGASHASGDVLVFLDSHCEVNRVWLEPLLHAIAKDPKMVVCPLIDVIDDRTLEYKPSPLVRGTFDWNLQFKWDNVFSYEMDGPEGSTKPIRSPAMSGGIFAIRRHYFNEIGQYDKDMDFWGRENLELSLRIWMCGGQLFIIPCSRVGHISKKQTGKPSTIISAMTHNYLRLVHVWLDEYKEQFFLRKPGLKYVTYGNIRERVELRKRLGCKSFQWYLDNVFPELEASVNSL

SEQ ID NO: 43 - protein, 735 aa, Homo sapiens
MHCGLLEEPDMDSTESWIERCLNESENKRYSSHTSLGNVSNDENEEKENNRASKPHSTPATLQWLEENYEIAEGVCIPRSALYMHYLDFCEKNDTQPVNAASFGKIIRQQFPQLTTRRLGTRGQSKYHYYGIAVKESSQYYDVMYSKKGAAWVSETGKKEVSKQTVAYSPRSKLGTLLPEFPNVKDLNLPASLPEEKVSTFIMMYRTHCQRILDTVIRANFDEVQSFLLHFWQGMPPHMLPVLGSSTVVNIVGVCDSILYKAISGVLMPTVLQALPDSLTQVIRKFAKQLDEWLKVALHDLPENLRNIKFELSRRFSQILRRQTSLNHLCQASRTVIHSADITFQMLEDWRNVDLNSITKQTLYTMEDSRDEHRKLITQLYQEFDHLLEEQSPIESYIEWLDTMVDRCVVKVAAKRQGSLKKVAQQFLLMWSCFGTRVIRDMTLHSAPSFGSFHLIHLMFDDYVLYLLESLHCQERANELMRAMKGEGSTAEVREEIILTEAAAPTPSPVPSFSPAKSATSVEVPPPSSPVSNPSPEYTGLSTTGAMQSYTWSLTYTVTTAAGSPAENSQQLPCMRNTHVPSSSVTHRIPVYPHREEHGYTGSYNYGSYGNQHPHPMQSQYPALPHDTAISGPLHYAPYHRSSAQYPFNSPTSRMEPCLMSSTPRLHPTPVTPRWPEVPSANTCYTSPSVHSARYGNSSDMYTPLTTRRNSEYEHMQHFPGFAYINGEASTGWAK

SEQ ID NO: 44 - protein, 168 aa, Homo sapiens
MSLTHRLHLCKYWGCAVSNVCRFWEGRPLPLMIVVPYTLPVSLPVGSCVIITGTPILTFVKDPQLEVNFYTGMDEDSDIAFQFRLHFGHPAIMNSCVFGIWRYEEKCYYLPFEDGKPFELCIYVRHKEYKVMVNGQRIYNFAHRFPPASVKMLQVFRDISLTRVLISD

SEQ ID NO: 45 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 46 - protein, 450 aa, Homo sapiens
MRECISIHVGQAGVQIGNACWELYCLEHGIQPDGQMPSDKTIGGGDDSFNTFFSETGAGKHVPRAVFVDLEPTVVDEVRTGTYRQLFHPEQLITGKEDAANNYARGHYTIGKEIVDLVLDRIRKLADLCTGLQGFLIFHSFGGGTGSGFASLLMERLSVDYGKKSKLEFAIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCMLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDLAALEKDYEEVGVDSVEAEAEEGEEY

SEQ ID NO: 47 - DNA, 544 bp, Artificial Sequence
  probe derived from SOX30
gaaagttttcagtcgtgatttgaggagttaaaaccaaatgcaatttatgtcttcataaaattttgattagtgaaactagagtctggatgtttcattgtaggaatatttaagttattaagtagtttaattttaatggctgaaatttgcatcaacatgtattattattactttatcctggaacatgcaaaatactgaagcctcacagttgtatgtgaggggaaaggggaaataaatctagcatagtgtgatttttattttatctcaggatacattttttaaatgattttttgtttgctttttatgtaatacttatggatgttgtcaatttttgatgtaacattttgaaagtattttgacaactcctagtgaacttggacttggttgctaaatttaacttacactaataaccaattataagttccaaatgtgttttaatggcacctgggtgattcttcagctaaatttagtcatttctgtttctaaatatttttatcattttaaaatattttttttccatttggcatacatcgttctttgttgtaat

SEQ ID NO: 48 - DNA, 289 bp, Artificial Sequence
  probe derived from SPATA22
gatcgtgaacttccgagactgattagaggccgagttcatagatgtgttggcaactatgaccagaaaaagaacattttccaatgtgtttctgtcagaccggcgtctgtttctgaacaaaaaactttccaggcatttgtcaaaattgcagatgttgagatgcagtattatattaatgtgatgaatgaaacttaagtagtgataaaaggaagtttagcataaattatagcagttttctgttattgcttaatttaccatctccatagttttatagctactattgtatttcact

SEQ ID NO: 49 - DNA, 386 bp, Artificial Sequence
  probe derived from MAEL
gaggccagcaatagtgtgacacccaaaatggttgtattggatgcagggcgttaccagaagctaagggttgggagttcaggattctctcatttcaactcttctaatgaggaacaaagatcaaacacacccattggtgactacccatctagggcaaaaatttctggccaaaacagcagcgttcggggaagaggaattacccgcttactagagagcatttccaattcttccagcaatatccacaaattctccaactgtgacacttcactctcaccttacatgtcccaaaaagatggatacaaatctttctcttccttatcttaatgatggtactcttttcaatttctgaaaacagtaacaggcccaacttccttcttactacagtcata

SEQ ID NO: 50 - DNA, 313 bp, Artificial Sequence
  probe derived from COX8C
ccctgtctgccgcggaaatggctgttggacttgtggtgttttttacnaccttcttaacaccagctgcatatgtgctaggcaacctgaagcagttcagaaggaattagatggaagatgatgttgaacagctgttaacgtccaaaaaactttcagaaaaagctgtgtttttgttaacgagcaaaattgcctagttgagttgatgcaaccattgtggtattcactttcctcatgtttatgatgaatattttgcacttttttagtactgtgcattatatagatgtatagtcaaaaatgttctgcttaagtgttaaat

SEQ ID NO: 51 - DNA, 521 bp, Artificial Sequence
  probe derived from TKTL1
ccttcattcatccctagttcggaaattcaagctaactacttaccctttaaactgtcactgcatatgcaagtaccgctctaatttttggatcattaaagggagttacacaacttttaagtgaaaaaaataggtaacaaaacaaccacctgatagtaagttttctgataagactatagataagtggtagaggtaatcaattcttccgaagtgtttccttcgtgaataactggtagaggtaatagttttttcaatgtatttccttcatgagtaaagaaaatgtggattgaagtatagattccagtagcctagtttccacagcacgataacaccatgacgcctactgctgttcccaccttgggattctgtgtgctgccatcccacctgcagctgccctggaattcccttcgctgtttgccttcatctccctccacgtttgagaggctgtcaggcagcagcgaaagcttgttaggatgtcctgtgctgcttgtgatgagagcctccacactgtactgttcaagtca

SEQ ID NO: 52 - DNA, 355 bp, Artificial Sequence
  probe derived from RBM46
agcacttgaagacaatggttatagtagatttgattaccaangatcactatctgtanctggagattagaacaattatatgaccagaagcatctaaccattatgtaaaaagaaatgatgagacaaaaagattaagatacaaattttgtgcagtactaaagaaaaagcagtctaccattgtggtccttgaaaataactatagatatttttgttatttgttagacacaaattataattttgttgttaatgtatttaagcattttatagttatgctttgtgtttttgatattctttgtattgttaataacaagtgttatgggtttttaatgttgaaatcatgtgttaatttttgtacttg

SEQ ID NO: 53 - DNA, 279 bp, Artificial Sequence
  probe derived from MAGEB6
tcacattcatggctgtttaaccaatctgaaagttacggtttgggaattaataaaacaaagtcatacaacacattttctttgtaattgagaactagataacatggtaacagagaattgattttcatatgaatcttaacnccacagtaaaatagttgacatcataatangaagagaaagaaaaggaaaaacagaaatgtaaaagttgtttaattcttggtttgcctaattcgttttcctatttcttttcatacaaataaaggatacctggatttatttagg

SEQ ID NO: 54 - DNA, 497 bp, Artificial Sequence
  probe derived from NBPF4
gcagagaaggcccagtgtgtccatccccagtgcggtgatactaggatggtcacttggttaaggaggggtctaggagctctgtcccttgtaaagacatcttatttgtaagtaatttggaaagtggtttgaaatagtataaatatcctgtattctagtgatcttcttcagaacattttatcaccaattaatcaccccgtctgtgtcagttattatatttaagtttgtacattgaaaattgtctatctcaaaatcttaccttatacttgcttttgctggcattcttngtaaaaaagatcattccctgcccaaattttaactttcatccaaaattaattttaatttctttttgctggcattctgttgtgaaaaagaatattctctgccccaattatnactttcatccaaaattaattttagtccatcagttaaaattttaanttttaaatctgtttaattaaaacatttcttgcctctcactctggactattggatttttt

SEQ ID NO: 55 - DNA, 417 bp, Artificial Sequence
  probe derived from C12orf37
ttcctcctgaccacaaaaagcacttatactctaggatgactgattccagcccagtggcctggcaagggtgaattacaccttgcatatcacactcttgacatttgtgtgcgctagcataagaattataattgaaacagggatttaagtatctcctctctaggtgcctaccctccttggactcaggtcaaatttattaaaggaagttttgtttctagataggttgtttgaaataaaataacagaatgttcaagtaacacagtgtacctacagcttttaacaaaattgaggacttgggtctcgaaacaatttcctttgattttcaggtattttatctataaaaagggagataaagcattagttcataggacagttatatgtttaaatgtgataatgtatattaaccaccttgcatgtatt

SEQ ID NO: 56 - DNA, 364 bp, Artificial Sequence
  probe derived from TPTE2P2
ctgaggttctattcatggtgagcaagtcttttttttngtttgtttcttcaagctctaacaagggtgcctactacatggcttttcagttancnccaaaataanatgtnacaattttttttnctattcttaggctttatctacaaagaaatgaattggataatcttcataaacaaaaaacntggaaaatttatcaaccagaatatgcagtagaganatattttnatgagaaatgacttaagttatgttgtaactggtagctgattaagtatagttcccngcaccccttctgggaaagaattatgttctttctaaccctgccacatagttatatgttctaaatcttccttgctggtacatctatatt

SEQ ID NO: 57 - DNA, 504 bp, Artificial Sequence
  probe derived from DPEP3
gggcagtcattggatctgagttcatcgggattggtggaaattatgacgggactggccggttccctcaggggctggaggatgtgtccacatacccagtcctgatagaggagttgctgagtcgtagctggagcgaggaagagcttcaaggtgtccttcgtggaaacctgctgcgggtcttcagacaagtggaaaaggtgagagaggagagcagggcgcagagccccgtggaggctgagtttccatatgggcaactgagcacatcctgccactcccacctcgtgcctcagaatggacaccaggctactcatctggaggtgaccaagcagccaaccaatcgggtcccctggaggtcctcaaatgcctccccataccttgttccaggccttgtggctgctgccaccatcccaaccttcacccagtggctctgctgacacagtcggtccccgcagaggtcactgtggcaaagcctcacaaagccccctctcctagttcattcacaagcat

SEQ ID NO: 58 - DNA, 446 bp, Artificial Sequence
  probe derived from C10orf82
aggtcactgaatttggctgtggcacgagatacactgtcatggccaaaaactgctacaaggacttcctggagatcacggagagggccaagaaggcacatctgaaaccatatgaagaaatatatggagttagctccacaaaaacttctgctccgtctccaaaagttttgcagcatgaagagctgctgccaaaatatcccgatttttctattccagatggaagctgccctgcccttggaaggcccctgagagaggaccccaaaactccgctgacatgtggctgtgctcagaggccaagtataccatgcagtgggaagatgtatctagagccactgtcctccgcaaagtatgcagaaggctagaagcgcagagtctcccaaggaggtgaactttaagtggggcttccaaaacctgccattctcatgttggaatcacgcccagtgagcaat

SEQ ID NO: 59 - DNA, 262 bp, Artificial Sequence
  probe derived from LOC440896
agctgagtgccacgtgctgacgtcactaagatcaatacagcaaactctgaaagatggacagagagacaggagatggtcctttataatgcagtgtgatctgtgctgcaatagagatggaagagtgttctgatcggctggaaaacaacgcacatgggaagctgtacagaaatgagtggggaaagttttggaagctagaagttcaaaacgaggtgttggcagcaccatgctctctcngaagatgctaggaagaatctgctccatg

SEQ ID NO: 60 - DNA, 503 bp, Artificial Sequence
  probe derived from CDNA clone IMAGE5265646
gataccaacatctaatccgccaagaaagaatttcagaattaaaatttgggtgtgttcttgcgctggggatttctctgacctaccttctgatagaactttgaacccgagtcaatagtcaatatactagttttttacttaaaactgtaacattttaatctaattttggggacgtgaaataaaactaatggnnnnnnnnagaaatttttatcactggnaataacctaattttgaaaacactgatggttagtttcttgaaatattaatattactacaagtcataagtaaaagcattctatcttaagtgagaaactacaaagttggataattactatttgagtttgtggcttggtttgaataaacacttgcttgttttaagtaaaagttcagctgaagtgacaatcaacctttaatcttgtaaagcttctgtgttagatattttctatctctaacatgccaaacatgcatattaaactgagtttttttgcatgcaatttctgtgccca

SEQ ID NO: 61 - DNA, 361 bp, Artificial Sequence
  probe derived from HIST1H3C
tccgcgcaagcagcttgctactaaagcagcccgtaagagcgctccggccaccggtggcgtgaagaaacctcatcgctaccgcccgggcaccgtggccttgcgcgaaatccgtcgctaccagaagtccaccgagctgctgatccggaagctgccgttccagcgcctggtgcgagaaatcgcccaggacttcaaaaccgacctgcgtttccagagctctgcggtgatggcgctgcaggaggcttgcgaggcctacctggtgggactcttcgaagacaccaatctgtgcgctattcacgctaaacgcgtcaccatcatgcccaaagatatccagctggcacgtcgcatccgtggggaaagggca

SEQ ID NO: 62 - DNA, 546 bp, Artificial Sequence
  probe derived from PIWIL1
attcaccggcttccttattttatatgtaaaaattaagattttatattttatcttcttgtttctcatagatattttgtgagcatttttttgtttattttgaagaaatgtggataagatacttggtagtataaaacagactctctgagangtatttgaaatgtgtttggagatttacttaaacgtactttcaggagtgagcaagtcctacttataaacctatattaactttatttttgagatacctgttttgaatttaaaggagataagaggcgtaaagtaggatgctcactacaaccataggtggggtttcagctcatatcttaaagataaaaggtactattatataacctatacacaagatacaggagaaaatatgcttgatttttatttggcaggggggctaggttgtatgggagtaaaaaaaacattgaaaatttttaaattgtccaaagaaacattttaagactctttaacaaaaaaggccatgagtaaatctctatattaacattactatttattttgttttggaactgggacatgattcta

SEQ ID NO: 63 - DNA, 521 bp, Artificial Sequence
  probe derived from C19orf41
ggccgtgcgtgatgggcatggaggggcctttcttccgggactacgcgctgaacgtgtttgtggggaaagtggagacaaatcaactggaccttgtggcgtcctttgtcaagaaccaaacgcagcacttaatgggtaactctctgaaagatgagcctctgctggaagagctggtgaccctcagggcgaatgtgatcaaggaattcaagaaagttttaatttcatatgaattaaaagcctgcaaccccaaactttgccgcttgctaaaagaagaggtgttggactgtttacattgccagaggatcactcccaagtgtatccacaaaaagtactgctttgtcgaccggcaaccccgcgtggccctgcagtaccagatggacagcaaatacccgaggaaccaggcgctgttgggcatcctcatttctgtgtctctggctgtctttgtcttcgtggtcatcgtggtctcggcttgtacatacagacaaaaccgaaaactcctgctgcagtaggacggtggtttgggg

SEQ ID NO: 64 - DNA, 486 bp, Artificial Sequence
  probe derived from RNF17
gaatttaatcctttatctatcttagtacaatttgttgattatggatcaactgcaaagctgacattaaacagactgtgccaaattccttctcatcttatgcggtatccagctcgagccataaaggttctcttggcagggtttaaacctcccttaagggatctaggggagacaagaataccatattgtcccaaatggagcatggaggcactgtgggctatgatagactgtcttcaaggaaaacaactctatgctgtgtccatggctccagcaccagaacagatagtgacattatatgacgatgaacagcatccagttcatatgccgttggtagaaatggggcttgcagataaagatgaataagtgcctaagtgtatacagtgagagcatctatagaagcctagaagaattctgttatgtttagactatgtcttatctttagactatttcaggcttaattttcctaacttgttcagccctagtgcttta

SEQ ID NO: 65 - DNA, 306 bp, Artificial Sequence
  probe derived from GALNTL5
ggaactttcactaaggatctggatgtgtggaggccaactctttnataatcccctgctctcgagtaggacatatcagtaagaaacaaactggaaaaccttctacaatcatcagtgctatgacacataactacctaagactggtgcacgtttggctggatgaatataaggagcagttttttcttcgaaagcctggtctgaaatatgtcacctacggaaatattcgcgagcgtgttgagttaaggaaacgactgggttgcaagtcatttcagtggtatttggataatgtcttcccagagttggaggcat

SEQ ID NO: 66 - DNA, 454 bp, Artificial Sequence
  probe derived from RFX4
gatttatggcattgagtatcacactcagctctgctgtgttaactttgtgaaactggatggaacaaactttaacttaccaagcaccaagtgtgaaagtgactttcacggttccttcataaaactataataatatccgacactttgatagaaaaaaattcaaagctgtgcctttgagcctatactatactgtgtatgtgtggaaataaaaatgtattgtacttttggagaattttttgtaggcatttttctgtcagatttgtagtaatttgtgaggtttgttagagattaatataggttttctttctgtattataaaatgcaccaagcaattatggtggacctattaccctatgggtaagaaataaatggaaatatgacatcggatgtttcagcaactgttctgtaaataaaatctttgatcacaccactcagtgtgataattgtgtctacagcta

SEQ ID NO: 67 - DNA, 505 bp, Artificial Sequence
  probe derived from LGALS14
agaacaatgtcatcactacccgtaccatacacactgcctgtttccttgcctgttggttcgtgcgtgataatcacagggacaccgatcctcacttttgtcaaggacccacagctggaggtgaatttctacactgggatggatgaggactcagatattgctttccaattccgactgcactttggtcatcctgcaatcatgaacagttgtgtgtttggcatatggagatatgaggagaaatgctactatttaccctttgaagatggcaaaccatttgagctgtgcatctatgtgcgtcacaaggaatacaaggtaatggtaaatggccaacgcatttacaactttgcccatcgattcccgccagcatctgtgaagatgctgcaagtcttcagagatatctccctgaccagagtgcttatcagcgattgagggagatgatcagactcctcattgttgaggaatccctctttctacctgaccatgggattcccagagcctactaacagaa

SEQ ID NO: 68 - DNA, 526 bp, Artificial Sequence
  probe derived from IGFBP1
aataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtcgataat

SEQ ID NO: 69 - DNA, 287 bp, Artificial Sequence
  probe derived from TUBA3C
tctccggcaggtgggcattaactaccagccccccacggtggtccctgggggagacctggccaaggtgcagcgggctgtgtgcatgctgagcaacaccacggccatcgcggaggcctgggctcgcctggaccataagttcgatctcatgtatgccaagcgggcctttgtgcactggtacgtgggagaaggcatggaggagggggagttctctgaggcccgcgaggacctggcagctctggagaaggattatgaagaggtgggcgtggattccgtggaagccgaggctg

SEQ ID NO: 70 - DNA, 1949 bp, Homo sapiens; identical to SEQ ID NO: 7

SEQ ID NO: 71 - DNA, 2145 bp, Homo sapiens
aaggcagggggcggggcgtctccgagcggcggggccaagggagggcacaacagctgctacctgaacagtttctgacccaacagttacccagcgccggactcgctgcgccccggcggctctagggacccccggcgcctacacttagctccgcgcccgagagaatgttggaccgacgacacaagacctcagacttgtgttattctagcagctgaacacaccccaggctcttctgaccggcagtggctctggaagcagtctggtgtatagagttatggattcactaccagattctactgtatgctcttgacaactatgaccacaatggtccacccacaaatgaattatcaggagtgaacccagaggcacgtatgaatgaaagtcctgatccgactgacctggcgggagtcatcattgagctcggccccaatgacagtccacagacaagtgaatttaaaggagcaaccgaggaggcacctgcgaaagaaagtgtgttagcacgactttccaagtttgaagttgaagatgctgaaaatgttgcttcatatgacagcaagattaagaaaattgtgcattcaattgtatcatcctttgcatttggactatttggagttttcctggtcttactggatgtcactctcatccttgccgacctaattttcactgacagcaaactttatattcctttggagtatcgttctatttctctagctattgccttattttttctcatggatgttcttcttcgagtatttgtagaaaggagacagcagtatttttctgacttatttaacattttagatactgccattattgtgattcttctgctggttgatgtcgtttacattttttttgacattaagttgcttaggaatattcccagatggacacatttacttcgacttctacgacttattattctgttaagaatttttcatctgtttcatcaaaaaagacaacttgaaaagctgataagaaggcgggtttcagaaaacaaaaggcgatacacaagggatggatttgacctagacctcacttacgttacagaacgtattattgctatgtcatttccatcttctggaaggcagtctttctatagaaatccaatcaaggaagttgtgcggtttctagataagaaacaccgaaaccactatcgagtctacaatctatgcagtgaaagagcttacgatcctaagcacttccataatagggtcgttagaatcatgattgatgatcataatgtccccactctacatcagatggtggttttcaccaaggaagtaaatgagtggatggctcaagatcttgaaaacatcgtagcgattcactgtaaaggaggcacagatagaacaggaactatggtttgtgccttccttattgcctctgaaatatgttcaactgcaaaggaaagcctgtattattttggagaaaggcgaacagataaaacccacagcgaaaaatttcagggagtagaaactccttctcagaagagatatgttgcatattttgcacaagtgaaacatctctacaactggaatctccctccaagacggatactctttataaaacacttcattatttattcgattcctcgttatgtacgtgatctaaaaatccaaatagaaatggagaaaaaggttgtcttttccactatttcattaggaaaatgttcggtacttgataacattacaacagacaaaatattaattgatgtattcgacggtctacctctgtatgatgatgtgaaagtgcagtttttctattcgaatcttcctacatactatgacaattgctcattttacttctggttgcacacatcttttattgaaaataacaggctttatctaccaaaaaatgaattggataatctacataaacaaaaagcacggagaatttatccatcagattttgccgtggagatactttttggcgagaaaatgacttccagtgatgttgtagctggatccgattaagtatagctcccccttccccttctgggaaagaattatgttctttccaaccctgccacatgttcatatatcctaaatctatcctaaatgttccttgaagtatttatttatgtttatatatgtttatatatgttcttcataaatctattacatatatatagataaaaaaaaaaaaaaa

SEQ ID NO: 72 - DNA, 2462 bp, Homo sapiens
gctctgaccgactggtcccctaaacggtggcggcggtttttggtcgttgggccccgggatttaggaccaacatttgaagacccgaaggggaactgcaaccatgaatgaagaaaatatagatggaacaaatggatgcagtaaagttcgaactggtattcagaatgaagcagcattacttgctttgatggaaaagactggttacaacatggttcaggaaaatggacaaaggaaatttggcggtcctcctccaggttgggaaggtccacctccacctagaggctgtgaagtttttgtaggaaaaatacctcgtgatatgtatgaagatgagttagttcctgtatttgaaagagctgggaagatatatgaatttcgacttatgatggaatttagtggtgaaaatcgaggttatgcttttgtgatgtacactacaaaagaagaagcccaattagccatcagaattcttaataattatgaaattcgaccagggaagtttattggtgtgtgtgtaagcctggataattgtagattatttattggagctattcccaaggaaaagaagaaagaagaaattttagatgaaatgaagaaagttacagaaggagttgtagatgtcattgtttatccaagtgcaactgataagaccaaaaatcgtggttttgcatttgtggaatatgaatctcacagagctgctgctatggcaaggaggaaactaattccaggaacattccaactatggggccacaccattcaggtagattgggctgacccagagaaagaggtggatgaggaaaccatgcagagagttaaagttctttatgtaagaaatttaatgatctcaactacagaggaaacaattaaagcagaattcaataaatttaagcctggtgcagttgaacgggtaaagaaacttagagattatgcttttgttcactttttcaaccgagaagatgcagtggctgccatgtctgttatgaatggaaaatgcattgatggagcaagtattgaggtaacactagctaaaccagtaaataaagaaaacacttggagacagcatcttaatggtcagattagtccaaattctgaaaatctgattgtgtttgctaacaaagaagagagccacccaaaaactctaggcaagctgccaactcttcctgctcgtctcaatggtcagcatagcccaagtccgcctgaagttgaaagatgcacttaccctttttatcctggaacaaagcttactccaattagtatgtattctttaaaatccaatcattttaattctgcagtaatgcatttggattattactgcaacaaaaataactgggcaccaccagaatattatttatattcaacaacaagtcaagatgggaaagtactcttggtgtataagatagttattcctgctattgcaaatggatcccagagttacttcatgccagacaaactctgtactacgttagaagatgcaaaggaactggcagcccagtttacattacttcatttggactacaatttccatcgcagctcaataaatagtctttcccctgttagtgctaccctctcttctgggactcccagcgtgcttccttatacttcaaggccttattcttatccaggctatcctttgtcaccaacaatatcacttgctaatggcagccatgttggacagcggctatgtatctccaatcaggcctccttcttctgaagaaaatactaacattagtatgaaaatttgtgtaaatttgtagtatgaaaacttgcaaattaaaatattgttttattttagaatcgggtttgcatatttggttttaaaaaggtatttattccaaagtactaaacatcagctataattcagaataacatggagttgtagaatttataaaaatgcaaagtttaaaaagttattcagtggtttctcttgataaaggtacagcaaactactattctttttaaacttctaggattttcttctactttctgagtgggcaatagaacctagtcatttatgttttttttttttttgcataattttactaaatagtatttcacaaatattaaagcacttgaagacaatggttatagtagatttgattaccaaggatcactatctgtactggagattagaacaattatatgaccagaagcatctaaccattatgtaaaaagaaatgatgagacaaaaagattaagatacaaattttgtgcagtactaaagaaaaagcagtctaccattgtggtccttgaaaataactatagatatttttgttatttgttagacacaaattataattttgttgttaatgtatttaagcattttatagttatgctttgtgtttttgatattctttgtattgttaataacaagtgttatgggtttttaatgttgaaatcatgtgttaatttttgtacttgaattcaaattttttgacattaaatatgtgatgcttctaaaaaaaaaaaaaaaa

SEQ ID NO: 73 - DNA, 459 bp, Homo sapiens; identical to SEQ ID NO: 15

SEQ ID NO: 74 - DNA, 2591 bp, Homo sapiens
actctttctctctcactctctctcttttcccacccttaagccaagtacagggatagttgtctcatcattggtggcttaaaatgatgtttttgaacaagaagacaccccatgggtacttttggtgactagcactatctctgtttttttccttttaaattcctgagctattgtttagcagtacacccttttatctccattgctactgaagctgaatgttacttgggtggaaagcataactgctttcttttctatgtccttaaaccctttgataatgttactgtttgagagtccctgaagccaggatattagaagagtctggcttgtctgaacagctgaactacgaaataatggagtagggcaggtgggtgggggagcagggcgttctgtcgataaacgagctcccttctttgcacacatagccagttaatcgggcattctgagatagtttggatggggagggggagcttctgagaatcgccagtgacagttaagtggcctattgttgacgtcctctgctgaacgacttggttggattcagcttctgccttacccccaccccctgtggattttctgttagattcatcatttgccattcaggcatcatctttcactttctccttccaacatgactgctttgtgtgctggcccctgctttactcctgctattcccaaactataaagggaactgtgtggggcttcagcaggactgagaaattgactctgctgtctgttgagaactctaataatgactgcggtgaccttcatgtctagcagaaaacccactgtctgtgctagcacaatgatgaccacctgaagacagagaaaagggaatcttgattgaattccttctcatacaatatatagttatttcctttgttctcctctccattctcttctcttccccttctccccatcgccactggaggactgatctcaaatgcagctgtgactaaaagttaatgccttttgaataataatacattgcattttgcaggctttaccaagccaattcactcaagttgtctcatctataccccttcaaaccctgtgagcctctaggtgctgtgctgtcctgaggcctgggccatggtgcccaaggaaagcccctgaagctcaccaggaggaagaagcatgcagggcactcctggaggcgggacgcgccctgggccatcccccgtggacaggcggacactcctggtcttcagctttatcctggcagcagctttgggccaaatgaatttcacaggggaccaggttcttcgagtcctggccaaagatgagaagcagctttcacttctcggggatctggagggcctgaaaccccagaaggtggacttctggcgtggcccagccaggcccagcctccctgtggatatgagagttcctttctctgaactgaaagacatcaaagcttatctggagtctcatggacttgcttacagcatcatgataaaggacatccaggtgctgctggatgaggaaagacaggccatggcgaaatcccgccggctggagcgcagcaccaacagcttcagttactcatcataccacaccctggaggagatatatagctggattgacaactttgtaatggagcattccgatattgtctcaaaaattcagattggcaacagctttgaaaaccagtccattcttgtcctgaagttcagcactggaggttctcggcacccagccatctggattgacactggaattcactcccgggagtggatcacccatgccaccggcatctggactgccaataagattgtcagtgattatggcaaagaccgtgtcctgacagacatactgaatgccatggacatcttcatagagctcgtcacaaaccctgatgggtttgcttttacccacagcatgaaccgcttatggcggaagaacaagtccatcagacctggaatcttctgcatcggcgtggatctcaacaggaactggaagtcgggttttggaggaaatggttctaacagcaacccctgctcagaaacttatcacgggccctcccctcagtcggagccggaggtggctgccatagtgaacttcatcacagcccatggcaacttcaaggctctgatctccatccacagctactctcagatgcttatgtacccttacggccgatcgctggatcccgtttcaaatcagagggagttgtacgatcttgccaaggatgcggtggaggccttgtataaggtccatgggatcgagtacatttttggcagcatcagcaccaccctctatgtggccagtgggatcaccgtcgactgggcctacgacagtggcatcaagtacgccttcagctttgagctccgggacactgggcagtatggcttcctgctgccggccacacagatcatccccacggcccaggagacgtggatggcgcttcggaccatcatggagcacaccctgaatcacccctactagcagcacgactgagggcaggaggctccatccttctccccaaggtctgtggctcctcccgaaacccaagttatgcatccccatccccatgccctcatcccgacctcttagaaaataaatacaagtttgaacaggcaaaaaaaaaaaaaaaa

SEQ ID NO: 75 - DNA, 3623 bp, Homo sapiens; identical to SEQ ID NO: 20

SEQ ID NO: 76 - DNA, 1504 bp, Homo sapiens; identical to SEQ ID NO: 23

SEQ ID NO: 77 - DNA, 1746 bp, Homo sapiens
agcaaccgccaaacgtagctgagtggctgggcctgcggccctccctgcaccggcggacgctcctctcagtcttggagtctcttcgcccaggtggctgtggatccggtacgggagttgccgccgcggtccaactccccgctgccgcccagcgcatccgctcgcaggagcgccggcggccagcagtgcgctctgcagcatgtcgctacatgcctgggagtgggaagaggaccccgcaagcatagagcccatctcctccatcactagcttttaccagtccacgagcgagtgtgacgtggaggaacacctgaaggccaaggccagggcccaggagtctgactctgaccgcccgtgcagcagcatcgagtcctcatctgagcctgccagcactttcagctccgacgtgccccacgtggtcccctgcaaattcaccatctcactggccttccctgtgaatatgggtcagaagggaaaatatgcaagtttgattgaaaaatataagaaacaccctaaaacagacagttctgttacaaagatgcgtcgtttttaccacattgagtatttccttctgccggacgatgaagaacctaaaaaagttgacatattgctatttccaatggtggccaaagtattcctggagtcaggagtaaagactgtgaagccgtggcacgaaggtgacaaagcctgggtgtcgtgggagcagacttttaatatcactgtgacaaaggaattattaaagaaaataaatttccacaaaatcaccttgaggctctggaacactaaagacaagatgtcaagaaaagtcagatattaccgattaaagactgccggcttcacagacgacgtgggagcttttcataagtcagaagtgagacatttggttttaaatcagagaaaattatctgaacagggcattgagaataccaacattgtcagagaagagtcgaaccaggaacatccgccaggaaaacaagaaaaaacagaaaaacacccaaagtctttgcaaggttctcaccaagcagagcccgaaacttcttccaagaacagtgaggaatatgagaagtccctcaaaatggacgattcttccacgattcaatggagtgtttcaagaacaccaaccatttctttggcaggagcaagcatgatggagatcaaggaattaattgagagtgaatcacttagcagcttaacaaacatattagacagacaaaggtctcaaattaaagggaaagattcagagggaagaaggaaaatccagaggagacataagaagcccctggcagaagaagaggcagaccccacgctgacaggccccaggaagcagagcgctttctccatccagctggccgtcatgccgctccttgccggtacccactgcttgccctgttcccagcagctcctgcttgtcttgtggccagaacggccataagcggatgctcctaatgacaaccctgctgcgtgccacctcatttcatcctcacagccacctcaggaaactcaggggaattggcttcatagtacagggggaaacaaaggcccagagagggtagctaccttgcctgagatcacacagctaggaaatgtgaagcctggacttgaacccagctctgtttgattccaaaatccaaggaagcctatgggaatattaaatgtcatcattgtgtttgttaagaagatgctgcttttaataaatgctttcctacagatttttacaaaaaaaaaaaaaaaaaaaagaaaaaaaaaaaaaaa

SEQ ID NO: 78 - DNA, 524 bp, Homo sapiens
ttttttttttcaaattttttaataaagcaaatggctgggaaaaaaggtgagaaactgctcgcttggatttaagaactgctgctacttgctgtgtgacctggactagttttttgtttgttagttttatttttatttttgtttttttgctgggctttaccttggttttaattttctgaacttcattctgctcatctgaaaaatgaaatactaatgtcttcatcttagtgtaatagggatgattaggtaaggtcaaatatatggaagcatcttgtaaactgtaaaggtatgtaaaaatgtgagggtttgttttttgtgtgtgtatgtgctcttaatgcccaagatgcagagtagaaattaggcagctgggatgccaaacagccaggatgcaacatattggagagaagagccagatatctgaggaggaagtcacaccatcattgccatccatgttgtgatagtcaacagaaggagtaaaaagagacatcagaggaatgaaagggaggaatgaggattgggaaggaacg

SEQ ID NO: 79 - DNA, 2837 bp, Homo sapiens
agtctcgcgggaagctccgttgtgggcgccccggctggtggctgagctcaggccttcaggcagaggggaggcgagggcggggcggtcacgtgagagcactgccgcggtgggttgtgggggtgctgcggcgccgtttgctttgccaaaccgacaaaagagagatgatggccaacgacgccaagcccgacgtgaagaccgtgcaggtgctgcgggacacagccaaccgcctgcggatccattccatcagggccacgtgtgcctctggttctggccagctcacgtcgtgctgcagtgcagcggaggtcgtgtctgtcctcttcttccacacgatgaagtataaacagacagacccagaacacccggacaacgaccggttcatcctctccaggggacatgctgctcctatcctctatgctgcttgggtggaggtgggtgacatcagtgaatctgacttgctgaacctgaggaaacttcacagcgacttggagagacaccctaccccccgattgccgtttgttgacgtggcaacagggtccctaggtcagggattaggtactgcatgtggaatggcttatactggcaagtaccttgacaaggccagctaccgggtgttctgccttatgggagatggcgaatcctcagaaggctctgtgtgggaggcttttgcttttgcctcccactacaacttggacaatctcgtggcggtcttcgacgtgaaccgcttgggacaaagtggccctgcaccccttgagcatggcgcagacatctaccagaattgctgtgaagcctttggatggaatacttacttagtggatggccatgatgtggaggccttgtgccaagcattttggcaagcaagtcaagtgaagaacaagcctactgctatagttgccaagaccttcaaaggtcggggtattccaaatattgaggatgcagaaaattggcatggaaagccagtgccaaaagaaagagcagatgcaattgtcaaattaattgagagtcagatacagaccaatgagaatctcataccaaaatcgcctgtggaagactcacctcaaataagcatcacagatataaaaatgacctccccacctgcttacaaagttggtgacaagatagctactcagaaaacatatggtttggctctggctaaactgggccgtgcaaatgaaagagttattgttctgagtggtgacacgatgaactccaccttttctgagatattcaggaaagaacaccctgagcgtttcatagagtgtattattgctgaacaaaacatggtaagtgtggcactaggctgtgctacacgtggtcgaaccattgcttttgctggtgcttttgctgccttttttactagagcattcgatcagctccgaatgggagccatttctcaagccaatatcaaccttattggttcccactgtggggtatccactggagaagatggagtctcccagatggccctggaggatctagccatgttccgaagcattcccaattgtactgttttctatccaagtgatgccatctcgacagagcatgctatttatctagccgccaataccaagggaatgtgcttcattcgaaccagccaaccagaaactgcagttatttataccccacaagaaaattttgagattggccaggccaaggtggtccgccacggtgtcaatgataaagtcacagtaattggagctggagttactctccatgaagccttagaagctgctgaccatctttctcaacaaggtatttctgtccgtgtcatcgacccatttaccattaaacccctggatgccgccaccatcatctccagtgcaaaagccacaggcggccgagttatcacagtggaggatcactacagggaaggtggcattggagaagctgtttgtgcagctgtctccagggagcctgatatccttgttcatcaactggcagtgtcaggagtgcctcaacgtgggaaaactagtgaattgctggatatgtttggaatcagtaccagacacattatagcagccgtaacacttactttaatgaagtaaactaggcttatttctaaaaagtcaagtctattggctttggcccaaaagcactggtatctttgtattaaattcatgtttattgtcacaaaaccattatttatacctatacagttgtactgtttcttttaaagcaaagccatttaacatctttcttcattcctaatttggaaattaaagtttacctttctgttaatctatgtataaatgttactctgagttattaatgtggattttaaaattgtaagcaatagaataggaaataaaacaactacctaatacaaatatttctgataagactacaaatatctgactgagctggggattaaagtagaggtaactgtatcttaaatgagtatgatttccttgtaagttaaaaaaattgaaatttaattgtagacttcaatagtccaagttttgaaggatgtttgagcttttgtataatgccatttatacctgcagttttacagataatgtttgactgcagttgccttggaaattcctccaaagtttgccttcatctctcctctacagtttggaggtgatggtgcagcagtggaacatctcttgatgcaccacactacttgtgttctgtgaagtgatgaaagtataactggttctagtttgcacactacacacatagttttgtgaagcttcagaaatgttttttcttttccttgtggccaaaccagtttgttaatctgattatattcatctgctaatgatactaaagttaatgtaataaagcatttaaaaatcagaaaaaaaaaaaaaaaaaaaaaaaaaaaaaaaaaaaaa

SEQ ID NO: 80 - DNA, 2345 bp, Homo sapiens
aaatgcagggcgcagcagccgctgcagtggagccggtaggcctggccggcgggctgaaaggaagtgcgagctgtccgcccagggccgggtatccgcccctgcaggctgtggaggggatgtcaggagactggctggcctcttttcttggcccccgactccttccagtctgacactgaagactttataagcttccccccgaccaccctccacgggctccactctccacgggcctgggcttgcgccgcttcgagatcagcctgggggtcgcgccctcctggtcttgtccacgaagcgccgttcttgggccgttaggagctgctgggaagggctctgataggcccactcctcttctccacccaggagatgagaaggagggcaggcctttttaatctgatcagaatgttaacccatctctccgccttgcggtagaacccctggatacattatttgccctctcgaaaggcaggctctgaatttgattcaggtatatttcttcatagctaaccagcacaatggaaaactcagggaaagcaaataaaaaggatacacatgacgggccaccaaaagaaattaaactgcctaccagtgaagcacttctagactatcaatgtcaaataaaggaagatgccgtggagcaattcatgtttcaaataaagacacttaggaaaaagaaccaaaaatatcatgaaagaaatagccgcttaaaagaagaacagatttggcacatacggcatctactaaaggaactgagtgaagagaaggcagagggattgccagttgtaacaagagaggatgttgaagaagcgatgaaggaaaaatggaagtttgaaagagaccaggaaaaaaacttgagagatatgcgcatgcaaataagtaatgctgagaaactatttcttgagaaactcagtgaaaaggaatattgggaggagtacaagaatgtagggagtgaacgacatgctaaactcattacctccttacaaaatgacatcaacacagttaaagagaatgcagagaaaatgtcagaacactataaaatcactctggaagatactagaaagaaaataatcaaggaaactttgttgcaactggaccaaaagaaggaatgggccacacagaatgctgtaaagctcattgacaagggcagttatctagagatctgggagaatgactggctcaaaaaagaggttgcaattcacaggaaggaagttgaagaattaaaaaatgctattcatgaactggaagcagaaaatttggtgcttattgatcaactatccaactgtagacttgtggatctcaagatacccaggtatccagtgctacattcctgtcccacctctaatcctcgtcatctgctgctgctgcctttggaatcatgtctaatctctgccaggcgttgctggcgactatatcttacccaagctgctggactagaagtgccacctgaagaaatgtctttggaattgccagaaacacatatagaagagaagtcagaattgcaacccacagaagtagaaagtagagacttgatgtcctcatcagatgagagcactatcttacatcttagtcatgaaaatagcatcgaagatctccagtatgtgaagatagataaagaggaaaactcaggcacagagtttggggacactgatatgaagtacttactatatgaggatgagaaggatttcaaggattatgtaaacttgggccccctgggagtgaagcttatgagtgtggagagcaagaaaatgcccattcattttcaagagaaggaaattccagtcaaactctataaagatgtcaggagcccagaaagccacatcacatataagatgatgaagtcttttctctaagacggaaagctgcaaaggaaacacaacttttccttataaatgttctttgggaactgaagtatatccgttgcccattttacttacactttggctcatttttaaaccagctgttatttctaaaggtcatatttacatttaaaatcaaaggtattcagctattcatttacttgcatggtatgagtgaccaaaacggaagcacgctttgtatttctacactgaagtattcagaagcatgacagtgggttcaaggtagtctctgaggttccttttcacacacaaaaaattcactgattaatctgtgattccagtatgaaatagttccattagaaatgtttctaagaaaaacttagaagtttgcatagcattgtctacacatctttccctctgaggatgctcaatgtgatagacagccagtctataatgcaagccaattctccgtagtttaaccctgtgtattagtctgttctcatgctgctaataaagacataattgaaactg

SEQ ID NO: 81 - DNA, 1670 bp, Homo sapiens
gggtcgtcatgatccggaccccattgtcggcctctgcccatcgcctgctcctcccaggctcccgcggccgacccccgcgcaacatgcagcccacgggccgcgagggttcccgcgcgctcagccggcggtatctgcggcgtctgctgctcctgctactgctgctgctgctgcggcagcccgtaacccgcgcggagaccacgccgggcgcccccagagccctctccacgctgggctcccccagcctcttcaccacgccgggtgtccccagcgccctcactaccccaggcctcactacgccaggcacccccaaaaccctggaccttcggggtcgcgcgcaggccctgatgcggagtttcccactcgtggacggccacaatgacctgccccaggtcctgagacagcgttacaagaatgtgcttcaggatgttaacctgcgaaatttcagccatggtcagaccagcctggacaggcttagagacggcctcgtgggtgcccagttctggtcagcctccgtctcatgccagtcccaggaccagactgccgtgcgcctcgccctggagcagattgacctcattcaccgcatgtgtgcctcctactctgaactcgagcttgtgacctcagctgaaggtctgaacagctctcaaaagctggcctgcctcattggcgtggagggtggtcactcactggacagcagcctctctgtgctgcgcagtttctatgtgctgggggtgcgctacctgacacttaccttcacctgcagtacaccatgggcagagagttccaccaagttcagacaccacatgtacaccaacgtcagcggattgacaagctttggtgagaaagtagtagaggagttgaaccgcctgggcatgatgatagatttgtcctatgcatcggacaccttgataagaagggtcctggaagtgtctcaggctcctgtgatcttctcccactcagctgccagagctgtgtgtgacaatttgttgaatgttcccgatgatatcctgcagcttctgaagaagaacggtggcatcgtgatggtgacactgtccatgggggtgctgcagtgcaacctgcttgctaacgtgtccactgtggcagatcactttgaccacatcagggcagtcattggatctgagttcatcgggattggtggaaattatgacgggactggccggttccctcaggggctggaggatgtgtccacatacccagtcctgatagaggagttgctgagtcgtagctggagcgaggaagagcttcaaggtgtccttcgtggaaacctgctgcgggtcttcagacaagtggaaaaggtgagagaggagagcagggcgcagagccccgtggaggctgagtttccatatgggcaactgagcacatcctgccactcccacctcgtgcctcagaatggacaccaggctactcatctggaggtgaccaagcagccaaccaatcgggtcccctggaggtcctcaaatgcctccccataccttgttccaggccttgtggctgctgccaccatcccaaccttcacccagtggctctgctgacacagtcggtccccgcagaggtcactgtggcaaagcctcacaaagccccctctcctagttcattcacaagcatatgctgagaataaacatgttacacatggaaaaaaaaaaaaaaaaaaa

SEQ ID NO: 82 - DNA, 1009 bp, Homo sapiens; identical to SEQ ID NO: 12

SEQ ID NO: 83 - DNA, 1406 bp, Homo sapiens
atcttaagaggcgttcctttttgcatagttcccatgagcatgagagaagaagcaatgcacgctccggcagattcctaggaaccaaatacctctgaggagcaccagatttcagcttatgggatgctttgattgctctgtggctgcatttaggagaaggaagctgcagtcatgcgtcatcactgccagcctcacatctcttgacagttaaagccttagggtggagcaagggaaaatttaaaataacaaatgaagcaaaagcaagaggtgatgttccaaagcagaggaaggctaagtttatatatacaaatgtcaagtgtgtatagtgcaaaactaggaccagttggtggaatctgtggtcaaaaacaaaagccttccttttttttttttcaaggcccagtcccaagacgcaagaccacttgcgccagcagcgtgcatcagcaagatagcaaaagcaggacgagagctgcccggaagacatctacctggccagaagacacctaccctggccggaagacatgtacccctgaagatagagaaagaggccatcgtgtactacgtagcagtcatgtcagactgggacacttcctgtttacagaggactataaaacccctgtcctgtcctcacttggggctgacgccatcttaggcctcagcccgcctgcagccaggcgttcgttaaaacagcatgttgctccacaccgccttgtattgtttgttggtcccactctctgggctcgaaccaatacaagcacctttcaagcagtatattcttcagtgtcttgatcctccaaataactctcttctaattcctcctgaccacaaaaagcacttatactctaggatgactgattccagcccagtggcctggcaagggtgaattacaccttgcatatcacactcttgacatttgtgtgcgctagcataagaattataattgaaacagggatttaagtatctcctctctaggtgcctaccctccttggactcaggtcaaatttattaaaggaagttttgtttctagataggttgtttgaaataaaataacagaatgttcaagtaacacagtgtacctacagcttttaacaaaattgaggacttgggtctcgaaacaatttcctttgattttcaggtattttatctataaaaagggagataaagcattagttcataggacagttatatgtttaaatgtgataatgtatattaaccaccttgcatgtattcaaatgtgttttgaaatctaacgtctacattttgatagtttaactgttctacataagtgacttacaacaggcattaaatattgtttggcattttcatatatctgtaactgtatcttaatctacaatgagcttaattttaagtgtagcataaaacagaaccttcaataaagtggtaatattagg

SEQ ID NO: 84 - DNA, 3416 bp, Homo sapiens
ctccgcgggagccgttggggctgttggcctcgggctgaggtgcaaggaccaggactagggcgagggcagcggtccaagaaatagaaaacaatgactgggagagcccgagccagagccagaggaagggcccgcggtcaggagacagcgcagctggtgggctccactgccagtcagcaacctggttatattcagcctaggcctcagccgccaccagcagagggggaattatttggccgtggacggcagagaggaacagcaggaggaacagccaagtcacaaggactccagatatctgctggatttcaggagttatcgttagcagagagaggaggtcgtcgtagagattttcatgatcttggtgtgaatacaaggcagaacctagaccatgttaaagaatcaaaaacaggttcttcaggcattatagtaaggttaagcactaaccatttccggctgacatcccgtccccagtgggccttatatcagtatcacattgactataacccactgatggaagccagaagactccgttcagctcttctttttcaacacgaagatctaattggaaagtgtcatgcttttgatggaacgatattatttttacctaaaagactacagcaaaaggttactgaagtttttagtaagacccggaatggagaggatgtgaggataacgatcactttaacaaatgaacttccacctacatcaccaacttgtttgcagttctataatattattttcaggaggcttttgaaaatcatgaatttgcaacaaattggacgaaattattataacccaaatgacccaattgatattccaagtcacaggttggtgatttggcctggcttcactacttccatccttcagtatgaaaacagcatcatgctctgcactgacgttagccataaagtccttcgaagtgagactgttttggatttcatgttcaacttttatcatcagacagaagaacataaatttcaagaacaagtttccaaagaactaataggtttagttgttcttaccaagtataacaataagacatacagagtggatgatattgactgggaccagaatcccaagagcacctttaagaaagccgacggctctgaagtcagcttcttagaatactacaggaagcaatacaaccaagagatcaccgacttgaagcagcctgtcttggtcagccagcccaagagaaggcggggccctggggggacactgccagggcctgccatgctcattcctgagctctgctatcttacaggtctaactgataaaatgcgtaatgattttaacgtgatgaaagacttagccgttcatacaagactaactccagagcaaaggcagcgtgaagtgggacgactcattgattacattcataaaaacgataatgttcaaagggagcttcgagactggggtttgagctttgattccaacttactgtccttctcaggaagaattttgcaaacagaaaagattcaccaaggtggaaaaacatttgattacaatccacaatttgcagattggtccaaagaaacaagaggtgcaccattaattagtgttaagccactagataactggctgttgatctatacgcgaagaaattatgaagcagccaattcattgatacaaaatctatttaaagttacaccagccatgggcatgcaaatgagaaaagcaataatgattgaagtggatgacagaactgaagcctacttaagagtcttacagcaaaaggtcacagcagacacccagatagttgtctgtctgttgtcaagtaatcggaaggacaaatacgatgctattaaaaaatacctgtgtacagattgccctaccccaagtcagtgtgtggtggcccgaaccttaggcaaacagcaaactgtcatggccattgctacaaagattgccctacagatgaactgcaagatgggaggagagctctggagggtggacatccccctgaagctcgtgatgatcgttggcatcgattgttaccatgacatgacagctgggcggaggtcaatcgcaggatttgttgccagcatcaatgaagggatgacccgctggttctcacgctgcatatttcaggatagaggacaggagctggtagatgggctcaaagtctgcctgcaagcggctctgagggcttggaatagctgcaatgagtacatgcccagccggatcatcgtgtaccgcgatggcgtaggagacggccagctgaaaacactggtgaactacgaagtgccacagtttttggattgtctaaaatccattggtagaggttacaaccctagactaacggtaattgtggtgaagaaaagagtgaacaccagattttttgctcagtctggaggaagacttcagaatccacttcctggaacagttattgatgtagaggttaccagaccagaatggtatgacttttttatcgtgagccaggctgtgagaagtggtagtgtttctcccacacattacaatgtcatctatgacaacagcggcctgaagccagaccacatacagcgcttgacctacaagctgtgccacatctattacaactggccaggtgtcattcgtgttcctgctccttgccagtacgcccacaagctggcttttcttgttggccagagtattcacagagagccaaatctgtcactgtcaaaccgcctttactacctctaacctgcagaagacgatgcagccgcttttctttttgaaatgactttgggatttttttaagcttttatttactttttttttaactgttatctttctggatgaaacttgggaaggggattaggagatctagcattttatttctagcattgctattcaccggcttccttattttatacgtaaaaattaagattttatattttatcttcttgtttctcatagatattttgtgagcatttttttgtttattttgaagaaatgtggataagatacttggtagtataaaacagactctctgagagtatttgaaatgtgtttggagatttacttaaacgtactttcaggagtgagcaagtcctacttataaacctatattaactttatttttgagatacctgttttgaatttaaaggagataagaggcgtaaagtaggatgctcactacaaccataggtggggtttcagctcatatcttaaagataaaaggtactattatataacctatacacaagatacaggagaaaatatgcttgatttttatttggcaggggggctaggttgtatgggagtaaaaaaaacattgaaaatttttaaattgtccaaagaaacattttaagactctttaacaaaaaaggccatgagtaaatctctatattaacattactatttattttgttttggaactgggacatgattctatttgttataaaataaaattgatgtgattgtcaccttaaaaaaaaaaa

SEQ ID NO: 85 - DNA, 1112 bp, Homo sapiens
tagcacttcatagactgctatcacgcattcatttttactagtacctattgaacactaagttccaggcactgggctaggcactgggatggtgtggtgagcaaaaactgaccagtcctgccgtggagtttgctgggggagacacatgttactcaaagaatcacactaaggatagcaatttatctcaaagctgcaagttcctgccatctacatgtgcccagagtccccaaaattgagtgtcaaacagaggctgggtgacctcctgtccaggattgctctcatcgtatgaatcgaagttttcatcttaacgtgtttttttctcctgagaataggccaaccaatcaatggctcagacagataagccaacatgcatcccgccggagctgccgaagatgctgaaggagtttgccaaagccgccattagggtgcagccgcaggacctcatccagtgggcagccgattattttgaggccctgtcccgtggagagacgcctccggtgagagagcggtctgagcgagtcgctttgtgtaaccgggcagagctaacacctgagctgttaaagatcctgcattctcaggttgctggcagactgatcatccgtgcagaggagctggcccagatgtggaaagtggtgaatctcccaacagatctgtttaatagtgtgatgaatgtgggtcgcttcacggaggagatcgagtggctgaagtttttagcccttgcttgcagcgctctgggagttactattaccaaaactctcaagatagtgtgtgaggtcttatcatgtgaccataatggtgggtcgccccggatcccgttcagcaccttccagtttctctacacgtatattgccaaagtggatggggagatctctgcatcacatgtcagcaggatgctaaactacatggaacaggaagtaattggccctgatggtataatcacagtgaatgactttacccaaaaccccagggttcagctggagtaaaagcacaattttggcaattttaaaggaagatacagagatgattgtacttcagaatgactgaaacccatataccacccaaaatccattttcttgtacaactggtacacactaataaacaattaaaaaaaaaaa

SEQ ID NO: 86 - DNA, 2818 bp, Homo sapiens
gttacctggtacgctggctgctacctccctcactcttgtcagagtcggagctacaggcagtgccttcagctctgagctcaggcatcccggtccctgtttttgcggttaaggactctaaagtgttgtgtcgtgttcatcaactttttctcaacttccctggctctacctcttctgccacaaacgtcagcatggtggtatctgccgaccctttgtccagcgagagggcagagatgaacatcctagaaatcaaccaggaattgcgctcgcagctggcagagagcaatcagcagttccgagacctcaaagagaaattccttataactcaagctactgcctactccctggccaaccagctgaagaaatacaagtgtgaagagtacaggaatcacctgcccccagagaggtgcagaagactgaagaaaaggaagtccctcaggactcactggaggaatgtgctgtcacttgttcaaatagtcacaacccttctaactccaaccagcctcacaggagcaccaaaatcacatttaaggaacacgaagtcgactctgctctggttgtagagagtgaacaccctcatgatgaagaggaggaagctctaaacattcccccagaaaatcaaaatgaccatgaggaggaggaggggaaagcgccagtgccccccagacaccatgacaagtccaactcttaccggcatcgtgaagtctctttcttggcattggatgaacagaaagtttgctccgctcaggatgttgccagggattactccaatcccaaatgggatgaaacctcacttggcttcctcgaaaagcaaagtgatcttgaagaggtgaaaggacaagaaacagttgctcccaggctcagcaggggaccgctgagagtggacaagcatgaaatcccccaggagtcactggatggatgttgcttgactccttccatccttcctgacctgactccctcctaccacccttattggagcactttgtactcttttgaagacaagcaagtcagcttggctcttgtagacaaaattaaaaaggatcaagaggagatagaagaccaaagcccaccatgccccaggctcagccaggagctgccagaggtgaaggagcaggaagtcccagaggactctgtgaatgaagtttacttgactccctcagttcaccatgacgtgtctgactgccaccagccttatagcagcaccttgtcctcattggaggatcagcttgcctgctctgctctggatgtagcctcccccaccgaggcggcctgtccccaagggacttggagtggagacttgagccaccaccagtcagaggtgcaagtttcacaggcacagctggaaccaagcaccctggtgcccagttgtctgcgactacagctggatcaagggttccactgtgggaacggcttggcccagcggggcctttcctccaccacctgcagcttctcagccaatgctgattctgggaaccaatggcccttccaagagctggttttagagccctctctggggatgaagaaccctccccagctggaagatgatgcacttgaaggctcagcaagcaacacacaagggcgtcaagtcactggccggattcgtgcctcccttgtcctgatactgaagaccatcagaagaagactcccgttcagcaagtggagactggcattcagattcgctggcccgcatgctgagagcgcagagataccaaatactgctggaaggacgcaaaggatggcaggatgaaagaatgtcacaaaaagcagcttttccacttgataaaaacaactaaaacagcaaagcaagtttaagtccaaacacaatactgcaggggtccttcactgaggattgaatttcagacacagaatactcttgatgacttcaagccactatgctcctttgatttgagaagccacattccatccccctccaattgtgatcaatacctagggagaccaatgcccagatggacaaatagcattgaccggcgttagccctgtttctcaattcccatcgtgtagagaacaggagtccgcagctgctggcaggagacagcatgtcagccgggactctgccagggcagagtatgagcaataccatgttcttgctgaaaacgcttagcctgagtttcataggcggtaaccctcagataactgcagaatgtagaacattgaacaggacaactgacatggacttgtttgtggaggacaggtcagctgtctggctcaatggtctacattctgaagttatctgaaaatgtcgtcatgattaaattcagcctaaacattttgccaggaactctgcagagtccatgctgtgagcttcctacctcagcccatctgcaggcagagaaggcccagtgtgtccatccccagtgcggtgatactaggatggtcacttggttaaggaggggtctaggagctctgtcccttgtaaagacatcttatttgtaagtaatttggaaagtggtttgaaatagtataaatatcctgtattctagtgatcttcttcagaacattttatcaccaattaatcaccccgtctgtgtcagttattatatttaagtttgtacattgaaaattgtctatctcaaaatcttaccttatacttgcttttgctggcattctttgtaaaaaagatcattccctgcccaaattttaactttcatccaaaattaattttaatttctttttgctggcattctgttgtgaaaaagaatattctctgccccaattataactttcatccaaaattaattttagtccatcagttaaaattttaaattttaaatctgtttaattaaaacatttcttgcctctc

SEQ ID NO: 87 - DNA, 3848 bp, Homo sapiens; identical to SEQ ID NO: 10

SEQ ID NO: 88 - DNA, 1000 bp, Homo sapiens
agggagacgcctagaagatggaggcccagattcttgagacgtttctctttctgatctagcaggagggacaaagagctcctccactccctcattccccaagaaggcccccagcctacccagtttccgtgaccattccgccctgggaaagcggcttcccagacctccttatctatttttcttgaatcatgagagatgaaattgcaacaacagttttctttgtcacaagattggtgaaaaaacatgataaactaagtaaacagcaaatagaagactttgcagaaaagctgatgacgatcttgtttgaaacatacagaagtcactggcactctgattgcccttctaaagggcaagccttcaggtgcatcaggataaacaacaatcagaataaagatcccattctagaaagggcatgtgtggaaagtaatgtagatttttctcacctgggacttccgaaggagatgaccatatgggtagatccctttgaagtatgctgtaggtatggtgagaaaaaccatccatttacagttgcttcttttaaaggcagatgggaggaatgggaactatatcaacaaatcagttatgccgttagtagagcctcatcagacgtttcctctggcacttcctgcgatgaagaaagttgtagcaaggaacctcgtgtcattcctaaagtcagcaatccgaagagtatttatcaggttgaaaacttgaaacagccctttcaatcttggttacaaatcccccgcaaaaagaatgtggtggacggccgtgttggcctcctgggaaacacttaccatggctcgcagaagcatcctaagtgttacaggcctgctatgcaccggctggacagaattttataacccacatctgggaatgaatttgcagcacctggtagaagaaggcaccttggaaggcactgccttgggcttccatggcaggaagatgagaagaaatcttcagggtgatttctggagcctgaaaagaataaaaaacaaaaccaaaa

SEQ ID NO: 89 - DNA, 2942 bp, Homo sapiens
agagcagcctcggaaccgagacgatgcgtgcgctccgcgaccgagccgggctcctcctctgcgtgctgctgctggcggcgctgctggaggcggcgctagggctccccgtgaagaagccgcggctccgcggaccacggcctgggagcctcacgaggctcgcagaggtctcagcctccccagatcctaggcctctgaaggaagaggaggaggcaccactgctccccagaacccacctgcaggcagagccacaccaacatggatgctggactgtcactgagccagcagccatgaccccaggcaacgccacccctcccaggaccccagaggttactccgttgcggctggagctgcagaagctgccgggattggccaacacaaccttgagtacccctaaccctgatacccaggcttcagcctccccagatcctaggcctctgagggaagaggaggaggcacgactgctccccagaacccacctgcaggcagagctacaccaacatggatgttggactgtcactgagccagcagccctgaccccagggaatgccacgcctcccaggacccaggaggttactcccttgctgctggagctgcagaagctgccagaattggtccacgcaaccttgagtacccctaaccctgataaccaggtgaccatcaaggtggtggaggacccccaggccgaggtgtcgatagacctgttggctgagcccagcaatcccccgccccaggatacccttagctggctgcccgccctctggtccttcctctggggagactacaaaggagaggaaaaagacagggccccaggggagaagggggaggaaaaggaggaagacgaggactatccttcagaggatatcgagggtgaggatcaagaggacaaagaggaagatgaggaagagcaggcgctctggttcaatggaactacagacaactgggaccagggctggctggcccccggggattgggtcttcaaggattctgtcagctacgactatgagcctcagaaggagtggagtccctggtctccctgcagtgggaactgcagcactggcaagcagcagaggactcggccctgtggctatggctgcactgccaccgagacccgtacctgtgacctgccctcctgtcctggcactgaggacaaggacaccttgggcctccccagtgaggagtggaagctcctggcccgcaatgctacggacatgcatgatcaagatgtggacagctgtgagaagtggctgaactgcaagagcgacttcctaatcaagtatctgagccagatgctgcgggacctgcccagctgcccgtgtgcctacccactggaggccatggacagccctgtgagcctacaggacgagcaccagggccgcagcttccggtggagggatgccagtggccctcgcgagcgcctggacatctaccagcccacggcgcgcttctgcctgcgttccatgctgtctggggagagcagcacactggccgcccagcactgctgctatgacgaggacagccggctgctgacccgtggcaagggcgccggcatgcccaacctcatcagcaccgacttctcacctaagctgcacttcaagttcgacacgacgccctggatcctgtgcaagggggactggagccgcctccacgctgtgctccctcccaacaacggccgagcctgcaccgacaaccccctggaggaggagtacctagcacagttgcaggaggccaaggagtactagtgacggggttgctgaacagacactgcagggagagggcaggcggctgctgctgttgcacgggagaactttcctcacccgcccctgcccagacagggtgaggaaagggctcccccagtgaggttggtccgaggctgtgtgccctctgccagcgaccccgaagcagatatctcagtggggttagtgagaaggttgaagggtatgtagggcccagggtgggtgtccctgggagccctggaaatgtgcatatgtgcatgtgtctgccggggcctccctctgctgcctgctgggaccctggccactcatttttctcctccttgggagctgggctcttctgccctggctctgcacataagtgttagccagcagctccagaaaaatcccgattcccgggatctgccacgagtcactcctactccaccctgatggccagcagaggaagggccactcttctcatgggcacagccatcctttgccgggggggcatccagcccgggtggccacccctccttatctctgggtggtgcacatgcccttctttccccactccctgccacgagccactgcacaggaggctatctgtagccccaagctgcctttctgttggacaccaactttagtcttgggctgcaagccagcccagctgaggcgaagtggactccaggcagggaatgggttgcccaattctggtccctttcctttgctcagccccctctgttctgctgattgtagggatgtgcagggctgggagttggcactccccccgagtggggaggtgacagcttgtcacagtagccaggcttgggtgggttcagcactagctcgggacggtgtgtcacacgtctatagtaaaccagttctctgggaggggaaaaaagccctgatttattgcatttgggcagcttctgtggtgtaaattctcccagcagtgtcccatgtcatgctgccagcatcactgaatgcactgaactcagagttgggaagagatgcacataatcgctctcccggcacacctcatgcctcttccctgcctccccattcccctggctgcacttccttgccttctatggggttgaaatattgaagtctcaactgtctctgttcacaagagccaccaaaagttaggggacttcagtcctagcccccagatggccgccctgaagctctctgggctcctcagcaataaagcactttattttcaaaaaa

SEQ ID NO: 90 - DNA, 2570 bp, Homo sapiens
aatcggtatatttactgattgtgtattggtgaattggtgctggctctcagttcccgcctcgcggcggggggggcctgaccacccctgcgatgggtgtcctaagagccagggggggaagaggggctggctgtcagtccccgcctcccggggggtgcctcccgcccctacgatggtggttccaagagtctgggggggaagaggggctggctttcagtacccgcctcgcggggggtgcctcccccaggtgcgatgggggtcctaagagccagggggtgaagaggggctggctctctgtcccctcgtcgcgggggttgcatcccccccctgtgatgggggtcccaagagccagggggggaagaagggctggctctcagtccccgcctcactagcaggggagttgctgccaaggccctcaaacatgggggccatcctttagaaaccctgtctagttgtttagagacataggccaccgacctcatccagggccccacagtttgggttaaaagtccacctgccatcttttctctctctgacacatacaatggaaaaggctttgtcagatcgggtaaccccagggctgaagctgccagaagtttttcctttaactcatgaaagacttgctgttgttaggatcccccttccaaaggttcccggtccccgacccctttgtgacctcatacaaaggcttggcttatactgcaaagtttgggatccacagtctacaaaaccccacagctcctgagaattctctcgcctgccttcggcccttaggctctggtagattgcaaataacatgctttctttctgttcccgggtggcttcggacccctgtcggatcggaaatcccaagtaaggtacctgccctgggcagatttgagctttcttcttggacacctaatacccacagtcctccaggctgaggtagattgcaaatgacctgctttctttctgttcctgggtgccttcggacccctgtcggatagtaaatcccaagtaaggtacctgccgtcagcagatttgagctttcttcttggacacctaatacccacagtcctccaggtgggtcctaaagttcataggatccgcgatgggggtcccaagccagggagggacgaggggctggctctcagtccccgcctcgcgggggggtgcctccccccccccctgcgatgagggtcctaagagccaggggggaagaggggatggctgtcagtccctccctcgtgggtggtacctccgccttctgcgatggtggtcctaagagccagggggagacgaggggctggctctcagtccccgcttcgcgaggggtgcctcccccccccgcgatgggggtcctaagagtctggggggaaagaggggctggctctcaatccccgcctcgcgggggtgcctcccccaggtgctatgtgggtcctaagagccaggggtgaagaggggctagctctctgtcccctcgtcacgggggttgcatcacccaccctgcgatggaggttccaagagccaagggggtaagaggggctggctctcagtccccgcctcgcggagggtgcctcccccaccactgcgatgggggtcccaagagccagggggggaagagggtctggctctccaccaccacaaaatggggggcctttatgttcaggttttgcccaagagtcagcttatttgcttcttgtactagcagggcagttgctgccaaggccctcaaacagggtgccatcctttagaaaccctgtctagttgttcagagacgtaggccaccggcctcatccagggccccacagtttgggttaaaagtccacctgccatcttttctctctctgacgcatacaatggaaaaggctttgtccgatcggatagccccagggctgaagctgccagaagtttttcctttaactcatgaaagacttgctgttgttgggatcccccttccaaaggttcccagtccccgccccctttgtgacatcatacaaagtcttggcttatactgcaaagtttgggatccacagtctacaaaaccccacagctcctgagaactctcttgcctgccttcggctcttaggctatagtagattccaaataacctgctttctttctgttcccgggtggcttcggacccctgtcggatcggaaatcccaagtaaggtacctgccgtcggcagatttgagctttcttcttggacacctaatacccacagtcctccaggctccagtagattgcaaatgacctgcttactttctgttcccgggctgcgttctgacacctgtcggatagtaaatcccaagtaaggtaccagccgtcggcagatttgagctttcttcttggacacctatacccacagtcctccagtgttttagacgcccagctgcacaacttgattgccttacaaatgacctgcttccaggatgcggaaattcctaatttcttctgtgacccttctcaactcccccatcttgcatgttgtgacaccttcaccaataacataatcatgtatttccctgctgtcatatttggttttcttcccatctctgggacccttttctcttactataaaattgtttcctcc

SEQ ID NO: 91 - DNA, 1149 bp, Homo sapiens
ccgcagccatgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccagctacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtagcaagggcttcccgctgcctccagacagcacctgggtcctcgccaccctaagccccgggacacctgttggagggcggatgggatctgcctagcctgggctggagtccttgctttgctgctgctgagctgccgggcaacctcagatgaccgacttttccctttgagcctcagtttctctagctgagaaatggagatgtactactctctcctttacctttacctttaccacagtgcagggctgactgaactgtcactgtgagatattttttattgtttaattagaaaagaattgttgttgggctgggcgcagtggatcgcacctgtaatcccagtcactgggaagccgacgtgggagggtagcttgaggccaggagctcgaaaccagtccgggccacacagcaagaccccatctctaaaaaattaatataaatataaaataaaaaaaaaaaaaa

SEQ ID NO: 92 - DNA, 1099 bp, Homo sapiens; identical to SEQ ID NO: 21

SEQ ID NO: 93 - DNA, 1021 bp, Homo sapiens
atgactggccgcaggcggccgggcgggctgtaacccgccgctgaactagcgcttctgtgtccagaggcttcggcctggccgccgtcgcctgtaagctacgaggaggagatttacgacttggccgggcgcagcaaaggccagactctgcgcgaacaggcgctgcgcaccaaccggcaggcacctggcgggcaccatcgcacggtggcgcagaagcccttcaatggccagcgccagctgcagccgcggccgcgcagtcgtcccacctgagcttgggcgaatgtggattgggaaagtcgacattaatcaactcattattcctcatagatttgtattctccagagtatccaggtccttctcacagaattaaaaagactgtacagctggcagcctgttatcgattacattgatagtaaatttgaggactacctaaatgcagaatcatgcgtgaacagacatcagatgcctgataacagggtgcagtgttgttcatacttcattgctccttcaggacatggacttaaaccattggatattgagtttatgaagcgtttgcatgaaaaagtgagtatcaccccacttattgccaaagcagacacactcacaccagaggaatgccaacagtttaaaaaacagttgaaaatggtgaacattgttattttacaattctaagaaatatgttgataaggtaacttttccggtcacacacaacagactgattttcctgtggaagacctcacaggagttctcttgaaccaggagactattttgagctgaaatacctggagccctctcaagctatttgggcctttcaatttgaaacccaggtttggatgattctaagctctatggcagagactgtaatcattttctcttcaccctcaggttggggactcaccctcagggttgggagtgactgctgtctctccagcccccaccaattacaccatgttctggttgttaaaatccagtgtatgcatgaaatgtaataaaagtatcttcgcagcaaaaaaaaaaaaaaaaa

SEQ ID NO: 94 - DNA, 551 bp, Homo sapiens
gagaggggtatacacagggaggccaggcagcctggagttagtcgaccgttgcgagacgttgagctgcggcagatgagtccaaagccgagagcctcgggacctccggccaaggccaaggagacaggaaagaggaagtcctcctctcagccgagccccagtggcccgaagaagaagactaccaaggtggccgagaagggagaagcagttcgtggagggagacgcgggaagaaaggggctgcgacaaagatggcggccgtgacggcacctgaggcggagagcgggccagcggcacccggccccagcgaccagcccagccaggagctccctcagcacgagctgccgccggaggagccagtgagcgaggggacccagcacgaccccctgagtcaggagagcgagctggaggaaccactgagtaaggggcgcccatctactcccctatctccctgagcagcaactaagtttaggcccagctgccagacctcagagatctcaccagcagggtgcttcccatgttgatgacaataaaatgaatgtgttgcaaaccgaaa

SEQ ID NO: 95 - DNA, 4399 bp, Homo sapiens
caacctttagacctagggcttactataactccagtatccacaaaggaggctgagcattcgacaaccctgagaaaaactgcagttcctccaaaacaccctgaagtgactcttgcaactccagaccatgtgcaggctcagcacacaaacctaactgaggtcacagtttaaactttggatctgaaacttaccacaattccacaacctactacagagaatatatttcctccaaccatggagaactcaaatcaacttccagaaccacctacggaggttgtagctcaacttccacctcgttatgaggtgacaattccaacacaaggtcaggatcaagctcagctttcaacactggccagtgtcacacttcaacctttggacctggggtttatcatcactccagaatccactacagaaattgaactttctccaaccatgcaggagaccccaactcagcctcctaaggaatttgtaccccaacctccagtatatcaagaggtgagtgttccaacaccgggtcaggatcaagctcagcatccaatgtcacctagcgttacagttcaacctctggacctggtggacttaccataactccagaacccactacagaggttgaacattctacacccctgaaaaagactacagttcctccaaagcaccctgagatgacacttccacatccacaccaggttcagactctacattcaaacctgattcaagtcacagttcaacctttgggtctgaaacttaccttaactctatggaggttgaatcctctatggaggttgaaccttctccaaccatgcagaagaccccaactcggcctccagagctacctaaggagtttgtagctcaaccgcctgtgtattattatcagatacccattccaacaccaagccaagatcaagctctgcccttctacagccccgatgactacagctcctcctccaaagcatcctgaagtgacacatccacctccagacaagaaccaggctcagcatccaaacctgactcaattcacagttcaatctttggacctggagcttaccataactacagaacctactacagaggttaaaacttctccaaccatggaggagacctcaactcagccttcagacctgggatttgccatagttccagaactcaccatagagactgaacattctacaggcctggacaagactacagctccacatccagaccaagttcagactcagcattgaaacctgactgaagtcacacatttcaccttctgaactagaacctactcagaattcactggtgcagtctgaaagttatgcccaaaataaggctttaactgcacaggaggaaccgaaggcctctacacgcaccaacatatgtgatctctatacctgcagagatgaaacactctcatgtattgatctcagcccaaagcagaggctccaccaagtgcctgtaccagagcccagcacctgcaatgacaccttcaccatcctgtgagaattgtctttcctcaattgttctgtgtcctgcctgacatgacagccttttcgtggaggccttcctgggcctcctttatctcaccaaaccgaactgacagcggactttctgctttcacctttcttgtcaattcttccttctcctggttctcctttactgttaggccccttctctggtcttttacttgtgattgctcttaacccttttcttatccactttcctttagccccatcacatcattgcttaacagcggctctcctcccattttcacttcaccctctttacagcagcctgtccctcttcccatctcagtgatgatgctctaagtggttaagagttgattctgtagccaggctgcctgggtttgaacccaggtctgtcatttattagcttggttaccctgagcaagttattcttctctgtgactcagtttcctcatctttaaactggggattatgctagttaccacgccataggattgttgtgagatttaagtgagtgcatacatgtattgcttacattggtgcctagcatatgtgggagtgttggctgctaacatgattactcagtcctttagttatgtccagaacgcatctttgtccctggctttctatctgtagcagtcgttttctgtcaacccttggccaagtatgatactgtcttcagaaatgaaaatgataggagggaagaaagagactaggcatgaaaaggaggtatatataatgaaatactacaagataatgcagaccatcggtgctaggattcaccagaatctgtgatccttgaggtgtggagatcagggaaagctacatcaataagctaaaacttacttgggacttaaagtgtagctataatttgttaaatagaaaacaaatgggagtacagtctaggcaaagtcatgattacaggtatggttgaaatttggtagacaaggctgcagctcagcctccagagaaccccagggaggtggactcttcctcaacccaattagagggcccagctcagacaccagagtgcactgaggagatgaaatattttgcccccagcaggggaccccagctgagcctccaggtcctcctgtggaggctgaaccttcccccagtcagcaggagcagccagctcagccttctgagttttctggggaggtggaattttctcagacccaggagacccccaactctgcctccagagtcttctatagagagtgtagctcaaactccactgaatcatgaagtgacagttcaaactcagggtgaggatcaagctcattataccttgccgagcattacagttaaacctgcagatgtagagattagcataacttcagagcctaccacggacactgactcttctccagcccagcaggcggccccaaaccagcatccagagcaggtgtaaccttctgcaacccaacaggaggccacaactgagcctccaggtcctcatgtgaatgctgaacattccccagtgagcaggagcagccaggtctgccttctgggttttctggagaagttgagtcctctctagcctgcaggagaccccagcccagcctccagaacatcatcaagtaacagttccacctcctggtcaccatcaagttcaatactgagatttgcccaatgtcactgttaagcctccaaatatgcagctcaccatagcaacacagcctactgcagaggtgggaactttgccagtccatcaggaggctacagctcagctctcagggccagttaatgatgtggaacattctgacatccagcatggggccccgcctctgcctacagagtcatcggaagagactggacctttaccagttcaacaggagacttcagttgaatctccagaacctactaaagatgagaacccctctccaatacagtaggaggctgcaggtgagcatccacagacccctgagtaggtcgagtcttctccaacccagcaagatgccccagctcagccttcagagctccctaatgaagttgtagctcaacctccagagcatcacagagtaatagtttctcctataagtcatgaggaagttcagcctccaacatttcaccatgtcattgttaagcctgtggatcacatggttaccatgactccagagttcacctatcaggtggaagttttaactcaacacagggccccagctcagcctttaatatcccctgagcagtttaaacatttgaaagaccagcaaaagattatcattcagcagctaaatacccctggaaatgatgaacttccgccaaatctatcaagagcccatgactccatctccaactcagctctcctcagacatttcatgcttatccaacgagtgtataaaaggcccaagaagacaaggttcagagagcttccggatagctgaacgcatggaggctgacaggacagtgaaggagaactcatccacgcgctgggcgagtggtgcaccccaactccacaggaacggaagctcctccagatcttgccttgtgttatctttccatctggctatttatttgcatcctttttaaatgtaagtaagtgcttccataagttccgtgagctcctccagcaaattaatcaaccccgaagagggtgggtcatggtaaccccaacttgaagccagctggtcagacattctggaagcccagactcgtgactggtgggaaggagggagcagttctgtggaactgattcctcaacctgtggtttctgaggctatttccaggtagatggtgtcacagttgaattaactggtggacacccggctgtgtccactgcagaactaattgcttacttggtgtgtgggaagaaacccctacatattttgtcacagaagtcttctgtattattatggtgtaagagaacaggaaaaatgcatgttgactgttttttccacactcccagtccacaaaagttttctccacttatgaac

SEQ ID NO: 96 - DNA, 1660 bp, Homo sapiens; identical to SEQ ID NO: 22

SEQ ID NO: 97 - DNA, 1953 bp, Homo sapiens
aggtgctcctgggtccgcgcgggtggcgggtgccgcgcacttatccgttggccagctgcgttccgggatcagctaccagacggtccctgagatgaccgggaaccgggctgggggaggatagagccgagactggaggatcgattggcacctccgcccacttttcccacaacgccttcccgaacgataggttggaaaggctcctggatccaaactcgctgagggccaggcaagaaaatgtcctcaaatttattgccaacactgaattctggaggtaaagtaaaagatggctcaaccaaagaggacaggccttataagatctttttcagagatctctttcttgtcaaagaaaatgaaatggcagcaaaggaaacggaaaaatttatgaaccgtaacatgaaagtctaccagaaaactactttttcatccagaatgaagagtcattcatacctgagccaactagctttctaccctaaaaggagtggtaggtcatttgaaaagtttgggccaggtcctgctccgattcctagattaatagaaggttccgacacaaaaaggactgtccatgaatttattaatgaccagagagacaggtttctgctcgagtatgctttgtcaaccaaaagaaacacaatcaaaaagtttgaaaaagacatagcaatgagggaacggcaactaaaaaaagcagagaaaaagctccaagatgatgcactggcctttgaagagttccttcgagaaaatgaccagagatctgtagacgctctgaaaatggcagcacaggaaacaataaacaaactccaaatgacagcagagctgaagaaagcaagcatggaggtacaagcagtgaaaagtgaaatagcaaaaacagaattcctcctcagggagtatatgaaatatggtttttttctgctgcaaatgtctccaaaacattggcaaatccagcaagcactaaaaagagcacaggcatcaaaaagtaaagcaaatatcatccttccaaaaatattagcaaaattatcattacattcaagtaacaaggaaggcatccttgaggagtccgggaggacagctgtcctttcagaagatgcttctcagggaagagacagccaaggaaagccaagcagaagcctgactcgcactccagagaaaaagaaatcaaacctggctgaaagtttcggttcagaagacagtttggaattccttttagatgatgaaatggacgttgatttggagccagcactttatttcaaggaaccagaggagttacttcaagtcctcagagagctggaagagcagaatcttactttgtttcaatattcccaagatgtagatgaaaatcttgaagaggtaaacaaaagagaaaaagttatacaggataaaacaaatagcaacatagagtttcttttggagcaagaaaaaatgcttaaagctaactgtgtgagagaagaagagaaagcagcagaattgcaattaaagtccaagctctttagctttggagaatttaattcagatgctcaggaaatactgatagactcacttagtaaaaagattactcaagtatacaaagtctgcattggagatgctgaggatgacggcctcaacccaattcaaaagctggtaaaagtagaatctcgcctggtagaactgtgtgacctcatcgaatccattcccaaagaaaatgtggaggcaattgagaggatgaaacagaaagaatggcggcaaaagtttcgtgatgagaaaatgaaagaaaaacaaagacaccaacaggaaaggctaaaagctgctctggaaaaagcagtagcacaaccaaagaaaaagttgggaagacaacttgtctttcattcaaaacctccatctggtaacaaacagcagctacctttagtcaatgaaacaaaaacaaaatcacaagaggaagaatatttttttacttgaataaaagcagtaagacattttattacc

SEQ ID NO: 98 - protein, 407 aa, Homo sapiens; identical to SEQ ID NO: 30

SEQ ID NO: 99 - protein, 533 aa, Homo sapiens
MNESPDPTDLAGVIIELGPNDSPQTSEFKGATEEAPAKESVLARLSKFEVEDAENVASYDSKIKKIVHSIVSSFAFGLFGVFLVLLDVTLILADLIFTDSKLYIPLEYRSISLAIALFFLMDVLLRVFVERRQQYFSDLFNILDTAIIVILLLVDVVYIFFDIKLLRNIPRWTHLLRLLRLIILLRIFHLFHQKRQLEKLIRRRVSENKRRYTRDGFDLDLTYVTERIIAMSFPSSGRQSFYRNPIKEVVRFLDKKHRNHYRVYNLCSERAYDPKHFHNRVVRIMIDDHNVPTLHQMVVFTKEVNEWMAQDLENIVAIHCKGGTDRTGTMVCAFLIASEICSTAKESLYYFGERRTDKTHSEKFQGVETPSQKRYVAYFAQVKHLYNWNLPPRRILFIKHFIIYSIPRYVRDLKIQIEMEKKVVFSTISLGKCSVLDNITTDKILIDVFDGLPLYDDVKVQFFYSNLPTYYDNCSFYFWLHTSFIENNRLYLPKNELDNLHKQKARRIYPSDFAVEILFGEKMTSSDVVAGSD

SEQ ID NO: 100 - protein, 533 aa, Homo sapiens; identical to SEQ ID NO: 29

SEQ ID NO: 101 - protein, 136 aa, Homo sapiens
MARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 102 - protein, 436 aa, Homo sapiens
MQGTPGGGTRPGPSPVDRRTLLVFSFILAAALGQMNFTGDQVLRVLAKDEKQLSLLGDLEGLKPQKVDFWRGPARPSLPVDMRVPFSELKDIKAYLESHGLAYSIMIKDIQVLLDEERQAMAKSRRLERSTNSFSYSSYHTLEEIYSWIDNFVMEHSDIVSKIQIGNSFENQSILVLKFSTGGSRHPAIWIDTGIHSREWITHATGIWTANKIVSDYGKDRVLTDILNAMDIFIELVTNPDGFAFTHSMNRLWRKNKSIRPGIFCIGVDLNRNWKSGFGGNGSNSNPCSETYHGPSPQSEPEVAAIVNFITAHGNFKALISIHSYSQMLMYPYGRSLDPVSNQRELYDLAKDAVEALYKVHGIEYIFGSISTTLYVASGITVDWAYDSGIKYAFSFELRDTGQYGFLLPATQIIPTAQETWMALRTIMEHTLNHPY

SEQ ID NO: 103 - protein, 735 aa, Homo sapiens; identical to SEQ ID NO: 43

SEQ ID NO: 104 - protein, 450 aa, Homo sapiens; identical to SEQ ID NO: 46

SEQ ID NO: 105 - protein, 409 aa, Homo sapiens
MSLHAWEWEEDPASIEPISSITSFYQSTSECDVEEHLKAKARAQESDSDRPCSSIESSSEPASTFSSDVPHVVPCKFTISLAFPVNMGQKGKYASLIEKYKKHPKTDSSVTKMRRFYHIEYFLLPDDEEPKKVDILLFPMVAKVFLESGVKTVKPWHEGDKAWVSWEQTFNITVTKELLKKINFHKITLRLWNTKDKMSRKVRYYRLKTAGFTDDVGAFHKSEVRHLVLNQRKLSEQGIENTNIVREESNQEHPPGKQEKTEKHPKSLQGSHQAEPETSSKNSEEYEKSLKMDDSSTIQWSVSRTPTISLAGASMMEIKELIESESLSSLTNILDRQRSQIKGKDSEGRRKIQRRHKKPLAEEEADPTLTGPRKQSAFSIQLAVMPLLAGTHCLPCSQQLLLVLWPERP

SEQ ID NO: 106 - protein, 110 aa, Homo sapiens
  misc_feature (110)..(110): Xaa can be any naturally occurring amino acid
EHIHTQKTNPHIFTYLYSLQDASIYLTLPNHPYYTKMKTLVFHFSDEQNEVQKIKTKVKPSKKTKIKIKLTNKKLVQVTQQVAAVLKSKRAVSHLFSQPFALLKNLKKKX

SEQ ID NO: 107 - protein, 626 aa, Homo sapiens
MMANDAKPDVKTVQVLRDTANRLRIHSIRATCASGSGQLTSCCSAAEVVSVLFFHTMKYKQTDPEHPDNDRFILSRGHAAPILYAAWVEVGDISESDLLNLRKLHSDLERHPTPRLPFVDVATGSLGQGLGTACGMAYTGKYLDKASYRVFCLMGDGESSEGSVWEAFAFASHYNLDNLVAVFDVNRLGQSGPAPLEHGADIYQNCCEAFGWNTYLVDGHDVEALCQAFWQASQVKNKPTAIVAKTFKGRGIPNIEDAENWHGKPVPKERADAIVKLIESQIQTNENLIPKSPVEDSPQISITDIKMTSPPAYKVGDKIATQKTYGLALAKLGRANERVIVLSGDTMNSTFSEIFRKEHPERFIECIIAEQNMVSVALGCATRGRTIAFAGAFAAFFTRAFDQLRMGAISQANINLIGSHCGVSTGEDGVSQMALEDLAMFRSIPNCTVFYPSDAISTEHAIYLAANTKGMCFIRTSQPETAVIYTPQENFEIGQAKVVRHGVNDKVTVIGAGVTLHEALEAADHLSQQGISVRVIDPFTIKPLDAATIISSAKATGGRVITVEDHYREGGIGEAVCAAVSREPDILVHQLAVSGVPQRGKTSELLDMFGISTRHIIAAVTLTLMK

SEQ ID NO: 108 - protein, 444 aa, Homo sapiens
MENSGKANKKDTHDGPPKEIKLPTSEALLDYQCQIKEDAVEQFMFQIKTLRKKNQKYHERNSRLKEEQIWHIRHLLKELSEEKAEGLPVVTREDVEEAMKEKWKFERDQEKNLRDMRMQISNAEKLFLEKLSEKEYWEEYKNVGSERHAKLITSLQNDINTVKENAEKMSEHYKITLEDTRKKIIKETLLQLDQKKEWATQNAVKLIDKGSYLEIWENDWLKKEVAIHRKEVEELKNAIHELEAENLVLIDQLSNCRLVDLKIPRYPVLHSCPTSNPRHLLLLPLESCLISARRCWRLYLTQAAGLEVPPEEMSLELPETHIEEKSELQPTEVESRDLMSSSDESTILHLSHENSIEDLQYVKIDKEENSGTEFGDTDMKYLLYEDEKDFKDYVNLGPLGVKLMSVESKKMPIHFQEKEIPVKLYKDVRSPESHITYKMMKSFL

SEQ ID NO: 109 - protein, 513 aa, Homo sapiens; identical to SEQ ID NO: 34

SEQ ID NO: 110 - protein, 154 aa, Homo sapiens; identical to SEQ ID NO: 35

SEQ ID NO: 111 - protein, 861 aa, Homo sapiens; identical to SEQ ID NO: 39

SEQ ID NO: 112 - protein, 212 aa, Homo sapiens
MAQTDKPTCIPPELPKMLKEFAKAAIRVQPQDLIQWAADYFEALSRGETPPVRERSERVALCNRAELTPELLKILHSQVAGRLIIRAEELAQMWKVVNLPTDLFNSVMNVGRFTEEIEWLKFLALACSALGVTITKTLKIVCEVLSCDHNGGSPRIPFSTFQFLYTYIAKVDGEISASHVSRMLNYMEQEVIGPDGIITVNDFTQNPRVQLE

SEQ ID NO: 113 - protein, 123 aa, Homo sapiens
MVVSADPLSSERAEMNILEINQELRSQLAESNQQFRDLKEKFLITQATAYSLANQLKKYKCEEYRNHLPPERCRRLKKRKSLRTHWRNVLSLVQIVTTLLTPTSLTGAPKSHLRNTKSTLLWL

SEQ ID NO: 114 - protein, 86 aa, Homo sapiens
ALLLPCSLISDCCASNQRDSVGVGPSKPGERAYDPKHFHNRVSRIMIDDHNVPTLREMVAFSKEVLEWMAQDSENIVVIHCKGGKE

SEQ ID NO: 115 - protein, 223 aa, Homo sapiens
MRDEIATTVFFVTRLVKKHDKLSKQQIEDFAEKLMTILFETYRSHWHSDCPSKGQAFRCIRINNNQNKDPILERACVESNVDFSHLGLPKEMTIWVDPFEVCCRYGEKNHPFTVASFKGRWEEWELYQQISYAVSRASSDVSSGTSCDEESCSKEPRVIPKVSNPKSIYQVENLKQPFQSWLQIPRKKNVVDGRVGLLGNTYHGSQKHPKCYRPAMHRLDRIL

SEQ ID NO: 116 - protein, 571 aa, Homo sapiens
MRALRDRAGLLLCVLLLAALLEAALGLPVKKPRLRGPRPGSLTRLAEVSASPDPRPLKEEEEAPLLPRTHLQAEPHQHGCWTVTEPAAMTPGNATPPRTPEVTPLRLELQKLPGLANTTLSTPNPDTQASASPDPRPLREEEEARLLPRTHLQAELHQHGCWTVTEPAALTPGNATPPRTQEVTPLLLELQKLPELVHATLSTPNPDNQVTIKVVEDPQAEVSIDLLAEPSNPPPQDTLSWLPALWSFLWGDYKGEEKDRAPGEKGEEKEEDEDYPSEDIEGEDQEDKEEDEEEQALWFNGTTDNWDQGWLAPGDWVFKDSVSYDYEPQKEWSPWSPCSGNCSTGKQQRTRPCGYGCTATETRTCDLPSCPGTEDKDTLGLPSEEWKLLARNATDMHDQDVDSCEKWLNCKSDFLIKYLSQMLRDLPSCPCAYPLEAMDSPVSLQDEHQGRSFRWRDASGPRERLDIYQPTARFCLRSMLSGESSTLAAQHCCYDEDSRLLTRGKGAGMPNLISTDFSPKLHFKFDTTPWILCKGDWSRLHAVLPPNNGRACTDNPLEEEYLAQLQEAKEY

SEQ ID NO: 117 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 118 - protein, 168 aa, Homo sapiens; identical to SEQ ID NO: 44

SEQ ID NO: 119 - protein, 125 aa, Homo sapiens
MSPKPRASGPPAKAKETGKRKSSSQPSPSGPKKKTTKVAEKGEAVRGGRRGKKGAATKMAAVTAPEAESGPAAPGPSDQPSQELPQHELPPEEPVSEGTQHDPLSQESELEEPLSKGRPSTPLSP

SEQ ID NO: 120 - protein, 123 aa, Homo sapiens
MKYFAPSRGPQLSLQVLLWRLNLPPVSRSSQLSLLSFLGRWNFLRPRRPPTLPPESSIESVAQTPLNHEVTVQTQGEDQAHYTLPSITVKPADVEISITSEPTTDTDSSPAQQAAPNQHPEQV

SEQ ID NO: 121 - protein, 259 aa, Homo sapiens; identical to SEQ ID NO: 45

SEQ ID NO: 122 - protein, 563 aa, Homo sapiens
MSSNLLPTLNSGGKVKDGSTKEDRPYKIFFRDLFLVKENEMAAKETEKFMNRNMKVYQKTTFSSRMKSHSYLSQLAFYPKRSGRSFEKFGPGPAPIPRLIEGSDTKRTVHEFINDQRDRFLLEYALSTKRNTIKKFEKDIAMRERQLKKAEKKLQDDALAFEEFLRENDQRSVDALKMAAQETINKLQMTAELKKASMEVQAVKSEIAKTEFLLREYMKYGFFLLQMSPKHWQIQQALKRAQASKSKANIILPKILAKLSLHSSNKEGILEESGRTAVLSEDASQGRDSQGKPSRSLTRTPEKKKSNLAESFGSEDSLEFLLDDEMDVDLEPALYFKEPEELLQVLRELEEQNLTLFQYSQDVDENLEEVNKREKVIQDKTNSNIEFLLEQEKMLKANCVREEEKAAELQLKSKLFSFGEFNSDAQEILIDSLSKKITQVYKVCIGDAEDDGLNPIQKLVKVESRLVELCDLIESIPKENVEAIERMKQKEWRQKFRDEKMKEKQRHQQERLKAALEKAVAQPKKKLGRQLVFHSKPPSGNKQQLPLVNETKTKSQEEEYFFT

(19) United States
(12) Patent Application Publication
(10) Pub. No.: US 2013/0267438 A1
(43) Pub. Date: Oct. 10, 2013

(54) USE OF SPECIFIC GENES FOR THE PROGNOSIS OF LUNG CANCER AND THE CORRESPONDING PROGNOSIS METHOD

(71) Applicants: Sophie Pison-Rousseaux, Saint Martin D'uriage (FR); Saadi Khochbin, Meylan (FR)

(72) Inventors: Sophie Pison-Rousseaux, Saint Martin D'uriage (FR); Saadi Khochbin, Meylan (FR)

(73) Assignee: Universite Joseph Fourier, Grenoble Cedex 09 (FR)

(21) Appl. No.: 13/880,423

(22) PCT Filed: Oct. 20, 2011
(86) PCT No.: PCT/EP2011/068375

§ 371 Date: Jun. 26, 2013

(30) Foreign Application Priority Data
Oct. 20, 2010 (EP) 10306142.0

(51) Int. Cl. C12Q 1/68; G01N 33/68

(52) U.S. Cl. CPC C12Q 1/6886; G01N 33/6884
USPC 506/9; 506/16; 506/18

(57) ABSTRACT

At least 13 genes chosen among a set of 28 genes for carrying out a method for identifying at least 66% of patients of those having a survival rate of at most about 20% at 30 months, among a population of patients afflicted by lung cancer having an estimated survival rate of at least 30% at 30 months based on the diagnosis of the lung cancer according to histopathological criteria.

The present invention relates to the use of specific genes for the prognosis of lung cancer and the corresponding prognosis method.

Lung cancer is a disease of uncontrolled cell growth in tissues of the lung. This growth may lead to metastasis, which is the invasion of adjacent tissues and infiltration beyond the lungs. The vast majority of primary lung cancers are carcinomas of the lung, derived from epithelial cells. Lung cancer, the most common cause of cancer-related death in men and women, is responsible for 1.3 million deaths worldwide annually, as of 2004. The most common symptoms are shortness of breath, coughing (including coughing up blood), and weight loss.

Due to the high prevalence of this type of tumors, there is a need to efficiently diagnose lung cancer. Moreover, it is important to propose a prognosis method that allows the pathologist to determine, when a patient is afflicted by lung tumors, the survival rate during a short and a long period, and consequently to propose an adapted therapy.

Presently, several clinical and pathological parameters help defining the prognosis, including histological subtypes, TNM stages (tumour size, presence of tumour cells in lymph nodes, presence of distant metastasis).

Classically, prognosis and diagnosis methods intend to detect the variation of expression of genes between the sample from a patient and a healthy control sample. However, with these methods, false positive results are frequent and indistinguishable from real positive samples.

Cancer Testis (CT) genes are genes that are expressed in testis cells, but not expressed in somatic non pathologic cells. In cancers, CT genes are deregulated and are expressed ectopically in somatic cells. They appear as good candidate for cancer diagnosis.

Some works have intended to identify a “general” strategy for diagnosing lung cancer, by detecting cancer testis gene expression.

For instance, the international application WO 2009/121878 discloses the use of a minimal group of CT genes for identifying any somatic or ovarian cancer. However, even if specific genes, or combinations of genes, can be used for diagnosing cancer, there is no indication that these genes can be used to establish a reliable prognosis during a short or a long period.

Recently, Gure et al. (Gure at al. Clin Cancer research, 2005, 11(22) p:8055-8061) have proposed that cancer testis genes are coordinately expressed in non-small cell lung cancers, and are markers of poor outcome. The study suggests that X-linked CT genes can be associated with worse prognosis, either by their expression, or by their increased expression.

Therefore, there is a need to provide prognosis marker that can give specific evolution of tumoral progression, and patient survival, for instance by using CT genes.

One aim of the invention is to a simple, rapid, easy-to-use and effective method for giving a prognosis of lung cancer.

Another aim of the invention is to provide a general prognosis method of lung tumor.

Another aim of the invention is to provide a kit for diagnosing lung cancer.

The present invention relates to the use of
at least 2 genes chosen among a set of 23 genes comprising or consisting of the nucleic acid sequences SEQ ID NO 1 to 23,
or fragments of said genes,
or complementary sequences of said genes,
or sequences having at least 80% homology with said genes or fragment thereof,
or protein coded by said genes,
or fragments of said proteins,
or antibodies directed against said proteins,
said at least 2 genes being such that
   at least one gene belongs to a first set A of 7 genes comprising or consisting of the nucleic acid sequences SEQ ID NO: 1-7
   at least one gene belongs to a second set B of 16 genes comprising or consisting of the nucleic acid sequences SEQ ID NO: 8-23
      for the implementation of a prognosis method, preferably in vitro, of the survival rate of a patient afflicted by a lung cancer, said prognosis being such that:
if none of the 23 genes of said set is expressed, the patient survival rate during a period of time from 30 to 120 months after the diagnosis of said lung cancer is from about 59% to about 78% or more, and
if at least one gene of at least one set A or B is expressed, the patient survival rate during a period of time from 30 to 120 months after the diagnosis of said lung cancer is about from about 3% to about 70%.

The present invention is based on the unexpected observation made by the Inventors that the expression of at least two genes of a group of 23 determined genes is sufficient to determine the survival rate of a patient afflicted by a lung cancer.

In other words, and as explained and exemplified hereafter, according to the invention the determination of the gene expression status on a ON/OFF basis of at least 2 genes chosen among a set of 23 genes allows to estimate at 30 months, or 60 months or 120 months after the diagnosis of a lung cancer, the probability of survival of an individual.

The diagnosis method proposed by the Inventors can also be carried out by detecting the expression of proteins expressed by at least two genes above mentioned, chosen among proteins coded by said 23 genes, also on a absence/presence basis.

Another aspect of the invention is that the diagnosis can also be carried out by determining the presence, of at least 2 specific antibodies specifically recognising at least 2 proteins coded by said at least 2 genes mentioned above. The above antibodies are specific of one protein, each protein being coded by one gene of the set of 23 genes.

A key aspect of the invention is the concept of ON/OFF for gene expression. The concept of ON/OFF expression can be extended to presence/absence of the proteins and antibodies detecting these proteins. This specific approach has the advantage of simplifying the analyses and making them independent of complex statistical tests to measure variations in expression levels applied to the majority of the existing tests.

The ON/OFF status of gene expression is established by determination of a threshold of gene expression allowing them to decide on the ON/OFF status of a gene such that:
if a gene is expressed at a level lower than the threshold, the gene is considered as not expressed or weakly expressed (defined as OFF), and
if a gene is expressed at a level upper to the threshold, the gene is considered as being expressed (defined as ON).

According to the invention, the prognosis is carried out as described hereafter:

The Inventors have identified 23 genes comprising or being constituted by the nucleic acid sequences SEQ ID NO 1 to 23, as being cancer testis genes (CT genes) that can be used to carry out the prognosis method according to the invention.

The above 23 genes have been identified as being liable to be “expressed” (form here by, “expressed” refers to the ON status and “not expressed” to the OFF status) in lung cancer cells, but not in healthy samples. In other words, the above 23 genes are such as
they are not expressed, or weakly expressed in healthy lung cells, and
they maybe expressed in lung tumor cells.

The difference between the absence of expression, or weak expression, and the expression determines its ON/OFF status, which is a key step of the invention.

Indeed, the Inventors have identified that the ON status of the above 23 genes is a key step to determine the prognosis of lung cancer.

On microarrays, the expression level of the above mentioned genes is determined by the fact that a threshold of expression has been identified by the Inventors allowing to determine expression (ON) and non-expression (OFF) of said genes. The threshold determination is detailed hereafter, in the Example section.

For the microarrays, the threshold enabling to determine the expression status of a gene (ON versus OFF) is calculated by using the signal mean value and distribution obtained from transcriptomic data (in the same technology) with the corresponding probes in a large number of somatic tissues (which do not express the genes).

A similar strategy enables determining a threshold for the presence/absence of the encoded proteins or antibodies. For each protein or antibody, the mean value and distribution of the signal intensities obtained in an appropriate number of control somatic tissues serves as a basis for calculating the threshold.

By “not expressed” it is defined in the invention the fact that the transcription of a gene is either not carried out, or is not detectable by common techniques known in the art, such as Quantitative RT-PCR, Northern blot or when microarrays data are considered.

By “weakly expressed”, it is defined in the invention that a gene is expressed at a low level, meaning that the values are within the range of those measured for healthy tissue samples by Q-RT-PCR and by Northern blots or below the threshold when microarray data are considered. These values are considered as false-positive expressions, due to probe cross hybridization for instance. All the expression falling in these categories are considered as “OFF”

By “expressed”, the invention defined that the transcript of a gene is detectable by the above known techniques while it is not detectable in healthy tissues or determined as being above the threshold when microarrays data are considered.

The 23 genes according to the invention have been classified by the Inventors in two sets:
a first set of 7 genes,
and a second set of 16 genes.

The first set, also called in the invention set A, of 7 genes consists of the genes comprising or constituted by the nucleic acid sequences SEQ ID NO: 1, SEQ ID NO: 2, SEQ ID NO: 3, SEQ ID NO: 4, SEQ ID NO: 5, SEQ ID NO: 6 and SEQ ID NO: 7.

The second set, also called in the invention set B, of 16 genes consists of the genes comprising or constituted by the nucleic acid sequences SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23.

The inventors have also defined subsets of each of the above sets A and B as follows:

Set A is divided into 4 subsets A1, A5, A6 and A7, said subset being such that:
subset A1 consists of the genes comprising or being constituted by the nucleic acid sequences SEQ ID NO: 1 and SEQ ID NO: 2,
subset A5 consists of the gene comprising or being constituted by the nucleic acid sequence SEQ ID NO: 3,
subset A6 consists of the genes comprising or being constituted by the nucleic acid sequence SEQ ID NO: 4, and SEQ ID NO: 5, and
subset A7 consists of the genes comprising or being constituted by the nucleic acid sequences SEQ ID NO: 6 and SEQ ID NO: 7
   Set A can also be divided into the following subsets:
subset A1 consists of the genes comprising or being constituted by the nucleic acid sequences SEQ ID NO: 1 and SEQ ID NO: 2,
subset A2 consists of the genes comprising or being constituted by the nucleic acid sequences SEQ ID NO: 1, SEQ ID NO: 2 and SEQ ID NO: 3,
subset A3 consists of the genes comprising or being constituted by the nucleic acid sequences SEQ ID NO: 1, SEQ ID NO: 2, SEQ ID NO: 3, SEQ ID NO: 4 and SEQ ID NO: 5, and
subset A4 consists of the genes comprising or being constituted by the nucleic acid sequences SEQ ID NO: 1, SEQ ID NO: 2, SEQ ID NO: 3, SEQ ID NO: 4, SEQ ID NO: 5, SEQ ID NO: 6 and SEQ ID NO: 7.
   Set B is divided into 3 subsets B1, B4 and B5, said subset being such that:
subset B1 consists of the genes comprising or being constituted by the nucleic acid sequences SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15,
subset B4 consists of the genes comprising or being constituted by the nucleic acid sequences SEQ ID NO: 16, SEQ ID NO: 17, SEQ ID NO: 18, SEQ ID NO: 19, SEQ ID NO: 20 and SEQ ID NO: 21, and
subset B5 consists of the genes comprising or being constituted by the nucleic acid sequences SEQ ID NO: 22, and SEQ ID NO: 23.
   Set B can also be divided into the following subsets:
subset B1 consists of the genes comprising or being constituted by the nucleic acid sequences SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15,
subset B2 consists of the genes comprising or being constituted by the nucleic acid sequences SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21, and
subset B3 consists of the genes comprising or being constituted by the nucleic acid sequences SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 and SEQ ID NO: 21, SEQ ID NO: 22, and SEQ ID NO: 23.

Thus, the prognosis method according to the invention is such that, when determining the expression status of 2 genes on a ON/OFF basis, belonging to the set of 23 genes comprising or consisting of nucleic acid sequences SEQ ID NO: 1 to 23,
if none of said at least 2 genes are expressed, the patient survival rate at 30 months, 60 months or 120 months following the diagnosis is from about 59% to about 78% or more, and
if at least one of said at least two genes is expressed, according to the defined expression threshold, the patient survival rate at 30 months, 60 months or 120 months following the diagnosis is from about 3% to about 70%.
   According to the invention, the above mentioned at least 2 genes are such that:
at least one of said at least two genes belongs to a first set A of 7 genes comprising or consisting of nucleic acid sequences SEQ ID NO: 1 to 7, and
at least one of said at least two genes belongs to a first set B of 16 genes comprising or consisting of nucleic acid sequences SEQ ID NO: 8 to 23.

Therefore, the prognosis method according to the invention can be carried out as by measuring at least the expression of the following 112 couples of genes: SEQ ID NO: 1+SEQ ID NO: 8, SEQ ID NO: 1+SEQ ID NO: 9, SEQ ID NO: 1+SEQ ID NO: 10, SEQ ID NO: 1+SEQ ID NO: 11, SEQ ID NO: 1+SEQ ID NO: 12, SEQ ID NO: 1+SEQ ID NO: 13, SEQ ID NO: 1+SEQ ID NO: 14, SEQ ID NO: 1+SEQ ID NO: 15, SEQ ID NO: 1+SEQ ID NO: 16, SEQ ID NO: 1+SEQ ID NO: 17, SEQ ID NO: 1+SEQ ID NO: 18, SEQ ID NO: 1+SEQ ID NO: 19, SEQ ID NO: 1+SEQ ID NO: 20, SEQ ID NO: 1+SEQ ID NO: 21, SEQ ID NO: 1+SEQ ID NO: 22, SEQ ID NO: 1+SEQ ID NO: 23, SEQ ID NO: 2+SEQ ID NO: 8, SEQ ID NO: 2+SEQ ID NO: 9, SEQ ID NO: 2+SEQ ID NO: 10, SEQ ID NO: 2+SEQ ID NO: 11, SEQ ID NO: 2+SEQ ID NO: 12, SEQ ID NO: 2+SEQ ID NO: 13, SEQ ID NO: 2+SEQ ID NO: 14, SEQ ID NO: 2+SEQ ID NO: 15, SEQ ID NO: 2+SEQ ID NO: 16, SEQ ID NO: 2+SEQ ID NO: 17, SEQ ID NO: 2+SEQ ID NO: 18, SEQ ID NO: 2+SEQ ID NO: 19, SEQ ID NO: 2+SEQ ID NO: 20, SEQ ID NO: 2+SEQ ID NO: 21, SEQ ID NO: 2+SEQ ID NO: 22, SEQ ID NO: 2+SEQ ID NO: 23, SEQ ID NO: 3+SEQ ID NO: 8, SEQ ID NO: 3+SEQ ID NO: 9, SEQ ID NO: 3+SEQ ID NO: 10, SEQ ID NO: 3+SEQ ID NO: 11, SEQ ID NO: 3+SEQ ID NO: 12, SEQ ID NO: 3+SEQ ID NO: 13, SEQ ID NO: 3+SEQ ID NO: 14, SEQ ID NO: 3+SEQ ID NO: 15, SEQ ID NO: 3+SEQ ID NO: 16, SEQ ID NO: 3+SEQ ID NO: 17, SEQ ID NO: 3+SEQ ID NO: 18, SEQ ID NO: 3+SEQ ID NO: 19, SEQ ID NO: 3+SEQ ID NO: 20, SEQ ID NO: 3+SEQ ID NO: 21, SEQ ID NO: 3+SEQ ID NO: 22, SEQ ID NO: 3+SEQ ID NO: 23, SEQ ID NO: 4+SEQ ID NO: 8, SEQ ID NO: 4+SEQ ID NO: 9, SEQ ID NO: 4+SEQ ID NO: 10, SEQ ID NO: 4+SEQ ID NO: 11, SEQ ID NO: 4+SEQ ID NO: 12, SEQ ID NO: 4+SEQ ID NO: 13, SEQ ID NO: 4+SEQ ID NO: 14, SEQ ID NO: 4+SEQ ID NO: 15, SEQ ID NO: 4+SEQ ID NO: 16, SEQ ID NO: 4+SEQ ID NO: 17, SEQ ID NO: 4+SEQ ID NO: 18, SEQ ID NO: 4+SEQ ID NO: 19, SEQ ID NO: 4+SEQ ID NO: 20, SEQ ID NO: 4+SEQ ID NO: 21, SEQ ID NO: 4+SEQ ID NO: 22, SEQ ID NO: 4+SEQ ID NO: 23, SEQ ID NO: 5+SEQ ID NO: 8, SEQ ID NO: 5+SEQ ID NO: 9, SEQ ID NO: 5+SEQ ID NO: 10, SEQ ID NO: 5+SEQ ID NO: 11, SEQ ID NO: 5+SEQ ID NO: 12, SEQ ID NO: 5+SEQ ID NO: 13, SEQ ID NO: 5+SEQ ID NO: 14, SEQ ID NO: 5+SEQ ID NO: 15, SEQ ID NO: 5+SEQ ID NO: 16, SEQ ID NO: 5+SEQ ID NO: 17, SEQ ID NO: 5+SEQ ID NO: 18, SEQ ID NO: 5+SEQ ID NO: 19, SEQ ID NO: 5+SEQ ID NO: 20, SEQ ID NO: 5+SEQ ID NO: 21, SEQ ID NO: 5+SEQ ID NO: 22, SEQ ID NO: 5+SEQ ID NO: 23, SEQ ID NO: 6+SEQ ID NO: 8, SEQ ID NO: 6+SEQ ID NO: 9, SEQ ID NO: 6+SEQ ID NO: 10, SEQ ID NO: 6+SEQ ID NO: 11, SEQ ID NO: 6+SEQ ID NO: 12, SEQ ID NO: 6+SEQ ID NO: 13, SEQ ID NO: 6+SEQ ID NO: 14, SEQ ID NO: 6+SEQ ID NO: 15, SEQ ID NO: 6+SEQ ID NO: 16, SEQ ID NO: 6+SEQ ID NO: 17, SEQ ID NO: 6+SEQ ID NO: 18, SEQ ID NO: 6+SEQ ID NO: 19, SEQ ID NO: 6+SEQ ID NO: 20, SEQ ID NO: 6+SEQ ID NO: 21, SEQ ID NO: 6+SEQ ID NO: 22, SEQ ID NO: 6+SEQ ID NO: 23, SEQ ID NO: 7+SEQ ID NO: 8, SEQ ID NO: 7+SEQ ID NO: 9, SEQ ID NO: 7+SEQ ID NO: 10, SEQ ID NO: 7+SEQ ID NO: 11, SEQ ID NO: 7+SEQ ID NO: 12, SEQ ID NO: 7+SEQ ID NO: 13, SEQ ID NO: 7+SEQ ID NO: 14, SEQ ID NO: 7+SEQ ID NO: 15, SEQ ID NO: 7+SEQ ID NO: 16, SEQ ID NO: 7+SEQ ID NO: 17, SEQ ID NO: 7+SEQ ID NO: 18, SEQ ID NO: 7+SEQ ID NO: 19, SEQ ID NO: 7+SEQ ID NO: 20, SEQ ID NO: 7+SEQ ID NO: 21, SEQ ID NO: 7+SEQ ID NO: 22 and SEQ ID NO: 7+SEQ ID NO: 23.

For instance, the prognosis method according to the invention can be carried out by determining the expression status of the above couple SEQ ID NO: 1+SEQ ID NO: 8, wherein
if neither SEQ ID NO: 1 nor SEQ ID NO: 8 is expressed, the patient survival rate at 30 months, 60 months or 120 months following the diagnosis is from about 59% to about 78% or more, and
if either SEQ ID NO: 1 or SEQ ID NO: 8 or both SEQ ID NO: 1+SEQ ID NO: 8 is(are) expressed, the patient survival rate at 30 months, 60 months or 120 months following the diagnosis is from about 3% to about 70%.

According to the invention, the terms “about X %” means that the percentage of survival proposed for the prognosis method have to be considered with a standard deviation corresponding to individual variability. This standard deviation is about 5%.

By “at least 2 genes/proteins/antibodies chosen among a set of 23 genes/proteins/antibodies”, it is defined in the invention: 2 genes/proteins/antibodies, or 3 genes/proteins/antibodies, or 4 genes/proteins/antibodies, or 5 genes/proteins/antibodies, or 6 genes/proteins/antibodies, or 7 genes/proteins/antibodies, or 8 genes/proteins/antibodies, or 9 genes/proteins/antibodies, or 10 genes/proteins/antibodies, or 11 genes/proteins/antibodies, or 12 genes/proteins/antibodies, or 13 genes/proteins/antibodies, or 14 genes/proteins/antibodies, or 15 genes/proteins/antibodies, or 16 genes/proteins/antibodies, or 17 genes/proteins/antibodies, or 18 genes/proteins/antibodies, or 19 genes/proteins/antibodies, or 20 genes/proteins/antibodies, or 21 genes/proteins/antibodies, or 22 genes/proteins/antibodies, or 23 genes/proteins/antibodies.

To summarise, the invention relates to the use of
at least 2 genes chosen among a set of 23 genes comprising or consisting of the nucleic acid sequences SEQ ID NO 1 to 23,
or fragments of said least 2 genes chosen among a set of 23 genes comprising or consisting of the nucleic acid sequences SEQ ID NO 1 to 23,
or complementary sequences of said least 2 genes chosen among a set of 23 genes comprising or consisting of the nucleic acid sequences SEQ ID NO 1 to 23,
or sequences having at least 80% homology with said genes or fragment thereof,
or proteins coded by said least 2 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
said
at least 2 genes being such that
   at least one gene belongs to a first set A of 7 genes comprising or consisting of the nucleic acid sequences SEQ ID NO: 1-7
   at least one gene belongs to a second set B of 16 genes comprising or consisting of the nucleic acid sequences SEQ ID NO: 8-23,
at least 2 proteins being such that
   at least one protein belongs to a first set AP of 7 proteins comprising or consisting of the amino acid sequences SEQ ID NO: 24-30,
   at least one protein belongs to a second set BP of 16 proteins comprising or consisting of the amino acid sequences SEQ ID NO: 31-46,
at least 2 antibodies directed against said 2 proteins being such that
   at least one antibody specifically recognises one protein that belongs to a first set AP of 7 proteins comprising or consisting of the amino acid sequences SEQ ID NO: 24-30,
   at least one antibody specifically recognises one protein that belongs to a second set BP of 16 proteins comprising or consisting of the amino acid sequences SEQ ID NO: 31-46,
      for the implementation of a prognosis method, preferably in vitro, of the survival rate of a patient afflicted by a lung cancer, said prognosis being such that:
      either
if none of the 23 genes of said set is expressed, the patient survival rate during a period of time from 30 to 120 months after the diagnosis of said lung cancer is from about 59% to about 78% or more, and
if at least one gene of at least one set A or B is expressed, the patient survival rate during a period of time from 30 to 120 months after the diagnosis of said lung cancer is about from about 3% to about 70%,
   or
if none of the 23 proteins of said set is expressed, the patient survival rate during a period of time from 30 to 120 months after the diagnosis of said lung cancer is from about 59% to about 78% or more, and
if at least one protein of at least one set AP or BP is expressed, the patient survival rate during a period of time from 30 to 120 months after the diagnosis of said lung cancer is about from about 3% to about 70%,
   or
if none of the antibodies directed against said 23 proteins of said set is expressed, the patient survival rate during a period of time from 30 to 120 months after the diagnosis of said lung cancer is from about 59% to about 78% or more, and
if at least one antibody directed against one protein of at least one set AP or BP is expressed, the patient survival rate during a period of time from 30 to 120 months after the diagnosis of said lung cancer is about from about 3% to about 70%,
   wherein said gene, protein or antibody is determined as being expressed when:
either it is expressed in a sample of a patient afflicted by a lung cancer but not in a control sample of an healthy individual,
or it is expressed above a threshold corresponding to a background signal observed in a series of reference healthy tissues for each detection method, and
said gene, protein or antibody is determined as being not expressed when:
it is neither expressed in a sample of a patient afflicted by a lung cancer nor in a control sample of an healthy individual,
or it is expressed in a sample of a patient afflicted by a lung cancer at a level substantially equal or inferior to the level in a control sample of an healthy individual.

According to the invention, “a control sample of an healthy individual” corresponds to a somatic tissue in which the CT gene is not expressed or weakly expressed as defined above, said somatic tissue originating from a person not afflicted by cancer.

In the invention “is expressed at a level above a threshold corresponding to a background signal observed in a series of reference healthy tissues for each detection method” means that the threshold, which corresponds to the key step of the invention, is determined by measuring the background signal in negative control samples of healthy tissues, in which there is no expression of CT genes.

This background signal depends upon the method used to carry out the invention. However, it is easy for a skilled person to measure such threshold whatever the method used, i.e.:
if the number of control samples is significantly statistically representative, e.g. at least 30 independent control samples, and the background signal of these control sample follows a normal distribution (Gaussian distribution), the threshold is determined by the mean+2 standard deviations of the background signal measured in the control samples,
If the number of control sample is not statistically representative, e.g. less than 30 independent control samples, or if the background signal of these control sample does not follow a normal distribution, the threshold is determined as being the maximal value of the background signal measured in the control samples.

According to the invention, the gene comprising or consisting of the nucleic acid sequence SEQ ID NO: 1 codes for the protein comprising or consisting of the amino acid sequence SEQ ID NO: 24, the gene comprising or consisting of the nucleic acid sequence SEQ ID NO: 2 codes for the protein comprising or consisting of the amino acid sequence SEQ ID NO: 25, the gene comprising or consisting of the nucleic acid sequence SEQ ID NO: 3 codes for the protein comprising or consisting of the amino acid sequence SEQ ID NO: 26, the gene comprising or consisting of the nucleic acid sequence SEQ ID NO: 4 codes for the protein comprising or consisting of the amino acid sequence SEQ ID NO: 27, the gene comprising or consisting of the nucleic acid sequence SEQ ID NO: 5 codes for the protein comprising or consisting of the amino acid sequence SEQ ID NO: 28, the gene comprising or consisting of the nucleic acid sequence SEQ ID NO: 6 codes for the protein comprising or consisting of the amino acid sequence SEQ ID NO: 29, the gene comprising or consisting of the nucleic acid sequence SEQ ID NO: 7 codes for the protein comprising or consisting of the amino acid sequence SEQ ID NO: 30, the gene comprising or consisting of the nucleic acid sequence SEQ ID NO: 8 codes for the protein comprising or consisting of the amino acid sequence SEQ ID NO: 31, the gene comprising or consisting of the nucleic acid sequence SEQ ID NO: 9 codes for the protein comprising or consisting of the amino acid sequence SEQ ID NO: 32, the gene comprising or consisting of the nucleic acid sequence SEQ ID NO: 10 codes for the protein comprising or consisting of the amino acid sequence SEQ ID NO: 33, the gene comprising or consisting of the nucleic acid sequence SEQ ID NO: 11 codes for the protein comprising or consisting of the amino acid sequence SEQ ID NO: 34, the gene comprising or consisting of the nucleic acid sequence SEQ ID NO: 12 codes for the protein comprising or consisting of the amino acid sequence SEQ ID NO: 35, the gene comprising or consisting of the nucleic acid sequence SEQ ID NO: 13 codes for the protein comprising or consisting of the amino acid sequence SEQ ID NO: 36, the gene comprising or consisting of the nucleic acid sequence SEQ ID NO: 14 codes for the protein comprising or consisting of the amino acid sequence SEQ ID NO: 37, the gene comprising or consisting of the nucleic acid sequence SEQ ID NO: 15 codes for the protein comprising or consisting of the amino acid sequence SEQ ID NO: 38, the gene comprising or consisting of the nucleic acid sequence SEQ ID NO: 16 codes for the protein comprising or consisting of the amino acid sequence SEQ ID NO: 39, the gene comprising or consisting of the nucleic acid sequence SEQ ID NO: 17 codes for the protein comprising or consisting of the amino acid sequence SEQ ID NO: 40, the gene comprising or consisting of the nucleic acid sequence SEQ ID NO: 18 codes for the protein comprising or consisting of the amino acid sequence SEQ ID NO: 41, the gene comprising or consisting of the nucleic acid sequence SEQ ID NO: 19 codes for the protein comprising or consisting of the amino acid sequence SEQ ID NO: 42, the gene comprising or consisting of the nucleic acid sequence SEQ ID NO: 20 codes for the protein comprising or consisting of the amino acid sequence SEQ ID NO: 43, the gene comprising or consisting of the nucleic acid sequence SEQ ID NO: 21 codes for the protein comprising or consisting of the amino acid sequence SEQ ID NO: 44, the gene comprising or consisting of the nucleic acid sequence SEQ ID NO: 22 codes for the protein comprising or consisting of the amino acid sequence SEQ ID NO: 45 and the gene comprising or consisting of the nucleic acid sequence SEQ ID NO: 23 codes for the protein comprising or consisting of the amino acid sequence SEQ ID NO: 46.

The 23 genes according to the invention code for 23 proteins. The 23 proteins have been classified by the Inventors in two sets:
a first set of 7 proteins,
and a second set of 16 proteins.

The first set, also called in the invention set AP, of 7 proteins consists of the proteins comprising or constituted by the amino acid sequences SEQ ID NO: 24, SEQ ID NO: 25, SEQ ID NO: 26, SEQ ID NO: 27, SEQ ID NO: 28, SEQ ID NO: 29 and SEQ ID NO: 30.

The second set, also called in the invention set BP, of 16 proteins consists of the proteins comprising or constituted by the amino acid sequences SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, and SEQ ID NO: 46.

The inventors have also defined subsets of each of the above sets AP and BP as follows:

Set AP is divided into 4 subsets AP1, AP5 A6 and AP7, said subset being such that:
subset AP1 consists of the proteins comprising or being constituted by the amino acid sequences SEQ ID NO: 24 and SEQ ID NO: 25,
subset AP5 consists of the protein comprising or being constituted by the amino acid sequence SEQ ID NO: 26,
subset AP6 consists of the proteins comprising or being constituted by the amino acid sequences SEQ ID NO: 27 and SEQ ID NO: 28, and
subset AP7 consists of the proteins comprising or being constituted by the amino acid sequences SEQ ID NO: 29 and SEQ ID NO: 30.
   Set AP can also be divided into the following subsets:
subset AP1 consists of the proteins comprising or being constituted by the amino acid sequences SEQ ID NO: 24 and SEQ ID NO: 25,
subset AP2 consists of the proteins comprising or being constituted by the amino acid sequences SEQ ID NO: 24, SEQ ID NO: 25 and SEQ ID NO: 26,
subset AP3 consists of the proteins comprising or being constituted by the amino acid sequences SEQ ID NO: 24, SEQ ID NO: 25, SEQ ID NO: 26, SEQ ID NO: 27 and SEQ ID NO: 28, and
subset AP4 consists of the proteins comprising or being constituted by the amino acid sequences SEQ ID NO: 24, SEQ ID NO: 25, SEQ ID NO: 26, SEQ ID NO: 27, SEQ ID NO: 28, SEQ ID NO: 29 and SEQ ID NO: 30.
   Set BP is divided into 3 subsets BP1, BP4 and BP5, said subset being such that:
subset BP1 consists of the proteins comprising or being constituted by the amino acid sequences SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38,
subset BP4 consists of the proteins comprising or being constituted by the amino acid sequences SEQ ID NO: 39, SEQ ID NO: 40, SEQ ID NO: 41, SEQ ID NO: 42, SEQ ID NO: 43 and SEQ ID NO: 44, and
subset BP5 consists of the proteins comprising or being constituted by the amino acid sequences SEQ ID NO: 45, and SEQ ID NO: 46.
   Set BP can also be divided into the following subsets:
subset BP1 consists of the proteins comprising or being constituted by the amino acid sequences SEQ ID NO: 31, SEQ ID NO: 32, SEQ ID NO: 33, SEQ ID NO: 34, SEQ ID NO: 35, SEQ ID NO: 36, SEQ ID NO: 37 and SEQ ID NO: 38,
subset BP2 consists of the proteins comprising or being constituted by the amino acid sequences SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43 and SEQ ID NO: 44, and
subset BP3 consists of the proteins comprising or being constituted by the amino acid sequences SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, and SEQ ID NO: 46.

In one advantageous embodiment, the invention relates to the use of
at least 2 genes chosen among a set of 23 genes comprising or consisting of the nucleic acid sequences SEQ ID NO 1 to 23,
or fragments of said genes,
or complementary sequences of said genes,
or sequences having at least 80% homology with said genes or fragment thereof,
or proteins coded by said genes,
or fragments of said proteins,
or antibodies directed against said proteins,
said at least 2 genes being such that
   at least one gene belongs to a subset A1 of a first set A of 7 genes comprising or consisting of the nucleic acid sequences SEQ ID NO: 1-7, said subset A1 comprising or consisting of nucleic acid sequences SEQ ID NO: 1 or 2
   at least one gene belongs to a subset B1 of a second set B of 16 genes comprising or consisting of the nucleic acid sequences SEQ ID NO: 8-23, said subset B1 comprising or consisting of nucleic acid sequences SEQ ID NO: 8 to 15
      for the implementation of a prognosis method, preferably in vitro, of the survival rate of a patient afflicted by a lung cancer, said prognosis being such that:
if none of the 23 genes of said set is expressed, the patient survival rate during a period of time from 30 to 120 months after the diagnosis of said lung cancer is from about 59% to about 78% or more, and
if at least one gene of at least one of the subset A1 or B1 is expressed, the patient survival rate during a period of time from 30 to 120 months after the diagnosis of said lung cancer is about from about 3% to about 70%.

In this embodiment of the invention, the prognosis method is carried out by using at least one of the following couples of genes:

SEQ ID NO: 1+SEQ ID NO: 8, SEQ ID NO: 1+SEQ ID NO: 9, SEQ ID NO: 1+SEQ ID NO: 10, SEQ ID NO: 1+SEQ ID NO: 11, SEQ ID NO: 1+SEQ ID NO: 12, SEQ ID NO: 1+SEQ ID NO: 13, SEQ ID NO: 1+SEQ ID NO: 14, SEQ ID NO: 1+SEQ ID NO: 15, SEQ ID NO: 2+SEQ ID NO: 8, SEQ ID NO: 2+SEQ ID NO: 9, SEQ ID NO: 2+SEQ ID NO: 10, SEQ ID NO: 2+SEQ ID NO: 11, SEQ ID NO: 2+SEQ ID NO: 12, SEQ ID NO: 2+SEQ ID NO: 13, SEQ ID NO: 2+SEQ ID NO: 14 and SEQ ID NO: 2+SEQ ID NO: 15.

The above 16 couples are sufficient to define a significant prognosis over 120 month of lung cancer.

Any other supplementary genes belonging to the group of 23 genes according to the invention can be used in order to affine the prognosis according to the invention.

In one advantageous embodiment, the invention relates to the use of
at least 2 genes chosen among a set of 23 genes comprising or consisting of the nucleic acid sequences SEQ ID NO 1 to 23,
or fragments of said genes,
or complementary sequences of said genes,
or sequences having at least 80% homology with said genes or fragment thereof,
or proteins coded by said genes,
or fragments of said proteins,
or antibodies directed against said proteins,
said at least 2 genes being such that
   at least one gene belongs to a subset A2 of a first set A of 7 genes comprising or consisting of the nucleic acid sequences SEQ ID NO: 1-7, said subset A1 comprising or consisting of nucleic acid sequences SEQ ID NO: 1 to 3
   at least one gene belongs to a subset B2 of a second set B of 16 genes comprising or consisting of the nucleic acid sequences SEQ ID NO: 8-23, said subset B1 comprising or consisting of nucleic acid sequences SEQ ID NO: 8 to 21
      for the implementation of a prognosis method, preferably in vitro, of the survival rate of a patient afflicted by a lung cancer, said prognosis being such that:
if none of the 23 genes of said set is expressed, the patient survival rate during a period of time from 30 to 120 months after the diagnosis of said lung cancer is from about 59% to about 78% or more, and
if at least one gene of at least one of the subset A2 or B2 is expressed, the patient survival rate during a period of time from 30 to 120 months after the diagnosis of said lung cancer is about from about 3% to about 70%.

In this embodiment of the invention, the prognosis method is carried out by using at least one of the following couples of genes:

SEQ ID NO: 1+SEQ ID NO: 8, SEQ ID NO: 1+SEQ ID NO: 9, SEQ ID NO: 1+SEQ ID NO: 10, SEQ ID NO: 1+SEQ ID NO: 11, SEQ ID NO: 1+SEQ ID NO: 12, SEQ ID NO: 1+SEQ ID NO: 13, SEQ ID NO: 1+SEQ ID NO: 14, SEQ ID NO: 1+SEQ ID NO: 15, SEQ ID NO: 1+SEQ ID NO: 16, SEQ ID NO: 1+SEQ ID NO: 17, SEQ ID NO: 1+SEQ ID NO: 18, SEQ ID NO: 1+SEQ ID NO: 19, SEQ ID NO: 1+SEQ ID NO: 20, SEQ ID NO: 1+SEQ ID NO: 21, SEQ ID NO: 2+SEQ ID NO: 8, SEQ ID NO: 2+SEQ ID NO: 9, SEQ ID NO: 2+SEQ ID NO: 10, SEQ ID NO: 2+SEQ ID NO: 11, SEQ ID NO: 2+SEQ ID NO: 12, SEQ ID NO: 2+SEQ ID NO: 13, SEQ ID NO: 2+SEQ ID NO: 14, SEQ ID NO: 2+SEQ ID NO: 15, SEQ ID NO: 2+SEQ ID NO: 16, SEQ ID NO: 2+SEQ ID NO: 17, SEQ ID NO: 2+SEQ ID NO: 18, SEQ ID NO: 2+SEQ ID NO: 19, SEQ ID NO: 2+SEQ ID NO: 20, SEQ ID NO: 2+SEQ ID NO: 21, SEQ ID NO: 3+SEQ ID NO: 8, SEQ ID NO: 3+SEQ ID NO: 9, SEQ ID NO: 3+SEQ ID NO: 10, SEQ ID NO: 3+SEQ ID NO: 11, SEQ ID NO: 3+SEQ ID NO: 12, SEQ ID NO: 3+SEQ ID NO: 13, SEQ ID NO: 3+SEQ ID NO: 14, SEQ ID NO: 3+SEQ ID NO: 15, SEQ ID NO: 3+SEQ ID NO: 16, SEQ ID NO: 3+SEQ ID NO: 17, SEQ ID NO: 3+SEQ ID NO: 18, SEQ ID NO: 3+SEQ ID NO: 19 and SEQ ID NO: 3+SEQ ID NO: 20.

In one advantageous embodiment, the invention relates to the use of
at least 2 genes chosen among a set of 23 genes comprising or consisting of the nucleic acid sequences SEQ ID NO 1 to 23,
or fragments of said genes,
or complementary sequences of said genes,
or sequences having at least 80% homology with said genes or fragment thereof,
or proteins coded by said genes,
or fragments of said proteins,
or antibodies directed against said proteins,
said at least 2 genes being such that
   at least one gene belongs to a subset A3 of a first set A of 7 genes comprising or consisting of the nucleic acid sequences SEQ ID NO: 1-7, said subset A1 comprising or consisting of nucleic acid sequences SEQ ID NO: 1 to 5
   at least one gene belongs to a subset B2 of a second set B of 16 genes comprising or consisting of the nucleic acid sequences SEQ ID NO: 8-23, said subset B1 comprising or consisting of nucleic acid sequences SEQ ID NO: 8 to 21
      for the implementation of a prognosis method, preferably in vitro, of the survival rate of a patient afflicted by a lung cancer, said prognosis being such that:
if none of the 23 genes of said set is expressed, the patient survival rate during a period of time from 30 to 120 months after the diagnosis of said lung cancer is from about 59% to about 78% or more, and
if at least one gene of at least one of the subset A3 or B2 is expressed, the patient survival rate during a period of time from 30 to 120 months after the diagnosis of said lung cancer is about from about 3% to about 70%.

In this embodiment of the invention, the prognosis method is carried out by using at least one of the following couples of genes:

SEQ ID NO: 1+SEQ ID NO: 8, SEQ ID NO: 1+SEQ ID NO: 9, SEQ ID NO: 1+SEQ ID NO: 10, SEQ ID NO: 1+SEQ ID NO: 11, SEQ ID NO: 1+SEQ ID NO: 12, SEQ ID NO: 1+SEQ ID NO: 13, SEQ ID NO: 1+SEQ ID NO: 14, SEQ ID NO: 1+SEQ ID NO: 15, SEQ ID NO: 1+SEQ ID NO: 16, SEQ ID NO: 1+SEQ ID NO: 17, SEQ ID NO: 1+SEQ ID NO: 18, SEQ ID NO: 1+SEQ ID NO: 19, SEQ ID NO: 1+SEQ ID NO: 20, SEQ ID NO: 1+SEQ ID NO: 21, SEQ ID NO: 1+SEQ ID NO: 22, SEQ ID NO: 1+SEQ ID NO: 23, SEQ ID NO: 2+SEQ ID NO: 8, SEQ ID NO: 2+SEQ ID NO: 9, SEQ ID NO: 2+SEQ ID NO: 10, SEQ ID NO: 2+SEQ ID NO: 11, SEQ ID NO: 2+SEQ ID NO: 12, SEQ ID NO: 2+SEQ ID NO: 13, SEQ ID NO: 2+SEQ ID NO: 14, SEQ ID NO: 2+SEQ ID NO: 15, SEQ ID NO: 2+SEQ ID NO: 16, SEQ ID NO: 2+SEQ ID NO: 17, SEQ ID NO: 2+SEQ ID NO: 18, SEQ ID NO: 2+SEQ ID NO: 19, SEQ ID NO: 2+SEQ ID NO: 20, SEQ ID NO: 2+SEQ ID NO: 21, SEQ ID NO: 2+SEQ ID NO: 22, SEQ ID NO: 2+SEQ ID NO: 23, SEQ ID NO: 3+SEQ ID NO: 8, SEQ ID NO: 3+SEQ ID NO: 9, SEQ ID NO: 3+SEQ ID NO: 10, SEQ ID NO: 3+SEQ ID NO: 11, SEQ ID NO: 3+SEQ ID NO: 12, SEQ ID NO: 3+SEQ ID NO: 13, SEQ ID NO: 3+SEQ ID NO: 14, SEQ ID NO: 3+SEQ ID NO: 15, SEQ ID NO: 3+SEQ ID NO: 16, SEQ ID NO: 3+SEQ ID NO: 17, SEQ ID NO: 3+SEQ ID NO: 18, SEQ ID NO: 3+SEQ ID NO: 19, SEQ ID NO: 3+SEQ ID NO: 20, SEQ ID NO: 3+SEQ ID NO: 21, SEQ ID NO: 3+SEQ ID NO: 22 and SEQ ID NO: 3+SEQ ID NO: 23.

In one advantageous embodiment, the invention relates to the use of
at least 2 proteins chosen among a set of 23 proteins coded by said 23 genes, said 23 proteins comprising or consisting of SEQ ID NO: 24 to 46,
or fragments of said proteins,
or antibodies directed against said proteins,
said at least 2 proteins being such that
   at least one gene belongs to a subset AP1 of a first set AP of 7 proteins comprising or consisting of the amino acid sequences SEQ ID NO: 24-30, said subset AP1 comprising or consisting of amino acid sequences SEQ ID NO: 24 or 25
   at least one protein belongs to a subset BP1 of a second set BP of 16 proteins comprising or consisting of the amino acid sequences SEQ ID NO: 31-46, said subset BP1 comprising or consisting of nucleic acid sequences SEQ ID NO: 31 to 38
      for the implementation of a prognosis method, preferably in vitro, of the survival rate of a patient afflicted by a lung cancer, said prognosis being such that:
if none of the 23 proteins of said set is expressed, the patient survival rate during a period of time from 30 to 120 months after the diagnosis of said lung cancer is from about 59% to about 78% or more, and
if at least one protein of at least one of the subset AP1 or BP1 is expressed, the patient survival rate during a period of time from 30 to 120 months after the diagnosis of said lung cancer is about from about 3% to about 70%.

In this embodiment of the invention, the prognosis method is carried out by using at least one of the following couples of proteins:

SEQ ID NO: 24+SEQ ID NO: 31, SEQ ID NO: 24+SEQ ID NO: 32, SEQ ID NO: 24+SEQ ID NO: 33, SEQ ID NO: 24+SEQ ID NO: 34, SEQ ID NO: 24+SEQ ID NO: 35, SEQ ID NO: 24+SEQ ID NO: 36, SEQ ID NO: 24+SEQ ID NO: 37, SEQ ID NO: 24+SEQ ID NO: 38, SEQ ID NO: 25+SEQ ID NO: 31, SEQ ID NO: 25+SEQ ID NO: 32, SEQ ID NO: 25+SEQ ID NO: 33, SEQ ID NO: 25+SEQ ID NO: 34, SEQ ID NO: 25+SEQ ID NO: 35, SEQ ID NO: 25+SEQ ID NO: 36, SEQ ID NO: 25+SEQ ID NO: 37 and SEQ ID NO: 25+SEQ ID NO: 38.

The above 16 couples are sufficient to defined a significant prognosis over 120 month of lung cancer.

In one advantageous embodiment, the invention relates to the use of
or at least 2 proteins chosen among a set of 23 proteins coded by said 23 genes, said 23 proteins comprising or consisting of SEQ ID NO: 24 to 46,
or fragments of said proteins,
or antibodies directed against said proteins,
said at least 2 proteins being such that
   at least one gene belongs to a subset AP2 of a first set AP of 7 proteins comprising or consisting of the amino acid sequences SEQ ID NO: 24-30, said subset AP2 comprising or consisting of amino acid sequences SEQ ID NO: 24 or 26
   at least one protein belongs to a subset BP2 of a second set BP of 16 proteins comprising or consisting of the amino acid sequences SEQ ID NO: 31-46, said subset BP2 comprising or consisting of nucleic acid sequences SEQ ID NO: 31 to 44
      for the implementation of a prognosis method, preferably in vitro, of the survival rate of a patient afflicted by a lung cancer, said prognosis being such that:
if none of the 23 proteins of said set is expressed, the patient survival rate during a period of time from 30 to 120 months after the diagnosis of said lung cancer is from about 59% to about 78% or more, and
if at least one protein of at least one of the subset AP2 or BP2 is expressed, the patient survival rate during a period of time from 30 to 120 months after the diagnosis of said lung cancer is about from about 3% to about 70%.

In one advantageous embodiment, the invention relates to the use of
or at least 2 proteins chosen among a set of 23 proteins coded by said 23 genes, said 23 proteins comprising or consisting of SEQ ID NO: 24 to 46,
or fragments of said proteins,
or antibodies directed against said proteins,
said at least 2 proteins being such that
   at least one gene belongs to a subset AP3 of a first set AP of 7 proteins comprising or consisting of the amino acid sequences SEQ ID NO: 24-30, said subset AP1 comprising or consisting of amino acid sequences SEQ ID NO: 24 or 28
   at least one protein belongs to a subset BP2 of a second set BP of 16 proteins comprising or consisting of the amino acid sequences SEQ ID NO: 31-46, said subset B1 comprising or consisting of nucleic acid sequences SEQ ID NO: 31 to 44
      for the implementation of a prognosis method, preferably in vitro, of the survival rate of a patient afflicted by a lung cancer, said prognosis being such that:
if none of the 23 proteins of said set is expressed, the patient survival rate during a period of time from 30 to 120 months after the diagnosis of said lung cancer is from about 59% to about 78% or more, and
if at least one protein of at least one of the subset AP3 or BP2 is expressed, the patient survival rate during a period of time from 30 to 120 months after the diagnosis of said lung cancer is about from about 3% to about 70%.

In one advantageous embodiment, the invention relates to the use of
at least 2 genes chosen among a set of 23 genes comprising or consisting of the nucleic acid sequences SEQ ID NO 1 to 23,
or fragments of said genes,
or complementary sequences of said genes,
or sequences having at least 80% homology with said genes or fragment thereof,
or proteins coded by said genes,
or fragments of said proteins,
or antibodies directed against said proteins,
said at least 2 genes being such that
   at least one gene belongs to a subset A5 of a first set A of 7 genes comprising or consisting of the nucleic acid sequences SEQ ID NO: 1-7, said subset A1 comprising or consisting of nucleic acid sequences SEQ ID NO: 3,
   at least one gene belongs to a subset B4 of a second set B of 16 genes comprising or consisting of the nucleic acid sequences SEQ ID NO: 8-23, said subset B1 comprising or consisting of nucleic acid sequences SEQ ID NO: 16 to 21,
      for the implementation of a prognosis method, preferably in vitro, of the survival rate of a patient afflicted by a lung cancer, said prognosis being such that:
if none of the 23 genes of said set is expressed, the patient survival rate during a period of time from 30 to 120 months after the diagnosis of said lung cancer is from about 59% to about 78% or more, and
if at least one gene of at least one of the subset A5 or B4 is expressed, the patient survival rate during a period of time from 30 to 120 months after the diagnosis of said lung cancer is about from about 3% to about 70%.

In one advantageous embodiment, the invention relates to the use of
at least 2 proteins chosen among a set of 23 proteins coded by said 23 genes, said 23 proteins comprising or consisting of SEQ ID NO: 24 to 46,
or fragments of said proteins,
or antibodies directed against said proteins,
said at least 2 proteins being such that
   at least one gene belongs to a subset AP5 of a first set AP of 7 proteins comprising or consisting of the amino acid sequences SEQ ID NO: 24-30, said subset AP5 comprising or consisting of amino acid sequences SEQ ID NO: 26
   at least one protein belongs to a subset BP4 of a second set BP of 16 proteins comprising or consisting of the amino acid sequences SEQ ID NO: 31-46, said subset BP4 comprising or consisting of nucleic acid sequences SEQ ID NO: 39 to 44
      for the implementation of a prognosis method, preferably in vitro, of the survival rate of a patient afflicted by a lung cancer, said prognosis being such that:
if none of the 23 proteins of said set is expressed, the patient survival rate during a period of time from 30 to 120 months after the diagnosis of said lung cancer is from about 59% to about 78% or more, and
if at least one protein of at least one of the subset AP5 or BP4 is expressed, the patient survival rate during a period of time from 30 to 120 months after the diagnosis of said lung cancer is about from about 3% to about 70%.

In one other advantageous embodiment, the invention relates to the use as mentioned above, wherein said prognosis method is such that:
if none of the 23 genes, or proteins, or antibodies, of said set is expressed, the patient survival rate during a period of time from 30 to 120 months after the diagnosis of said lung cancer is from about 59% to about 78% or more,
if none of the genes or proteins of the set B or BP, or of the subsets B1, BP1, B2, or BP2, or antibodies, is expressed and at least one gene or protein, or antibody, of the set A or AP, or of the subset A1, AP1, A2, AP2, A3 or AP3 is expressed, the patient survival rate during a period of time from 30 to 120 months after the diagnosis of said lung cancer is from about 27% to about 70%, and
if at least one gene, or protein, of the set B or BP, or of the subsets B1, BP1, B2 or BP2 or antibody, is expressed, the patient survival rate during a period of time from 30 to 120 months after the diagnosis of said lung cancer is from about 3% to about 55%.

In one other advantageous embodiment, the invention relates to the use as mentioned above, wherein said prognosis method is such that:
if none of the 23 genes, of said set is expressed, the patient survival rate during a period of time from 30 to 120 months after the diagnosis of said lung cancer is from about 59% to about 78% or more,
if none of the genes of the set B, or of the subsets B1 or B2 is expressed and at least one gene of the set A or of the subset A1, A2, or A3 is expressed, the survival rate during a period of time from 30 to 120 months after the diagnosis of said lung cancer is from about 27% to about 70%, and
if at least one gene of the set B or of the subsets B1, or B2 is expressed, survival rate during a period of time from 30 to 120 months after the diagnosis of said lung cancer is from about 3% to about 55%.

In one other advantageous embodiment, the invention relates to the use as mentioned above, wherein said prognosis method is such that:
if none of the 23 proteins, of said set is expressed, the patient survival rate during a period of time from 30 to 120 months after the diagnosis of said lung cancer is from about 59% to about 78% or more,
if none of the proteins, of the BP, or of the subsets BP1 or BP2, is expressed and at least one protein, of the set AP, or of the subset AP1, AP2, or AP3 is expressed, the patient survival rate during a period of time from 30 to 120 months after the diagnosis of said lung cancer is from about 27% to about 70%, and
if at least one protein, of the set BP, or of the subsets BP1 or BP2 is expressed, the survival rate during a period of time from 30 to 120 months after the diagnosis of said lung cancer is from about 3% to about 55%.

In one other advantageous embodiment, the invention relates to the above defined use, wherein said prognosis method is such that:
if none of the 23 genes or proteins, or antibodies, of said set is expressed, the patient survival rate during a period of time from 30 to 120 months after the diagnosis of said lung cancer is from about 59% to about 78% or more,
if
   none of the genes or proteins of the set B or BP, or of the subset B1, BP1, B2 or BP2, or antibodies, is expressed and at least 3 genes or proteins of the set A or AP, or of the subset A1, AP1, A2, AP2, A3 or AP3, or antibodies, is expressed, or
   at least 1 gene or protein of the set B or BP, or of the subset B1, BP1, B2 or BP2, or antibody, is expressed and from none to 2 genes or proteins of the set A or AP, or of the subset A1, AP1, A2, AP2, A3 or AP3, or antibodies, is expressed, or
   at least 2 genes or proteins of the set B or BP, or of the subset B1, BP1, B2 or BP2, or antibodies, is expressed and no gene or protein, of the set A or AP, or of the subset A1, AP1, A2, AP2, A3 or AP3, or antibodies, is expressed,
the patient survival rate during a period of time from 30 to 120 months after the diagnosis of said lung cancer is from about 27% to about 55%, and
if
   one gene or protein, of the genes or proteins of the set B or BP, or of the subset B1, BP1, B2 or BP2, or antibodies, is expressed and at least 3 genes or proteins of the set A or AP, or of the subset A1, AP1, A2, AP2, A3 or AP3, or antibodies, are expressed, or
   at least 2 genes or proteins of the set B or BP, or of the subset B1, BP1, B2 or BP2, or antibodies, are expressed and at least 1 gene or protein of the set A or AP, or of the subset A1, AP1, A2, AP2, A3 or AP3, or antibodies, are expressed,
the patient survival rate during a period of time from 30 to 120 months after the diagnosis of said lung cancer is from about 3% to about 13%.

In the invention “from none to 2” corresponds to none, or one or two.

By analogy, in the invention, “from none to X”, X varying from 3 to 23, corresponds to none, or one, or two, or three, or four, or five, or six, or seven . . . or twenty three.

In one other advantageous embodiment, the invention relates to the above defined use, wherein said prognosis method is such that:
if none of the 23 genes of said set is expressed, the patient survival rate during a period of time from 30 to 120 months after the diagnosis of said lung cancer is from about 59% to about 78% or more,
if
   none of the genes of the set B or of the subset B1or B2, is expressed and at least 3 genes of the set A, or of the subset A1, A2 or A3 is expressed, or
   at least 1 gene of the set B, or of the subset B1 or B2, is expressed and from none to 2 genes of the set A or of the subset A1, A2, or A3, is expressed, or
   at least 2 genes of the set B or of the subset B1or B2 is expressed and no gene of the set A or of the subset A1, A2, or A3, is expressed,
the patient survival rate during a period of time from 30 to 120 months after the diagnosis of said lung cancer is from about 27% to about 55%, and
if
   one gene of the genes of the set B or of the subset B1 or B2, is expressed and at least 3 genes of the set A or of the subset A1, A2, or A3, are expressed, or
   at least 2 genes of the set B or of the subset B1, or B2 are expressed and at least 1 gene of the set A or of the subset A1, A2, or A3, are expressed,
the patient survival rate during a period of time from 30 to 120 months after the diagnosis of said lung cancer is from about 3% to about 13%.

In one other advantageous embodiment, the invention relates to the above defined use, wherein said prognosis method is such that:
if none of the 23 proteins, of said set is expressed, the patient survival rate during a period of time from 30 to 120 months after the diagnosis of said lung cancer is from about 59% to about 78% or more,
if
   none of the proteins of the set BP, or of the subset BP1 or BP2, is expressed and at least 3 proteins, of the set AP, or of the subset AP1, AP2 or AP3, is expressed, or
   at least 1 protein of the set BP, or of the subset BP1 or BP2, is expressed and from none to 2 proteins, of the set AP, or of the subset AP1, AP2 or AP3, is expressed, or
   at least 2 proteins of the set BP, or of the subset BP1 or BP2, is expressed and no protein of the set AP, or of the subset AP1, AP2, or AP3, is expressed,
the patient survival rate during a period of time from 30 to 120 months after the diagnosis of said lung cancer is from about 27% to about 55%, and
if
   one protein of the proteins of the set BP, or of the subset BP1 or BP2, is expressed and at least 3 proteins of the set AP, or of the subset AP1, AP2 or AP3, are expressed, or
   at least 2 proteins of the set BP, or of the subset BP1 or BP2, are expressed and at least 1 protein of the set AP, or of the subset AP1, AP2 or AP3, are expressed,
the patient survival rate during a period of time from 30 to 120 months after the diagnosis of said lung cancer is from about 3% to about 13%.

In one another advantageous embodiment, the invention relates to the use as defined above, wherein said prognosis method is such that:
if none of the 23 genes or proteins of said set, or antibodies, is expressed the patient survival rate during a period of time from 30 to 120 months after the diagnosis of said lung cancer is from about 59% to about 78% or more,
if none of the genes or proteins of the set B or BP, or of the subsets B1, BP1, B2 or BP2, or antibodies, is expressed and one or two genes or proteins of the set A or AP, or of the subsets A1, AP1, A2, AP2, A3 or AP3, or antibodies, is expressed, the patient survival rate during a period of time from 30 to 120 months after the diagnosis of said lung cancer is from about 38% to about 70%,
if
   none of the genes or proteins of the set B or BP, or of the subsets B1, BP1, B2 or BP2, or antibodies, is expressed and at least 3 genes or proteins of the first set A or AP or of the subsets A1, AP1, A2, AP2, A3 or AP3, or antibodies, is expressed, or
   at least 1 gene or protein of the set B or BP, or of the subsets B1, BP1, B2 or BP2, or antibody, is expressed and from none to 2 genes or proteins of the set A or AP, or of the subsets A1, AP1, A2, AP2, A3 or AP3, or antibodies, is expressed, or
   at least 2 genes or proteins of the set B or BP, or of the subsets B1, BP1, B2 or BP2, or antibodies, is expressed and no gene or protein of the set A or AP, or of the subsets A1, AP1, A2, AP2, A3 or AP3, or antibody, is expressed,
the patient survival rate during a period of time from 30 to 120 months after the diagnosis of said lung cancer is from about 27% to about 55%, and
if
   one gene or protein of the genes or proteins, or antibodies, of the set B or BP, or of the subsets B 1, BP1, B2 or BP2, or antibody, is expressed and at least 3 genes or proteins of the set A or AP, or of the subsets A1, AP1, A2, AP2, A3 or AP3, or antibodies, are expressed, or
   at least 2 genes or proteins of the set B or BP, or of the subsets B1, BP1, B2 or BP2, or antibodies, are expressed and at least 1 gene or protein of the set A or AP, or of the subsets A1, AP1, A2, AP2, A3 or AP3, or antibody, is expressed
the patient survival rate during a period of time from 30 to 120 months after the diagnosis of said lung cancer is from about 3% to about 13%.

In one another advantageous embodiment, the invention relates to the use as defined above, wherein said prognosis method is such that:
if none of the 23 genes of said set is expressed, the patient survival rate during a period of time from 30 to 120 months after the diagnosis of said lung cancer is from about 59% to about 78% or more,
if none of the genes of the set B or of the subsets B1 or B2, is expressed and one or two genes of the set A or of the subsets A1, A2 or A3, is expressed, the patient survival rate is from about 38% to about 70%,
if
   none of the genes of the set B or of the subsets B1 or B2, is expressed and at least 3 genes of the first set is expressed, or
   at least 1 gene of the set B or of the subsets B1 or B2, is expressed and from none to 2 genes of the set A or of the subsets A1, A2 or A3, is expressed, or
   at least 2 genes of the set B or of the subsets B1 or B2, is expressed and no gene of the set A or of the subsets A1, A2 or A3, is expressed,
the patient survival rate during a period of time from 30 to 120 months after the diagnosis of said lung cancer is from about 27% to about 55%, and
if
   one gene of the genes of the set B or of the subsets B1 or B2, is expressed and at least 3 genes of the set A or of the subsets A1, A2 or A3, are expressed, or
   at least 2 genes of the set B or of the subsets B1 or B2, are expressed and at least 1 gene of the set A or of the subsets A1, A2 or A3, are expressed
the patient survival rate during a period of time from 30 to 120 months after the diagnosis of said lung cancer is from about 3% to about 13%.

In one another advantageous embodiment, the invention relates to the use as defined above, wherein said prognosis method is such that:
if none of the 23 proteins of said set is expressed, the patient survival rate during a period of time from 30 to 120 months after the diagnosis of said lung cancer is from about 59% to about 78% or more,
if none of the proteins of the set BP or of the subsets BP1 or BP2, is expressed and one or two proteins of the set AP or of the subsets AP1, AP2 or AP3, is expressed, the patient survival rate during a period of time from 30 to 120 months after the diagnosis of said lung cancer is from about 38% to about 70%,
if
   none of the proteins of the set BP or of the subsets BP1 or BP2, is expressed and at least 3 proteins of the first set is expressed, or
   at least 1 protein of the set BP or of the subsets BP1 or BP2, is expressed and from none to 2 proteins of the set AP or of the subsets AP1, AP2 or AP3, is expressed, or
   at least 2 proteins of the set BP or of the subsets BP1 or BP2, is expressed and no protein of the set AP or of the subsets AP1, AP2 or AP3, is expressed,
the patient survival rate during a period of time from 30 to 120 months after the diagnosis of said lung cancer is from about 27% to about 55%, and
if
   one protein of the proteins of the set BP or of the subsets BP1 or BP2, is expressed and at least 3 proteins of the set AP or of the subsets AP1, AP2 or AP3, are expressed, or
   at least 2 proteins of the set BP or of the subsets BP1 or BP2, are expressed and at least 1 protein of the set AP or of the subsets AP1, AP2 or AP3, are expressed
the patient survival rate during a period of time from 30 to 120 months after the diagnosis of said lung cancer is from about 3% to about 13%.

In another embodiment, the invention relates to the use as defined above, wherein during a period of time of 120 months from the diagnosis of said lung cancer
if none of the 23 genes or proteins of said set, or antibodies, is expressed, the patient survival rate is from about 59% or more, and
if at least one gene or protein of the genes or proteins of said set A or AP or B or BP, or of the subsets A1, AP1, A2, AP2, A3, AP3 or B1, BP1, B2 or BP2, or antibody, is expressed, the patient survival rate is about from about 3% to about 38%.

In another embodiment, the invention relates to the use as defined above, wherein during a period of time of 120 months from the diagnosis of said lung cancer
if none of the 23 genes of said set is expressed, the patient survival rate is from about 59% or more, and
if at least one gene of the genes of said set A or B, or of the subsets A1, A2, A3 or B1 or B2 is expressed, is expressed, the patient survival rate is about from about 3% to about 38%.

In another embodiment, the invention relates to the use as defined above, wherein during a period of time of 120 months from the diagnosis of said lung cancer
if none of the 23 proteins of said set is expressed, the patient survival rate is from about 59% or more, and
if at least one protein of the proteins of said set AP or BP, or of the subsets AP1, AP2, AP3 or BP1, BP2 is expressed, is expressed, the patient survival rate is about from about 3% to about 38%.

In an advantageous embodiment, the invention relates to the use as previously defined, wherein during a period of time of 120 months from the diagnosis of said lung cancer
if none of the 23 genes or proteins of said set, or antibodies, is expressed, the patient survival rate is about 59% or more,
if none of the genes or proteins of the set B or BP, or of the subsets B1, BP1, B2 or BP2, or antibodies, is expressed and one or two genes or proteins of the set A or AP, or of subsets A1, AP1, A2, AP2, A3, or AP3, or antibodies, is expressed, the patient survival rate is about 38%, and
if at least one gene or protein of the set B or BP, or of the subset B1, BP1, B2 or BP2, or antibody, is expressed, the patient survival rate is about 3% to about 27%.

In an advantageous embodiment, the invention relates to the use as previously defined, wherein during a period of time of 120 months from the diagnosis of said lung cancer
if none of the 23 genes of said set is expressed, the patient survival rate is about 59% or more,
if none of the genes of the set B, or of the subsets B1, or B2 is expressed and one or two genes of the set A, or of subsets A1, A2, or A3, is expressed, the patient survival rate is about 38%, and
if at least one gene of the set B, or of the subset B1, or B2, is expressed, the patient survival rate is about 3% to about 27%.

In an advantageous embodiment, the invention relates to the use as previously defined, wherein during a period of time of 120 months from the diagnosis of said lung cancer
if none of the 23 proteins of said set is expressed, the patient survival rate is about 59% or more,
if none of the proteins of the set BP, or of the subsets BP1 or BP2, is expressed and one or two proteins of the set AP, or of subsets AP1, AP2 or AP3 is expressed, the patient survival rate is about 38%, and
if at least one protein of the set B or BP, or of the subset B1, BP1, B2 or BP2, is expressed, the patient survival rate is about 3% to about 27%.

In an advantageous embodiment, the invention relates to the use as previously defined, wherein during a period of time of 120 months from the diagnosis of said lung cancer
if none of the 23 genes or proteins of said set, or antibodies, is expressed, the patient survival rate is about 59% or more,
if
   none of the genes or proteins of the set B or BP, or of subsets B1, BP1, B2 or BP2, or antibodies, is expressed and at least 3 genes or proteins of the set A or AP, or of subsets A1, AP1, A2, AP2, A3 or AP3, or antibodies, is expressed, or
   at least 1 gene or protein of the set B or BP, or of subsets B1, BP1, B2 or BP2, or antibody, is expressed and from none to 2 genes or proteins of the set A or AP, or of subsets A1, AP1, A2, AP2, A3 or AP3, or antibodies, is expressed, or
   at least 2 genes or proteins of the set B or BP, or of subsets B1, BP1, B2 or BP2, or antibodies, is expressed and no gene or protein of the set A or AP, or of subsets A1, AP1, A2, AP2, A3 or AP3, or antibody, is expressed,
the patient survival rate is about 27%, and
if
   one gene or protein of the genes or proteins, or antibodies, of the set B or BP, or of subsets B1, BP1, B2 or BP2, or antibody, is expressed and at least 3 genes or proteins of the set A or AP or of subsets A1, AP1, A2, AP2, A3 or AP3, or antibodies, are expressed, or
   at least 2 genes or proteins, of the set B or BP, or of subsets B1, BP1, B2 or BP2, or antibodies are expressed and at least 1 gene or protein of the set A or AP, or of subsets A1, AP1, A2, AP2, A3 or AP3, or antibody, are expressed
the patient survival rate is about 3%.

In an advantageous embodiment, the invention relates to the use as previously defined, wherein during a period of time of 120 months from the diagnosis of said lung cancer
if none of the 23 genes of said set is expressed, the patient survival rate is about 59% or more,
if
   none of the genes of the set B, or of subsets B1 or B2, is expressed and at least 3 genes of the set A, or of subsets A1, A2 or A3, is expressed, or
   at least 1 gene of the set B, or of subsets B1 or B2, is expressed and from none to 2 genes of the set A, or of subsets A1, A2 or A3, is expressed, or
   at least 2 genes of the set B, or of subsets B1 or B2, is expressed and no gene of the set A, or of subsets A1, A2, or A3, is expressed,
the patient survival rate is about 27%, and
if
   one gene of the genes of the set B, or of subsets B1 or B2, is expressed and at least 3 genes of the set A, or of subsets A1, A2, or A3, are expressed, or
   at least 2 genes of the set B, or of subsets B 1, or B2, are expressed and at least 1 gene of the set A, or of subsets A1, A2, or A3, are expressed
the patient survival rate is about 3%.

In an advantageous embodiment, the invention relates to the use as previously defined, wherein during a period of time of 120 months from the diagnosis of said lung cancer
if none of the 23 proteins of said set is expressed, the patient survival rate is about 59% or more,
if
   none of the proteins of the set BP, or of subsets BP1 or BP2, is expressed and at least 3 proteins of the set AP, or of subsets AP1, AP2 or AP3, is expressed, or
   at least 1 protein of the set BP, or of subsets BP1 or BP2, is expressed and from none to 2 proteins of the set AP, or of subsets AP1, AP2 or AP3, is expressed, or
   at least 2 proteins of the set BP, or of subsets BP1 or BP2, is expressed and no protein of the set AP, or of subsets AP1, AP2, or AP3, is expressed,
the patient survival rate is about 27%, and
if
   one protein of the proteins of the set BP, or of subsets BP1 or BP2, is expressed and at least 3 proteins of the set AP, or of subsets AP1, AP2, or AP3, are expressed, or
   at least 2 proteins of the set BP, or of subsets BP1, or BP2, are expressed and at least 1 protein of the set AP, or of subsets AP1, AP2, or AP3, are expressed
the patient survival rate is about 3%.

In an advantageous embodiment, the invention relates to the use as previously defined, wherein said prognosis method is such that:
if none of the 23 genes or proteins of said set, or antibodies, is expressed, the patient survival rate is about 59% or more,
if none of the genes or proteins of the set B or BP, or of subsets B1, BP1, B2 or BP2, or antibodies, is expressed and one or two genes or proteins of the set A or AP, or of subsets A1, AP1, A2, AP2, A3 or AP3, or antibodies, is expressed, the patient survival rate is about 38%,
if
   none of the genes or proteins of the set B or BP, or of subsets B1, BP1, B2 or BP2, or antibodies, is expressed and at least 3 genes or proteins of the set A or AP, or of subsets A1, AP1, A2, AP2, A3 or AP3, or antibodies, is expressed, or
   at least 1 gene or protein of the set B or BP, or of subsets B1, BP1, B2 or BP2, or antibody, is expressed and from none to 2 genes or proteins of the set A or AP, or of subsets A1, AP1, A2, AP2, A3 or AP3, or antibodies, is expressed, or
   at least 2 genes or proteins of the set B or BP, or of subsets B1, BP1, B2 or BP2, or antibodies, is expressed and no gene or protein of the set A or AP, or of subsets A1, AP1, A2, AP2, A3 or AP3, or antibody, is expressed,
the patient survival rate is about 27%, and
if
   one gene or protein of the genes or proteins, or antibodies, of the set B or BP, or of subsets B1, BP1, B2 or BP2, or antibody, is expressed and at least 3 genes or proteins of the set A or AP, or of subsets A1, AP1, A2, AP2, A3 or AP3, or antibodies, are expressed, or
   at least 2 genes or proteins of the set B or BP, or of subsets B1, BP1, B2 or BP2, or antibodies, are expressed and at least 1 gene or protein of the set A or AP, or of subsets A1, AP1, A2, AP2, A3 or AP3, or antibody, is expressed
the patient survival rate is about 3%.

In an advantageous embodiment, the invention relates to the use as previously defines, wherein said prognosis method is such that:
if none of the 23 genes of said set is expressed, the patient survival rate is about 59% or more,
if none of the genes of the set B, or of subsets B1 or B2, is expressed and one or two genes of the set A, or of subsets A1, A2 or A3, is expressed, the patient survival rate is about 38%,
if
   none of the genes of the set B, or of subsets B1 or B2, is expressed and at least 3 genes of the set A, or of subsets A1, A2, or A3, is expressed, or
   at least 1 gene of the set B, or of subsets B1 or B2, is expressed and from none to 2 genes of the set A, or of subsets A1, A2, or A3 is expressed, or
   at least 2 genes of the set B, or of subsets B1, or B2, is expressed and no gene of the set A, or of subsets A1 A2, or A3, is expressed,
the patient survival rate is about 27%, and
if
   one gene of the genes of the set B, or of subsets B1 or B2, is expressed and at least 3 genes of the set A, or of subsets A1, A2, or A3, are expressed, or
   at least 2 genes of the set B, or of subsets B1 or B2, are expressed and at least 1 gene of the set A, or of subsets A1, A2, A3, is expressed
the patient survival rate is about 3%.

In an advantageous embodiment, the invention relates to the use as previously defines, wherein said prognosis method is such that:
if none of the 23 proteins of said set is expressed, the patient survival rate is about 59% or more,
if none of the proteins of the set BP, or of subsets BP1 or BP2, is expressed and one or two proteins of the set AP, or of subsets AP1, AP2, or AP3, is expressed, the patient survival rate is about 38%,
if
   none of the proteins of the set BP, or of subsets BP1 or BP2, is expressed and at least 3 proteins of the set AP, or of subsets AP1, AP2 or AP3, is expressed, or
   at least 1 protein of the set BP, or of subsets BP1 or BP2, is expressed and from none to 2 proteins, of the set AP, or of subsets AP1, AP2 or AP3 is expressed, or
   at least 2 proteins of the set BP, or of subsets BP1 or BP2, is expressed and no protein, of the set AP, or of subsets AP1, AP2 or AP3, is expressed,
the patient survival rate is about 27%, and
if
   one protein of the proteins of the set BP, or of subsets BP1 or BP2, is expressed and at least 3 proteins of the set AP, or of subsets AP1, AP2 or AP3, are expressed, or
   at least 2 proteins of the set BP, or of subsets BP1 or BP2, are expressed and at least 1 protein of the set AP, or of subsets AP1, AP2 or AP3, is expressed
the patient survival rate is about 3%.

In another embodiment, the invention relates to the use as defined above, wherein during a period of time of 60 months from the diagnosis of said lung cancer
if none of the 23 genes or proteins of said set, or antibodies, is expressed, the patient survival rate is from about 66% or more, and
if at least one gene or protein of the genes or proteins of said set A or AP or B or BP, or of the subsets A1, AP1, A2, AP2, A3, AP3 or B1, BP1, B2 or BP2, or antibody, is expressed, the patient survival rate is about from about 3% to about 54%.

In another embodiment, the invention relates to the use as defined above, wherein during a period of time of 60 months from the diagnosis of said lung cancer
if none of the 23 genes of said set is expressed, the patient survival rate is from about 66% or more, and
if at least one gene of the genes of said set A or B, or of the subsets AP1, AP2, AP3 or BP1 or BP2 is expressed, is expressed, the patient survival rate is about from about 3% to about 54%.

In another embodiment, the invention relates to the use as defined above, wherein during a period of time of 60 months from the diagnosis of said lung cancer
if none of the 23 proteins of said set is expressed, the patient survival rate is from about 66% or more, and
if at least one protein of the proteins of said set AP or BP, or of the subsets AP1, AP2, AP3 or BP1, BP2 is expressed, is expressed, the patient survival rate is about from about 3% to about 54%.

In an advantageous embodiment, the invention relates to the use as previously defined, wherein during a period of time of 60 months from the diagnosis of said lung cancer
if none of the 23 genes or proteins of said set, or antibodies, is expressed, the patient survival rate is about 66% or more,
if none of the genes or proteins of the set B or BP, or of the subsets B1, BP1, B2 or BP2, or antibodies, is expressed and one or two genes or proteins of the set A or AP, or of subsets A1, AP1, A2, AP2, A3, or AP3, or antibodies, is expressed, the patient survival rate is about 54%, and
if at least one gene or protein of the set B or BP, or of the subset B1, BP1, B2 or BP2, or antibody, is expressed, the patient survival rate is about 3% to about 36%.

In an advantageous embodiment, the invention relates to the use as previously defined, wherein during a period of time of 60 months from the diagnosis of said lung cancer
if none of the 23 genes of said set is expressed, the patient survival rate is about 66% or more,
if none of the genes of the set B, or of the subsets B1, or B2 is expressed and one or two genes of the set A, or of subsets A1, A2, or A3, is expressed, the patient survival rate is about 54%, and
if at least one gene of the set B, or of the subset B1, or B2, is expressed, the patient survival rate is about 3% to about 36%.

In an advantageous embodiment, the invention relates to the use as previously defined, wherein during a period of time of 60 months from the diagnosis of said lung cancer
if none of the 23 proteins of said set is expressed, the patient survival rate is about 66% or more,
if none of the proteins of the set BP, or of the subsets BP1 or BP2, is expressed and one or two proteins of the set AP, or of subsets AP1, AP2 or AP3 is expressed, the patient survival rate is about 54%, and
if at least one protein of the set B or BP, or of the subset B1, BP1, B2 or BP2, is expressed, the patient survival rate is about 3% to about 36%.

In an advantageous embodiment, the invention relates to the use as previously defined, wherein during a period of time of 60 months from the diagnosis of said lung cancer
if none of the 23 genes or proteins of said set is expressed, or antibodies, the patient survival rate is about 66% or more,
if
   none of the genes or proteins of the set B or BP, or of subsets B1, BP1, B2 or BP2, or antibodies, is expressed and at least 3 genes or proteins of the set A or AP, or of subsets A1, AP1, A2, AP2, A3 or AP3, or antibodies, is expressed, or
   at least 1 gene or protein of the set B or BP, or of subsets B1, BP1, B2 or BP2, or antibody, is expressed and from none to 2 genes or proteins of the set A or AP, or of subsets A1, AP1, A2, AP2, A3 or AP3, or antibodies, is expressed, or
   at least 2 genes or proteins of the set B or BP, or of subsets B1, BP1, B2 or BP2, or antibodies, is expressed and no gene or protein of the set A or AP, or of subsets A1, AP1, A2, AP2, A3 or AP3, or antibody, is expressed,
the patient survival rate is about 36%, and
if
   one gene or protein of the genes or proteins of the set B or BP, or of subsets B1, BP1, B2 or BP2, or antibody, is expressed and at least 3 genes or proteins of the set A or AP or of subsets A1, AP1, A2, AP2, A3 or AP3, or antibodies, are expressed, or
   at least 2 genes or proteins of the set B or BP, or of subsets

B1, BP1, B2 or BP2, or antibodies, are expressed and at least 1 gene or protein of the set A or AP, or of subsets A1, AP1, A2, AP2, A3 or AP3, or antibody, are expressed
the patient survival rate is about 3%.

In an advantageous embodiment, the invention relates to the use as previously defined, wherein during a period of time of 60 months from the diagnosis of said lung cancer
if none of the 23 genes of said set is expressed, the patient survival rate is about 66% or more,
if
   none of the genes of the set B, or of subsets B1 or B2, is expressed and at least 3 genes of the set A, or of subsets A1, A2 or A3, is expressed, or
   at least 1 gene of the set B, or of subsets B1 or B2, is expressed and from none to 2 genes of the set A, or of subsets A1, A2 or A3, is expressed, or
   at least 2 genes of the set B, or of subsets B1 or B2, is expressed and no gene of the set A, or of subsets A1, A2, or A3, is expressed,
the patient survival rate is about 36%, and
if
   one gene of the genes of the set B, or of subsets B1 or B2, is expressed and at least 3 genes of the set A, or of subsets A1, A2, or A3, are expressed, or
   at least 2 genes of the set B, or of subsets B 1, or B2, are expressed and at least 1 gene of the set A, or of subsets A1, A2, or A3, are expressed
the patient survival rate is about 3%.

In an advantageous embodiment, the invention relates to the use as previously defined, wherein during a period of time of 60 months from the diagnosis of said lung cancer
if none of the 23 proteins of said set is expressed, the patient survival rate is about 66% or more,
if
   none of the proteins of the set BP, or of subsets BP1 or BP2, is expressed and at least 3 proteins of the set AP, or of subsets AP1, AP2 or AP3, is expressed, or
   at least 1 protein of the set BP, or of subsets BP1 or BP2, is expressed and from none to 2 proteins of the set AP, or of subsets AP1, AP2 or AP3, is expressed, or
   at least 2 proteins of the set BP, or of subsets BP1 or BP2, is expressed and no protein of the set AP, or of subsets AP1, AP2, or AP3, is expressed,
the patient survival rate is about 36%, and
if
   one protein of the proteins of the set BP, or of subsets BP1 or BP2, is expressed and at least 3 proteins of the set AP, or of subsets AP1, AP2, or AP3, are expressed, or
   at least 2 proteins of the set BP, or of subsets BP1, or BP2, are expressed and at least 1 protein of the set AP, or of subsets AP1, AP2, or AP3, are expressed
the patient survival rate is about 3%.

In an advantageous embodiment, the invention relates to the use as previously defined, wherein said prognosis method is such that:
if none of the 23 genes or proteins of said set, or antibodies, is expressed, the patient survival rate is about 66% or more,
if none of the genes or proteins of the set B or BP, or of subsets B1, BP1, B2 or BP2, or antibodies, is expressed and one or two genes or proteins of the set A or AP, or of subsets A1, AP1, A2, AP2, A3 or AP3, or antibodies is expressed, the patient survival rate is about 54%,
if
   none of the genes or proteins of the set B or BP, or of subsets B1, BP1, B2 or BP2, or antibodies, is expressed and at least 3 genes or proteins of the set A or AP, or of subsets A1, AP1, A2, AP2, A3 or AP3, or antibodies, is expressed, or
   at least 1 gene or protein of the set B or BP, or of subsets B1, BP1, B2 or BP2, or antibody, is expressed and from none to 2 genes or proteins of the set A or AP, or of subsets A1, AP1, A2, AP2, A3 or AP3, or antibodies, is expressed, or
   at least 2 genes or proteins of the set B or BP, or of subsets B1, BP1, B2 or BP2, or antibodies, is expressed and no gene or protein of the set A or AP, or of subsets A1, AP1, A2, AP2, A3 or AP3, or antibody, is expressed,
the patient survival rate is about 36%, and
if
   one gene or protein of the genes or proteins of the set B or BP, or of subsets B1, BP1, B2 or BP2, or antibody, is expressed and at least 3 genes or proteins of the set A or AP, or of subsets A1, AP1, A2, AP2, A3 or AP3, or antibodies, are expressed, or
   at least 2 genes or proteins of the set B or BP, or of subsets B1, BP1, B2 or BP2, or antibodies, are expressed and at least 1 gene or protein of the set A or AP, or of subsets A1, AP1, A2, AP2, A3 or AP3, or antibody, is expressed
the patient survival rate is about 3%.

In an advantageous embodiment, the invention relates to the use as previously defined, wherein said prognosis method is such that:
if none of the 23 genes of said set is expressed, the patient survival rate is about 66% or more,
if none of the genes of the set B, or of subsets B1 or B2, is expressed and one or two genes of the set A, or of subsets A1, A2 or A3, is expressed, the patient survival rate is about 54%,
if
   none of the genes of the set B, or of subsets B1 or B2, is expressed and at least 3 genes of the set A, or of subsets A1, A2, or A3, is expressed, or
   at least 1 gene of the set B, or of subsets B1 or B2, is expressed and from none to 2 genes of the set A, or of subsets A1, A2, or A3 is expressed, or
   at least 2 genes of the set B, or of subsets B1, or B2, is expressed and no gene of the set A, or of subsets A1 A2, or A3, is expressed,
the patient survival rate is about 36%, and
if
   one gene of the genes of the set B, or of subsets B1 or B2, is expressed and at least 3 genes of the set A, or of subsets A1, A2, or A3, are expressed, or
   at least 2 genes of the set B, or of subsets B1 or B2, are expressed and at least 1 gene of the set A, or of subsets A1, A2, A3, is expressed
the patient survival rate is about 3%.

In an advantageous embodiment, the invention relates to the use as previously defined, wherein said prognosis method is such that:
if none of the 23 proteins of said set is expressed, the patient survival rate is about 66% or more,
if none of the proteins of the set BP, or of subsets BP1 or BP2, is expressed and one or two proteins of the set AP, or of subsets AP1, AP2, or AP3, is expressed, the patient survival rate is about 54%,
if
   none of the proteins of the set BP, or of subsets BP1 or BP2, is expressed and at least 3 proteins of the set AP, or of subsets AP1, AP2 or AP3, is expressed, or
   at least 1 protein of the set BP, or of subsets BP1 or BP2, is expressed and from none to 2 proteins, of the set AP, or of subsets AP1, AP2 or AP3 is expressed, or
   at least 2 proteins of the set BP, or of subsets BP1 or BP2, is expressed and no protein, of the set AP, or of subsets AP1, AP2 or AP3, is expressed,
the patient survival rate is about 36%, and
if
   one protein of the proteins of the set BP, or of subsets BP1 or BP2, is expressed and at least 3 proteins of the set AP, or of subsets AP1, AP2 or AP3, are expressed, or
   at least 2 proteins of the set BP, or of subsets BP1 or BP2, are expressed and at least 1 protein of the set AP, or of subsets AP1, AP2 or AP3, is expressed
the patient survival rate is about 3%.

In another embodiment, the invention relates to the use as defined above, wherein during a period of time of 30 months from the diagnosis of said lung cancer
if none of the 23 genes or proteins of said set, or antibodies, is expressed, the patient survival rate is from about 78% or more, and
if at least one gene or protein of the genes or proteins of said set A or AP or B or BP, or of the subsets A1, AP1, A2, AP2, A3, AP3 or B1, BP1, B2 or BP2, or antibody, is expressed, the patient survival rate is about from about 13% to about 70%.

In another embodiment, the invention relates to the use as defined above, wherein during a period of time of 30 months from the diagnosis of said lung cancer
if none of the 23 genes of said set is expressed, the patient survival rate is from about 78% or more, and
if at least one gene of the genes of said set A or B, or of the subsets AP1, AP2, AP3 or BP1 or BP2 is expressed, is expressed, the patient survival rate is about from about 13% to about 70%.

In another embodiment, the invention relates to the use as defined above, wherein during a period of time of 30 months from the diagnosis of said lung cancer
if none of the 23 proteins of said set is expressed, the patient survival rate is from about 78% or more, and
if at least one protein of the proteins of said set AP or BP, or of the subsets AP1, AP2, AP3 or BP1, BP2 is expressed, is expressed, the patient survival rate is about from about 13% to about 70%.

In an advantageous embodiment, the invention relates to the use as previously defined, wherein during a period of time of 30 months from the diagnosis of said lung cancer
if none of the 23 genes or proteins of said set, or antibodies, is expressed, the patient survival rate is about 78% or more,
if none of the genes or proteins of the set B or BP, or of the subsets B1, BP1, B2 or BP2, or antibodies, is expressed and one or two genes or proteins of the set A or AP, or of subsets A1, AP1, A2, AP2, A3, or AP3, or antibodies, is expressed, the patient survival rate is about 70%, and
if at least one gene or protein of the set B or BP, or of the subset B1, BP1, B2 or BP2, or antibody, is expressed, the patient survival rate is about 13% to about 55%.

In an advantageous embodiment, the invention relates to the use as previously defined, wherein during a period of time of 30 months from the diagnosis of said lung cancer
if none of the 23 genes of said set is expressed, the patient survival rate is about 78% or more,
if none of the genes of the set B, or of the subsets B1, or B2 is expressed and one or two genes of the set A, or of subsets A1, A2, or A3, is expressed, the patient survival rate is about 70%, and
if at least one gene of the set B, or of the subset B1, or B2, is expressed, the patient survival rate is about 13% to about 55%.

In an advantageous embodiment, the invention relates to the use as previously defines, wherein during a period of time of 30 months from the diagnosis of said lung cancer
if none of the 23 proteins of said set is expressed, the patient survival rate is about 78% or more,
if none of the proteins of the set BP, or of the subsets BP1 or BP2, is expressed and one or two proteins of the set AP, or of subsets AP1, AP2 or AP3 is expressed, the patient survival rate is about 70%, and
if at least one protein of the set B or BP, or of the subset B1, BP1, B2 or BP2, is expressed, the patient survival rate is about 13% to about 55%.

In an advantageous embodiment, the invention relates to the use as previously defined, wherein during a period of time of 30 months from the diagnosis of said lung cancer
if none of the 23 genes or proteins of said set, or antibodies, is expressed, the patient survival rate is about 78% or more,
if
   none of the genes or proteins of the set B or BP, or of subsets B1, BP1, B2 or BP2, or antibodies, is expressed and at least 3 genes or proteins of the set A or AP, or of subsets A1, AP1, A2, AP2, A3 or AP3, or antibodies, is expressed, or
   at least 1 gene or protein of the set B or BP, or of subsets B1, BP1, B2 or BP2, or antibody, is expressed and from none to 2 genes or proteins of the set A or AP, or of subsets A1, AP1, A2, AP2, A3 or AP3, or antibodies, is expressed, or
   at least 2 genes or proteins of the set B or BP, or of subsets B1, BP1, B2 or BP2, or antibodies, is expressed and no gene or protein of the set A or AP, or of subsets A1, AP1, A2, AP2, A3 or AP3, or antibody, is expressed,
the patient survival rate is about 55%, and
if
   one gene or protein of the genes or proteins, or antibodies, of the set B or BP, or of subsets B1, BP1, B2 or BP2, or antibody, is expressed and at least 3 genes or proteins of the set A or AP or of subsets A1, AP1, A2, AP2, A3 or AP3, or antibodies, are expressed, or
   at least 2 genes or proteins, of the set B or BP, or of subsets B1, BP1, B2 or BP2, or antibodies are expressed and at least 1 gene or protein of the set A or AP, or of subsets A1, AP1, A2, AP2, A3 or AP3, or antibody, are expressed
the patient survival rate is about 13%.

In an advantageous embodiment, the invention relates to the use as previously defined, wherein during a period of time of 30 months from the diagnosis of said lung cancer
if none of the 23 genes of said set is expressed, the patient survival rate is about 78% or more,
if
   none of the genes of the set B, or of subsets B1 or B2, is expressed and at least 3 genes of the set A, or of subsets A1, A2 or A3, is expressed, or
   at least 1 gene of the set B, or of subsets B1 or B2, is expressed and from none to 2 genes of the set A, or of subsets A1, A2 or A3, is expressed, or
   at least 2 genes of the set B, or of subsets B1 or B2, is expressed and no gene of the set A, or of subsets A1, A2, or A3, is expressed,
the patient survival rate is about 55%, and
if
   one gene of the genes of the set B, or of subsets B1 or B2, is expressed and at least 3 genes of the set A, or of subsets A1, A2, or A3, are expressed, or
   at least 2 genes of the set B, or of subsets B 1, or B2, are expressed and at least 1 gene of the set A, or of subsets A1, A2, or A3, are expressed
the patient survival rate is about 13%.

In an advantageous embodiment, the invention relates to the use as previously defined, wherein during a period of time of 30 months from the diagnosis of said lung cancer
if none of the 23 proteins of said set is expressed, the patient survival rate is about 78% or more,
if
   none of the proteins of the set BP, or of subsets BP1 or BP2, is expressed and at least 3 proteins of the set AP, or of subsets AP1, AP2 or AP3, is expressed, or
   at least 1 protein of the set BP, or of subsets BP1 or BP2, is expressed and from none to 2 proteins of the set AP, or of subsets AP1, AP2 or AP3, is expressed, or
   at least 2 proteins of the set BP, or of subsets BP1 or BP2, is expressed and no protein of the set AP, or of subsets AP1, AP2, or AP3, is expressed,
the patient survival rate is about 55%, and
if
   one protein of the proteins of the set BP, or of subsets BP1 or BP2, is expressed and at least 3 proteins of the set AP, or of subsets AP1, AP2, or AP3, are expressed, or
   at least 2 proteins of the set BP, or of subsets BP1, or BP2, are expressed and at least 1 protein of the set AP, or of subsets AP1, AP2, or AP3, are expressed
the patient survival rate is about 13%.

In an advantageous embodiment, the invention relates to the use as previously defined, wherein said prognosis method is such that:
if none of the 23 genes or proteins of said set, or antibodies, is expressed, the patient survival rate is about 78% or more,
if none of the genes or proteins of the set B or BP, or of subsets B1, BP1, B2 or BP2, or antibodies, is expressed and one or two genes or proteins of the set A or AP, or of subsets A1, AP1, A2, AP2, A3 or AP3, or antibodies, is expressed, the patient survival rate is about 70%,
if
   none of the genes or proteins of the set B or BP, or of subsets B1, BP1, B2 or BP2, or antibodies, is expressed and at least 3 genes or proteins, of the set A or AP, or of subsets A1, AP1, A2, AP2, A3 or AP3, or antibodies, is expressed, or
   at least 1 gene or protein of the set B or BP, or of subsets B1, BP1, B2 or BP2, or antibody, is expressed and from none to 2 genes or proteins of the set A or AP, or of subsets A1, AP1, A2, AP2, A3 or AP3, or antibodies, is expressed, or
   at least 2 genes or proteins of the set B or BP, or of subsets B1, BP1, B2 or BP2, or antibodies, is expressed and no gene or protein of the set A or AP, or of subsets A1, AP1, A2, AP2, A3 or AP3, or antibody, is expressed,

the patient survival rate is about 55%, and
if
   one gene or protein of the genes or proteins, or antibodies, of the set B or BP, or of subsets B1, BP1, B2 or BP2, or antibody, is expressed and at least 3 genes or proteins of the set A or AP, or of subsets A1, AP1, A2, AP2, A3 or AP3, or antibodies, are expressed, or
   at least 2 genes or proteins of the set B or BP, or of subsets B1, BP1, B2 or BP2, or antibodies, are expressed and at least 1 gene or protein of the set A or AP, or of subsets A1, AP1, A2, AP2, A3 or AP3, or antibody, is expressed,
the patient survival rate is about 13%.

In an advantageous embodiment, the invention relates to the use as previously defined, wherein said prognosis method is such that:
if none of the 23 genes of said set is expressed, the patient survival rate is about 78% or more,
if none of the genes of the set B, or of subsets B1 or B2, is expressed and one or two genes of the set A, or of subsets A1, A2 or A3, is expressed, the patient survival rate is about 70%,
if
   none of the genes of the set B, or of subsets B1 or B2, is expressed and at least 3 genes of the set A, or of subsets A1, A2, or A3, is expressed, or
   at least 1 gene of the set B, or of subsets B1 or B2, is expressed and from none to 2 genes of the set A, or of subsets A1, A2, or A3 is expressed, or
   at least 2 genes of the set B, or of subsets B1, or B2, is expressed and no gene of the set A, or of subsets A1 A2, or A3, is expressed,
the patient survival rate is about 55%, and
if
   one gene of the genes of the set B, or of subsets B1 or B2, is expressed and at least 3 genes of the set A, or of subsets A1, A2, or A3, are expressed, or
   at least 2 genes of the set B, or of subsets B1 or B2, are expressed and at least 1 gene of the set A, or of subsets A1, A2, A3, is expressed
the patient survival rate is about 13%.

In an advantageous embodiment, the invention relates to the use as previously defined, wherein said prognosis method is such that:
if none of the 23 proteins of said set is expressed, the patient survival rate is about 78% or more,
if none of the proteins of the set BP, or of subsets BP1 or BP2, is expressed and one or two proteins of the set AP, or of subsets AP1, AP2, or AP3, is expressed, the patient survival rate is about 70%,
if
   none of the proteins of the set BP, or of subsets BP1 or BP2, is expressed and at least 3 proteins of the set AP, or of subsets AP1, AP2 or AP3, is expressed, or
   at least 1 protein of the set BP, or of subsets BP1 or BP2, is expressed and from none to 2 proteins, of the set AP, or of subsets AP1, AP2 or AP3 is expressed, or
   at least 2 proteins of the set BP, or of subsets BP1 or BP2, is expressed and no protein, of the set AP, or of subsets AP1, AP2 or AP3, is expressed,
the patient survival rate is about 55%, and
if
   one protein of the proteins of the set BP, or of subsets BP1 or BP2, is expressed and at least 3 proteins of the set AP, or of subsets AP1, AP2 or AP3, are expressed, or
   at least 2 proteins of the set BP, or of subsets BP1 or BP2, are expressed and at least 1 protein of the set AP, or of subsets AP1, AP2 or AP3, is expressed
the patient survival rate is about 13%.

In one another advantageous embodiment, the invention relates to the use as defined above, wherein said lung tumor has been previously histologically classified.

In one another advantageous embodiment, the invention relates to the use as defined above, wherein said histologically classified tumor belongs to the set consisting of: ADK, SQC, BAS, and LCNE, wherein ADK corresponds to adenocarcinoma, SQC corresponds to Squamous cell carcinoma, BAS corresponds to Basaloid tumours and LCNE corresponds to Large Cell Neuroendocrine.

The invention also relates to a prognosis method, preferably in vitro, of the survival rate of a patient afflicted by a lung tumour, from a biological sample containing said lung tumor, at a time from 30 to 120 months after the diagnosis of said lung cancer, as defined above,

said method comprising a step of measuring, in said biological sample, the expression of
at least 2 genes chosen among a set of 23 genes comprising or consisting of the nucleic acid sequences SEQ ID NO 1 to 23,
or fragments of said least 2 genes chosen among a set of 23 genes comprising or consisting of the nucleic acid sequences SEQ ID NO 1 to 23,
or complementary sequences of said least 2 genes chosen among a set of 23 genes comprising or consisting of the nucleic acid sequences SEQ ID NO 1 to 23,
or sequences having at least 80% homology with said genes or fragment thereof,
or proteins coded by said least 2 genes chosen among a set of 23 genes comprising or consisting of the nucleic acid sequences SEQ ID NO 1 to 23, said proteins comprising or consisting in amino acid sequences SEQ ID NO 24 to 46
or fragments of said proteins comprising or consisting in amino acid sequences SEQ ID NO 24 to 46,
or antibodies directed against said proteins comprising or consisting in amino acid sequences SEQ ID NO 24 to 46,
said
at least 2 genes being such that
   at least one gene belongs to a first set A of 7 genes comprising or consisting of the nucleic acid sequences SEQ ID NO: 1-7
   at least one gene belongs to a second set B of 16 genes comprising or consisting of the nucleic acid sequences SEQ ID NO: 8-23,
at least 2 proteins being such that
   at least one protein belongs to a first set AP of 7 proteins comprising or consisting of the amino acid sequences SEQ ID NO: 24-30,
   at least one protein belongs to a second set BP of 16 proteins comprising or consisting of the amino acid sequences SEQ ID NO: 31-46,
at least 2 antibodies directed against said 2 proteins being such that
   at least one antibody specifically recognises one protein that belongs to a first set AP of 7 proteins comprising or consisting of the amino acid sequences SEQ ID NO: 24-30,
   at least one antibody specifically recognises one protein that belongs to a second set BP of 16 proteins comprising or consisting of the amino acid sequences SEQ ID NO: 31-46,
      said method being such that:
      either
if none of the 23 genes of said set is expressed, the patient survival rate during a period of time from 30 to 120 months after the diagnosis of said lung cancer is from about 59% to about 78% or more, and
if at least one gene of at least one set A or B is expressed, the patient survival rate during a period of time from 30 to 120 months after the diagnosis of said lung cancer is about from about 3% to about 70%,
   or
if none of the 23 proteins of said set is expressed, the patient survival rate during a period of time from 30 to 120 months after the diagnosis of said lung cancer is from about 59% to about 78% or more, and
if at least one protein of at least one set AP or BP is expressed, the patient survival rate during a period of time from 30 to 120 months after the diagnosis of said lung cancer is about from about 3% to about 70%,
   or
if none of the antibodies directed against said 23 proteins of said set is expressed, the patient survival rate during a period of time from 30 to 120 months after the diagnosis of said lung cancer is from about 59% to about 78% or more, and
if at least one antibody directed against one protein of at least one set AP or BP is expressed, the patient survival rate during a period of time from 30 to 120 months after the diagnosis of said lung cancer is about from about 3% to about 70%.

In one advantageous embodiment, the invention relates to a prognosis method, as defined above,

said method comprising a step of measuring, in said biological sample, the expression of
at least 2 genes chosen among a set of 23 genes comprising or consisting of the nucleic acid sequences SEQ ID NO 1 to 23,
or fragments of said genes
or complementary sequences of said genes
or sequences having at least 80% homology with said genes or fragment thereof,
or protein coded by said genes,
or fragments of said proteins,
or antibodies directed against said proteins,
said at least 2 genes being such that
   at least one gene belongs to a first set A of 7 genes, or of a subset A1, A2 or A3 as defined above, said set comprising or consisting of the nucleic acid sequences SEQ ID NO: 1-7
   at least one gene belongs to a second set B of 16 genes, or of a subset B1 or B2 as defined above, said set comprising or consisting of the nucleic acid sequences SEQ ID NO: 8-23
      said prognosis method being such that
if none of the 23 genes of said set is expressed, the patient survival rate is from about 59% to about 78%, or more, and
if at least one gene of said set A or B, or of subsets A1, A2, A3, B1 or B2 is expressed, the patient survival rate from about 3% to about 70%.

In another advantageous embodiment, the invention relates to a prognosis method previously defined, wherein the step of measuring is carried out by using a technique chosen among the set consisting of:
Quantitative PCR,
DNA CHIP, and
Northern blot.

In another advantageous embodiment, the invention relates to a prognosis method as previously defined, wherein the step of measuring is carried out by using nucleic acid molecules consisting of from 15 to 100 nucleotides molecules being complementary to said at least 2 genes.

In one advantageous embodiment, the invention relates to a prognosis method as defined above, wherein the step of measuring is carried out by DNA CHIP using
at least one nucleic acid probe comprising or being constituted by the nucleic acid sequences SEQ ID NO: 47 to SEQ ID NO: 53, and
at least one nucleic acid probe comprising or being constituted by the nucleic acid sequences SEQ ID NO: 54 to SEQ ID NO: 69.

In another advantageous embodiment, the invention relates to the method as defined above, wherein the step of measuring is carried out by DNA CHIP using at least 2, preferably at least 3 nucleic acid probes as defined above.

An advantageous embodiment of the invention relates to the above method wherein the nucleic acid probes comprising or being constituted by the nucleic acid sequences SEQ ID NO: 47 to 69 are used, together.

In the invention the correspondence between the genes and the nucleic acid probes are as follows: SEQ ID NO:1 is able to be detected by the nucleic acid probe comprising or being constituted by the nucleic acid sequence SEQ ID NO: 47, SEQ ID NO:2 is able to be detected by the nucleic acid probe comprising or being constituted by the nucleic acid sequence SEQ ID NO: 48, SEQ ID NO:3 is able to be detected by the nucleic acid probe comprising or being constituted by the nucleic acid sequence SEQ ID NO: 49, SEQ ID NO:4 is able to be detected by the nucleic acid probe comprising or being constituted by the nucleic acid sequence SEQ ID NO: 50, SEQ ID NO:5 is able to be detected by the nucleic acid probe comprising or being constituted by the nucleic acid sequence SEQ ID NO: 51, SEQ ID NO:6 is able to be detected by the nucleic acid probe comprising or being constituted by the nucleic acid sequence SEQ ID NO: 52, SEQ ID NO:7 is able to be detected by the nucleic acid probe comprising or being constituted by the nucleic acid sequence SEQ ID NO: 53, SEQ ID NO:8 is able to be detected by the nucleic acid probe comprising or being constituted by the nucleic acid sequence SEQ ID NO: 54, SEQ ID NO:9 is able to be detected by the nucleic acid probe comprising or being constituted by the nucleic acid sequence SEQ ID NO: 55, SEQ ID NO:10 is able to be detected by the nucleic acid probe comprising or being constituted by the nucleic acid sequence SEQ ID NO: 56, SEQ ID NO:11 is able to be detected by the nucleic acid probe comprising or being constituted by the nucleic acid sequence SEQ ID NO: 57, SEQ ID NO:12 is able to be detected by the nucleic acid probe comprising or being constituted by the nucleic acid sequence SEQ ID NO: 58, SEQ ID NO:13 is able to be detected by the nucleic acid probe comprising or being constituted by the nucleic acid sequence SEQ ID NO: 59, SEQ ID NO:14 is able to be detected by the nucleic acid probe comprising or being constituted by the nucleic acid sequence SEQ ID NO: 60, SEQ ID NO:15 is able to be detected by the nucleic acid probe comprising or being constituted by the nucleic acid sequence SEQ ID NO: 61, SEQ ID NO:16 is able to be detected by the nucleic acid probe comprising or being constituted by the nucleic acid sequence SEQ ID NO: 62, SEQ ID NO:17 is able to be detected by the nucleic acid probe comprising or being constituted by the nucleic acid sequence SEQ ID NO: 63, SEQ ID NO:18 is able to be detected by the nucleic acid probe comprising or being constituted by the nucleic acid sequence SEQ ID NO: 64, SEQ ID NO:19 is able to be detected by the nucleic acid probe comprising or being constituted by the nucleic acid sequence SEQ ID NO: 65, SEQ ID NO:20 is able to be detected by the nucleic acid probe comprising or being constituted by the nucleic acid sequence SEQ ID NO: 66, SEQ ID NO:21 is able to be detected by the nucleic acid probe comprising or being constituted by the nucleic acid sequence SEQ ID NO: 67, SEQ ID NO:22 is able to be detected by the nucleic acid probe comprising or being constituted by the nucleic acid sequence SEQ ID NO: 68, SEQ ID NO:23 is able to be detected by the nucleic acid probe comprising or being constituted by the nucleic acid sequence SEQ ID NO: 69.

In one another advantageous embodiment, the invention relates to a prognosis method, preferably in vitro, of the survival rate of a patient afflicted by a lung tumour, from a biological sample containing said lung tumor, at a time from 30 to 120 months after the diagnosis of said lung cancer,

said method comprising a step of measuring, in said biological sample, the expression of
at least 2 proteins chosen among a set of 23 proteins coded by 23 genes comprising or consisting of the nucleic acid sequences SEQ ID NO 1 to 23,
or fragments of said proteins,
said at least 2 proteins being such that
   at least one protein belongs to a first set AP of 7 proteins, each 7 proteins being coded by one of 7 genes comprising or consisting of the nucleic acid sequences SEQ ID NO: 1-7
   at least one protein belongs to a second set BP of 16 proteins, each 16 proteins being coded by one of 16 genes comprising or consisting of the nucleic acid sequences SEQ ID NO: 8-23
      said prognosis method being such that
if none of the 23 proteins is expressed, the patient survival rate is from about 59% to about 78%, or more, and
if at least one protein of said set AP or BP is expressed, the patient survival rate from about 3% to about 70%.

In one another advantageous embodiment, the invention relates to a prognosis method, wherein the step of measuring is carried out by using a technique chosen among the set consisting of:
western Blot,
ELISA,
Immunofluorescence, and
Immunohistochemistry.

In one another advantageous embodiment, the invention relates to a prognosis method, wherein the step of measuring is carried out by using antibodies directed against said at least 2 proteins coded by said at least two genes.

In one another advantageous embodiment, the invention relates to a prognosis method, further comprising a step of comparison of said measured expression to the expression in at least one control sample.

According to the invention, the above mentioned gene, proteins or antibody expression can be compared with the expression level of the same genes, proteins and antibodies measured in
a control sample corresponding to a sample originating from an healthy individual, and/or
a positive sample corresponding to a sample of an individual expressing the above mentioned gene, protein, or antibodies.

The invention also concerns a kit comprising a DNA CHIP comprising at least the nucleic acid probes comprising or being constituted by the nucleic acid sequences SEQ ID NO: 47 to 69.

The invention also relates to the use of
at least 13 genes chosen among a set of 28 genes comprising or consisting of the nucleic acid sequences SEQ ID NO 70 to 97,
or fragments of said least 13 genes chosen among a set of 28 genes comprising or consisting of the nucleic acid sequences SEQ ID NO 70 to 97,
or complementary sequences of said least 13 genes chosen among a set of 28 genes comprising or consisting of the nucleic acid sequences SEQ ID NO 70 to 97,
or sequences having at least 80% homology with said genes or fragment thereof,
or proteins coded by said least 13 genes chosen among a set of 28 genes comprising or consisting of the nucleic acid sequences
or fragments of said proteins,
or antibodies directed against said proteins,
said at least 13 genes being such that
   12 genes comprise or consist of the nucleic acid sequences SEQ ID NO: 70 to 81, and
   at least one gene belongs to a subset of 16 genes comprising or consisting of the nucleic acid sequences SEQ ID NO: 82-97,
      for carrying out a method for identifying at least 66% of patients of those having a survival rate of at most about 20% at 30 months, among a population of patients afflicted by lung cancer having an estimated survival rate of about at least about 30% at 30 months based on the diagnosis of said lung cancer according to histopathological criteria.

Lung cancer is one of the most frequent cancer in humans and is the most frequent cause of mortality by cancer in human.

Classically, when diagnosed, lung tumors are classified according to the TNM classification (tumor size, node positivity and metastasis) by clinicians and into histopathological subtypes by histopathologists. TNM corresponds to the clinical criteria according to the invention.

Based on the TNM analysis, it is possible to establish for a patient a survival probability, in percent, at 30 months, 60 months and 120 months from the diagnosis.

At about 60 months, patients afflicted by lung tumors have a survival rate of about 50% (shown in FIG. 41).

In the invention, terms “survival rate” can be uniformally replaced by “survival probability” or “survival estimate”

The present invention is based on the unexpected observation made by the Inventors that the expression of at least 13 genes of a group of 28 determined genes is sufficient to discriminate at least ⅔ of the patients having a very poor prognosis, i.e. a survival rate very low, said patients being non identified by the clinical or histopatological criteria.

According to the invention as explained and exemplified hereafter, the determination of the gene expression status on a ON/OFF basis of at least 13 genes chosen among a set of 28 genes as defined above.

A key aspect of the invention is the concept of ON/OFF for gene expression. The concept of ON/OFF expression can be extended to presence/absence of the proteins and antibodies detecting these proteins. This specific approach has the advantage of simplifying the analyses and making them independent of complex statistical tests to measure variations in expression levels applied to the majority of the existing tests.

The ON/OFF status of gene expression is established by determination of a threshold of gene expression allowing them to decide on the ON/OFF status of a gene such that:
if a gene is expressed at a level lower than the threshold, the gene is considered as not expressed (defined as OFF), and
if a gene is expressed at a level upper to the threshold, the gene is considered as being expressed (defined as ON).

The above 28 genes have been identified as being liable to be “expressed” (form here by, “expressed” refers to the ON status and “not expressed” to the OFF status) in lung cancer cells, but not in healthy samples. In other words, the above 28 genes are such as
they are not expressed, in healthy lung cells, and
they maybe expressed in lung tumor cells.

The difference between the absence of expression and the expression determines its ON/OFF status, which is a key step of the invention.

Indeed, the Inventors have identified that the ON status of the above 28 genes is a key step to determine the prognosis of lung cancer.

On microarrays, the expression level of the above mentioned genes is determined by the fact that a threshold of expression has been identified by the Inventors allowing to determine expression (ON) and non-expression (OFF) of said genes. The threshold determination is detailed hereafter, in the Example section.

For the microarrays, the threshold enabling to determine the expression status of a gene (ON versus OFF) is calculated by using the signal mean value and distribution obtained from transcriptomic data (in the same technology) with the corresponding probes in a large number of somatic tissues (which do not express the genes).

A similar strategy enables determining a threshold for the presence/absence of the encoded proteins or antibodies. For each protein or antibody, the mean value and distribution of the signal intensities obtained in an appropriate number of control somatic tissues serves as a basis for calculating the threshold.

By “not expressed” it is defined in the invention the fact that the transcription of a gene is either not carried out, or is not detectable by common techniques known in the art, such as Quantitative RT-PCR, Northern blot or when microarrays data are considered.

By “expressed”, the invention defined that the transcript of a gene is detectable by the above known techniques while it is not detectable in healthy tissues or determined as being above the threshold when microarrays data are considered.

In the invention, terms “carrying out” and “implementation” are used uniformly.

The subgroup of 13 determined genes chosen among a group of 28 determined genes identified by the Inventors allows the identification of at least ⅔, or 66%, of patients having a prognosis to be alive 30 months after the diagnosis of their lung tumor of about at most 20%. Said patients with the above bad/poor prognosis are not detected by the histopathological methods, such as the TNM method.

Actually, the subgroup of 13 determined genes chosen among a group of 28 determined genes identified by the Inventors allows to separate 3 distinct populations:

P1 and P2 populations: patients having a survival rate of about at least 20% after 30 months from the diagnosis of their lung tumors, and
P3 population: patients having a survival rate of about at most 20% after 30 months from the diagnosis of their lung tumors.

This is illustrated in FIG. 42.

The patients of the P3 population have to be identified in order to treat them very rapidly, when possible, in view of the agressivity of their tumors, and to inform them that they have a very poor prognosis.

The above explanation applies when lung tumors are analysed independently from any further status.

The method according to the invention, the use as mentioned above also applies when tumors are detected at early stage (in the invention called “T1N0”) or at late stage (in the invention called “T+N+”).

According to the TNM classification, the survival rate over the months are represented in FIG. 43.

When applying the method as described above, in each of the T1N0 and T+N+, 3 populations can be defined: i.e. the above mentioned P1, P2 and P3 populations.

The repartition of P1, P2 and P3 populations is represented in FIG. 44 for T1N0 and in FIG. 45 for T+N+.

The same applies when tumors are identified according to their histological status, chosen among SQC (squamous cell cancer), LCNE (Large Cell Neuroendocrine tumour) and BAS (basaloid tumour).

According to the histopathological classification, the survival rates over the months are represented in FIG. 46.

When applying the method as described above, in each of the BAS, SQC or LCNE tumors, 3 populations can be defined: i.e. the above mentioned P1, P2 and P3 populations.

The repartition of P1, P2 and P3 populations is represented in FIG. 47 for BAS, in FIG. 48 for SQC and in FIG. 49 for LCNE.

According to the invention, it is sufficient to use at least 13 genes of the group of 28 genes comprising or consisting of SEQ ID NO: 70 to 97, to identify at least 66% of patients having a poor prognosis as defined above, said 13 genes being such that:
12 of these 13 genes comprise or consist of the nucleic acid sequences SEQ ID NO: 70-81, and
at least one gene chosen among the genes comprising or consisting of the nucleic acid sequences SEQ ID NO: 82-97.

In the invention, it is possible to use, at least 13 genes as mentioned above, or fragments of said at least 13 genes, or complementary sequences of said at least 13 genes or fragments thereof. For the purpose of the invention, the term “gene” refers to the transcriptional product of the genes, also called cDNA, or to the genomic counterpart.

The invention also relates to the use of at least 13 proteins, said proteins being coded by said at least 13 genes.

The correspondence between the genes and the proteins of the invention is as follows:

| Gene name | DNA: SEQ ID NO | Protein: SEQ ID NO: |
|---|---|---|
| MAGEB6 | 70 | 98 |
| TPTE/TPTE2 | 71 | 99 |
| RBM46 | 72 | 100 |
| HIST1H3A; HIST1H3C | 73 | 101 |
| CPA5 | 74 | 102 |
| RFX4 | 75 | 103 |
| TUBA3C/TUBA3D | 76 | 104 |
| KIAA1257 | 77 | 105 |
| ARHGEF40 | 78 | 106 |
| TKTL2 | 79 | 107 |
| CCDC83 | 80 | 108 |
| DPEP3 | 81 | 109 |
| C10orf82 | 82 | 110 |
| C12orf37 | 83 | — |
| PIWIL1 | 84 | 111 |
| ROPN1 | 85 | 112 |
| NBPF4/NBPF6 | 86 | 113 |
| LOC220115 | 87 | 114 |
| BTG4 | 88 | 115 |
| ISM2 | 89 | 116 |
| OR7E156P | 90 | — |
| EBI3 | 91 | 117 |
| LGALS14 | 92 | 118 |
| LOC441601 | 93 | — |
| VCY/VCY1B | 94 | 119 |
| FLJ43944 | 95 | 120 |
| IGFBP1 | 96 | 121 |
| CCDC38 | 97 | 122 |

There is, in the invention, 16 minimal sets of 13 genes that can be used to identify said at least 66% of patients belonging to the P3 population:
SEQ ID NO: 70, 71, 72, 73, 74, 75, 76, 77, 78, 79, 80 and 81, plus SEQ ID NO: 82,
SEQ ID NO: 70, 71, 72, 73, 74, 75, 76, 77, 78, 79, 80 and 81, plus SEQ ID NO: 83,
SEQ ID NO: 70, 71, 72, 73, 74, 75, 76, 77, 78, 79, 80 and 81, plus SEQ ID NO: 84,
SEQ ID NO: 70, 71, 72, 73, 74, 75, 76, 77, 78, 79, 80 and 81, plus SEQ ID NO: 85,
SEQ ID NO: 70, 71, 72, 73, 74, 75, 76, 77, 78, 79, 80 and 81, plus SEQ ID NO: 86,
SEQ ID NO: 70, 71, 72, 73, 74, 75, 76, 77, 78, 79, 80 and 81, plus SEQ ID NO: 87,
SEQ ID NO: 70, 71, 72, 73, 74, 75, 76, 77, 78, 79, 80 and 81, plus SEQ ID NO: 88,
SEQ ID NO: 70, 71, 72, 73, 74, 75, 76, 77, 78, 79, 80 and 81, plus SEQ ID NO: 89,
SEQ ID NO: 70, 71, 72, 73, 74, 75, 76, 77, 78, 79, 80 and 81, plus SEQ ID NO: 90,
SEQ ID NO: 70, 71, 72, 73, 74, 75, 76, 77, 78, 79, 80 and 81, plus SEQ ID NO: 91,
SEQ ID NO: 70, 71, 72, 73, 74, 75, 76, 77, 78, 79, 80 and 81, plus SEQ ID NO: 92,
SEQ ID NO: 70, 71, 72, 73, 74, 75, 76, 77, 78, 79, 80 and 81, plus SEQ ID NO: 93,
SEQ ID NO: 70, 71, 72, 73, 74, 75, 76, 77, 78, 79, 80 and 81, plus SEQ ID NO: 94,
SEQ ID NO: 70, 71, 72, 73, 74, 75, 76, 77, 78, 79, 80 and 81, plus SEQ ID NO: 95,
SEQ ID NO: 70, 71, 72, 73, 74, 75, 76, 77, 78, 79, 80 and 81, plus SEQ ID NO: 96,
SEQ ID NO: 70, 71, 72, 73, 74, 75, 76, 77, 78, 79, 80 and 81, plus SEQ ID NO: 97,

According to the invention, “the use of at least 13 genes of the group of 28 genes” means that 13, 14, 15, 16, 17, 18, 19, 20, 21, 22, 23, 24, 25, 26, 27 or 28 genes are used.

When at least 14 genes are considered, or more, the skilled person could easily combine SEQ ID NO: 70, 71, 72, 73, 74, 75, 76, 77, 78, 79, 80 and 81 plus two genes from those of the list SEQ ID NO: 82, 83, 84, 85, 86, 87, 88, 89, 90, 91, 92, 93, 94, 95, 96 and 97.

In one advantageous embodiment, the invention relates to the use of at least 13 proteins coded by said least 13 genes chosen among a set of 25 proteins
or fragments of said proteins,
or antibodies directed against said proteins,
said at least 13 proteins genes being such that
   12 proteins comprise or consist of the nucleic acid sequences SEQ ID NO: 98 to 110, and
   at least one protein belongs to a subset of 13 genes comprising or consisting of the nucleic acid sequences SEQ ID NO: 111-122,
      for carrying out a method for identifying at least 66% of patients of those having a survival rate of at most about 20% at 30 months, among a population of patients afflicted by lung cancer having an estimated survival rate of at least about 30% at 30 months based on the diagnosis of said lung cancer according to histopathological criteria.

Advantageously, using the above 13 gene, i.e. SEQ ID NO: 70-81+anyone of gene chosen among SEQ ID NO: 82-97, allows identification from about 66% to about 70% of patients of those having a survival rate of at most about 20% at 30 months

In one advantageous embodiment, the invention relates to the use defined above, of
at least 13 genes chosen among a set of 28 genes comprising or consisting of the nucleic acid sequences SEQ ID NO 70 to 97,
or fragments of said least 13 genes chosen among a set of 28 genes comprising or consisting of the nucleic acid sequences SEQ ID NO 70 to 97,
or complementary sequences of said least 13 genes chosen among a set of 28 genes comprising or consisting of the nucleic acid sequences SEQ ID NO 70 to 97,
or sequences having at least 80% homology with said genes or fragments thereof,
said at least 13 genes being such that
   12 genes comprise or consist of the nucleic acid sequences SEQ ID NO: 70 to 81, and
   at least one gene belongs to a subset of 16 genes comprising or consisting of the nucleic acid sequences SEQ ID NO: 82-97,
      for carrying out a method for identifying at least 66% of patients of those having a survival rate of at most about 20% at 30 months, among a population of patients afflicted by lung cancer having an estimated survival rate of at least about 30% at 30 months based on the diagnosis of said lung cancer according to histopathological criteria.

In the invention, all the percentages are expressed as “about X %”. This means that said percent are the X value±(plus or minus) the variation of 5% of the value X.

Thus “about 66% percent” encompass the interval 66−5%≦66≦66+5%, i.e from 62.7% to 69.3%.

From this explanation, the skilled person knows how to correctly determine the advantageous intervals of the invention.

In another advantageous embodiment, the invention relates to the use defined above, wherein said at least one gene belongs to a subset of 16 genes comprising or consisting of the nucleic acid sequences SEQ ID NO: 82-97, is at least the gene comprising or consisting of the nucleic acid sequences SEQ ID NO: 82,

for carrying out a method for identifying at least 70% of patients having a survival rate of at most about 20% at 30 months.

In another advantageous embodiment, the invention relates to the use defined above, of at least 18 genes
said at least 18 genes being such that
   12 genes comprise or consist of the nucleic acid sequences SEQ ID NO: 70 to 81, and
   at least 6 genes belongs to a subset of 16 genes comprising or consisting of the nucleic acid sequences SEQ ID NO: 82-97, said at least 6 genes comprising or consisting of the nucleic acid sequences SEQ ID NO:82-85 and 87-88,
      for carrying out a method for identifying at least 83% of patients of those having a survival rate of at most about 20% at 30 months.

In another advantageous embodiment, the invention relates to the use defined above, of at least 21 genes,
said at least 21 genes being such that
   12 genes comprise or consist of the nucleic acid sequences SEQ ID NO: 70 to 81, and
   at least 9 gene belongs to a subset of 16 genes comprising or consisting of the nucleic acid sequences SEQ ID NO: 82-97, said at least 9 genes preferably comprising or consisting of the nucleic acid sequences SEQ ID NO: 82-88 and 91-92,
      for carrying out a method for identifying at least 89% of patients having a survival rate of at most about 20% at 30 months.

In another advantageous embodiment, the invention relates to the use defined above, of at least 26 genes
said at least 26 genes being such that
   12 genes comprise or consist of the nucleic acid sequences SEQ ID NO: 70 to 81, and
   at least 14 gene belongs to a subset of 16 genes comprising or consisting of the nucleic acid sequences SEQ ID NO: 82-97, said at least 14 genes preferably comprising or consisting of the nucleic acid sequences SEQ ID NO: 82-92 and 93-96,
      for carrying out a method for identifying at least 97% of patients of those having a survival rate of at most about 20% at 30 months.

In another advantageous embodiment, the invention relates to the use defined above, of at least 27 genes
said at least 26 genes being such that
   12 genes comprise or consist of the nucleic acid sequences SEQ ID NO: 70 to 81, and
   at least 15 gene belongs to a subset of 16 genes comprising or consisting of the nucleic acid sequences SEQ ID NO: 82-97
      for carrying out a method for identifying at least 97% of patients of those having a survival rate of at most about 20% at 30 months.

In another advantageous embodiment, the invention relates to the use defined above, of 28 genes comprising or consisting of the nucleic acid sequences SEQ ID NO: 70-97 for carrying out a method for identifying 100% of patients of those having a survival rate of at most about 20% at 30 months.

Hereafter are indicated the advantageous groups of genes according to the invention, along with the percentage of patients belonging to the P3 group they allow to detect.

| GENES | % |
|---|---|
| 13genes = SEQ ID NO: 70-81 + SEQ ID NO: 82 | 70.8% |
| 13genes = SEQ ID NO: 70-81 + SEQ ID NO: 83 | 66.7% |
| 13genes = SEQ ID NO: 70-81 + SEQ ID NO: 84 | 64.6% |
| 13genes = SEQ ID NO: 70-81 + SEQ ID NO: 85 | 66.7% |
| 13genes = SEQ ID NO: 70-81 + SEQ ID NO: 86 | 64.6% |
| 13genes = SEQ ID NO: 70-81 + SEQ ID NO: 87 | 66.7% |
| 13genes = SEQ ID NO: 70-81 + SEQ ID NO: 88 | 66.7% |
| 13genes = SEQ ID NO: 70-81 + SEQ ID NO: 89 | 66.7% |
| 13genes = SEQ ID NO: 70-81 + SEQ ID NO: 90 | 66.7% |
| 13genes = SEQ ID NO: 70-81 + SEQ ID NO: 91 | 66.7% |
| 13genes = SEQ ID NO: 70-81 + SEQ ID NO: 92 | 66.7% |
| 13genes = SEQ ID NO: 70-81 + SEQ ID NO: 93 | 64.6% |
| 13genes = SEQ ID NO: 70-81 + SEQ ID NO: 94 | 64.6% |
| 13genes = SEQ ID NO: 70-81 + SEQ ID NO: 95 | 66.7% |
| 13genes = SEQ ID NO: 70-81 + SEQ ID NO: 96 | 66.7% |
| 13genes = SEQ ID NO: 70-81 + SEQ ID NO: 97 | 66.7% |
| 14 genes: SEQ ID NO: 70-83 | 72.9% |
| 15 genes: SEQ ID NO: 70-84 | 72.9% |
| 16 genes: SEQ ID NO: 70-85 | 75.0% |
| 17 genes: SEQ ID NO: 70-86 | 75.0% |
| 18 genes: SEQ ID NO: 70-87 | 81.3% |
| 19 genes: SEQ ID NO: 70-88 | 87.5% |
| 20 genes: SEQ ID NO: 70-89 | 87.5% |
| 21 genes: SEQ ID NO: 70-90 | 87.5% |
| 22 genes: SEQ ID NO: 70-91 | 89.6% |
| 23 genes: SEQ ID NO: 70-92 | 93.8% |
| 24 genes: SEQ ID NO: 70-93 | 93.8% |
| 25 genes: SEQ ID NO: 70-94 | 95.8% |
| 26 genes: SEQ ID NO: 70-95 | 97.9% |
| 27 genes: SEQ ID NO: 70-96 | 100.0% |
| All 28 genes SEQ ID NO: 70-97 | 100.0% |
| 13genes = SEQ ID NO: 70-81 + SEQ ID NO: 82 | 70.8% |
| 14genes = SEQ ID NO: 70-81 + SEQ ID NO: 82 + SEQ ID NO: 83 | 72.9% |
| 14genes = SEQ ID NO: 70-81 + SEQ ID NO: 82 + SEQ ID NO: 84 | 70.8% |
| 14genes = SEQ ID NO: 70-81 + SEQ ID NO: 82 + SEQ ID NO: 85 | 72.9% |
| 14genes = SEQ ID NO: 70-81 + SEQ ID NO: 82 + SEQ ID NO: 86 | 70.8% |
| 14genes = SEQ ID NO: 70-81 + SEQ ID NO: 82 + SEQ ID NO: 87 | 72.9% |
| 14genes = SEQ ID NO: 70-81 + SEQ ID NO: 82 + SEQ ID NO: 88 | 72.9% |
| 14genes = SEQ ID NO: 70-81 + SEQ ID NO: 82 + SEQ ID NO: 89 | 72.9% |
| 14genes = SEQ ID NO: 70-81 + SEQ ID NO: 82 + SEQ ID NO: 90 | 72.9% |
| 14genes = SEQ ID NO: 70-81 + SEQ ID NO: 82 + SEQ ID NO: 91 | 72.9% |
| 14genes = SEQ ID NO: 70-81 + SEQ ID NO: 82 + SEQ ID NO: 92 | 72.9% |
| 14genes = SEQ ID NO: 70-81 + SEQ ID NO: 82 + SEQ ID NO: 93 | 70.8% |
| 14genes = SEQ ID NO: 70-81 + SEQ ID NO: 82 + SEQ ID NO: 94 | 70.8% |
| 14genes = SEQ ID NO: 70-81 + SEQ ID NO: 82 + SEQ ID NO: 95 | 72.9% |
| 14genes = SEQ ID NO: 70-81 + SEQ ID NO: 82 + SEQ ID NO: 96 | 72.9% |
| 14genes = SEQ ID NO: 70-81 + SEQ ID NO: 82 + SEQ ID NO: 97 | 72.9% |
| 14genes = SEQ ID NO: 70-83 | 72.9% |
| 15genes = SEQ ID NO: 70-83 + SEQ ID NO: 84 | 72.9% |
| 15genes = SEQ ID NO: 70-83 + SEQ ID NO: 85 | 75.0% |
| 15genes = SEQ ID NO: 70-83 + SEQ ID NO: 86 | 72.9% |
| 15genes = SEQ ID NO: 70-83 + SEQ ID NO: 87 | 75.0% |
| 15genes = SEQ ID NO: 70-83 + SEQ ID NO: 88 | 77.1% |
| 15genes = SEQ ID NO: 70-83 + SEQ ID NO: 89 | 75.0% |
| 15genes = SEQ ID NO: 70-83 + SEQ ID NO: 90 | 75.0% |
| 15genes = SEQ ID NO: 70-83 + SEQ ID NO: 91 | 75.0% |
| 15genes = SEQ ID NO: 70-83 + SEQ ID NO: 92 | 75.0% |
| 15genes = SEQ ID NO: 70-83 + SEQ ID NO: 93 | 72.9% |
| 15genes = SEQ ID NO: 70-83 + SEQ ID NO: 94 | 72.9% |
| 15genes = SEQ ID NO: 70-83 + SEQ ID NO: 95 | 75.0% |
| 15genes = SEQ ID NO: 70-83 + SEQ ID NO: 96 | 75.0% |
| 15genes = SEQ ID NO: 70-83 + SEQ ID NO: 97 | 72.9% |
| 15genes = SEQ ID NO: 70-83 + SEQ ID NO: 88 | 77.1% |
| 16genes = SEQ ID NO: 70-83 + SEQ ID NO: 88 + SEQ ID NO: 84 | 77.1% |
| 16genes = SEQ ID NO: 70-83 + SEQ ID NO: 88 + SEQ ID NO: 85 | 81.3% |
| 16genes = SEQ ID NO: 70-83 + SEQ ID NO: 88 + SEQ ID NO: 86 | 77.1% |
| 16genes = SEQ ID NO: 70-83 + SEQ ID NO: 88 + SEQ ID NO: 87 | 79.2% |
| 16genes = SEQ ID NO: 70-83 + SEQ ID NO: 88 + SEQ ID NO: 89 | 77.1% |
| 16genes = SEQ ID NO: 70-83 + SEQ ID NO: 88 + SEQ ID NO: 90 | 79.2% |
| 16genes = SEQ ID NO: 70-83 + SEQ ID NO: 88 + SEQ ID NO: 91 | 79.2% |
| 16genes = SEQ ID NO: 70-83 + SEQ ID NO: 88 + SEQ ID NO: 92 | 79.2% |
| 16genes = SEQ ID NO: 70-83 + SEQ ID NO: 88 + SEQ ID NO: 93 | 77.1% |
| 16genes = SEQ ID NO: 70-83 + SEQ ID NO: 88 + SEQ ID NO: 94 | 79.2% |
| 16genes = SEQ ID NO: 70-83 + SEQ ID NO: 88 + SEQ ID NO: 95 | 79.2% |
| 16genes = SEQ ID NO: 70-83 + SEQ ID NO: 88 + SEQ ID NO: 96 | 79.2% |
| 16genes = SEQ ID NO: 70-83 + SEQ ID NO: 88 + SEQ ID NO: 97 | 77.1% |
| 16genes = SEQ ID NO: 70-83 + SEQ ID NO: 85 + SEQ ID NO: 88 | 81.3% |
| 17genes = SEQ ID NO: 70-83 + SEQ ID NO: 85 + SEQ ID NO: 88 + | 81.3% |
| SEQ ID NO: 84 | |
| 17genes = SEQ ID NO: 70-83 + SEQ ID NO: 85 + SEQ ID NO: 88 + | 81.3% |
| SEQ ID NO: 86 | |
| 17genes = SEQ ID NO: 70-83 + SEQ ID NO: 85 + SEQ ID NO: 88 + | 83.3% |
| SEQ ID NO: 87 | |
| 17genes = SEQ ID NO: 70-83 + SEQ ID NO: 85 + SEQ ID NO: 88 + | 81.3% |
| SEQ ID NO: 89 | |
| 17genes = SEQ ID NO: 70-83 + SEQ ID NO: 85 + SEQ ID NO: 88 + | 81.3% |
| SEQ ID NO: 90 | |
| 17genes = SEQ ID NO: 70-83 + SEQ ID NO: 85 + SEQ ID NO: 88 + | 83.3% |
| SEQ ID NO: 91 | |
| 17genes = SEQ ID NO: 70-83 + SEQ ID NO: 85 + SEQ ID NO: 88 + | 83.3% |
| SEQ ID NO: 92 | |
| 17genes = SEQ ID NO: 70-83 + SEQ ID NO: 85 + SEQ ID NO: 88 + | 81.3% |
| SEQ ID NO: 93 | |
| 17genes = SEQ ID NO: 70-83 + SEQ ID NO: 85 + SEQ ID NO: 88 + | 83.3% |
| SEQ ID NO: 94 | |
| 17genes = SEQ ID NO: 70-83 + SEQ ID NO: 85 + SEQ ID NO: 88 + | 83.3% |
| SEQ ID NO: 95 | |
| 17genes = SEQ ID NO: 70-83 + SEQ ID NO: 85 + SEQ ID NO: 88 + | 83.3% |
| SEQ ID NO: 96 | |
| 17genes = SEQ ID NO: 70-83 + SEQ ID NO: 85 + SEQ ID NO: 88 + | 81.3% |
| SEQ ID NO: 97 | |
| 17genes = SEQ ID NO: 70-83 + SEQ ID NO: 85 + SEQ ID NO: 87-88 | 83.3% |
| 18genes = SEQ ID NO: 70-83 + SEQ ID NO: 85 + SEQ ID NO: 87-88 + | 85.4% |
| SEQ ID NO: 84 | |
| 18genes = SEQ ID NO: 70-83 + SEQ ID NO: 85 + SEQ ID NO: 87-88 + | 85.4% |
| SEQ ID NO: 86 | |
| 18genes = SEQ ID NO: 70-83 + SEQ ID NO: 85 + SEQ ID NO: 87-88 + | 83.3% |
| SEQ ID NO: 89 | |
| 18genes = SEQ ID NO: 70-83 + SEQ ID NO: 85 + SEQ ID NO: 87-88 + | 83.3% |
| SEQ ID NO: 90 | |
| 18genes = SEQ ID NO: 70-83 + SEQ ID NO: 85 + SEQ ID NO: 87-88 + | 85.4% |
| SEQ ID NO: 91 | |
| 18genes = SEQ ID NO: 70-83 + SEQ ID NO: 85 + SEQ ID NO: 87-88 + | 85.4% |
| SEQ ID NO: 92 | |
| 18genes = SEQ ID NO: 70-83 + SEQ ID NO: 85 + SEQ ID NO: 87-88 + | 85.4% |
| SEQ ID NO: 93 | |
| 18genes = SEQ ID NO: 70-83 + SEQ ID NO: 85 + SEQ ID NO: 87-88 + | 85.4% |
| SEQ ID NO: 94 | |
| 18genes = SEQ ID NO: 70-83 + SEQ ID NO: 85 + SEQ ID NO: 87-88 + | 85.4% |
| SEQ ID NO: 95 | |
| 18genes = SEQ ID NO: 70-83 + SEQ ID NO: 85 + SEQ ID NO: 87-88 + | 85.4% |
| SEQ ID NO: 96 | |
| 18genes = SEQ ID NO: 70-83 + SEQ ID NO: 85 + SEQ ID NO: 87-88 + | 83.3% |
| SEQ ID NO: 97 | |
| 18genes = SEQ ID NO: 70-83 + SEQ ID NO: 84-85 + SEQ ID NO: 87-88 | 85.4% |
| 19genes = SEQ ID NO: 70-83 + SEQ ID NO: 84-85 + SEQ ID NO: 87-88 + | 87.5% |
| SEQ ID NO: 86 | |
| 19genes = SEQ ID NO: 70-83 + SEQ ID NO: 84-85 + SEQ ID NO: 87-88 + | 85.4% |
| SEQ ID NO: 90 | |
| 19genes = SEQ ID NO: 70-83 + SEQ ID NO: 84-85 + SEQ ID NO: 87-88 + | 85.4% |
| SEQ ID NO: 91 | |
| 19genes = SEQ ID NO: 70-83 + SEQ ID NO: 84-85 + SEQ ID NO: 87-88 + | 87.5% |
| SEQ ID NO: 92 | |
| 19genes = SEQ ID NO: 70-83 + SEQ ID NO: 84-85 + SEQ ID NO: 87-88 + | 87.5% |
| SEQ ID NO: 92 | |
| 19genes = SEQ ID NO: 70-83 + SEQ ID NO: 84-85 + SEQ ID NO: 87-88 + | 87.5% |
| SEQ ID NO: 93 | |
| 19genes = SEQ ID NO: 70-83 + SEQ ID NO: 84-85 + SEQ ID NO: 87-88 + | 87.5% |
| SEQ ID NO: 94 | |
| 19genes = SEQ ID NO: 70-83 + SEQ ID NO: 84-85 + SEQ ID NO: 87-88 + | 87.5% |
| SEQ ID NO: 95 | |
| 19genes = SEQ ID NO: 70-83 + SEQ ID NO: 84-85 + SEQ ID NO: 87-88 + | 87.5% |
| SEQ ID NO: 96 | |
| 19genes = SEQ ID NO: 70-83 + SEQ ID NO: 84-85 + SEQ ID NO: 87-88 + | 85.4% |
| SEQ ID NO: 97 | |
| 19genes = SEQ ID NO: 70-88 | 87.5% |
| 20genes = SEQ ID NO: 70-88 + SEQ ID NO: 89 | 87.5% |
| 20genes = SEQ ID NO: 70-88 + SEQ ID NO: 90 | 87.5% |
| 20genes = SEQ ID NO: 70-88 + SEQ ID NO: 91 | 89.6% |
| 20genes = SEQ ID NO: 70-88 + SEQ ID NO: 92 | 89.6% |
| 20genes = SEQ ID NO: 70-88 + SEQ ID NO: 93 | 87.5% |
| 20genes = SEQ ID NO: 70-88 + SEQ ID NO: 94 | 89.6% |
| 20genes = SEQ ID NO: 70-88 + SEQ ID NO: 95 | 89.6% |
| 20genes = SEQ ID NO: 70-88 + SEQ ID NO: 96 | 89.6% |
| 20genes = SEQ ID NO: 70-88 + SEQ ID NO: 97 | 87.5% |
| 20genes = SEQ ID NO: 70-88 + SEQ ID NO: 91 | 89.6% |
| 21genes = SEQ ID NO: 70-88 + SEQ ID NO: 91 + SEQ ID NO: 89 | 89.6% |
| 21genes = SEQ ID NO: 70-88 + SEQ ID NO: 91 + SEQ ID NO: 90 | 89.6% |
| 21genes = SEQ ID NO: 70-88 + SEQ ID NO: 91 + SEQ ID NO: 92 | 91.7% |
| 21genes = SEQ ID NO: 70-88 + SEQ ID NO: 91 + SEQ ID NO: 93 | 89.6% |
| 21genes = SEQ ID NO: 70-88 + SEQ ID NO: 91 + SEQ ID NO: 94 | 91.7% |
| 21genes = SEQ ID NO: 70-88 + SEQ ID NO: 91 + SEQ ID NO: 95 | 91.7% |
| 21genes = SEQ ID NO: 70-88 + SEQ ID NO: 91 + SEQ ID NO: 96 | 91.7% |
| 21genes = SEQ ID NO: 70-88 + SEQ ID NO: 91 + SEQ ID NO: 97 | 89.6% |
| 21genes = SEQ ID NO: 70-88 + SEQ ID NO: 91 + SEQ ID NO: 92 | 91.7% |
| 22genes = SEQ ID NO: 70-88 + SEQ ID NO: 91-92 + SEQ ID NO: 89 | 91.7% |
| 22genes = SEQ ID NO: 70-88 + SEQ ID NO: 91-92 + SEQ ID NO: 90 | 91.7% |
| 22genes = SEQ ID NO: 70-88 + SEQ ID NO: 91-92 + SEQ ID NO: 93 | 91.7% |
| 22genes = SEQ ID NO: 70-88 + SEQ ID NO: 91-92 + SEQ ID NO: 94 | 93.8% |
| 22genes = SEQ ID NO: 70-88 + SEQ ID NO: 91-92 + SEQ ID NO: 95 | 93.8% |
| 22genes = SEQ ID NO: 70-88 + SEQ ID NO: 91-92 + SEQ ID NO: 96 | 93.8% |
| 22genes = SEQ ID NO: 70-88 + SEQ ID NO: 91-92 + SEQ ID NO: 97 | 91.7% |
| 22genes = SEQ ID NO: 70-88 + SEQ ID NO: 91-92 + SEQ ID NO: 94 | 93.8% |
| 23genes = SEQ ID NO: 70-88 + SEQ ID NO: 91-92 + SEQ ID NO: 94 + | 93.8% |
| SEQ ID NO: 89 | |
| 23genes = SEQ ID NO: 70-88 + SEQ ID NO: 91-92 + SEQ ID NO: 94 + | 93.8% |
| SEQ ID NO: 90 | |
| 23genes = SEQ ID NO: 70-88 + SEQ ID NO: 91-92 + SEQ ID NO: 94 + | 93.8% |
| SEQ ID NO: 93 | |
| 23genes = SEQ ID NO: 70-88 + SEQ ID NO: 91-92 + SEQ ID NO: 94 + | 95.8% |
| SEQ ID NO: 95 | |
| 23genes = SEQ ID NO: 70-88 + SEQ ID NO: 91-92 + SEQ ID NO: 94 + | 95.8% |
| SEQ ID NO: 96 | |
| 23genes = SEQ ID NO: 70-88 + SEQ ID NO: 91-92 + SEQ ID NO: 94 + | 93.8% |
| SEQ ID NO: 97 | |
| 23genes = SEQ ID NO: 70-88 + SEQ ID NO: 91-92 + SEQ ID NO: 94-95 | 95.8% |
| 24genes = SEQ ID NO: 70-88 + SEQ ID NO: 91-92 + SEQ ID NO: 94-95 + | 95.8% |
| SEQ ID NO: 89 | |
| 24genes = SEQ ID NO: 70-88 + SEQ ID NO: 91-92 + SEQ ID NO: 94-95 + | 95.8% |
| SEQ ID NO: 90 | |
| 24genes = SEQ ID NO: 70-88 + SEQ ID NO: 91-92 + SEQ ID NO: 94-95 + | 95.8% |
| SEQ ID NO: 93 | |
| 24genes = SEQ ID NO: 70-88 + SEQ ID NO: 91-92 + SEQ ID NO: 94-95 + | 97.9% |
| SEQ ID NO: 96 | |
| 24genes = SEQ ID NO: 70-88 + SEQ ID NO: 91-92 + SEQ ID NO: 94-95 + | 95.8% |
| SEQ ID NO: 97 | |
| 25genes = SEQ ID NO: 70-88 + SEQ ID NO: 91-92 + SEQ ID NO: 94-96 + | 97.9% |
| SEQ ID NO: 89 | |
| 25genes = SEQ ID NO: 70-88 + SEQ ID NO: 91-92 + SEQ ID NO: 94-96 + | 97.9% |
| SEQ ID NO: 90 | |
| 25genes = SEQ ID NO: 70-88 + SEQ ID NO: 91-92 + SEQ ID NO: 94-96 + | 97.9% |
| SEQ ID NO: 93 | |
| 25genes = SEQ ID NO: 70-88 + SEQ ID NO: 91-92 + SEQ ID NO: 94-96 + | 97.9% |
| SEQ ID NO: 97 | |
| 25genes = = SEQ ID NO: 70-89 + SEQ ID NO: 91-92 + SEQ ID NO: 94-96 | 97.9% |
| 25genes = SEQ ID NO: 70-88 + SEQ ID NO: 90-92 + SEQ ID NO: 94-96 | 97.9% |
| 25genes = SEQ ID NO: 70-88 + SEQ ID NO: 91-96 | 97.9% |
| 25genes = SEQ ID NO: 70-88 + SEQ ID NO: 91-92 + SEQ ID NO: 94-97 | 97.9% |
| 26 genes = SEQ ID NO: 70-92 + SEQ ID NO: 94-96 | 100.0% |
| 26 genes = SEQ ID NO: 70-89 + SEQ ID NO: 91-96 | 97.9% |
| 26 genes = SEQ ID NO: 70-89 + SEQ ID NO: 91-92 + SEQ ID NO: 94-97 | 97.9% |
| 26 genes = SEQ ID NO: 70-92 + SEQ ID NO: 94-96 | 100.0% |
| 26 genes = SEQ ID NO: 70-88 + SEQ ID NO: 90-96 | 97.9% |
| 26 genes = SEQ ID NO: 70-88 + SEQ ID NO: 90-92 + SEQ ID NO: 94-97 | 97.9% |
| 26 genes = SEQ ID NO: 70-89 + SEQ ID NO: 91-96 | 97.9% |
| 26 genes = SEQ ID NO: 70-88 + SEQ ID NO: 90-96 | 97.9% |
| 26 genes = SEQ ID NO: 70-88 + SEQ ID NO: 91-97 | 97.9% |
| 26 genes = SEQ ID NO: 70-89 + SEQ ID NO: 91-97 | 97.9% |
| 26 genes = SEQ ID NO: 70-88 + SEQ ID NO: 90-97 | 97.9% |
| 26 genes = SEQ ID NO: 70-89 + SEQ ID NO: 91-96 | 97.9% |
| 27genes SEQ ID NO: 70-96 | 100.0% |
| 27 genes SEQ ID NO: 70-89 and SEQ ID NO: 91-97 | 97.9% |
| 28 genes SEQ ID NO: 70-97 | 100% |

The invention also relates to a method, preferably in vitro, for identifying patient afflicted by a lung cancer having of the survival rate of at most about 20% at 30 months, among a population of patients afflicted by lung cancer having an estimated survival rate of at least about 30% at 30 months based on the diagnosis of said lung cancer according to histopathological criteria,

said method allowing the identification of at least 66% of patient of those afflicted by a lung cancer having of the survival rate of at most about 20% at 30 months,
said method comprising
a step of measuring, in a biological sample of said patients, the expression of
   at least 13 genes chosen among a set of 28 genes comprising or consisting of the nucleic acid sequences SEQ ID NO 70 to 97,
   or fragments of said genes
   or complementary sequences of said genes
said at least 13 genes being such that
   12 genes comprise or consist of the nucleic acid sequences SEQ ID NO: 70 to 81, and
   at least one gene belongs to a subset of 16 genes comprising or consisting of the nucleic acid sequences SEQ ID NO: 82-97, and
a step of identifying biological samples expressing said at least 13 genes.

Advantageously, the invention relates to the method defined above, wherein said at least 13 genes being such that
12 genes comprise or consist of the nucleic acid sequences SEQ ID NO: 70 to 81, and
at least one gene belongs to a subset of 16 genes comprising or consisting of the nucleic acid sequences SEQ ID NO: 82-97, said at least one gene comprising or consisting of the nucleic acid sequences SEQ ID NO: 82,
   said method allowing the identification of at least 70% of patients of those afflicted by a lung cancer having of the survival rate of at most about 20% at 30 months.

In one advantageous embodiment, the invention relates to a method, according to the above definition, said method comprising a step of measuring, in a biological sample of said patients, the expression of at least 18 genes chosen among a set of 28 genes comprising or consisting of the nucleic acid sequences SEQ ID NO 70 to 97,

said at least 18 genes being such that
12 genes comprise or consist of the nucleic acid sequences SEQ ID NO: 70 to 81, and
at least 6 genes belongs to a subset of 16 genes comprising or consisting of the nucleic acid sequences SEQ ID NO: 82-97, said at least 6 genes comprising or consisting of the nucleic acid sequences SEQ ID NO:82-85 and 87-88,
   said method allowing the identification of at least 83% of patients of those having a survival rate of at most about 20% at 30 months.

In one advantageous embodiment, the invention relates to a method, according to the above definition, said method comprising a step of measuring, in a biological sample of said patients, the expression of at least 21 genes chosen among a set of 28 genes comprising or consisting of the nucleic acid sequences SEQ ID NO 70 to 97,

said at least 21 genes being such that
12 genes comprise or consist of the nucleic acid sequences SEQ ID NO: 70 to 81, and
at least 9 gene belongs to a subset of 16 genes comprising or consisting of the nucleic acid sequences SEQ ID NO: 82-97, said at least 9 genes preferably comprising or consisting of the nucleic acid sequences SEQ ID NO: 82-88 and 91-92,
   said method allowing identifying at least 89% of patients having a survival rate of at most about 20% at 30 months.

In one advantageous embodiment, the invention relates to a method, according to the above definition, said method comprising a step of measuring, in a biological sample of said patients, the expression of at least 26 genes chosen among a set of 28 genes comprising or consisting of the nucleic acid sequences SEQ ID NO 70 to 97,

said at least 26 genes being such that
12 genes comprise or consist of the nucleic acid sequences SEQ ID NO: 70 to 81, and
at least 14 gene belongs to a subset of 16 genes comprising or consisting of the nucleic acid sequences SEQ ID NO: 82-97, said at least 14 genes preferably comprising or consisting of the nucleic acid sequences SEQ ID NO: 82-92 and 93-96,
   said method allowing identifying at least 97% of patients of those having a survival rate of at most about 20% at 30 months.

In one advantageous embodiment, the invention relates to a method, according to the above definition, said method comprising a step of measuring, in a biological sample of said patients, the expression of at least 27 genes chosen among a set of 28 genes comprising or consisting of the nucleic acid sequences SEQ ID NO 70 to 97,

said at least 26 genes being such that
12 genes comprise or consist of the nucleic acid sequences SEQ ID NO: 70 to 81, and
at least 15 gene belongs to a subset of 16 genes comprising or consisting of the nucleic acid sequences SEQ ID NO: 82-97,
   said method allowing identifying at least 97% of patients of those having a survival rate of at most about 20% at 30 months.

In one advantageous embodiment, the invention relates to a method, according to the above definition, said method comprising a step of measuring, in a biological sample of said patients, the expression of 28 genes chosen comprising or consisting of the nucleic acid sequences SEQ ID NO 70 to 97,

said method allowing the identification of 100% of patient afflicted by a lung cancer having of the survival rate of at most about 20% at 30 months
said method comprising
   a step of measuring, in a biological sample of said patients, the expression of 28 comprising or consisting of the nucleic acid sequences SEQ ID NO 70 to 97, and
   a step of identifying biological samples expressing said 28 genes.

In another advantageous embodiment, the invention relates to a method as previously defined, wherein the step of measuring is carried out by using nucleic acid molecules consisting of from 15 to 100 nucleotides molecules being complementary to said at least 13 genes, or said at least 18 genes, or said at least 21 genes, or said at least 26 genes, or said 28 genes.

The skilled person can easily carry out the above method by choosing the appropriate means allowing the detection of the genes as defined above.

The above method applies mutatis mutandis using the proteins coded by the at least 13 genes as defined above, or by using antibodies recognizing the proteins coded by the at least 13 genes as defined above.

The invention is illustrated by the following 65 figures and the two examples.

### LEGEND TO THE FIGURES

FIG. 1 represents the cumulative survival rate (Kaplan Mayer curve) over 60 months of patients expressing (A) or not (B) the gene SEQ ID NO: 4 (gene 1161). Y-axis represents cumulative survival in %, X-axis represents time in months.

FIG. 2 represents the cumulative survival rate (Kaplan Mayer curve) over 60 months of patients expressing (A) or not (B) the gene SEQ ID NO: 4 (gene 1161) and or the gene SEQ ID NO: 6 (gene 391). Y-axis represents cumulative survival in %, X-axis represents time in months.

FIG. 3 represents the cumulative survival rate (Kaplan Mayer curve) over 60 months of patients expressing (A) or not (B) the gene SEQ ID NO: 4 (gene 1161) and/or the gene SEQ ID NO: 6 (gene 391) and/or the gene SEQ ID NO: 2 (gene 35). Y-axis represents cumulative survival in %, X-axis represents time in months.

FIG. 4 represents the cumulative survival rate (Kaplan Mayer curve) over 60 months of patients expressing (A) or not (B) at least one of the following genes: SEQ ID NO: 4 (gene 1161), SEQ ID NO: 6 (gene 391), SEQ ID NO: 2 (gene 35) and SEQ ID NO: 1(gene 442). Y-axis represents cumulative survival in %, X-axis represents time in months.

FIG. 5 represents the cumulative survival rate (Kaplan Mayer curve) over 60 months of patients expressing (A) or not (B) at least one of the following genes: SEQ ID NO: 4 (gene 1161), SEQ ID NO: 6 (gene 391), SEQ ID NO: 2 (gene 35), SEQ ID NO: 1(gene 442) and SEQ ID NO 5 (gene 102). Y-axis represents cumulative survival in %, X-axis represents time in months.

FIG. 6 represents the cumulative survival rate (Kaplan Mayer curve) over 60 months of patients expressing (A) or not (B) at least one of the following genes: SEQ ID NO: 4 (gene 1161), SEQ ID NO: 6 (gene 391), SEQ ID NO: 2 (gene 35), SEQ ID NO: 1(gene 442), SEQ ID NO 5 (gene 102) and SEQ ID NO:7 (gene 390). Y-axis represents cumulative survival in %, X-axis represents time in months.

FIG. 7 represents the cumulative survival rate (Kaplan Mayer curve) over 60 months of patients expressing (A) or not (B) at least one of the genes: SEQ ID NO: 1 to 7. Y-axis represents cumulative survival in %, X-axis represents time in months.

FIG. 8 represents the cumulative survival rate (Kaplan Mayer curve) over 60 months of patients expressing one or two (B), or three or more (A) or no (C) genes: SEQ ID NO: 1 to 7. Y-axis represents cumulative survival in %, X-axis represents time in months.

FIG. 9 represents the cumulative survival rate (Kaplan Mayer curve) over 60 months of patients expressing one (C) or two (B), or three or more (A) or no (D) genes: SEQ ID NO: 1 to 7. Y-axis represents cumulative survival in %, X-axis represents time in months.

FIG. 10 represents the cumulative survival rate (Kaplan Mayer curve) over 60 months of patients expressing (A) or not (B) the gene SEQ ID NO: 16 (gene 125). Y-axis represents cumulative survival in %, X-axis represents time in months.

FIG. 11 represents the cumulative survival rate (Kaplan Mayer curve) over 60 months of patients expressing (A) or not (B) at least one of the following genes: SEQ ID NO: 16 (gene 125) and SEQ ID NO: 22 (gene 117). Y-axis represents cumulative survival in %, X-axis represents time in months.

FIG. 12 represents the cumulative survival rate (Kaplan Mayer curve) over 60 months of patients expressing (A) or not (B) at least one of the following genes: SEQ ID NO: 16 (gene 125), SEQ ID NO: 22 (gene 117) and SEQ ID NO:19 (766). Y-axis represents cumulative survival in %, X-axis represents time in months.

FIG. 13 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766) and SEQ ID NO: 17(gene 144). Y-axis represents cumulative survival in %, X-axis represents time in months.

FIG. 14 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144) and SEQ ID NO: 12 (gene 108). Y-axis represents cumulative survival in %, X-axis represents time in months.

FIG. 15 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108) and SEQ ID NO: 8 (gene 222). Y-axis represents cumulative survival in %, X-axis represents time in months.

FIG. 16 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222) and SEQ ID NO: 17 (gene 72). Y-axis represents cumulative survival in %, X-axis represents time in months.

FIG. 17 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72) and SEQ ID NO: 10 (gene 1165). Y-axis represents cumulative survival in %, X-axis represents time in months.

FIG. 18 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165) and SEQ ID NO: 21 (gene 487). Y-axis represents cumulative survival in %, X-axis represents time in months.

FIG. 19 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487) and SEQ ID NO: 9(gene 1261). Y-axis represents cumulative survival in %, X-axis represents time in months.

FIG. 20 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9(gene 1261) and SEQ ID N NO: 13 (gene 205). Y-axis represents cumulative survival in %, X-axis represents time in months.

FIG. 21 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9(gene 1261), SEQ ID N NO: 13 (gene 205) and SEQ ID NO: 18 (gene 437). Y-axis represents cumulative survival in %, X-axis represents time in months.

FIG. 22 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9(gene 1261), SEQ ID N NO: 13 (gene 205), SEQ ID NO: 18 (gene 437) and SEQ ID NO:15 (gene 1328). Y-axis represents cumulative survival in %, X-axis represents time in months.

FIG. 23 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9(gene 1261), SEQ ID N NO: 13 (gene 205), SEQ ID NO: 18 (gene 437), SEQ ID NO:15 (gene 1328) and SEQ ID NO: 14 (gene 1188). Y-axis represents cumulative survival in %, X-axis represents time in months.

FIG. 24 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9(gene 1261), SEQ ID N NO: 13 (gene 205), SEQ ID NO: 18 (gene 437), SEQ ID NO:15 (gene 1328), SEQ ID NO: 14 (gene 1188) and SEQ ID NO: 20 (gene 436). Y-axis represents cumulative survival in %, X-axis represents time in months.

FIG. 25 represents the cumulative survival rate (Kaplan Mayer curve) over 60 months of patients expressing (A) or not (B) at least one of the following genes: SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9(gene 1261), SEQ ID N NO: 13 (gene 205), SEQ ID NO: 18 (gene 437), SEQ ID NO:15 (gene 1328), SEQ ID NO: 14 (gene 1188), SEQ ID NO: 20 (gene 436) and SEQ ID NO: 23 (gene 135). Y-axis represents cumulative survival in %, X-axis represents time in months.

FIG. 26 represents the cumulative survival rate (Kaplan Mayer curve) over 60 months of patients expressing one (B), or two or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487) and SEQ ID NO: 9 (gene 1261). Y-axis represents cumulative survival in %, X-axis represents time in months.

FIG. 27 represents the cumulative survival rate (Kaplan Mayer curve) over 60 months of patients expressing one (B), or two or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9 (gene 1261) and SEQ ID N NO: 13 (gene 205). Y-axis represents cumulative survival in %, X-axis represents time in months.

FIG. 28 represents the cumulative survival rate (Kaplan Mayer curve) over 60 months of patients expressing one (B), or two or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9 (gene 1261), SEQ ID N NO: 13 (gene 205) and SEQ ID NO: 18 (gene 437). Y-axis represents cumulative survival in %, X-axis represents time in months.

FIG. 29 represents the cumulative survival rate (Kaplan Mayer curve) over 60 months of patients expressing one (B), or two or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9 (gene 1261), SEQ ID N NO: 13 (gene 205), SEQ ID NO: 18 (gene 437) and SEQ ID NO:15 (gene 1328). Y-axis represents cumulative survival in %, X-axis represents time in months.

FIG. 30 represents the cumulative survival rate (Kaplan Mayer curve) over 60 months of patients expressing one (B), or two or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9 (gene 1261), SEQ ID N NO: 13 (gene 205), SEQ ID NO: 18 (gene 437), SEQ ID NO:15 (gene 1328) and SEQ ID NO: 14 (gene 1188). Y-axis represents cumulative survival in %, X-axis represents time in months.

FIG. 31 represents the cumulative survival rate (Kaplan Mayer curve) over 60 months of patients expressing one (B), or two or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9 (gene 1261), SEQ ID N NO: 13 (gene 205), SEQ ID NO: 18 (gene 437), SEQ ID NO:15 (gene 1328), SEQ ID NO: 14 (gene 1188) and SEQ ID NO: 20 (gene 436). Y-axis represents cumulative survival in %, X-axis represents time in months.

FIG. 32 represents the cumulative survival rate (Kaplan Mayer curve) over 60 months of patients expressing one (B), or two or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9 (gene 1261), SEQ ID N NO: 13 (gene 205), SEQ ID NO: 18 (gene 437), SEQ ID NO:15 (gene 1328), SEQ ID NO: 14 (gene 1188), SEQ ID NO: (gene 436) and SEQ ID NO: 23 (gene 135). Y-axis represents cumulative survival in %, X-axis represents time in months.

FIG. 33 represents the cumulative survival rate (Kaplan Mayer curve) over 60 months of patients expressing one (D), or two (B) or three or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9 (gene 1261), SEQ ID N NO: 13 (gene 205), SEQ ID NO: 18 (gene 437), SEQ ID NO:15 (gene 1328), SEQ ID NO: 14 (gene 1188) and SEQ ID NO: 20 (gene 436). Y-axis represents cumulative survival in %, X-axis represents time in months.

FIG. 34 represents the cumulative survival rate (Kaplan Mayer curve) over 60 months of patients expressing one (C), or two (B) or three or more (A) or no (C) genes chosen among SEQ ID NO: 16 (gene 125), SEQ ID NO: 22 (gene 117), SEQ ID NO:19 (gene 766), SEQ ID NO: 17(gene 144), SEQ ID NO: 12 (gene 108), SEQ ID NO: 8 (gene 222), SEQ ID NO: 17 (gene 72), SEQ ID NO: 10 (gene 1165), SEQ ID NO: 21 (gene 487), SEQ ID NO: 9 (gene 1261), SEQ ID N NO: 13 (gene 205), SEQ ID NO: 18 (gene 437), SEQ ID NO:15 (gene 1328), SEQ ID NO: 14 (gene 1188), SEQ ID NO: 20 (gene 436) and SEQ ID NO: 23 (gene 135). Y-axis represents cumulative survival in %, X-axis represents time in months.

FIG. 35 represents the cumulative survival rate (Kaplan Mayer curve) over 60 months of patients expressing no (B), or at least one gene SEQ ID NO:1-23. Y-axis represents cumulative survival in %, X-axis represents time in months.

FIG. 36 represents the cumulative survival rate (Kaplan Mayer curve) over 60 months of patients expressing no (C),

or none of the genes SEQ ID NO: 8-23 and at least 3 genes SEQ ID NO: 1-7 are expressed, or at least 1 gene SEQ ID NO: 8-23 is expressed and from none to 2 genes SEQ ID NO: 1-7 is expressed, or at least 2 genes SEQ ID NO: 8-23 are expressed and no gene SEQ ID NO: 1-7 is expressed (B),
or one gene SEQ ID NO: 8-23 is expressed and at least 3 genes SEQ ID NO: 1-7 are expressed, or at least 2 genes SEQ ID NO: 8-23 are expressed and at least 1 gene SEQ ID NO: 1-7 is expressed (A).
Y-axis represents cumulative survival in %, X-axis represents time in months.

FIG. 37 represents the cumulative survival rate (Kaplan Mayer curve) over 60 months of patients expressing no (D),

or if none of the genes SEQ ID NO: 8-23 is expressed and one or two genes SEQ ID NO: 1-7 is expressed (C)
or none of the genes SEQ ID NO: 8-23 and at least 3 genes SEQ ID NO: 1-7 are expressed, or at least 1 gene SEQ ID NO: 8-23 is expressed and from none to 2 genes SEQ ID NO: 1-7 is expressed, or at least 2 genes SEQ ID NO: 8-23 are expressed and no gene SEQ ID NO: 1-7 is expressed (B),
or one gene SEQ ID NO: 8-23 is expressed and at least 3 genes SEQ ID NO: 1-7 are expressed, or at least 2 genes SEQ ID NO: 8-23 are expressed and at least 1 gene SEQ ID NO: 1-7 is expressed (A).
Y-axis represents cumulative survival in %, X-axis represents time in months.

FIG. 38 represents the cumulative survival rate (Kaplan Mayer curve) over 60 months of patients expressing no genes SEQ ID NO:1-23 (A) or expressing LDHC gene (B).

FIG. 39 represents the cumulative survival rate (Kaplan Mayer curve) over 60 months of patients expressing no genes SEQ ID NO:1-23 (A) or expressing MAGEA5 gene (B).

FIG. 40 represents the cumulative survival rate (Kaplan Mayer curve) over 60 months of patients expressing no genes SEQ ID NO:1-23 (A) or expressing MAGEB18 gene (B).

FIGS. 41-65 refer to the at least 13 genes chosen among 28 genes comprising or consisting of SEQ ID NO: 70-97.

FIG. 41 represents the global survival probability over 5 years of 300 lung cancer patients.

FIG. 42 represents the global survival probability over 5 years of 300 lung cancer patients by using the invention.

FIG. 43 represents the global survival probability over 5 years of patients with T1N0 (early stages) or advanced stages (T+N+) of lung cancer according to the TNM classification.

FIG. 44 represents the global survival probability over 5 years of patients with T1N0 (early stages) of lung cancer by using the invention.

FIG. 45 represents the global survival probability over 5 years of patients with advanced stages (T+N+) of lung cancer by using the invention.

FIG. 46 represents the respective global survival probabilities over 5 years of patients with BAS, SQC and LCNE lung cancer according to the histopathological classification.

FIG. 47 represents the global survival probability over 5 years of patients with BAS lung cancer by using the invention.

FIG. 48 represents the global survival probability over 5 years of patients with SQC lung cancer by using the invention.

FIG. 49 represents the global survival probability over 5 years of patients with LCNE lung cancer by using the invention.

FIG. 50 represents the global survival probability over 30 months of 300 lung cancer patients by using 13 genes of invention.

FIG. 51 represents the global survival probability over 30 months of 300 lung cancer patients by using 14 genes of invention

FIG. 52 represents the global survival probability over 30 months of 300 lung cancer patients by using 15 genes of invention

FIG. 53 represents the global survival probability over 30 months of 300 lung cancer patients by using 16 genes of invention

FIG. 54 represents the global survival probability over 30 months of 300 lung cancer patients by using 17 genes of invention

FIG. 55 represents the global survival probability over 30 months of 300 lung cancer patients by using 18 genes of invention

FIG. 56 represents the global survival probability over 30 months of 300 lung cancer patients by using 19 genes of invention

FIG. 57 represents the global survival probability over 30 months of 300 lung cancer patients by using 20 genes of invention

FIG. 58 represents the global survival probability over 30 months of 300 lung cancer patients by using 21 genes of invention

FIG. 59 represents the global survival probability over 30 months of 300 lung cancer patients by using 22 genes of invention

FIG. 60 represents the global survival probability over 30 months of 300 lung cancer patients by using 23 genes of invention

FIG. 61 represents the global survival probability over 30 months of 300 lung cancer patients by using 24 genes of invention

FIG. 62 represents the global survival probability over 30 months of 300 lung cancer patients by using 25 genes of invention

FIG. 63 represents the global survival probability over 30 months of 300 lung cancer patients by using 26 genes of invention

FIG. 64 represents the global survival probability over 30 months of 300 lung cancer patients by using 27 genes of invention

FIG. 65 represents the global survival probability over 30 months of 300 lung cancer patients by using 28 genes of invention

### EXAMPLES

### Example 1

The invention describes a group of 23 genes, which can be used to establish the survival prognosis of lung tumour patients. All these genes are actively repressed and silent in normal adult somatic cells, since their expression is strictly restricted to placenta or male germinal cells. The inventors have demonstrated that the aberrant expression in malignant cells of at least one of these genes is associated with significantly poorer prognosis for lung cancer patients. Moreover, the detection of the expression of several combinations of these genes allows predicting prognosis in lung tumour patients with higher significance and accuracy than with individual genes.

The invention has led to the identification of 23 genes, whose aberrant expression was found associated with poor prognosis in lung tumour patients.

According to their expression and prognosis value in lung cancer patients, these 23 genes were divided into two groups
A group of 7 genes, whose aberrant expression in lung cancer is relatively frequent (>7% of cases of our series). The expression of each individual one of these seven genes is associated with a significantly reduced survival probability (global or disease free survival over five years significantly reduced, logrank test p<0.07) of all lung cancer patients (without considering histological subtypes).
A group of 16 genes, whose aberrant expression in lung cancer is relatively rare (<7% of cases of our series). The expression of each individual one of these seven genes is associated with a significantly reduced survival probability (global or disease free survival over five years significantly reduced, logrank test p<0.07) of all lung cancer patients (without considering histological subtypes).

The lists of these genes and their individual association with survival rates in patients from a series of 271 lung tumours are shown in the following table 1.

TABLE 1

| | SEQ | SEQ | | | |
|---|---|---|---|---|---|
| | ID NO | ID NO | | Logrank | Nb lung |
| GeneID: | gene | protein | Gene name | p value | cancer |
| 442 | 1 | 24 | SOX30 | 0.047 | 61 |
| 35 | 2 | 25 | SPATA22 | 0.02 | 22 |
| 295 | 3 | 26 | MAEL | 0.069 | 27 |
| 1161 | 4 | 27 | COX8C | 0.004 | 40 |
| 102 | 5 | 28 | TKTL1 | 0.059 | 40 |
| 391 | 6 | 29 | RBM46 | 0.007 | 29 |
| 390 | 7 | 30 | MAGEB6 | 0.062 | 68 |
| 222 | 8 | 31 | NBPF4 | 0.0008 | 8 |
| 1261 | 9 | 32 | C12orf37 | 0.013 | 9 |
| 1165 | 10 | 33 | TPTE2P3 | 0.003 | 5 |
| 72 | 11 | 34 | DPEP3 | 0.002 | 9 |
| 108 | 12 | 35 | C10orf82 | 0.0005 | 6 |
| 205 | 13 | 36 | LOC440896 | 0.021 | 9 |
| 1188 | 14 | 37 | CDNA clone | 0.04 | 6 |
| | | | IMAGE: 5265646 | | |
| 1328 | 15 | 38 | HIST1H3C | 0.029 | 16 |
| 125 | 16 | 39 | PIWIL1 | <0.0001 | 7 |
| 144 | 17 | 40 | C19orf41 | <0.0001 | 4 |
| 437 | 18 | 41 | RNF17 | 0.026 | 7 |
| 766 | 19 | 42 | GALNTL5 | <0.0001 | 5 |
| 436 | 20 | 43 | RFX4 | 0.06 | 15 |
| 487 | 21 | 44 | LGALS14 | 0.009 | 5 |
| 117 | 22 | 45 | IGFBP1 | <0.0001 | 6 |
| 135 | 23 | 46 | TUBA3C | 0.077 | 9 |

The correspondence between the gene ID number and the corresponding SEQ ID is represented as follows:

Gene Num ID: 442 corresponds to SEQ ID NO: 1, Gene Num ID: 35 corresponds to SEQ ID NO: 2, Gene Num ID: 295 corresponds to SEQ ID NO: 3, Gene Num ID: 1161 corresponds to SEQ ID NO: 4, Gene Num ID: 102 corresponds to SEQ ID NO: 5, Gene Num ID: 391 corresponds to SEQ ID NO: 6, Gene Num ID: 390 corresponds to SEQ ID NO: 7, Gene Num ID: 222 corresponds to SEQ ID NO: 8, Gene Num ID: 1261 corresponds to SEQ ID NO: 9, Gene Num ID: 1165 corresponds to SEQ ID NO: 10, Gene Num ID: 72 corresponds to SEQ ID NO: 11, Gene Num ID: 108 corresponds to SEQ ID NO: 12, Gene Num ID: 205 corresponds to SEQ ID NO: 13, Gene Num ID: 1188 corresponds to SEQ ID NO: 14, Gene Num ID: 1328 corresponds to SEQ ID NO: 15, Gene Num ID: 125 corresponds to SEQ ID NO: 16, Gene Num ID: 144 corresponds to SEQ ID NO: 17, Gene Num ID: 437 corresponds to SEQ ID NO: 18, Gene Num ID: 766 corresponds to SEQ ID NO: 19, Gene Num ID: 436 corresponds to SEQ ID NO: 20, Gene Num ID: 487 corresponds to SEQ ID NO: 21, Gene Num ID: 117 corresponds to SEQ ID NO: 22 and Gene Num ID: 135 corresponds to SEQ ID NO: 23.

Logrank p value corresponds to the significance of difference in cumulative global survival probabilities over 5 years between patients expressing the gene and those not expressing the gene. Nb lung cancer corresponds to the number of lung cancer patients expressing the gene (/271).

Each of these genes can be used individually to establish a prognosis in lung cancer patients: any patient expressing any one of the 23 genes (of the group of 7 or the group of 16) has significantly lower chances of survival compared to the patients not expressing the gene (see table 2a).

| | | | | | | | | | | p-value | p-value | |
|---|---|---|---|---|---|---|---|---|---|---|---|---|
| | | | | | % alive | | | | | 2.5 years | 5 years | p-value |
| | | | Total | Nb alive | 30 | Nb alive | % alive | Nb alive | % alive | (Logrank | (Logrank | 10 years |
| | Gene | Classes | nb | 30 month | month | 60 month | 60 month | 120 month | 120 month | Test) | Test) | (Logrank Test) |
| Combi7genes | 1161 | 0 | 231 | 152 | 66 | 118 | 51 | 95 | 41 | 0.006 | 0.004 | 0.006 |
| | | 1 | 40 | 19 | 48 | 13 | 33 | 10 | 25 | | | |
| | 391 | 0 | 242 | 157 | 65 | 124 | 51 | 100 | 41 | 0.079 | 0.007 | 0.007 |
| | | 1 | 29 | 14 | 48 | 7 | 24 | 5 | 17 | | | |
| | 35 | 0 | 249 | 160 | 64 | 125 | 50 | 101 | 41 | 0.135 | 0.02 | 0.007 |
| | | 1 | 22 | 11 | 50 | 6 | 27 | 4 | 18 | | | |
| | 442 | 0 | 210 | 135 | 64 | 110 | 52 | 89 | 42 | 0.527 | 0.047 | 0.04 |
| | | 1 | 61 | 36 | 59 | 21 | 34 | 16 | 26 | | | |
| | 102 | 0 | 231 | 152 | 66 | 118 | 51 | 94 | 41 | 0.054 | 0.059 | 0.138 |
| | | 1 | 40 | 19 | 48 | 13 | 33 | 11 | 28 | | | |
| | 390 | 0 | 203 | 132 | 65 | 103 | 51 | 85 | 42 | 0.085 | 0.062 | 0.021 |
| | | 1 | 68 | 39 | 57 | 28 | 41 | 20 | 29 | | | |
| | 295 | 0 | 244 | 158 | 65 | 122 | 50 | 100 | 41 | 0.08 | 0.069 | 0.027 |
| | | 1 | 27 | 13 | 48 | 9 | 33 | 5 | 19 | | | |
| Combi16genes | 125 | 0 | 264 | 171 | 65 | 131 | 50 | 105 | 40 | <0.0001 | <0.0001 | <0.0001 |
| | | 1 | 7 | 0 | 0 | 0 | 0 | 0 | 0 | | | |
| | 117 | 0 | 265 | 171 | 65 | 131 | 49 | 105 | 40 | <0.0001 | <0.0001 | <0.0001 |
| | | 1 | 6 | 0 | 0 | 0 | 0 | 0 | 0 | | | |
| | 766 | 0 | 266 | 171 | 64 | 131 | 49 | 105 | 39 | <0.0001 | <0.0001 | <0.0001 |
| | | 1 | 5 | 0 | 0 | 0 | 0 | 0 | 0 | | | |
| | 144 | 0 | 267 | 170 | 64 | 130 | 49 | 104 | 39 | <0.0001 | <0.0001 | <0.0001 |
| | | 1 | 4 | 1 | 25 | 1 | 25 | 1 | 25 | | | |
| | 108 | 0 | 265 | 170 | 64 | 131 | 49 | 105 | 40 | 0.004 | 0.0005 | 0.0005 |
| | | 1 | 6 | 1 | 17 | 0 | 0 | 0 | 0 | | | |
| | 222 | 0 | 263 | 169 | 64 | 131 | 50 | 105 | 40 | 0.018 | 0.0008 | 0.0008 |
| | | 1 | 8 | 2 | 25 | 0 | 0 | 0 | 0 | | | |
| | 72 | 0 | 262 | 169 | 65 | 130 | 50 | 104 | 40 | 0.003 | 0.002 | 0.003 |
| | | 1 | 9 | 2 | 22 | 1 | 11 | 1 | 11 | | | |
| | 1165 | 0 | 266 | 170 | 64 | 131 | 49 | 105 | 39 | 0.011 | 0.003 | 0.003 |
| | | 1 | 5 | 1 | 20 | 0 | 0 | 0 | 0 | | | |
| | 487 | 0 | 266 | 170 | 64 | 131 | 49 | 105 | 39 | 0.032 | 0.009 | 0.009 |
| | | 1 | 5 | 1 | 20 | 0 | 0 | 0 | 0 | | | |
| | 1261 | 0 | 262 | 169 | 65 | 130 | 50 | 104 | 40 | 0.015 | 0.013 | 0.034 |
| | | 1 | 9 | 2 | 22 | 1 | 11 | 1 | 11 | | | |
| | 205 | 0 | 262 | 167 | 64 | 129 | 49 | 103 | 39 | 0.072 | 0.021 | 0.053 |
| | | 1 | 9 | 4 | 44 | 2 | 22 | 2 | 22 | | | |
| | 437 | 0 | 264 | 169 | 64 | 129 | 49 | 103 | 39 | 0.006 | 0.026 | 0.026 |
| | | 1 | 7 | 2 | 29 | 2 | 29 | 2 | 29 | | | |
| | 1328 | 0 | 255 | 164 | 64 | 127 | 50 | 104 | 41 | 0.068 | 0.029 | 0.007 |
| | | 1 | 16 | 7 | 44 | 4 | 25 | 1 | 6 | | | |
| | 1188 | 0 | 265 | 169 | 64 | 130 | 49 | 104 | 39 | 0.041 | 0.04 | 0.058 |
| | | 1 | 6 | 2 | 33 | 1 | 17 | 1 | 17 | | | |
| | 436 | 0 | 256 | 166 | 65 | 126 | 49 | 101 | 39 | 0.002 | 0.06 | 0.134 |
| | | 1 | 15 | 5 | 33 | 5 | 33 | 4 | 27 | | | |
| | 135 | 0 | 262 | 168 | 64 | 129 | 49 | 103 | 39 | 0.05 | 0.077 | 0.231 |
| | | 1 | 9 | 3 | 33 | 2 | 22 | 2 | 22 | | | |

Table 2a represents the data for all the 23 genes according to the invention
Gene = gene identifier(s) of individual genes or combinations of genes
Classes = Lung Kc patient not expressing (=0) or expressing (=1) the gene;
In the case of combinations of several genes, some patients could express one gene only or at least one gene of the combination (=1), 2 genes only or 2 genes or more of the combination (=2) etc . . .
Total Nb = total number of patients of this class (considering the whole Brambilla study with 271 cases, including all histological types)
% or nb alive 30, 60, 120 month = % or number of patients of this class alive after 30, 60, 120 month
p-value corresponding to the significance of the difference in cumulative global survival probabilities between the different classes of patients over 2.5, 5 and 10 years (Logrank Test, considering survival curves of all the classes of patients).

The inventors have also shown that using combinations of these genes allows a more accurate prognosis. Examples of these combinations and their use to establish a prognosis are shown below.

In order to establish a prognosis in lung cancer patients, the aberrant expression of these genes can be used as follows:
Combinations of all or several of the genes of the group of 7 genes and/or of the group of 16 genes (examples of combinations, see tables 2b and 2c)
Any one gene of the group of 7 genes (preferably) or of the group of 16 genes (see table 2a)

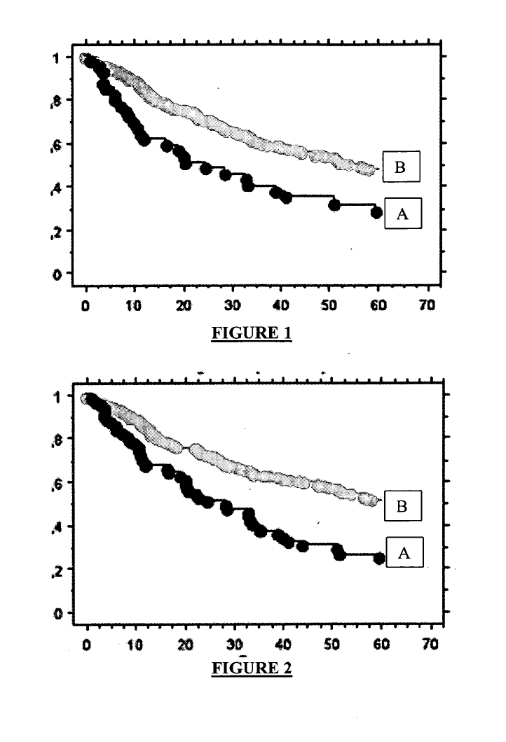

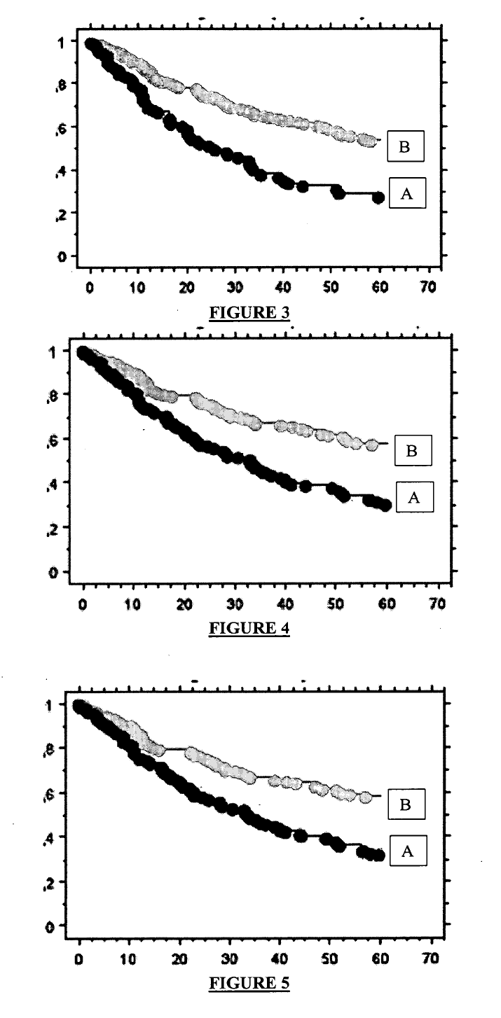

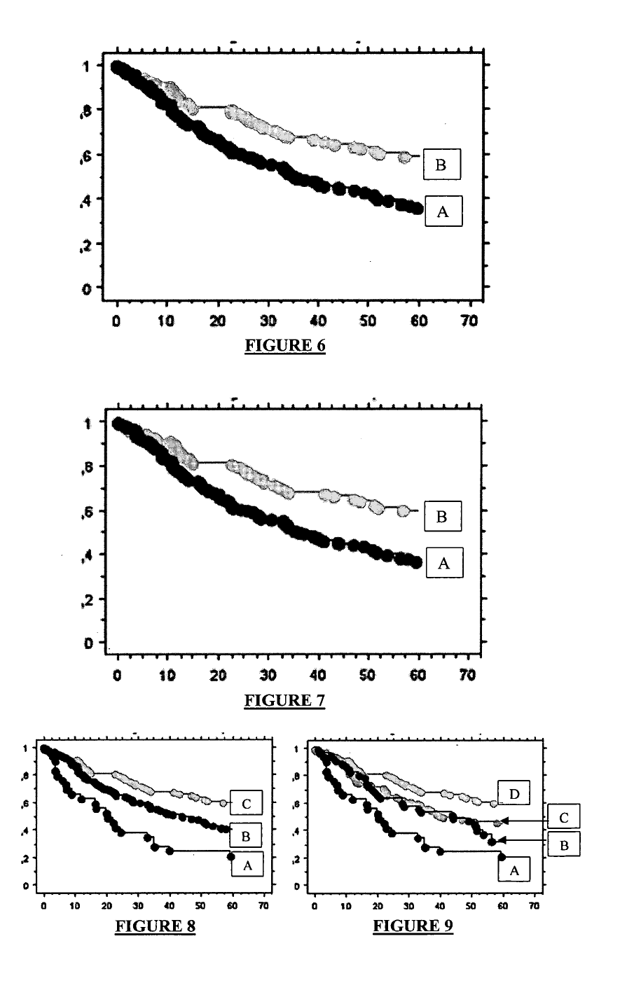

TABLE 2b

| | | | Nb | % | | | | | p-value | p-value | p-value | |
|---|---|---|---|---|---|---|---|---|---|---|---|---|
| | | Total | alive | alive | Nb alive | % alive | Nb alive | % alive | 2.5 years | 5 years | 10 years | Corresponding |
| genes | Classes | Nb | 30 m | 30 m | 60 m | 60 m | 120 m | 120 m | (Logrank Test) | (Logrank Test) | (Logrank Test) | FIG. |
| 1161 | 0 | 231 | 152 | 66 | 118 | 51 | 95 | 41 | 0.006 | 0.0038 | 0.006 | FIG. 1 |
| | 1 | 40 | 19 | 48 | 13 | 33 | 10 | 25 | | | | |
| 1161 + | 0 | 209 | 141 | 67 | 114 | 55 | 92 | 44 | 0.003 | <0.0001 | 0.0001 | FIG. 2 |
| 391 | >=1 | 62 | 30 | 48 | 17 | 27 | 13 | 21 | | | | |
| 1161 + | 0 | 193 | 134 | 69 | 108 | 56 | 88 | 46 | 0.0002 | <0.0001 | <0.0001 | FIG. 3 |
| 391 + | >=1 | 78 | 37 | 47 | 23 | 29 | 17 | 22 | | | | |
| 35 | | | | | | | | | | | | |
| 1161 + | 0 | 155 | 110 | 71 | 93 | 60 | 76 | 49 | 0.002 | <0.0001 | <0.0001 | FIG. 4 |
| 391 + | >=1 | 116 | 61 | 53 | 38 | 33 | 29 | 25 | | | | |
| 35 + | 0 | 155 | 110 | 71 | 93 | 60 | 76 | 49 | 0.008 | <0.0001 | <0.0001 | |
| 442 | 1 | 86 | 45 | 52 | 29 | 34 | 23 | 27 | | | | |
| | >=2 | 30 | 16 | 53 | 9 | 30 | 6 | 20 | | | | |
| 1161 + | 0 | 145 | 103 | 71 | 88 | 61 | 72 | 50 | 0.005 | <0.0001 | <0.0001 | FIG. 5 |
| 391 + | >=1 | 126 | 68 | 54 | 43 | 34 | 33 | 26 | | | | |
| 35 + | 0 | 145 | 103 | 71 | 88 | 61 | 72 | 50 | 0.006 | 0.0001 | 0.0002 | |
| 442 + | 1 | 74 | 44 | 59 | 28 | 38 | 21 | 28 | | | | |
| 102 | >=2 | 52 | 24 | 46 | 15 | 29 | 12 | 23 | | | | |
| 1161 + | 0 | 113 | 81 | 72 | 69 | 61 | 59 | 52 | 0.009 | 0.0006 | 0.0001 | FIG. 6 |
| 391 + | >=1 | 158 | 90 | 57 | 62 | 39 | 46 | 29 | | | | |
| 35 + | 0 | 113 | 81 | 72 | 69 | 61 | 59 | 52 | 0.007 | 0.0002 | 0.0001 | |
| 442 + | 1 | 84 | 53 | 63 | 40 | 48 | 29 | 35 | | | | |
| 102 + | >=2 | 74 | 37 | 50 | 22 | 30 | 27 | 36 | | | | |
| 390 | 0 | 113 | 81 | 72 | 69 | 61 | 59 | 52 | 0.004 | <0.0001 | <0.0001 | |
| | 1 | 84 | 53 | 63 | 40 | 48 | 29 | 35 | | | | |
| | 2 | 53 | 29 | 55 | 18 | 34 | 14 | 26 | | | | |
| | >=3 | 21 | 8 | 38 | 4 | 19 | 3 | 14 | | | | |
| | 0 | 113 | 81 | 72 | 69 | 61 | 59 | 52 | 0.002 | <0.0001 | <0.0001 | |
| | 1&2 | 137 | 82 | 60 | 58 | 42 | 43 | 31 | | | | |
| | >=3 | 21 | 8 | 38 | 4 | 19 | 3 | 14 | | | | |
| 1161 + | 0 | 105 | 76 | 72 | 65 | 62 | 57 | 54 | 0.008 | 0.0005 | <0.0001 | FIG. 7 |
| 391 + | >=1 | 166 | 95 | 57 | 66 | 40 | 48 | 29 | | | | |
| 35 + | 0 | 105 | 76 | 72 | 65 | 62 | 57 | 54 | 0.008 | 0.0002 | <0.0001 | |
| 442 + | 1 | 87 | 55 | 63 | 42 | 48 | 29 | 33 | | | | |
| 102 + | >=2 | 79 | 40 | 51 | 24 | 30 | 19 | 24 | | | | |
| 390 + | 0 | 105 | 76 | 72 | 65 | 62 | 57 | 54 | 0.002 | <0.0001 | <0.0001 | FIG. 9 |
| 295 | 1 | 87 | 55 | 63 | 42 | 48 | 29 | 33 | | | | |
| | 2 | 50 | 29 | 58 | 18 | 36 | 15 | 30 | | | | |
| | >=3 | 29 | 11 | 38 | 6 | 21 | 4 | 14 | | | | |
| | 0 | 105 | 76 | 72 | 65 | 62 | 57 | 54 | 0.0006 | <0.0001 | <0.0001 | FIG. 8 |
| | 1&2 | 137 | 84 | 61 | 60 | 44 | 44 | 32 | | | | |
| | >=3 | 29 | 11 | 38 | 6 | 21 | 4 | 14 | | | | |

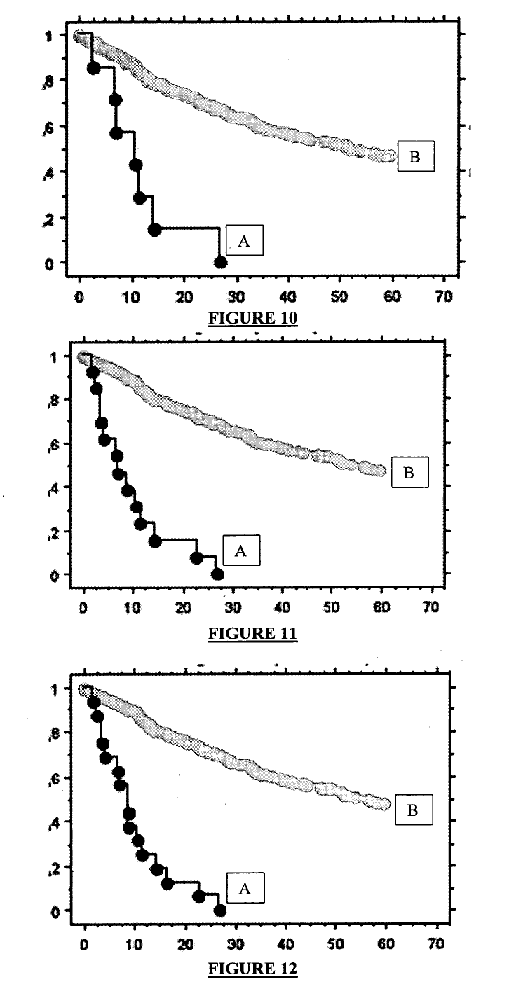

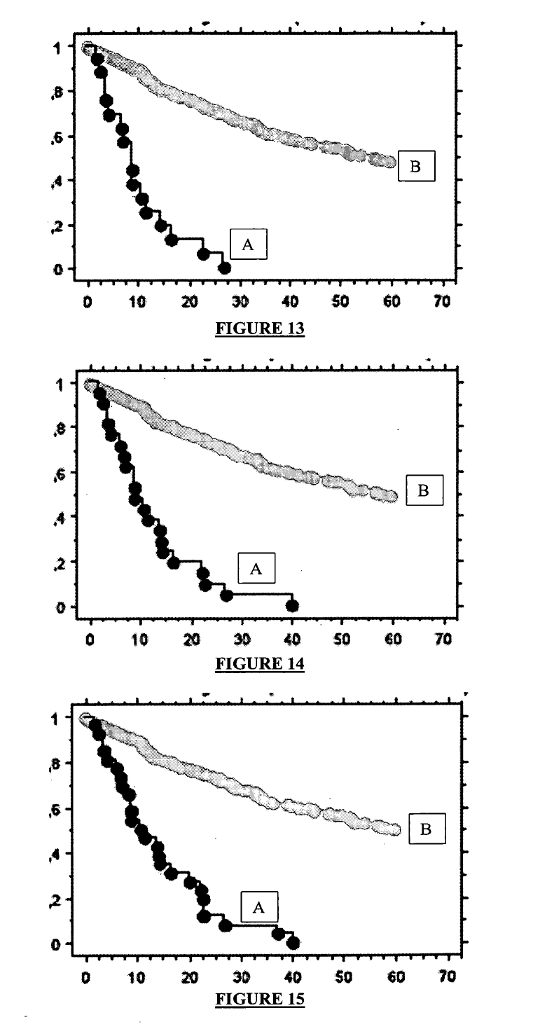

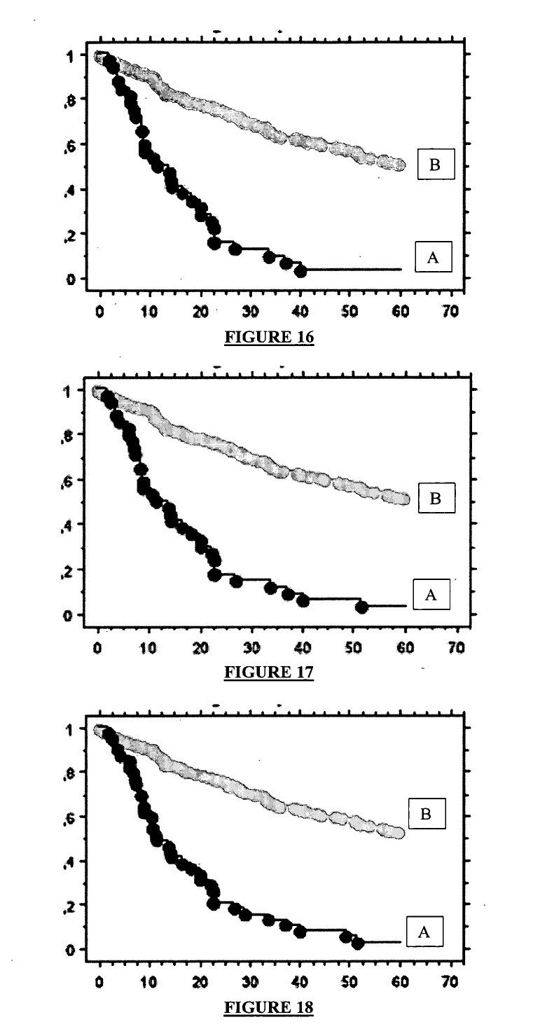

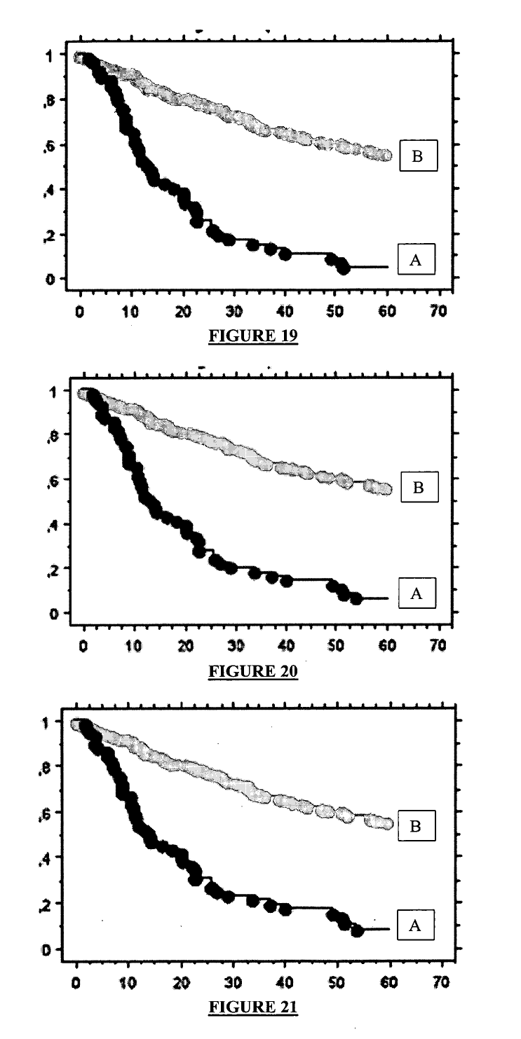

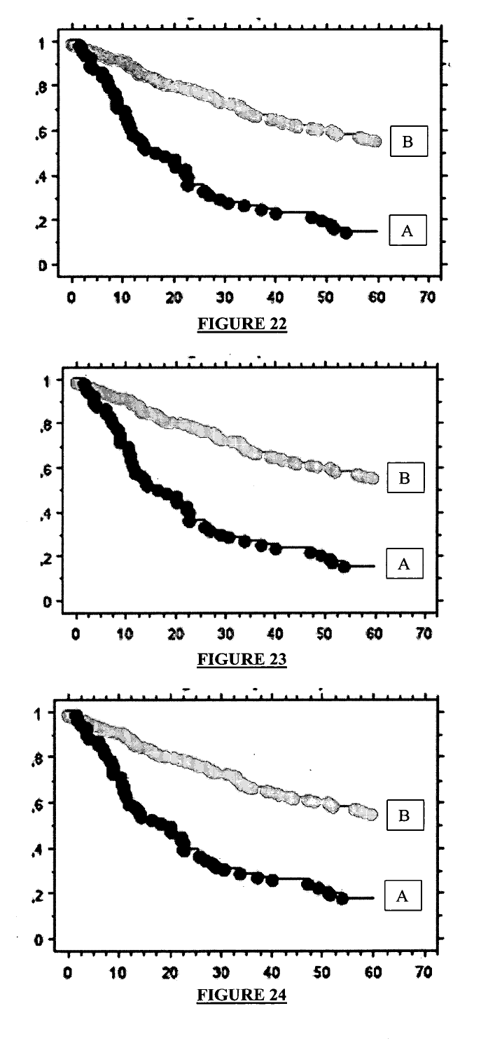

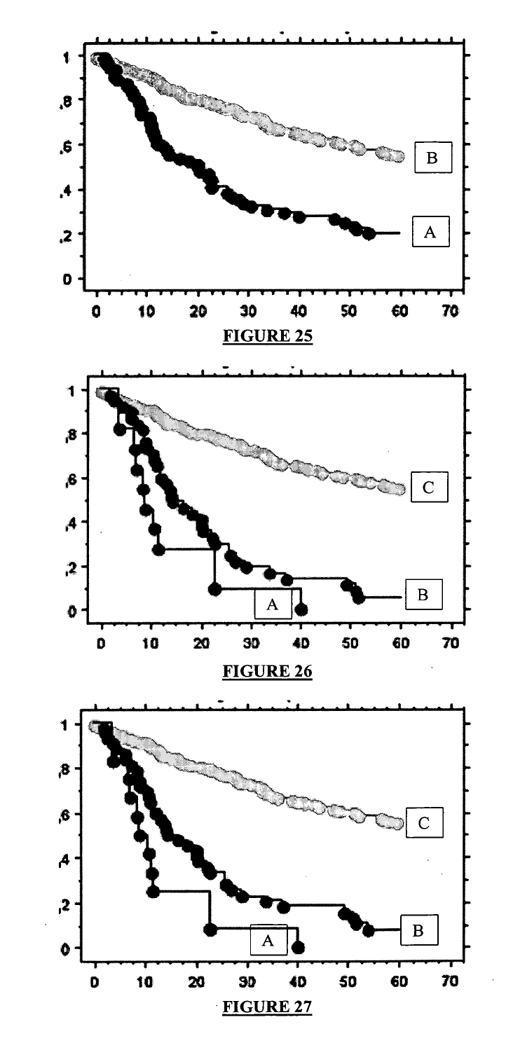

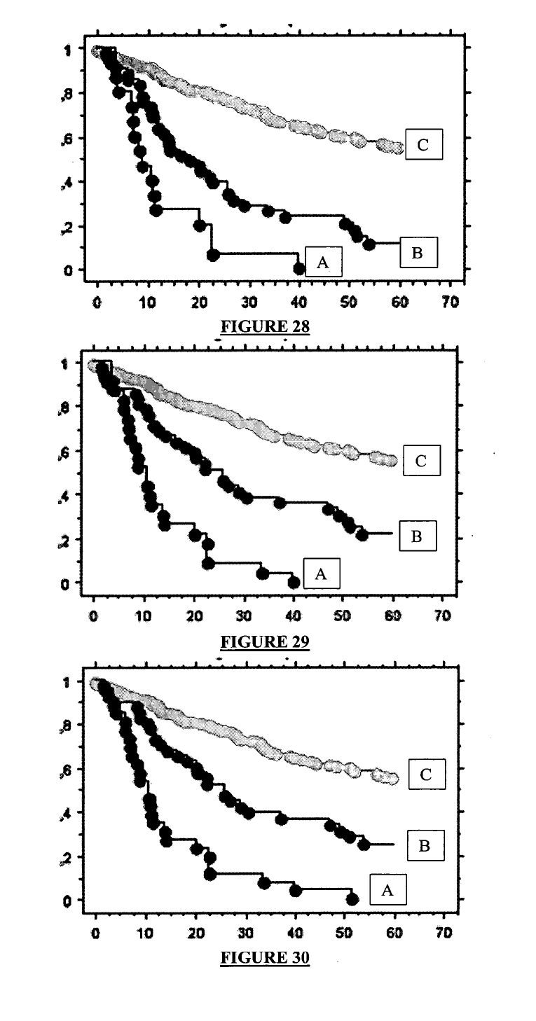

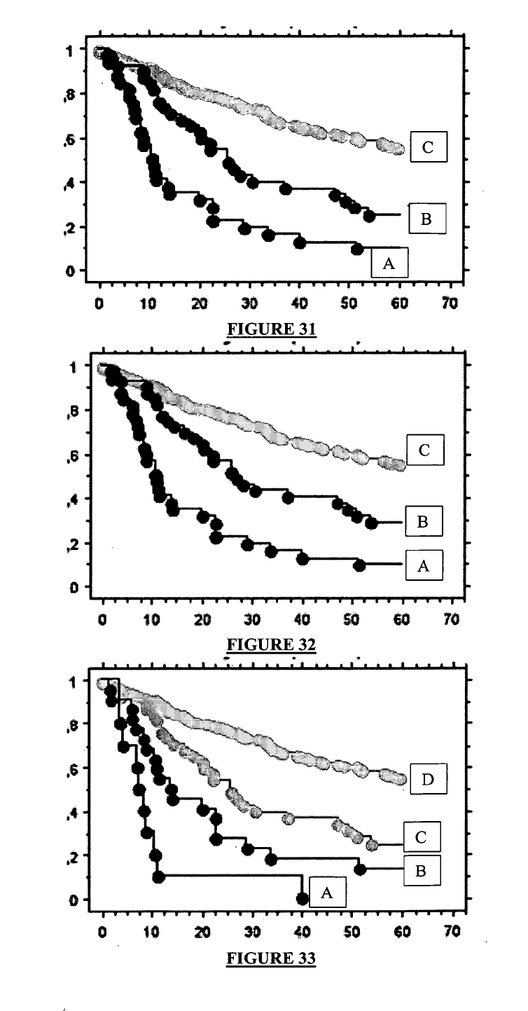

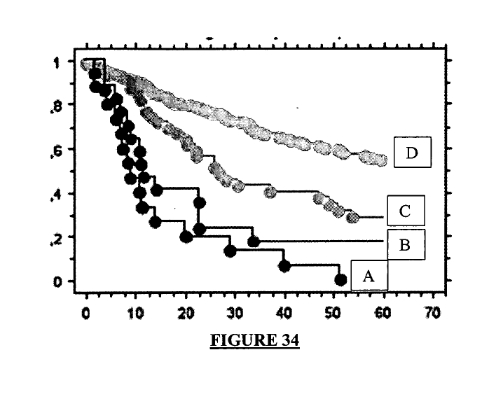

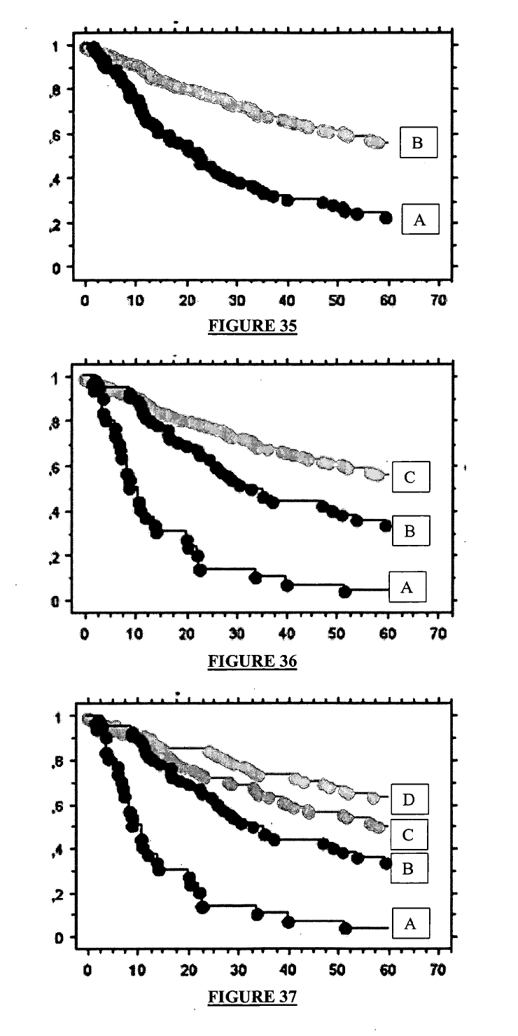

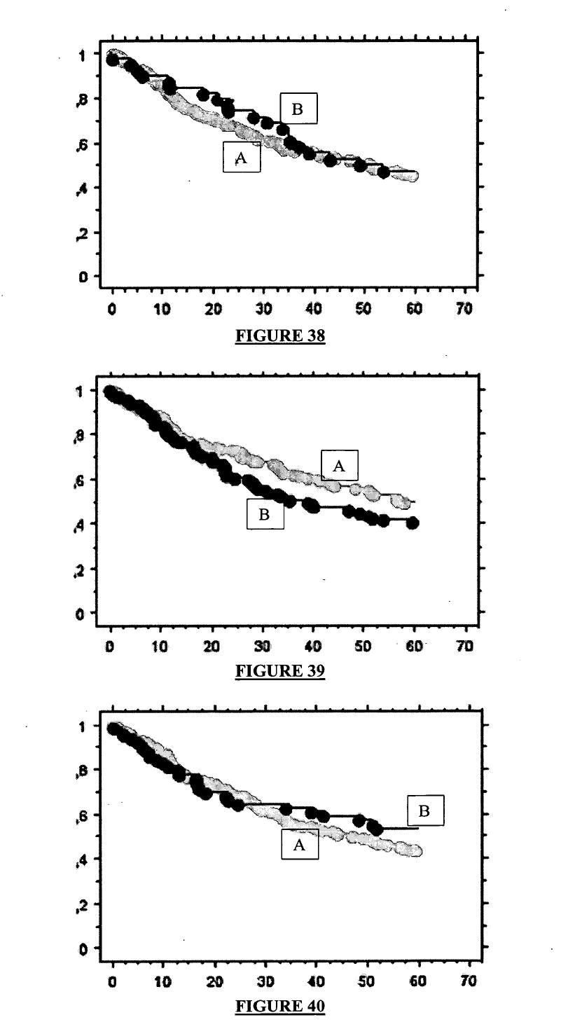

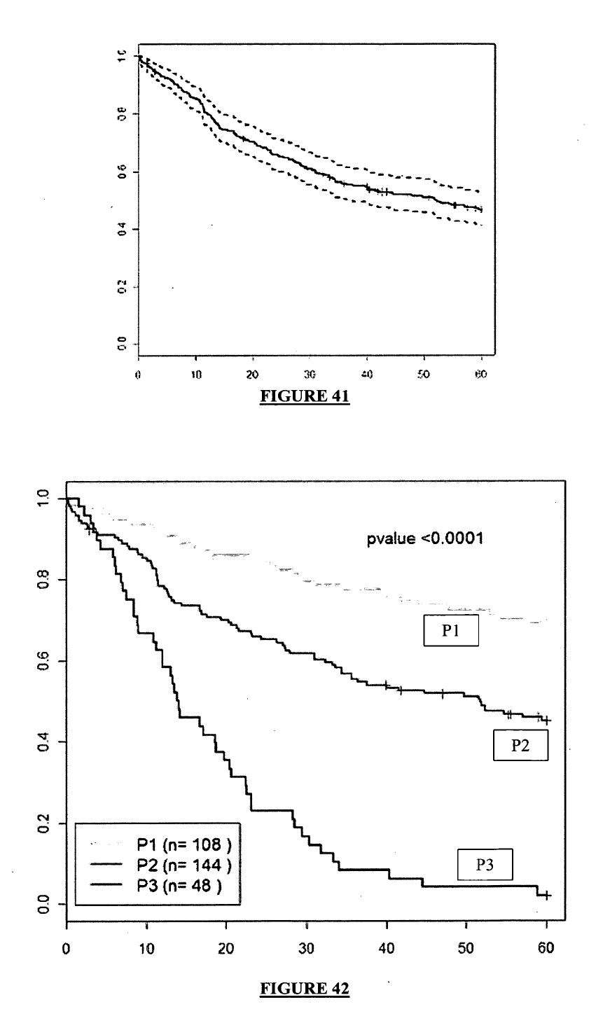

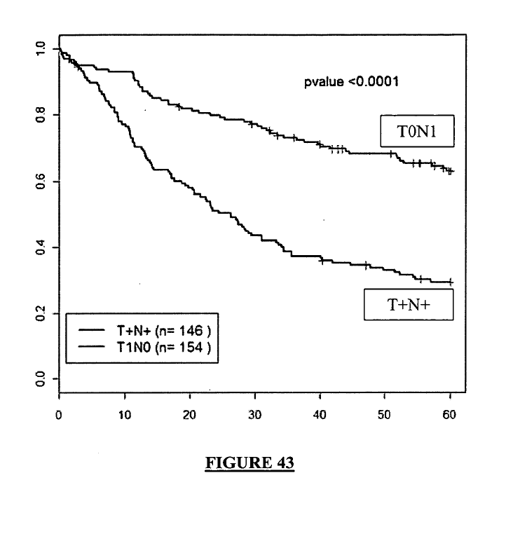

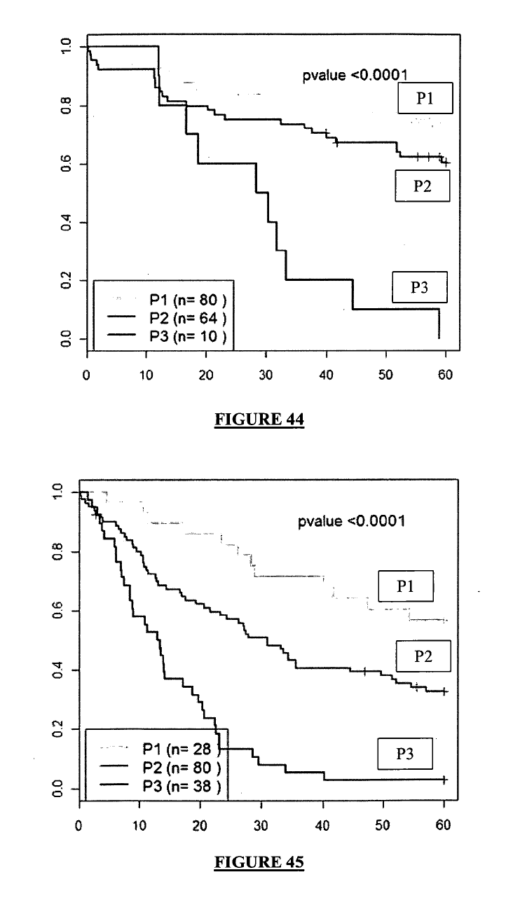

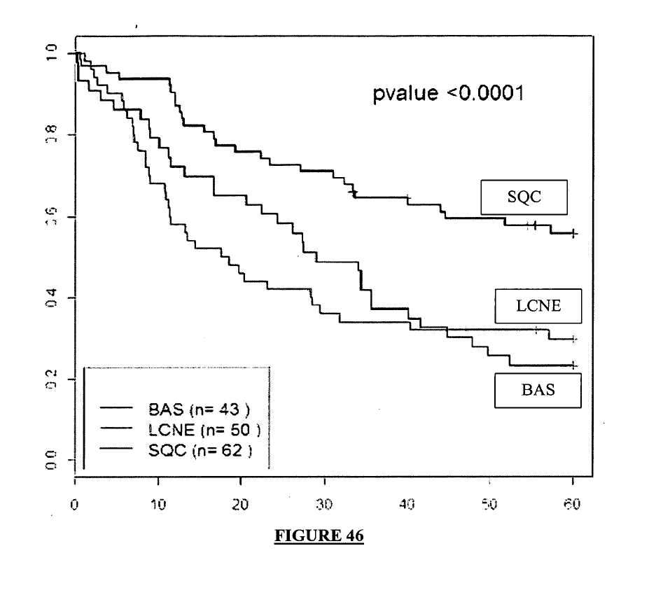

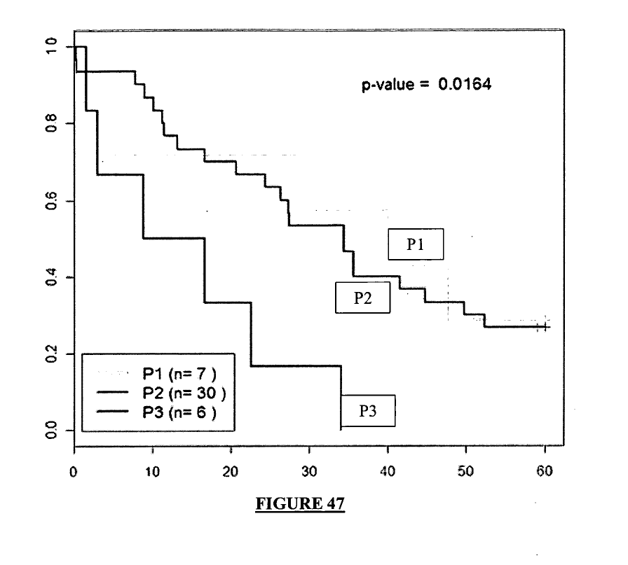

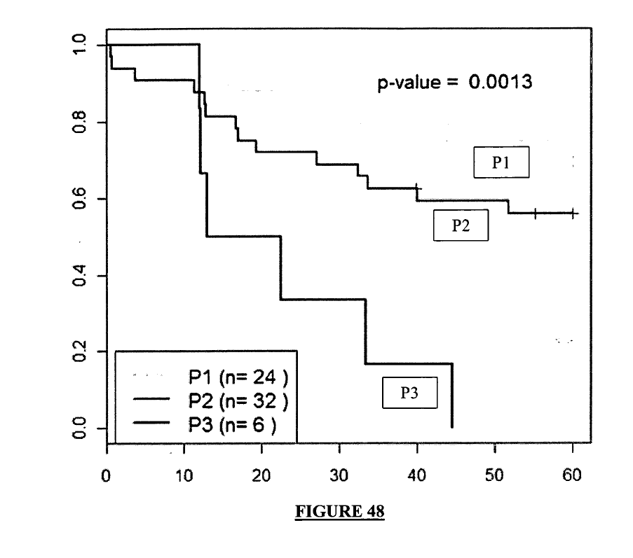

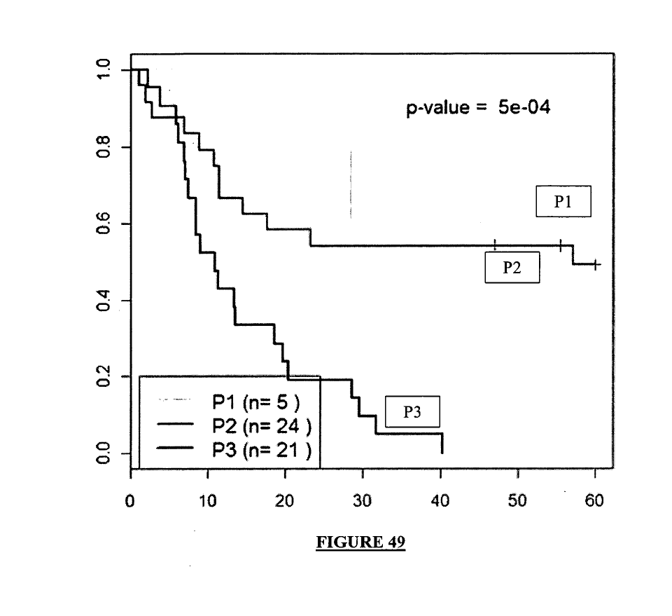

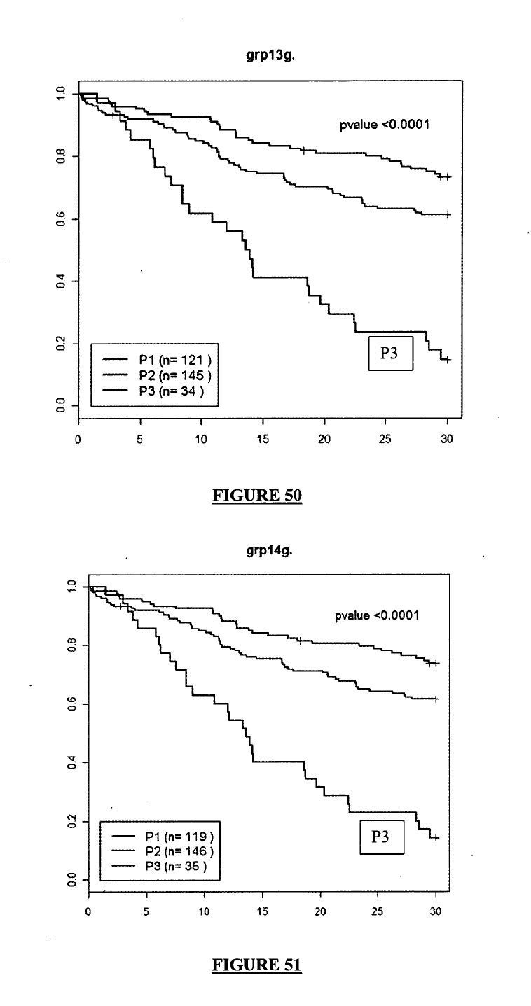

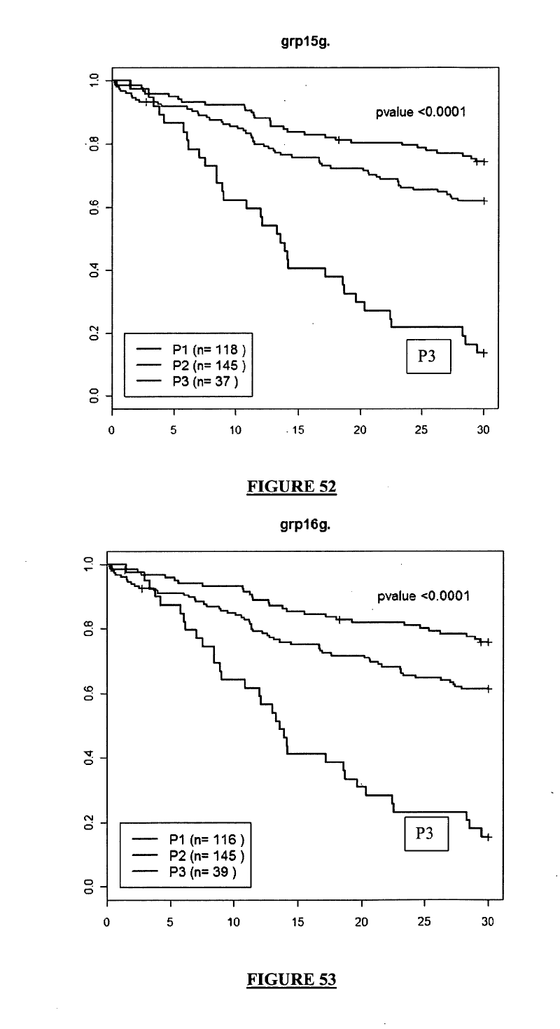

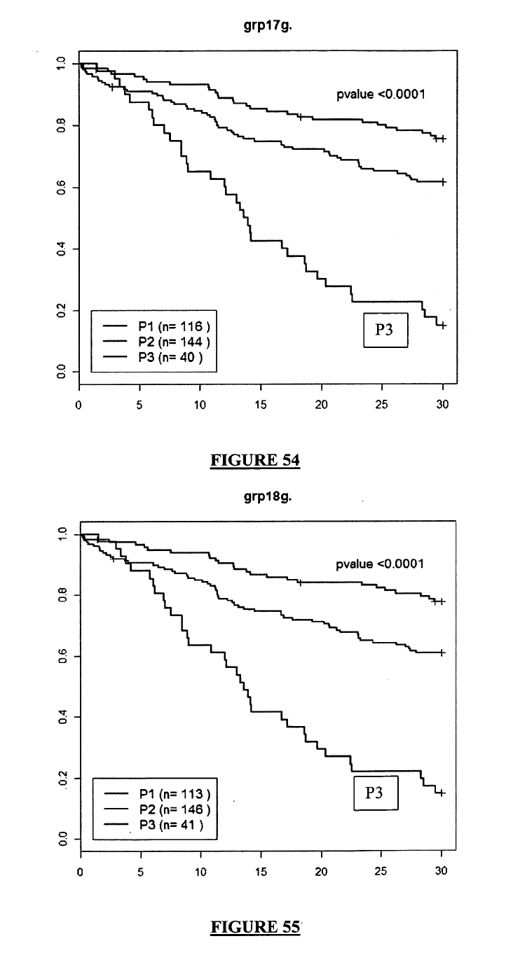

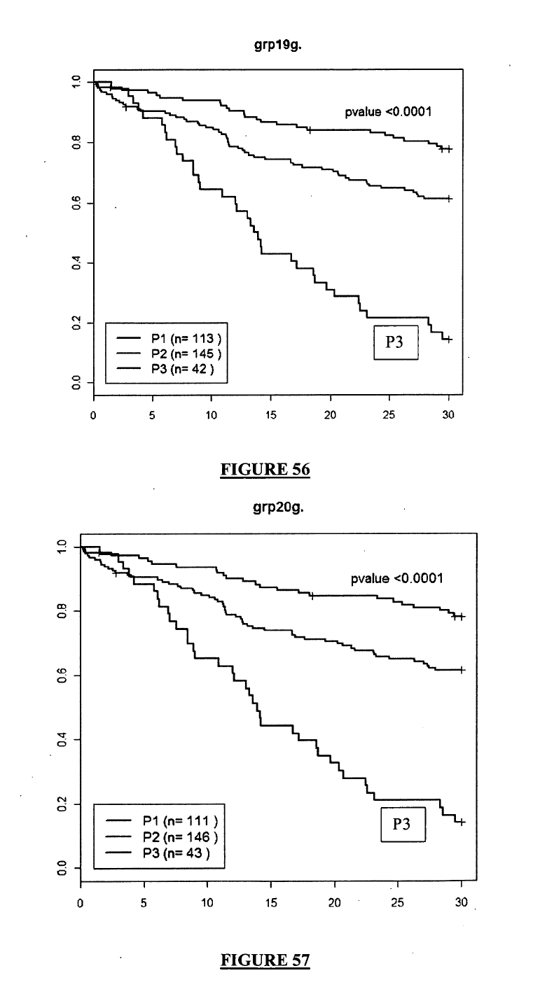

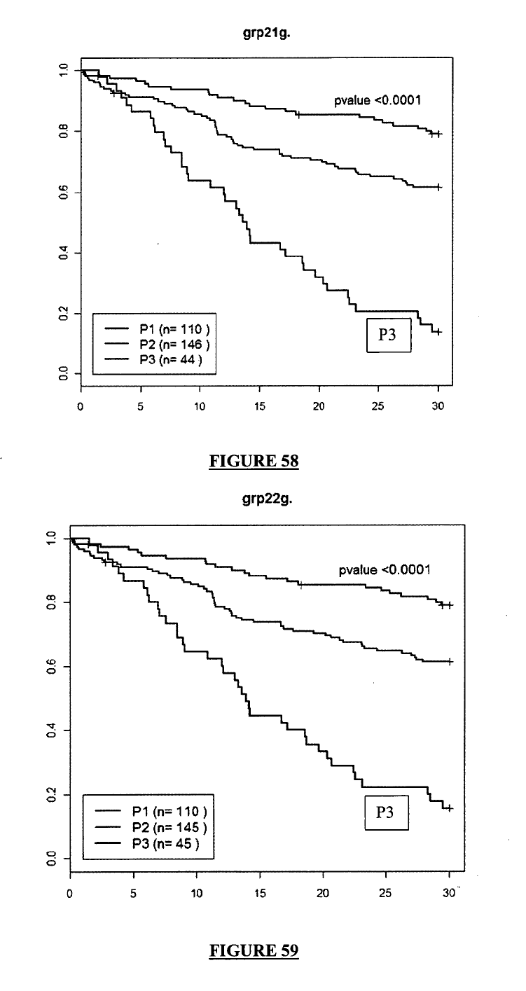

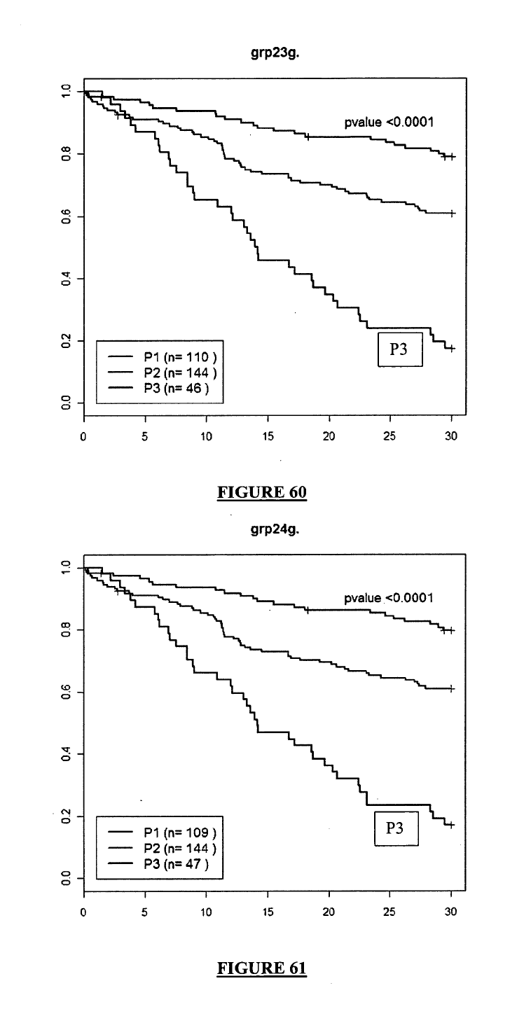

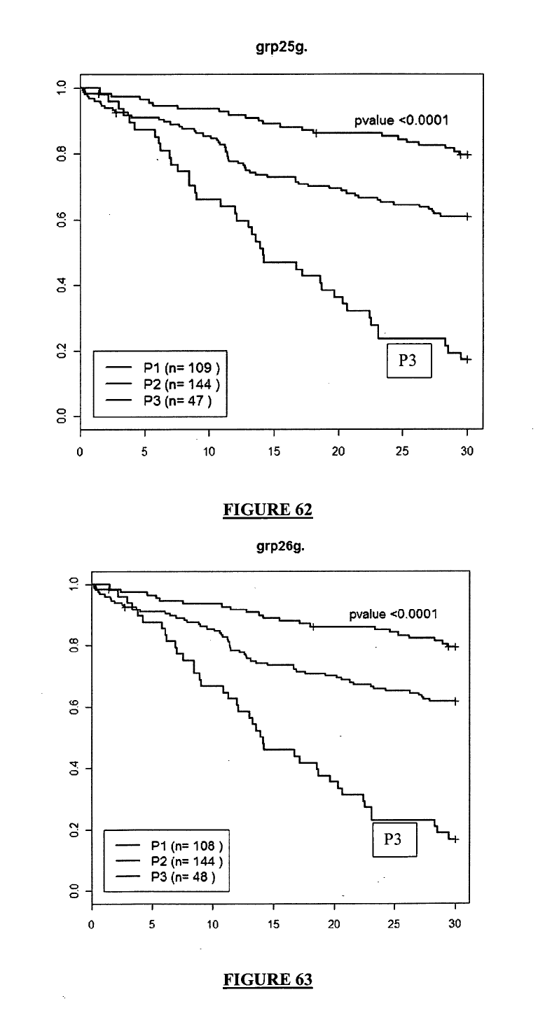

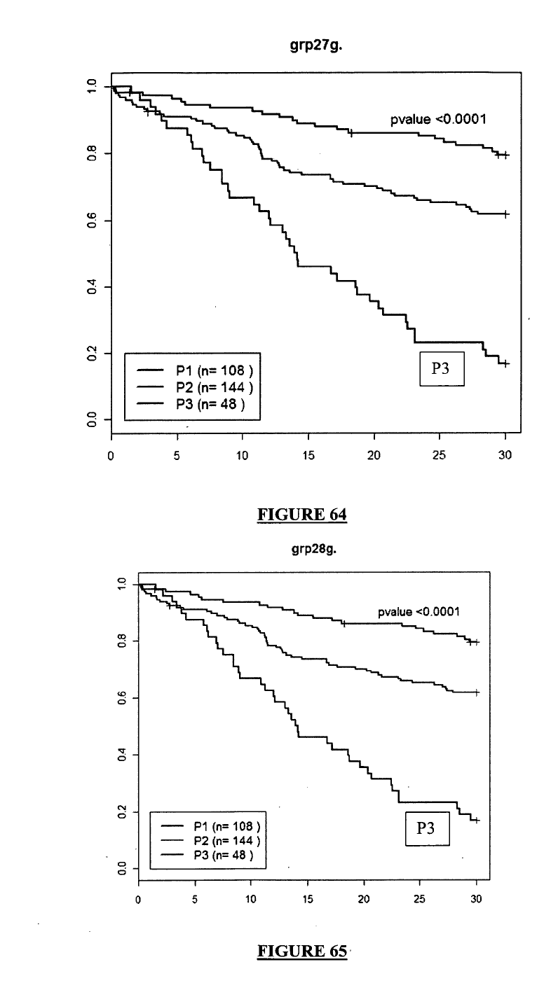

TABLE 2c

| | | | | | | | | | p-value | p-value | p-value | |
|---|---|---|---|---|---|---|---|---|---|---|---|---|
| | | | | | | | | | 2.5 years | 5 years | 10 years | |
| | | | Nb alive | % alive | Nb alive | % alive | Nb alive | % alive | (Logrank | (Logrank | (Logrank | Corresponding |
| Genes | Classes | Total Nb | 30 m | 30 m | 60 m | 60 m | 120 m | 120 m | Test) | Test) | Test) | FIG. |
| 125 | 0 | 264 | 171 | 65 | 131 | 50 | 105 | 40 | <0.0001 | <0.0001 | <0.0001 | FIG. 10 |
| | 1 | 7 | 0 | 0 | 0 | 0 | 0 | 0 | | | | |
| 125 + | 0 | 258 | 171 | 66 | 131 | 51 | 105 | 41 | <0.0001 | <0.0001 | <0.0001 | FIG. 11 |
| 117 | >=1 | 13 | 0 | 0 | 0 | 0 | 0 | 0 | | | | |
| 125 + | 0 | 255 | 171 | 67 | 131 | 51 | 105 | 41 | <0.0001 | <0.0001 | <0.0001 | FIG. 12 |
| 117 + | >=1 | 16 | 0 | 0 | 0 | 0 | 0 | 0 | | | | |
| 766 | | | | | | | | | | | | |
| 125 + | 0 | 254 | 170 | 67 | 130 | 51 | 104 | 41 | <0.0001 | <0.0001 | <0.0001 | FIG. 13 |
| 117 + | >=1 | 17 | 1 | 6 | 1 | 6 | 0 | 0 | | | | |
| 766 + | | | | | | | | | | | | |
| 144 | | | | | | | | | | | | |
| 125 + | 0 | 249 | 169 | 68 | 130 | 52 | 104 | 42 | <0.0001 | <0.0001 | <0.0001 | FIG. 14 |
| 117 + | >=1 | 22 | 2 | 9 | 1 | 5 | 0 | 0 | | | | |
| 766 + | | | | | | | | | | | | |
| 144 + | | | | | | | | | | | | |
| 108 | | | | | | | | | | | | |
| 125 + | 0 | 244 | 168 | 69 | 130 | 53 | 104 | 43 | <0.0001 | <0.0001 | <0.0001 | FIG. 15 |
| 117 + | >=1 | 27 | 3 | 11 | 1 | 4 | 0 | 0 | | | | |
| 766 + | | | | | | | | | | | | |
| 144 + | | | | | | | | | | | | |
| 108 + | | | | | | | | | | | | |
| 222 | | | | | | | | | | | | |
| 125 + | 0 | 238 | 166 | 70 | 129 | 54 | 103 | 43 | <0.0001 | <0.0001 | <0.0001 | FIG. 16 |
| 117 + | >=1 | 33 | 5 | 15 | 2 | 6 | 2 | 6 | | | | |
| 766 + | | | | | | | | | | | | |
| 144 + | | | | | | | | | | | | |
| 108 + | | | | | | | | | | | | |
| 222 + | | | | | | | | | | | | |
| 72 | | | | | | | | | | | | |
| 125 + | 0 | 236 | 165 | 70 | 129 | 55 | 103 | 44 | <0.0001 | <0.0001 | <0.0001 | FIG. 17 |
| 117 + | >=1 | 35 | 6 | 17 | 2 | 6 | 2 | 6 | | | | |
| 766 + | | | | | | | | | | | | |
| 144 + | | | | | | | | | | | | |
| 108 + | | | | | | | | | | | | |
| 222 + | | | | | | | | | | | | |
| 72 + | | | | | | | | | | | | |
| 1165 | | | | | | | | | | | | |
| 125 + | 0 | 231 | 164 | 71 | 129 | 56 | 103 | 45 | <0.0001 | <0.0001 | <0.0001 | FIG. 18 |
| 117 + | >=1 | 40 | 7 | 18 | 2 | 5 | 2 | 5 | | | | |
| 766 + | | | | | | | | | | | | |
| 144 + | | | | | | | | | | | | |
| 108 + | | | | | | | | | | | | |
| 222 + | | | | | | | | | | | | |
| 72 + | | | | | | | | | | | | |
| 1165 + | | | | | | | | | | | | |
| 487 | | | | | | | | | | | | |
| 125 + | 0 | 222 | 162 | 73 | 128 | 58 | 102 | 46 | <0.0001 | <0.0001 | <0.0001 | FIG. 19 |
| 117 + | >=1 | 49 | 9 | 18 | 3 | 6 | 3 | 6 | | | | |
| 766 + | 0 | 222 | 162 | 73 | 128 | 58 | 102 | 46 | <0.0001 | <0.0001 | <0.0001 | FIG. 26 |
| 144 + | 1 | 38 | 8 | 21 | 3 | 8 | 3 | 8 | | | | |
| 108 + | >=2 | 11 | 1 | 9 | 0 | 0 | 0 | 0 | | | | |
| 222 + | | | | | | | | | | | | |
| 72 + | | | | | | | | | | | | |
| 1165 + | | | | | | | | | | | | |
| 487 + | | | | | | | | | | | | |
| 1261 | | | | | | | | | | | | |
| 125 + | 0 | 216 | 159 | 74 | 126 | 58 | 100 | 46 | <0.0001 | <0.0001 | <0.0001 | FIG. 20 |
| 117 + | >=1 | 55 | 12 | 22 | 5 | 9 | 5 | 9 | | | | |
| 766 + | 0 | 216 | 159 | 74 | 126 | 58 | 100 | 46 | <0.0001 | <0.0001 | <0.0001 | FIG. 27 |
| 144 + | 1 | 43 | 11 | 26 | 5 | 12 | 5 | 12 | | | | |
| 108 + | >=2 | 12 | 1 | 8 | 0 | 0 | 0 | 0 | | | | |
| 222 + | | | | | | | | | | | | |
| 72 + | | | | | | | | | | | | |
| 1165 + | | | | | | | | | | | | |
| 487 + | | | | | | | | | | | | |
| 1261 + | | | | | | | | | | | | |
| 205 | | | | | | | | | | | | |
| 125 + | 0 | 214 | 157 | 73 | 124 | 58 | 98 | 46 | <0.0001 | <0.0001 | <0.0001 | FIG. 21 |
| 117 + | >=1 | 57 | 14 | 25 | 7 | 12 | 7 | 12 | | | | |
| 766 + | 0 | 214 | 157 | 73 | 124 | 58 | 98 | 46 | <0.0001 | <0.0001 | <0.0001 | FIG. 28 |
| 144 + | 1 | 42 | 13 | 31 | 7 | 17 | 7 | 17 | | | | |
| 108 + | >=2 | 15 | 1 | 7 | 0 | 0 | 0 | 0 | | | | |
| 222 + | | | | | | | | | | | | |
| 72 + | | | | | | | | | | | | |
| 1165 + | | | | | | | | | | | | |
| 487 + | | | | | | | | | | | | |
| 1261 + | | | | | | | | | | | | |
| 205 + | | | | | | | | | | | | |
| 437 | | | | | | | | | | | | |
| 125 + | 0 | 206 | 151 | 73 | 120 | 58 | 97 | 47 | <0.0001 | <0.0001 | <0.0001 | FIG. 22 |
| 117 + | >=1 | 65 | 20 | 31 | 11 | 17 | 8 | 12 | | | | |
| 766 + | 0 | 206 | 151 | 73 | 120 | 58 | 97 | 47 | <0.0001 | <0.0001 | <0.0001 | FIG. 29 |
| 144 + | 1 | 42 | 18 | 43 | 11 | 26 | 8 | 19 | | | | |
| 108 + | >=2 | 23 | 2 | 9 | 0 | 0 | 0 | 0 | | | | |
| 222 + | | | | | | | | | | | | |
| 72 + | | | | | | | | | | | | |
| 1165 + | | | | | | | | | | | | |
| 487 + | | | | | | | | | | | | |
| 1261 + | | | | | | | | | | | | |
| 205 + | | | | | | | | | | | | |
| 437 + | | | | | | | | | | | | |
| 1328 | | | | | | | | | | | | |
| 125 + | 0 | 204 | 150 | 74 | 119 | 58 | 96 | 47 | <0.0001 | <0.0001 | <0.0001 | FIG. 23 |
| 117 + | >=1 | 67 | 21 | 31 | 12 | 18 | 9 | 13 | | | | |
| 766 + | 0 | 204 | 150 | 74 | 119 | 58 | 96 | 47 | <0.0001 | <0.0001 | <0.0001 | FIG. 30 |
| 144 + | 1 | 41 | 18 | 44 | 12 | 29 | 9 | 22 | | | | |
| 108 + | >=2 | 26 | 3 | 12 | 0 | 0 | 0 | 0 | | | | |
| 222 + | | | | | | | | | | | | |
| 72 + | | | | | | | | | | | | |
| 1165 + | | | | | | | | | | | | |
| 487 + | | | | | | | | | | | | |
| 1261 + | | | | | | | | | | | | |
| 205 + | | | | | | | | | | | | |
| 437 + | | | | | | | | | | | | |
| 1328 + | | | | | | | | | | | | |
| 1188 | | | | | | | | | | | | |
| 125 + | 0 | 201 | 148 | 74 | 117 | 58 | 94 | 47 | <0.0001 | <0.0001 | <0.0001 | FIG. 24 |
| 117 + | >=1 | 70 | 23 | 33 | 14 | 20 | 11 | 16 | | | | |
| 766 + | 0 | 201 | 148 | 74 | 117 | 58 | 94 | 47 | <0.0001 | <0.0001 | <0.0001 | FIG. 31 |
| 144 + | 1 | 38 | 17 | 45 | 11 | 29 | 9 | 24 | | | | |
| 108 + | >=2 | 32 | 6 | 19 | 3 | 9 | 2 | 6 | | | | |
| 222 + | 0 | 201 | 148 | 74 | 117 | 58 | 94 | 47 | <0.0001 | <0.0001 | <0.0001 | FIG. 33 |
| 72 + | 1 | 38 | 17 | 45 | 11 | 29 | 9 | 24 | | | | |
| 1165 + | 2 | 22 | 5 | 23 | 3 | 14 | 2 | 9 | | | | |
| 487 + | >=3 | 10 | 1 | 10 | 0 | 0 | 0 | 0 | | | | |
| 1261 + | | | | | | | | | | | | |
| 205 + | | | | | | | | | | | | |
| 437 + | | | | | | | | | | | | |
| 1328 + | | | | | | | | | | | | |
| 1188 + | | | | | | | | | | | | |
| 436 | | | | | | | | | | | | |
| 125 + | 0 | 199 | 146 | 73 | 115 | 58 | 92 | 46 | <0.0001 | <0.0001 | <0.0001 | FIG. 25 |
| 117 + | >=1 | 72 | 25 | 35 | 16 | 22 | 13 | 18 | | | | |
| 766 + | 0 | 199 | 146 | 73 | 115 | 58 | 92 | 46 | <0.0001 | <0.0001 | <0.0001 | FIG. 32 |
| 144 + | 1 | 40 | 19 | 48 | 13 | 33 | 11 | 28 | | | | |
| 108 + | >=2 | 32 | 6 | 19 | 3 | 9 | 2 | 6 | | | | |
| 222 + | 0 | 199 | 146 | 73 | 115 | 58 | 92 | 46 | <0.0001 | <0.0001 | <0.0001 | FIG. 34 |
| 72 + | 1 | 40 | 19 | 48 | 13 | 33 | 11 | 28 | | | | |
| 1165 + | 2 | 17 | 4 | 24 | 3 | 18 | 2 | 12 | | | | |
| 487 + | >=3 | 15 | 2 | 13 | 0 | 0 | 0 | 0 | | | | |
| 1261 + | | | | | | | | | | | | |
| 205 + | | | | | | | | | | | | |
| 437 + | | | | | | | | | | | | |
| 1328 + | | | | | | | | | | | | |
| 1188 + | | | | | | | | | | | | |
| 436 + | | | | | | | | | | | | |
| 135 | | | | | | | | | | | | |

### Detailed Procedure and Examples

### I—Methodological Approach

The following procedure allowed identifying the genes according to the invention.

### Overview

The expression of the 497 testis- and placenta-specific genes was studied in a series of 271 lung cancer samples by extracting the corresponding expression data from genome-wide transcriptomic data (the latter were obtained by C. and E. Brambilla supported by the Ligue CIT program (Carte d'Identite des Tumeurs)).

For each gene, the patients were divided into two groups, those expressing the gene (calculated as described below), and those not expressing the gene (following the procedure for the determination of the ON/OFF gene expression status).

For each of the 497 genes of the list, the global and disease free survival probabilities were compared between the patients expressing the gene (ON) and those not expressing the gene (OFF). This was done considering the whole period of the study, as well as for 10 years (120 months), 5 years (60 months) and 2.5 years (30 months) of follow-up. This was performed considering all patients of the study (n=271), as well as each of the main histological subtypes of this population (ADK=adenocarcinoma; BAS=basaloid; LCNE=Large cell neuroendocrine; SQC=squamous cell tumour).

The genes whose ON status allowed discriminating the patients with good or bad prognosis with a significance corresponding to a p value=<0.07 (Logrank p value, obtained when comparing cumulative global survival and/or disease free survival over 5 years between patients expressing or not expressing the gene) were selected as candidate prognosis markers.

For all these genes, the correlation between their expression (ON status) and prognosis was validated in at least one of the two following lung published cancer transcriptomic studies with survival clinical data using the same Affymetrix technology (website GEO: http://www.ncbi.nlm.nih.gov/geo, respectively GSE4576 and GSE8894).

These studies were selected as external populations of lung cancer patients in order to validate our survival data obtained by analysing the transcriptomic data of the Brambilla study.

### Detailed Procedure

### 1—Establishment of a List of Placenta and Testis Restricted Genes and Analysis of Their Aberrant Expression in Lung Cancer Patients

1a—A list of 497 human genes whose expression was restricted to placenta or male germ cells was established as mentioned in the international application WO/2009/121878.

These genes are never expressed in normal adult somatic tissues (adult somatic tissues comprise all tissues except germinal cells, foetal tissues and placenta).

1b—Expression data were extracted from a series of 112 normal adult somatic tissues randomly selected from a genome wide study of normal human tissues (GSE3526 on GEO, this study was chosen because it uses the same probes and measurement technology to detect gene expression as the Brambilla study: Affymetrix Human Genome U133 Plus 2.0 Array). The CEL files (raw data) from the control samples were downloaded from GEO. They were entered in the Genespring software and normalized (RMA algorithm) simultaneously with the CEL files from the Brambilla study.
1c—For each of the 497 genes/probes, the mean hybridization intensity signal value of the 112 control samples+2sd was defined as the threshold for expression.

This threshold was used to distinguish between the cancer samples expressing the gene ON) and those not expressing the gene (OFF).

The measurement of expression of the genes using Affymetrix microarrays involves the hybridization of fluorescence labeled cDNAs from each tissue sample on microarrays containing gene-specific probes, the fluorescence intensity signal corresponding to each probe of the microarray is measured and changed into a raw value. The absolute value of the fluorescence intensity signal is highly variable and probe-dependent (different probes corresponding to the same gene can give different intensities of fluorescence). Therefore, on the basis of these absolute fluorescence intensity values it is generally not possible to determine whether a gene is expressed or not, and commonly people use this technique to assess variations of expression between samples (see below for more details).

In the invention, the definition of a precise threshold for expression was possible because the selected 497 genes are NOT expressed in any normal adult somatic tissue (according to the original criteria for their selection). Therefore the signal values obtained in the 112 normal adult somatic control samples give a high confidence set of values corresponding to the background noise signal, which allow further analyses.

A threshold signal value for expression could not have been defined for genes, which do not have a restricted expression pattern. Indeed in all these types of transcriptomic experiments the background noise signal value is highly dependent on the sequence of the probe. For instance several probes representative of the same gene generally give different signal values (although these signal values should normally vary between samples in the same direction). In the case of non-restricted genes (most genes have a pattern of expression, which is not restricted to germinal cells or placenta), it is therefore impossible to use these signal values as “absolute” indicators of the presence or absence of expression. However, one can compare expression levels between two groups of tissues (=expression in group of tissues A is significantly higher/lower than expression in group of tissues B). Therefore, in this particular study, since we have previously demonstrated that all the studied 497 genes are NOT expressed in normal adult somatic tissues, we were able to define a threshold differentiating expression (ON) and non-expression (OFF). This is a specific key feature of our approach.

1d—Based on this threshold, the expression of each of the 497 genes in each of the samples was defined as negative (OFF) or positive (ON) as follow. In each cancer sample, if the normalised signal value was above this threshold, the gene was considered as aberrantly expressed in this sample (gene ON), if it was under this threshold, it was considered as not expressed (gene OFF).
1e—From the Brambilla study (271 cases of lung cancer), the Inventors found that 130 of the 497 genes were aberrantly expressed in at least 1% of these lung cancer cases.
2—Correlation Between the Expression of Each Individual Gene (of the List of 130 Genes) and the Prognosis for Survival in the Lung Cancer Patients, and Selection of 23 Genes Individually Associated to the Prognosis of all Lung Cancer Cases (without Considering Histological Subtypes; Named after “Global Prognosis Genes”).
2a—As a first step, using each of the 130 genes individually, we compared the global survival over a period of five years between the groups of patients expressing the gene (yes) versus those not expressing the gene (no). A Logrank Mantel-Cox test was performed and a p value was calculated. This analysis was performed first with the whole population of lung cancer patients of the Brambilla study (n=271), second with each one of the following populations: ADK cases (n=91), BAS cases (n=46), LCNE cases (n=47), SQC cases (n=62).
2b—A total of 23 genes were selected, whose individual expression was significantly associated with a poorer prognosis (as measured by the cumulative global survival and/or disease-free survival over five years; p<0.05) in the Brambilla lung cancer study, as well as in at least one of the external validation populations.

The expression of any one of 23 genes in lung cancer is significantly associated with a poor prognosis when considering all histological subtypes.

2c—A detailed quantitative evaluation of the prognosis is given in table 2a, using each of the 23 genes associated with poor prognosis in all lung cancer types. The Kaplan Meyer survival curves obtained using each of these genes can be visualized by clicking on the link in the last column of the table.
2d—These 23 genes were then divided into two groups
A group of 7 genes, whose aberrant expression in lung cancer is relatively frequent (>10% of cases of our series). The expression of each individual one of these seven genes is associated with a significantly reduced survival probability (global or disease free survival over five years significantly reduced, logrank test p<0.07) of all lung cancer patients (without considering histological subtypes).
A group of 16 genes, whose aberrant expression in lung cancer is relatively rare (<10% of cases of our series). The expression of each individual one of these seven genes is associated with a significantly reduced survival probability (global or disease free survival over five years significantly reduced, logrank test p<0.07) of all lung cancer patients (without considering histological subtypes).
   3—Association of Several or all of 23 Genes of the Groups of 7 Genes or 16 Genes Allows a More Accurate Prognosis in Lung Cancer Patients than the Use of Each of Them
   3a—Different associations of these 23 genes were tested for the correlation between the expression of at least one gene of a given association of genes and the prognosis.
   3b—The 7 genes more frequently expressed were classified by increasing Logrank p value as follows: 1161; 391; 35; 442; 102; 390; 295. Following the same order, subgroups of the 1rst of these genes, the 1rst+2^{nd} of these genes, the 1rst+2^{nd}+3^{rd} of these genes, etc. . . . and finally the seven genes, were respectively tested for their prognosis prediction value. The distribution of patients according to the number of the genes of the group aberrantly expressed was studied, and relevant groups of patients were compared for their survival probability. The detailed quantitative evaluation of the prognosis using these subgroups of the seven genes is given in table 2b.
   3c—Similarly, the 16 genes rarely expressed were classified by increasing p values as follow: 125; 117; 766; 144; 108; 222; 72; 1165; 487; 1261; 205; 437; 1328; 1188; 436; 135. Following the same order, subgroups of the 1rst of these genes, the 1rst+2^{nd} of these genes, the 1rst+2^{nd}+3^{rd} of these genes, etc. . . . and finally the sixteen genes, were respectively tested for their prognosis prediction value. The distribution of patients according to the number of the genes of the group aberrantly expressed was studied, and relevant groups of patients were compared for their survival probability. The detailed quantitative evaluation of the prognosis using these subgroups of the sixteen genes is given table 2c.
   3d—Using the expression data of all 23 genes, the distribution of the 271 lung cancer patients according to the number of genes aberrantly expressed from the group of 7 genes and from the group of 16 genes respectively, was studied. Nine groups of patients were constituted according to these criteria, and the survival Kaplan Meyer curves were compared between these nine groups of patients. Finally the 271 lung cancer patients were classified into three/four prognosis subgroups: P1a, P1b, P2 and P3. The P1a, P1b, P2 and P3 definition is indicated in the Table 4 below.

### II—Example of the Combination Using all the Genes of the Group of 7 Genes and of the Group of 16 Genes to Establish the Prognosis of Lung Cancer

Number of lung cancer patients distributed among the different groups according to the number of genes expressed from the “group7genes” (combination of 7 genes) and the “group16genes” (combination of 16 genes) (Table 3).

The groups P1a, P1b, P2 and P3 are defined as follows:

P1A corresponds to patient samples in which no gene of the group of 7 genes or of 16 genes are expressed.
P2B corresponds to patient samples in which 1 or 2 genes of the group of 7 genes are expressed but no genes of the group of 16 genes are expressed.
P2 corresponds to patient samples in which
either 3 or more genes of the group of 7 genes are expressed, but no genes of the group of 16 genes are expressed,
or at least 1 gene of the group of 16 genes is expressed but no genes of the group of 7 genes is expressed,
or one gene of the group of 16 gene is expressed, and 1 or 2 genes of the group of 7 genes is expressed.
   P3 corresponds to patient samples in which
either 2 or more genes of the group of 16 genes are expressed, and 1 or 2 genes of the group of 7 genes are expressed,
or at least 3 genes of the group of 7 genes is expressed and at least 1 gene of the group of 16 genes are expressed.

TABLE 3
represents the number of patient samples expressing the
indicated number of genes of the group of 7 and 16 genes.

| Combination | Combination of 16genes | | |
|---|---|---|---|
| of 7genes | 0 | 1 | >=2 |
| 0 | 87 | 12 | 6 |
| 1 or 2 | 99 | 24 | 14 |
| >=3 | 13 | 4 | 12 |

TABLE 4
represents the 4 prognosis subgroups.

| Combination | Combination of 16genes | | |
|---|---|---|---|
| of 7genes | 0 | 1 | >=2 |
| 0 | P1A | P2 | P2 |
| 1 or 2 | P1B | P2 | P3 |
| >=3 | P2 | P3 | P3 |

The following table 5 recapitulates the data of table 3 and table 4.

| | | Nb of patients |
|---|---|---|
| P1A | Combi7genes: 0 and Combi16genes: 0 | 87 |
| Total P1A | | 87 |
| P1B | Combi7genes: 1&2 and Combi16genes: 0 | 99 |
| Total P1B | | 99 |
| P2 | Combi7genes: 0 and Combi16genes: 1 | 12 |
| P2 | Combi7genes: 0 and Combi16genes: >=2 | 6 |
| P2 | Combi7genes: 1&2 and Combi16genes: 1 | 24 |
| P2 | Combi7genes: >=3 and Combi16genes: 0 | 13 |
| Total P2 | | 55 |
| P3 | Combi7genes: >=3 and Combi16genes: >=2 | 12 |
| P3 | Combi7genes: >=3 and Combi16genes: 1 | 4 |
| P3 | Combi7genes: 1&2 and Combi16genes: >=2 | 14 |
| Total P3 | | 30 |
| All groups | | 271 |

### III—Examples of CT Genes Whose Aberrant Expression in Cancer is not Correlated with Prognosis

To enforce the specificity of the present prognosis method, the Inventors have evaluated the prognosis impact of 3 CT genes, identified as cancer marker.

The results are shown in FIGS. 38-40.

These results demonstrate that 3 genes, well known as cancer markers, do not give any significant information about the survival rate of patients afflicted by lung tumors.

Therefore, the combination of 7 and 16 genes (i.e. 23 genes) according to the invention provides a very specific and useful method for prognosis lung tumors.

### Example 2

### The Ectopic Activation of 28 Tissue-Restricted Genes in Lung Tumors is a Strong and Independent Predictor of Poor Prognosis

Having found that the “off-context” expression of normally silent genes systematically occurs in cancer, the Inventors next investigated whether these genes could represent useful biomarkers by considering one cancer type, lung cancer. Lung cancer is one of the most frequent cancers in humans and is the most frequent cause of mortality by cancer in men. In the context of a clinical research program, the Inventors constituted a cohort of 300 lung cancer cases (recruited in the Grenoble University Hospital, France), who received surgery, including 154 early clinical stage patients (T1N0) according to the TNM classification (tumor size, node positivity and metastasis). For each of these cases genome-wide transcriptomic analysis was performed on pre-treatment diagnostic tumor samples, and pathological and clinical data recorded, including global and disease-free survival over a period of 5 to 10 years.

Applying the strategy described above, the Inventors could detect aberrant expressions of TSPS genes in all, including the 154 cases of early-stage T1N0, lung tumor samples of their series. Moreover, a series of nine paired tumor and corresponding non-tumoral lung samples confirmed that these genes are activated specifically in the tumors and not in the non-tumoral lung.

This screen identified 28 TSPS genes, whose aberrant expression was individually associated with a lower survival probability in the lung cancer patients of our series (log-rank test p-values<0.05 and Hazard Ratios>1.5). The Inventors then tested these 28 genes in combination as predictor of prognosis. Using the optimal and simplest combination, the Inventors assigned patients into two groups:
none of the 28 genes expressed and
at least one of the 28 genes expressed.

The Inventors then further refined this latter group by distinguishing tumors expressing one or two genes from tumors expressing three genes or more. Finally tumors were stratified into three groups:

P1, expressing none of the 28 genes,
P2, expressing 1 or 2 and
P3 expressing 3 and more of the 28 genes.

Highly significant differences in overall survival probabilities between these three groups were found. Additionally, the prognostic power of this 28-gene classifier was independent of other parameters, including clinical stage (TNM classification) and histological subtype. In particular, this 28-gene group was a very efficient predictor for overall survival of early stage patients.

A multivariate analysis confirmed that the 28 genes combination of the invention was the strongest prognostic parameter associated with overall survival (p<0.0001).

A comparison of the clinical outcomes between P1 and P3 patients allowed the Inventors to confirm that the tumors classified “P3” presented a particularly aggressive phenotype.

Indeed most patients with these tumors quickly relapsed and/or developed metastases, which was generally followed by short-term fatal outcome.

The following tables summarize the results:

Tables A-C indicates the number of patients, their survival rate at 30 months according to the method disclosed in the invention.

TABLE A
The table indicates the number of P3 patients and their survival rate at 30 months among the 300 pateinets having lung cancer,
for the indicated group of genes.

| | grp13g | | | grp14g | | | grp15g | | | grp16g | | |
|---|---|---|---|---|---|---|---|---|---|---|---|---|
| | Nb | % | p- | Nb | % | p- | Nb | % | p- | Nb | % | p- |
| Pc_grp | patients | Survival | value | patients | Survival | value | patients | Survival | value | patients | Survival | value |
| P1 | 121 | 73.55 | <0.0001 | 119 | 73.95 | <0.0001 | 118 | 74.58 | <0.0001 | 116 | 75.86 | <0.0001 |
| P2 | 145 | 61.38 | | 146 | 61.64 | | 145 | 62.07 | | 145 | 61.38 | |
| P3 | 34 | 14.71 | | 35 | 14.29 | | 37 | 13.51 | | 39 | 15.38 | |
| | grp17g | | | grp18g | | | grp19g | | | grp20g | | |
| | Nb | % | p- | Nb | % | p- | Nb | % | p- | Nb | % | p- |
| Pc_grp | patients | Survival | value | patients | Survival | value | patients | Survival | value | patients | Survival | value |
| P1 | 116 | 75.86 | <0.0001 | 113 | 77.88 | <0.0001 | 113 | 77.88 | <0.0001 | 111 | 78.38 | <0.0001 |
| P2 | 144 | 61.81 | | 146 | 60.96 | | 145 | 61.38 | | 146 | 61.64 | |
| P3 | 40 | 15.00 | | 41 | 14.63 | | 42 | 14.29 | | 43 | 13.95 | |
| | grp21g | | | grp22g | | | grp23g | | | grp24g | | |
| | Nb | % | p- | Nb | % | p- | Nb | % | p- | Nb | % | p- |
| Pc_grp | patients | Survival | value | patients | Survival | value | patients | Survival | value | patients | Survival | value |
| P1 | 110 | 79.09 | <0.0001 | 110 | 79.09 | <0.0001 | 110 | 79.09 | <0.0001 | 109 | 79.82 | <0.0001 |
| P2 | 146 | 61.64 | | 145 | 61.38 | | 144 | 61.11 | | 144 | 61.11 | |
| P3 | 44 | 13.64 | | 45 | 15.56 | | 46 | 17.39 | | 47 | 17.02 | |
| | grp25g | | | grp26g | | | grp27g | | | grp28g | | |
| | Nb | % | p- | Nb | % | p- | Nb | % | p- | Nb | % | p- |
| Pc_grp | patients | Survival | value | patients | Survival | value | patients | Survival | value | patients | Survival | value |
| P1 | 109 | 79.82 | <0.0001 | 108 | 79.63 | <0.0001 | 108 | 79.63 | <0.0001 | 108 | 79.63 | <0.0001 |
| P2 | 144 | 61.11 | | 144 | 61.81 | | 144 | 61.81 | | 144 | 61.81 | |
| P3 | 47 | 17.02 | | 48 | 16.67 | | 48 | 16.67 | | 48 | 16.67 | |

TABLE B
The table indicates the number of P3 patients and their survival rate at 30 months among
the patients having T+N+ lung cancer, for the indicated group of genes.

| | grp15g | | | grp16g | | | grp17g | | | grp18g | | |
|---|---|---|---|---|---|---|---|---|---|---|---|---|
| | Nb | % | | Nb | % | | Nb | % | | Nb | % | |
| Pcgrp | patients | Survival | p-value | patients | Survival | p-value | patients | Survival | p-value | patients | Survival | p-value |
| P1 | 37 | 54.05 | <0.0001 | 36 | 55.56 | <0.0001 | 35 | 57.14 | <0.0001 | 33 | 60.61 | <0.0001 |
| P2 | 80 | 51.25 | | 81 | 50.62 | | 80 | 51.25 | | 81 | 50.62 | |
| P3 | 29 | 10.34 | | 29 | 10.34 | | 31 | 9.68 | | 32 | 9.38 | |
| | grp17g | | | grp18g | | | grp19g | | | grp20g | | |
| | Nb | % | | Nb | % | | Nb | % | | Nb | % | |
| Pcgrp | patients | Survival | p-value | patients | Survival | p-value | patients | Survival | p-value | patients | Survival | p-value |
| P1 | 33 | 60.61 | <0.0001 | 30 | 66.67 | <0.0001 | 30 | 66.67 | <0.0001 | 30 | 66.67 | <0.0001 |
| P2 | 81 | 50.62 | | 83 | 49.40 | | 82 | 50.00 | | 81 | 50.62 | |
| P3 | 32 | 9.38 | | 33 | 9.09 | | 34 | 8.82 | | 35 | 8.57 | |
| | grp21g | | | grp22g | | | grp23g | | | grp24g | | |
| | Nb | % | | Nb | % | | Nb | % | | Nb | % | |
| Pcgrp | patients | Survival | p-value | patients | Survival | p-value | patients | Survival | p-value | patients | Survival | p-value |
| P1 | 29 | 68.97 | <0.0001 | 29 | 68.97 | <0.0001 | 29 | 68.97 | <0.0001 | 28 | 71.43 | <0.0001 |
| P2 | 81 | 50.62 | | 81 | 50.62 | | 81 | 50.62 | | 81 | 50.62 | |
| P3 | 36 | 8.33 | | 36 | 8.33 | | 36 | 8.33 | | 37 | 8.11 | |
| | grp25g | | | grp26g | | | grp27g | | | grp28g | | |
| | Nb | % | | Nb | % | | Nb | % | | Nb | % | |
| Pcgrp | patients | Survival | p-value | patients | Survival | p-value | patients | Survival | p-value | patients | Survival | p-value |
| P1 | 28 | 71.43 | <0.0001 | 28 | 71.43 | <0.0001 | 28 | 71.43 | <0.0001 | 28 | 71.43 | <0.0001 |
| P2 | 81 | 50.62 | | 80 | 51.25 | | 80 | 51.25 | | 80 | 51.25 | |
| P3 | 37 | 8.11 | | 38 | 7.89 | | 38 | 7.89 | | 38 | 7.89 | |

TABLE C
The table indicates the number of P3 patients and their survival rate at 30 months among the patients having BAS
lung cancer, for the indicated group of genes.

| grp15g | | | grp16g | | |
|---|---|---|---|---|---|
| Nb | % | p- | Nb | % | p- |
| patients | Survival | value | patients | Survival | value |
| 7 | 57.14286 | 0.145 | 7 | 57.14 | 0.145 |
| 31 | 51.6129 | | 31 | 51.61 | |
| 5 | 20 | | 5 | 20.00 | |

| | grp17g | | | grp18g | | | grp19g | | | grp20g | | |
|---|---|---|---|---|---|---|---|---|---|---|---|---|
| | Nb | % | p- | Nb | % | p- | Nb | % | p- | Nb | % | p- |
| Pcgrp | patients | Survival | value | patients | Survival | value | patients | Survival | value | patients | Survival | value |
| P1 | 7 | 57.14 | 0.089 | 7 | 57.14 | 0.089 | 7 | 57.14 | 0.089 | 7 | 57.14 | 0.089 |
| P2 | 30 | 53.33 | | 30 | 53.33 | | 30 | 53.33 | | 30 | 53.33 | |
| P3 | 6 | 16.67 | | 6 | 16.67 | | 6 | 16.67 | | 6 | 16.67 | |
| | grp21g | | | grp22g | | | grp23g | | | grp24g | | |
| | Nb | % | p- | Nb | % | p- | Nb | % | p- | Nb | % | p- |
| Pcgrp | patients | Survival | value | patients | Survival | value | patients | Survival | value | patients | Survival | value |
| P1 | 7 | 57.14 | 0.089 | 7 | 57.14 | 0.089 | 7 | 57.14 | 0.089 | 7 | 57.14 | 0.089 |
| P2 | 30 | 53.33 | | 30 | 53.33 | | 30 | 53.33 | | 30 | 53.33 | |
| P3 | 6 | 16.67 | | 6 | 16.67 | | 6 | 16.67 | | 6 | 16.67 | |
| | grp25g | | | grp26g | | | grp27g | | | grp28g | | |
| | Nb | % | p- | Nb | % | p- | Nb | % | p- | Nb | % | p- |
| Pcgrp | patients | Survival | value | patients | Survival | value | patients | Survival | value | patients | Survival | value |
| P1 | 7 | 57.14 | 0.089 | 7 | 57.14 | 0.089 | 7 | 57.14 | 0.089 | 7 | 57.14286 | 0.089 |
| P2 | 30 | 53.33 | | 30 | 53.33 | | 30 | 53.33 | | 30 | 53.33333 | |
| P3 | 6 | 16.67 | | 6 | 16.67 | | 6 | 16.67 | | 6 | 16.66667 | |

FIGS. 50-65 represent respectively the percentage of survival of patients over the time (in months) when using from at least 13 to 28 genes according to the invention. P3 population (and curves) are indicated.

The population is the population of 300 patients afflicted by lung cancer.

FIGS. 45 and 47 represent respectively the percentage of survival of patients over the time (in months) when using the 28 genes according to the invention. P3 population (and curves) are indicated, in e T+N+ population and BAS population.

1. An element consisting of:
at least 13 genes chosen among a set of 28 genes comprising or consisting of the nucleic acid sequences SEQ ID NO 70 to 97,
or fragments of said least 13 genes chosen among a set of 28 genes comprising or consisting of the nucleic acid sequences SEQ ID NO 70 to 97,
or complementary sequences of said least 13 genes chosen among a set of 28 genes comprising or consisting of the nucleic acid sequences SEQ ID NO 70 to 97,
or sequences having at least 80% homology with said genes or fragment thereof,
or proteins coded by said least 13 genes chosen among a set of 28 genes comprising or consisting of the nucleic acid sequences
or fragments of said proteins,
or antibodies directed against said proteins,
said at least 13 genes being such that
12 genes comprise or consist of the nucleic acid sequences SEQ ID NO: 70 to 81, and
at least one gene belongs to a subset of 16 genes comprising or consisting of the nucleic acid sequences SEQ ID NO: 82-97,
said element suitable for carrying out a method for identifying at least 66% of patients of those having a survival rate of at most about 20% at 30 months, among a population of patients afflicted by lung cancer having an estimated survival rate of at least 30% at 30 months based on the diagnosis of said lung cancer according to histopathological criteria.

2. The element according to claim 1, of
at least 13 genes chosen among a set of 28 genes comprising or consisting of the nucleic acid sequences SEQ ID NO 70 to 97,
or fragments of said least 13 genes chosen among a set of 28 genes comprising or consisting of the nucleic acid sequences SEQ ID NO 70 to 97,
or complementary sequences of said least 13 genes chosen among a set of 28 genes comprising or consisting of the nucleic acid sequences SEQ ID NO 70 to 97,
or sequences having at least 80% homology with said genes or fragments thereof,
said at least 13 genes being such that
12 genes comprise or consist of the nucleic acid sequences SEQ ID NO: 70 to 81, and
at least one gene belongs to a subset of 16 genes comprising or consisting of the nucleic acid sequences SEQ ID NO: 82-97,
for carrying out a method for identifying at least 66% of patients of those having a survival rate of at most about 20% at 30 months, among a population of patients afflicted by lung cancer having an estimated survival rate of at least 30% at 30 months based on the diagnosis of said lung cancer according to histopathological criteria.

3. The element according to claim 2, wherein said at least one gene belongs to a subset of 16 genes comprising or consisting of the nucleic acid sequences SEQ ID NO: 82-97, is at least the gene comprising or consisting of the nucleic acid sequences SEQ ID NO: 82,
for carrying out a method for identifying at least 70% of patients having a survival rate of at most about 20% at 30 months.

4. The element according to claim 2, of at least 18 genes
said at least 18 genes being such that
a. 12 genes comprise or consist of the nucleic acid sequences SEQ ID NO: 70 to 81, and
b. at least 6 genes belongs to a subset of 16 genes comprising or consisting of the nucleic acid sequences SEQ ID NO: 82-97, said at least 6 genes comprising or consisting of the nucleic acid sequences SEQ ID NO:82-85 and 87-88,
for carrying out a method for identifying at least 83% of patients of those having a survival rate of at most about 20% at 30 months.

5. The element according to claim 2, of at least 21 genes,
said at least 21 genes being such that
12 genes comprise or consist of the nucleic acid sequences SEQ ID NO: 70 to 81, and
at least 9 gene belongs to a subset of 16 genes comprising or consisting of the nucleic acid sequences SEQ ID NO: 82-97, said at least 9 genes preferably comprising or consisting of the nucleic acid sequences SEQ ID NO: 82-88 and 91-92,
for carrying out a method for identifying at least 89% of patients having a survival rate of at most about 20% at 30 months.

6. The element according to claim 2, of at least 26 genes
said at least 26 genes being such that
12 genes comprise or consist of the nucleic acid sequences SEQ ID NO: 70 to 81, and
at least 14 gene belongs to a subset of 16 genes comprising or consisting of the nucleic acid sequences SEQ ID NO: 82-97, said at least 14 genes preferably comprising or consisting of the nucleic acid sequences SEQ ID NO: 82-92 and 93-96,
for carrying out a method for identifying at least 97% of patients of those having a survival rate of at most about 20% at 30 months.

7. The element according to claim 2, of 28 genes comprising or consisting of the nucleic acid sequences SEQ ID NO: 70-97 for carrying out a method for identifying 100% of patients of those having a survival rate of at most about 20% at 30 months.

8. Method, preferably in vitro, for identifying patient afflicted by a lung cancer having of the survival rate of at most about 20% at 30 months, among a population of patients afflicted by lung cancer having an estimated survival rate of at least 30% at 30 months based on the diagnosis of said lung cancer according to histopathological criteria,
said method allowing the identification of at least 66% of patient of those afflicted by a lung cancer having of the survival rate of at most about 20% at 30 months,
said method comprising
a step of measuring, in a biological sample of said patients, the expression of
at least 13 genes chosen among a set of 28 genes comprising or consisting of the nucleic acid sequences SEQ ID NO 70 to 97,
or fragments of said genes
or complementary sequences of said genes
said at least 13 genes being such that
12 genes comprise or consist of the nucleic acid sequences SEQ ID NO: 70 to 81, and
at least one gene belongs to a subset of 16 genes comprising or consisting of the nucleic acid sequences SEQ ID NO: 82-97, and
a step of identifying biological samples expressing said at least 13 genes.

9. Method, according to claim 8, wherein said at least 13 genes being such that
12 genes comprise or consist of the nucleic acid sequences SEQ ID NO: 70 to 81, and
at least one gene belongs to a subset of 16 genes comprising or consisting of the nucleic acid sequences SEQ ID NO: 82-97, said at least one gene comprising or consisting of the nucleic acid sequences SEQ ID NO: 82,
said method allowing the identification of at least 70% of patients of those afflicted by a lung cancer having of the survival rate of at most about 20% at 30 months.

10. Method, according to claim 8, said method comprising a step of measuring, in a biological sample of said patients, the expression of at least 18 genes chosen among a set of 28 genes comprising or consisting of the nucleic acid sequences SEQ ID NO 70 to 97,
said at least 18 genes being such that
12 genes comprise or consist of the nucleic acid sequences SEQ ID NO: 70 to 81, and
at least 6 genes belongs to a subset of 16 genes comprising or consisting of the nucleic acid sequences SEQ ID NO: 82-97, said at least 6 genes comprising or consisting of the nucleic acid sequences SEQ ID NO:82-85 and 87-88,
said method allowing the identification of at least 83% of patients of those having a survival rate of at most about 20% at 30 months.

11. Method, according to claim 8, said method comprising a step of measuring, in a biological sample of said patients, the expression of at least 21 genes chosen among a set of 28 genes comprising or consisting of the nucleic acid sequences SEQ ID NO 70 to 97,
said at least 21 genes being such that
12 genes comprise or consist of the nucleic acid sequences SEQ ID NO: 70 to 81, and
at least 9 gene belongs to a subset of 16 genes comprising or consisting of the nucleic acid sequences SEQ ID NO: 82-97, said at least 9 genes preferably comprising or consisting of the nucleic acid sequences SEQ ID NO: 82-88 and 91-92,
said method allowing identifying at least 91% of patients having a survival rate of at most about 20% at 30 months.

12. Method, according to claim 8, said method comprising a step of measuring, in a biological sample of said patients, the expression of at least 26 genes chosen among a set of 28 genes comprising or consisting of the nucleic acid sequences SEQ ID NO 70 to 97,
said at least 26 genes being such that
12 genes comprise or consist of the nucleic acid sequences SEQ ID NO: 70 to 81, and
at least 14 gene belongs to a subset of 16 genes comprising or consisting of the nucleic acid sequences SEQ ID NO: 82-97, said at least 14 genes preferably comprising or consisting of the nucleic acid sequences SEQ ID NO: 82-92 and 93-96,
said method allowing identifying 100% of patients of those having a survival rate of at most about 20% at 30 months.

13. Method, according to claim 8, said method comprising a step of measuring, in a biological sample of said patients, the expression of 28 genes chosen comprising or consisting of the nucleic acid sequences SEQ ID NO 70 to 97,
said method allowing identifying 100% of patients of those having a survival rate of at most about 20% at 30 months.
said method allowing the identification of 100% of patient afflicted by a lung cancer having of the survival rate of at most about 20% at 30 months
said method comprising
a step of measuring, in a biological sample of said patients, the expression of 28 comprising or consisting of the nucleic acid sequences SEQ ID NO 70 to 97, and
a step of identifying biological samples expressing said 28 genes.
